SEQUENCE LISTING (19 sequences)

SEQ ID NO: 1 - DNA, 7418 bp, Homo sapiens
  source 1..7418: mol_type = other DNA
tgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaa

SEQ ID NO: 2 - DNA, 2396 bp, Homo sapiens
  source 1..2396: mol_type = other DNA
agtagcaccaccgccttccaagtttccccttgtggatgcgcggccccgcggctctgctcctcccggcgcagaggggccgggagaggccacaggagcggacctggcacgggatttctgaggaacgggagaagactggcgcccgacccgctctggagggtcggtgaacgatgaagggccggcggcggcgacgccgagagtactgcaagttcgcgctgctgttggtgctgtacacgctggtgctgttgctcgtcccctccgtattggacggcggccgcgacggggacaagggcgccgagcactgccccggcctgcagcgcagcctgggagtgtggagcctggaggcggcggcggccggcgaacgcgagcagggagcggaggcgcgggccgccgaggaagggggcgcgaaccagtctcctcggttcccaagcaacctcagcggcgctgtcggggaggcagtgtctcgcgagaagcagcacatctacgtgcatgccacctggcgcaccggctcgtccttcctgggcgaactctttaaccagcacccggacgttttctacttgtatgagcccatgtggcatctatggcaggcgctgtatccgggcgacgccgagagcttgcagggcgcgctgcgcgacatgctgcgttcgctcttccgctgcgacttctccgtgctgcggctgtacgcgccgccgggggaccccgctgcgcgcgccccggacacggccaatcttaccacggccgccctcttccgctggcggactaacaaggtcatctgctcgccgccactgtgtcctggcgcaccccgtgcccgggccgaggtgggcctcgtcgaggacaccgcctgcgagcgcagctgcccacccgtggcgatacgcgccctggaggccgagtgccgaaagtacccggtggtggtcatcaaggacgtgcgcctgctcgatctgggcgtgctggtgcccctgttgcgtgatccaggcctcaacctgaaggtggtgcagcttttccgcgacccgagggcggtgcacaactcgcgcctcaagtctaggcagggactgctgcgcgagagcatccaggtgctgcgcacccgccagaggggcgaccgcttccaccgtgtgctgctggcgcacggcgtgggtgctcgccccgggggccagtctcgcgcgctgcccgccgcgccgcgcgccgatttcttcctgaccggtgcgctcgaggtgatctgcgaagcctggctgcgcgatctgcttttcgcgcgcggcgcgcccgcctggctgcggcgccgctacctgaggctgcgctatgaggacctggtgcggcagccacgcgcccagctgcgccgcctgctgcgcttctccgggctacgcgcgctcgcagcgctcgatgccttcgcgctcaacatgactcgcggcgcggcctacggcgccgaccggcccttccacctgtcagcgcgcgacgcccgggaggcggtgcacgcctggcgcgagcgcctgagccgagagcaggtgcgccaggtggaggccgcctgcgctccagccatgcgtctgctcgcctaccctcgcagcggagaggagggcgacgcggagcagcccagggaaggggagacgccgctggagatggatgccgacggcgccacgtagcctcccatccctgtccccggcacggatccgggtcagtcaccacgaacaggggcactcggcatgctgccccagcactggagaagcagcgctgtgggggcaatctgtcacactctcagagtctgggacttgacttgctaccaacaactgctgtgcaattctgctgagcaggaatatcatgagctgttcaataatgacggacgcattggttgagatgaagtttccagtaaggaagtgacagtgcaatgtggatatttatggctgtaaaataggaagagctttagttcccaggctgaacctgccactgctggagccatttcaacaaggcatcctcacaacaaagaagagatgtgatttggtaccatttcacaccagcaggtgtctggacgaaaacatcaatgtgaataagggccaagtgcagtcctgtcttgattaaattacttaataatattattaaataataataggtctgggcagtattgtttttaacctgactcatccagctgtccttcaaatagctccgtctccctctacccagaactgatttttaaaaagaagtaatttttctccctgggctgggaaaaccctaatgaactgaaacacacttttactttaaaatttttctgtctggcgtttttgtaatcatactattaaatgactctggagtcatgttaatgacaggatttgttttgtttggatgcagttcaattgcatggtttgggtaaaagctagcctacatacaaaggaatatgaagactgtggaagaaactg

SEQ ID NO: 3 - DNA, 1792 bp, Homo sapiens
  source 1..1792: mol_type = other DNA
gacgaacttgtgtcctgcgtcaacagtgtgtggaacgttggcgacctcggcgctcgagctgcactccgctgggctcttggcagcaccctgtgctctgagggtgtcttgcccgccgggcgccgtggctcacgcctgtaatcccaacactttgggaggctgaggcgggcggatcacttgaggtcaagagttcaagaccagcctggccaacatgattctactctaaaagtcaccaggaaaaattcagctccactggtcctggctctaagtctactagcaaaatcttccagagaagtggaaaagccatcagccaatgtgttagactatcatttccatgaccttacacctatcaaaaaggcactattaccaggcttatagaattctaaaaaagttggaaaccattttccattataaaagaaattcttccaaatcacacaaaacactctgacacttctccaattccccctatcatggggctggcagcagccgctttgcgtcctgaatgtcatgacagtgatgataacagtaatgatgataacagcaatgatgaagtctcacatttaaaattttcaagtcactcatcacatcccaagtcactcccttcattaccttttatttatactggagtcatcctgtgtgcctcagtcagaccattttgtatgtacaatgaccgacaatgagatttaacatgcagtttgcagatcctcccttctctgggtcacctgaaaataagccacaaacaaaggaatgaaattgttgatgaaaagggaagaggcaagactccatatgaagaatacaactgactcacctgtctgtacatgcctctgtgtgtgtgttttatattggtgctggtgaagatagttctttatgctgatgcttaataaggtgaaatgtgtctattttcttccattggcccaggcacagatgcgattataattgctaagattatttgcatattggcttttaatgatgcatttaaaaatattcatcccatttactgacttatgcaggcaatgtacttttgtaaatgtgggccagcatcatgcttaaattcccaaaccactatttatttaaaactcatcacttcagtttttataccctttctcccccataagggaaaaaatatgattctgactgtataagcaccaactaaacagaacacaaaacatggcagacgataaaaacatattttcacaaatgagacatgtcattttaccatcgaggagagaaacaaaataagctggttttcaagttccatatcttgtgtgctcctttcagctaaatgcccttacatctgtaagcccttgggacaggttaaggcttttattattattttaaagtcttcataactacaacctagcatccaaagtgaaacagatgctattggatatgagtagcatatgcaatggaaatattgacattgccttcccgacagtcatgagggatagaccataggctttttggtaacctatattttctaggcccaaagcagggaatgagatggcagggaaattcagggtaaactccaaatttacataaagttatagaagaatatagatcatgagtagtggctagcaccttcctgacttccagaaaatggtggtgccgatattgaacaacagtttggagaaaatgtaaaatgtttacatttacatttacagtttagtctttgaacaacagtttggagaaaatgtaaaatgaaaatttgttaacgaacttgtttttaaaaagcacctagactccatataagagtacttgtcctatgttattttcataaactacatttgtgattgtt

SEQ ID NO: 4 - RNA, 562 nt, Homo sapiens
  source 1..562: mol_type = genomic RNA
aacactcaataaagctcttctttgtcttgttcaccttccacttgtctgcataggcaggatttggcaccactggcagtgacactgtaacattttgggggctcatctgggatctccaaaagggtgaagaaagagaggattaatgagaaaaacaaattagacggcatagcctagatgcagaaagtctaactccaaaattcatgaaccttccaaaatttaccatgttgtccctcttcacttggaactctagtgacacacaaagatcaacacctccacatttgcccccagtgataggccttggtagcttaactggttgtgatatcataggaaatgactgcatgcatgtagaaggatgacaaagatggagaataaccagagtgcataagagggtaagatggaaggtatgaggaagtggtgattaccagaacagaaaagagagaaaaagtcaaaagggagaaaggggaggaaagcaaactaaatctcaggaaacattgtgaaaaaatagtgagaagtgagaaactcttccagcatagtaactgtattcaacaggtatgtctatgctgtg

SEQ ID NO: 5 - DNA, 2065 bp, Homo sapiens
  source 1..2065: mol_type = genomic DNA
gagacgactctcgctctgtcgcccagactggggtgcagtggtgtgatctcggctcactgcaagctccgcctcctgggttcacaccactctcctgcctcagcctcccgagtagctgggactacaggcacctggctaattttctgtatttttagtagagatggggtttcactgtgttagccaggatggtctcgatctcctgacctcgagatccgcccaccttggcctcccaaagtgctgggattacaggcatgagctactatgcctggccacatttttttaagttttgaatcatgcaaaccttttttagaatttcagaataagaatataaaaaatcacatcctaacactgaatatcaacagaaacaaatgtatctaactatatatataactctaactagatacccttaatggactatagtctaagtgcaaaaacaatgtccaaaaaaaaaaaacagttgtactaagtagtttgctgttggtagcagtattagtattgttatgctgaaactattttgtacgtattgttggataaagtgaataagcaagtattgctgagaacctgggttttcactgtgagagaggggagatgcatatatagaatgtgagaagtgatgaagaaccttgtagtggtggacttgaattggagctatgagactaaattcgtgattttgaaaaaatatgatttcctaacactatccttagaaaagtcctaaaaataacgatgctccgttaaaagtgagcatgtccaacacccatatttggtttctaaatgctattccccacttaaaagaaccagaacttctcagagagatggctgattacaggtctggggcaggcaagataacaagatgaccctggaaattttggtgtgcataaagcaagggagtcttcaaacaccaatgacattatatcaaaaggacataggaagtagcttgaaagggaaccactgataagcttgggacaatttgagcatgaaaaaaagacagtggcagaaactgattagaactcattaattaaaacataatgggttattttccattaatgccatgtgttagattaatttaaaaaacatgaaatccacagaaatattaaaaagtgggaaagggaaatcactagacgaaaagttaagaagataatattggccaggagcggtggctcacacctgtaatcccagcactttgggaggccgaggtgggtggatcacgaggtcaggagatcaagacatattggccaacatggtgaaaccccatctctagtaacatacaaaaaattgctgggcatggtggcacgtgcctgtaatcccagctactcaggaggctgaggcagggcaatcgcttgaacccaggaggcagaggttgcagtaagctgagatcgcgccactgcactcgagcctggtgacagagcaagactccaacaacaacaacaacaacaacaacaacaacaacaaaagataataatcacatggtctgtaaatatcactacacagattaattaaaaagggaaaatgttcctttacaatgaggagagctagaagactacaccttaagcatgttcaaatttagcaaaaccagtaataaaacctgactttattgccacctgatgtgatacaataaatacacatcatcacccacaccaaatacttcagcacaaaaaggaatatcataatacctacaactttcaaatggttcagtaaatgtggcaaaatgccaacaactggtgagtaaaagtgaagagtataaggatgttaattgtactagtttttcaacttttctatagatttggaattttgtaaaataagagttggggaagaaaaagtgtgtaggatgatttgaggacagaattgactgggtgatagttattaaagattccaccgttttaatttcaccatataaaaattatgccacactactaaaacaactaaacctgattaaatttttaccttcaactgagactactaaatgtgacaatgccaccccaaaagcggaaggcatagtaattaagccgaagttctagcaataaattttagtgggtcttagtcactctggtaagaaatgtaatacattaat

SEQ ID NO: 6 - DNA, 2367 bp, Homo sapiens
  source 1..2367: mol_type = genomic DNA
ggagtggggaggcggcaagaggacctgcggcaggccctcttcggcagtctctccggcccggtttccctcggcgtgctactgtgcgctcgatccagcaccatggggaagcgggacaatcgggtggcctatatgaacccaatagcaatggcgagatcaaggggtccaatccagtcttcagggccaacaatacaggattatctgaatcgaccaaggcctacctgggaagaagtaaaagagcaactagaaaagaaaaagaaaggctccaaggctttggctgaatttgaagaaaaaatgaatgagaactggaagaaagaactggaaaaacacagggagaaattgttaagtggaagtgagagctcatccaaaaaaagacagagaaagaaaaaagaaaagaagaaatctggtaggtattcatcttcttcttcatcaagctctgattcttccagcagttcttctgattctgaagatgaggataagaaacaaggaaaacggagaaagaaaaagaagaaccgttcacataaatcttctgaaagctccatgtcagaaactgaatcagacagtaaggatagtttaaaaaagaaaaagaagtcaaaagatggaactgagaaagaaaaggatattaaaggactcagcaaaaagagaaagatgtattctgaagataaacctttatcatctgagtccttgtcagaatcagagtatattgaggaggtgcgagcaaaaaagaagaaaagcagtgaagaacgagaaaaagcaacagaaaaaacaaaaaagaaaaagaagcataagaaacacagtaagaagaagaaaaagaaggctgctagttcaagtcctgactcaccataacattaagaaaaatcaggattcccttataaagaaagtgcaatgtctgaggaaatttcaactgtgaaaactacaacatatttactaaaatgcatgaattttcttgtttttggaattattcctggactattcagtagccactcagatgccactgtgtgaaagggccataaatgttgcctgctgcttgaacatctatttttttctcttccagtgcttgataactctgggagataatacactgcagtcgtactagtggttaagatatttgggaataaaattaatacttttgactagaagcgtctaaggataaaccaacagaaattgaatctggatacatctttaagatgtaatcagaaatgaccagatgactctagttagaatttttgaaggagggattacattaatatttcaaaacccttactctgtagataagtgtattttaattttttcccctcgtatacttttatttacctggggaaggagcttttagggttggggggtggtttgctatctctttagctagcagaatagtgtgcctttgatcctcacacatcctgtattatggacacagtagccatgcttcacggggaggtcagagctggctaccagcagtcttgccctttactgagcttagtgtcatctttggatgctgtcatatgctgctttgagtgaaccagagaaacagccatttgcagcatgagaaagccccaaaagctctgggatttacctccactttagtaataatgaatattttttagcattagaatgtgttatgtcatttgaattaattttgactacactttggcttgggagaggaattattttaaatagacattggtactttttgaacttgatagctaaagattctaaaatgcatgttttatactaagttttaaccagtcaggaaaattttatgtaactagtgatagtttatttttttgtatgaattttgtttaggctgcaatgtttagcttttgttaactcctcactcttgctgtcttaagttcattactatgtttaatggcctacttgccaagatatttagcatgtaaaaagcagggttttgattaaaaaaaaaaaacggcttcatattgaagctgagacttacaataacaagttgagtggcaagcctggtatgctgtgtcttattgccagaatcttagtaaatgtaatgttttaaaagtttgttgtctttgtatattaataacaaattatgacaagttaagtttaaataagagttacattattgctctccctgtgtcatattttactaagattttgtgcctcatgtctactgctgaattgtttactaggaatgttaaaaggaattgataattcattttcattttacatttttatataatcaggtaacatgacatacaacttgatgtacaattttgcctgttacaaagggtgtgctaattatagtggtatatgcacagaacatgttggcatgacaacaggctgaataaatagctattttacttaaaaatagctgtgttcttatcagctccctttggactgcaa

SEQ ID NO: 7 - DNA, 2759 bp, Homo sapiens
  source 1..2759: mol_type = genomic DNA
agattagcagggtgactttcaccctgcaacagtcacgctggagtactctggtgaaggatcaagaatattaccagttttctgatgaagaaaggaatatatttggtcaccagcaatctgcctttgaccatcctcaaagccaagtttttcatcgctgtctagaaaaatacttcattcagaggacacatatgtcctctttgaatgtctcctttttgggagcagtaggagaaagaaacttcaagatgtagaaagaattaatagtgtaacggaaagaaagaaggcaaaaggaagtaaatgtggaggccatagaaatagtaataagaataaagaagaaaaacaggaaagaagagggaaaagccatagaaaataacagccagagcgagtaggaggagacagagagaacgagagagagaaaagctgataaattcctgagaaaagtttctcttcttaagaattctaggtcaagaagtaaaatatggcacagagcattgaaaggaggcaacggatgcccagtgcaagattctgaagaagcaggaattcagcccgatgggagtccgagttcatgtcgtggcggcctcagccctgctgtatttcatcctgctttctgggacgagatgtgaggaaaactgtggtaatcctgaacattgcctgaccacagactgggtacatctctggtatatatggttgctagtggtaattggcgcgctgcttctcctgtgtggcctgacgtccctgtgcttccgctgctgctgtctgagccgccagcaaaatggggaagatgggggcccaccaccctgtgaagtgaccgtcattgctttcgatcacgacagcactctccagagcactatcacatctctgcagtcggtgtttggccctgcagctcggaggatcctggctgtggctcactcccacagctccctgggccagctgccctcctctttggacaccctcccagggtatgaagaagctcttcacatgagtcgcttcacagtagccatgtgcgggcagaaagcacctgatctacccccagtacctgaagaaaagcagctgcctccaacagagaaggagtcgactcgaatagttgactcttggaactgatgagagctgtcattttataaataggagtggagtgatgtccagagtctgtgggaaaatggaacacatacttttctaaccctcagaagttttaagatggcatctaacaccatcattctatgggaaagatggttcttactcttcgttcacaggcctttatatcttccgatacagaatgctctaattgggaactctaattttgtatccaatggccaaaatctgcaagtaatctctagccacactgattactactaaaccaggaaagcatcaaggtgtcttgaattcctttaactattgagtgcatatagaattcctgtacccacatgatactgcaagttgtgtctctctctgtcagcgaatccactgcggttaactggaaaagaaagacaacagtgtcagcacagccatcgacattaatgcactgaatgcatgcatctttcctcctgagacagcaatcgattttacaccgaatgacaatgatcatcttagacagcacaacatacccactcggatatctaaaagctagggatggcattgctgatatgggcaaagagaacacagtatagtatttaagtgccaaatatcagtctttctttctctctggtcctacccctcagcagtatgaaaaactccatactgtgcagtcacagttggattaattcttcagttcctccgcactgcaaacacatatatgtgcgcacatgcatgtatacctgcaccctgttttaactctaaaggaatagtgttgctttacttctttcctgttttgcctggaccacttaaagccacaacacctctatagtgacacacgctagtctctagtggtggccctcactgccacctagaggagccatggtggaaaacacactctctcctttgagcctatctgcacatctctcgagttcttggagcaaaaactaaatgctgaactaagcctggttgagatgcttcccatggaccatgccgcagcacagtgctaatctatccacaaaacataccacctcccaaagtattattattggaaaatcgaggaagtgacgcacatttagggaaaaactactcaccttagaaaagtcactgaaatccttttttttttttttgagatggagttttgctcttgtagcccaggctgggatgcaatggcatggtctcagctcactgtaacctccacctcccggattcaagcaattcttctgcctcagcttcccgactagctgggattacagctgcctgccaccgtgcccagctaatttttgtatttttagtggagagggggtttcaccatgttggccagtctggtctagaactcctgacgtcaggtgatccgcccaccttggcctcccaaagtgctggaattagaggcctgaccccctgctcctggcctgaaatctttaaagccgttttttccctaaaaaatgggaaataataacacctcagaaggtttttgtgaagatcaaagaagctaaatatatgtggcatgatttgtaaagtgttatgcatatgtatgttattcttcctactgtcttctaaccttcccttgcctgctatgacttatctgagagccatgttcccatttatctttttgccaactatgttactgttgtcacacctgaaatggctttgtttttatcaataaatacttgttgattgcggtaaacagcaacaga

SEQ ID NO: 8 - DNA, 189678 bp, Homo sapiens
  source 1..189678: mol_type = genomic DNA
gaattccacttcttgaaatttgttctttaaatacactgcacaggggcataaggatgttcccgctagcatgacttatagtagagacaatctagatgtcctcattaggggttagttctttaagttagaacaaagtgacacattatgagtgggttaaaaagagagatttgtatatttggacattcttaatcaaacaatcaagaattgctgattgaaaaaattatatatacatatttaaattaggctgttgcaaaagtaattgcggtttttgccaattacttttgcaccaacctaatatatgtacacaatccatatatatgtatatatatttaatgtccacaggaaaaaaatctaagaggacatccaccaccttgttggttaggttctaaagtccacttgtgaggtaaaagatcctgtatagaaattttgtattgaaattcctaaagaagacactatacaattcacaacatattctgaagtaaggactatggagttggattgctgaagtcaactgctgggtctattgcctgactttgcaatatcaactggtcacccacctaaagcaaacagagtagctataaatgtctatgagggtcagagacaaaagacgtggggggtagaaaagggataaacacgtatgtgcctactttgtccctggtactttgtgtatgaggaaagtaaaacccagagatgaacctgtgctaggtaatcaaaggtctcaggtacaaccggaacttggtctggcttcaaagctgtactttttctttttttgttgcttttattatttttaatgtattatttctttgtgttgggaacattcaaaatttgctcttccggctatttggaatatacaatacattgtctttaattatagtcaccctacagtgctgtagaacctagaatctattccttctatctaaaagctgcacttcttttttgtttgtttgttttttgagatggagtctcactctgtcacccaggctggagtgcaatggcgcgatctctgctcactgcaaactctgcctcccgggcccaagcaattcttctgcctcagcctcccaggtagctgggattacaggtgcccaccaccacacctggctaacttttgtgtttttagtagagacagggtttcaccatattggccaggctggtctcgaactcctgacctcaggtgatcctcccacgtgagcctcccaaagtgctgggattataggaatgagccaccacgcccagcctaaaagctgcacttctttactgatgcaaggtcttgctgcctttctgtggaggaagctccagatccatgagttttctgaattagagagcttagaaagacatgaggaagagaaaggaagcctggggaaatagaggtgggtggtgtctatctgaaacaaaagtgatcttgagtttcaaattaggtccaagactggttcaagcatcatttataggaattcgtccctaaaagtatgttccctacacacttgcattccagtatcagttctataactcaagagatttactttgcccaccaacacagaaacctttagataccctttaattgtccttcttactttactttcggaaaggagactcagattagagaaacaaagtcttcagaagagttgaatgctttctccaaactgcttatcaaagtgacatggggacacaatagcctggttggcagcctcaacagtgctggagagacttggtgagggcactagcactaagctccaccaccaactccattaattcccaactcaagcagttcagtcttgcatcaattttcccaaattcctgtttttttttttttttttttggatggagtctccctctgttgcccaggctggagtgcagtggtgcgatctcggctcactgcaacctctgcctcctgggttcaagcgattctcctgcctcagcctcctgagtagcaagtagtggggactacaggcgtccgccaccaagcatggctaatttttgtatttttagtacagacagggtttcaccatgttggccaggctggtctccaactcctgatctcaggtgatacacccgcctcggcctcccaaagtgctgggattacaggcgtgagccactgcacccagcacctaaatcacttttaaaagagagagaactgaaaaggacctagccagaccgtctttatttaaacaactaagattttttgccttacataccatggtcattattttcaaacattctccaatatttttcctcaataaaatctcccagcagccagggtgggcacagttggtgagctggtaacccgggctggccaagctgcatagcacggctttctctatctgaccagcctgcaacataaggccctacttaaggctttttcagtttgcttttgtttttgttttttttctttaggcaacctaacattatgaatgtaactccctaaggtttaaatgtcatgtgtaataggaacagtgtttgggggcaattaaggcaatgctgtttcggtgccgggaatccctcagatcacagagctgcctccatggtgtgggcgtcacctctaggctatgaccatggcccgtggagctgcttccgtccatggtgtgggcatcacctccaccacagagctgcttcgatggttaggtgtcagctcctgtctgtgactaaggtgagccatcaggctccccctagaagggaggaggatatgatttaggagaagtgagaaagaggtgtctgaaggagtgttggaaagagtaaagactgggatagaacctgtgtccaaggtgcctgccagcctccattcactttgactattgagggtcaattgggaaaggggggtgaggtgggaagtgggtttaagactagctagctagttacagggtgcacccccctcaacacaggagggatgtctctgggtctgaataaggagttggctcttctcttctcttcatgtctcatgcagggttgagcactgaggaaaggggtgtcaagacaaaacagccagatgctgtcccagggctcccctcattctgcctgtcaaggtttccaggagatccacaagtttcccagttgcatgcacttgcacggtgggtgtccatttaacatccctcgaatcagcagtgcccagtcaaatcttagcgggccggtgaacggcatacacagaatatggagagaacctatggcttctttctaccaaacagcacagcagcaaccctgtacctacctaaacacaagattgcaaaggagtagcgggtaagaagaagaaacagtaatgatggaagacagactacatttgaaaagattcaaaccctgttggcaaacttaagctgtttgccaacttctttttatcttctgtttgctgtgtttacctgatttgaagctccttctttttttttgagacggatctcgctctgtcgcccaggctggagtgcaatggcacaatctcggctcactgcaacctctgcctcctgggttcaagcgattctcctgcctcagcctcctgagtagctgggactacaggcgctcgccaccatgcctggctaatttttgtacttttagtaaagatggggtttcaccatattggccaggctggtctcgaactcctgaccttgtgatccgcccgccttggcctcccaaagtgctgggattacaggtgtgagccactgcgcctggccaaagctccttctttattatttgccacaagacataagcacagccgtaggtagacccagggtcatctagttatccacaatgtgaaggggcagcagcactgggcacagttttaatcactgcatttactagaatgtttccagaaaagaattgcaagacaattaaagaggtatatggaagattggcatctagaacagagaaactgaaagggaaatgtggctctgcctggaagggagtgcaaagaggataatgagacagtgttgaactcaggaagcctgtctgtggagtccagccttgacagtcctgtaattatcgacaaaaccactcttttctctggactgtaattgcctcaccataaaatgaaatcactgggcttggcatgagaggataagtgatacaatctttaacttgacagtacatattaaactccgttagaatgtacacacttcttaacccaacatgaccacttcttggaataaattacttataagaaaataactgggggctacacaaagttctatacaagagaatacttattgtgggactgtttggaataccatatagaaataggcttgtttgtcaaattataggtcactggttaaataaatgcagatcagctctcaaaattcacatcaccaaagccagttctgtgagggttttcaacttacaagatgatcagttcaccaaaattggtatcttttaattatttcagatttacgtaatgtcatattagatgggatagttttaacagcttaagtttataacagttggtattagttgtggtgctttcatagcttaagagtatagtaattcttttttaaaaataatttcaacttttattttaggttcaggaatacatgtgcaggtttgttacatgggtatgttgcaagatgctgaggtttggggtacagatgatcccaccatccaggtagtgagcatagtaccaaatagttttttggtgaatgtctccccttctccctcccccactttagtagtctcccgtgtctattgctcctatctttacatccatatgtattcaatgcctagctcccattcataagtgagagcctgcaatttggttttctgttcctgcattaattcacttaggataatggcttccagctgcatccatgttgctgcccaggacatgatttcattttttatggctgtgtagtattccctggtatataaatgcgagtataataattcttatcaaatataattgttttaggcctggtgtggtggctcatgcctgtaatcccagcactttgggaggcccgggcaggaggatcacctgaggttaggagttcgagaccagcctggccaacatggcgaaaccctgtctctactaaaaatacaaaaattagccaggcgtggtggtgcatgcttgtgatcccagctactcaggagctgaggcaggagaattgcttgaacttgagaggtggaggttgctgtgagccaagatagcaccactgtactccaggatgggcaaaagagtgaggctccatctaaaaaaaaacaaaaacaaacaaacaaaaaatatatatataattgttttaaaagattttgacaaatgttctattttttagaaagctaaggattattgagtcttcttagaactatattaatctgtcactagtaattagtagcttaagtttaatgtgagatatggttgtttctgtagtttcctaattcacttcttgctttacctctccttacatttttagaattttaatttttaaaaaattcttcaaccttaatgtcatcttttattacatttgagattcaagtcttctgcttctttctggtatccctatgcacaggaacacttcctttccttatttcaggatggttctatcctttcaactcaaatggttttgcaaacagctgccaggttaacacatcctaatttcaccttctcagaaactaaagtttaagcatgtcatatgtgttggaaatgatttggtcacttacaatttctttttgaggcttataaatttaaattttattttttctaattgcagaaacaatgttgatatgtcatttgaagaactagcaaaagcacattaaaaatgcataaagttttttaaataaaaataatattcaagtagttaaaagaaacaagtcttctggtacggtgtaacatttgagaaaacatcaaattgcccttctggtaactggcttttggaaaattgatctagaacatgatacttcaacacaaaagaatactaagtgactattaaattttaatacatatatatttattgacataaaaagatattcacaatatatttaagtgaaaaaatgtaaaaacagtatatatagcatgatatcattttgtgagggaaaaggtacaaatgaatagaaaaaagtctgaaaagaaataggctaaactaccgcaatgttcatctacagctatttttggttttgcccttgcaaatttttaattttcccatcatatataccttatttaaaaggtatattttatatacatatatgtataattttatatatgtataaaatcagagtttcgaggcccttaggttttgtgctcagttccccttagtctaaaattttaaaaatgttttccattgtgtacaaattatgtatacaagtatcaatgtattttattataatacataatacatagacaagttatacatgtacatatagtacaataggtaaacaatatacatatttatgtacaacataaggttatgtataggatacattacatataatatgtgctatgtacgagttacatattatattttaaaatataatatattttatttaaaaatataatatattttattatatataaaaagtaacatatacaagttatatatatataacttatatatatatacacacacgtatatgtatatgacagaaaaataaaaaagtttttaaaattttgaagcaaaataagataatctgaacccatggcctaaggtccttcaaactctaattgtccgtgggagtaagaacttagagagtagccatgcatgggggctcacatctctaatcccagtgcactggaggccaagctgggaggattactggagcctaagagtttggggcttcagtgtgaggtaaaagaaacaggccctgccaaggtcctgggatgagctgatggccacagcaggagctggtatgaccggaatgaagccagcagggggaggaggaagaggcaagaccaggtcttggtcccccatcaacatgctgcaagaagcctccaccacgtgccgcatccccatttgcataactcttagttgagcaatttcacacagaggaagacaacagggcattgctgaaacaagtggggttttttggttttgtttctcttaacaataaaagggaacactatgaaaacaagatgagctcatggaaacttacgtaaggtagggaaatagaccatttaaaggaccagaaggggacattcaaagcagagtctgaattatgtatatccagccaaccaaggagagttattaaacatcaactttgtttccagattcagatttttatgatcaacaatccagccacccatattcagaaatacatataatagtgtatttatgtcccactttaagagttgtcagcttatctaaatgcatgtaacataatagaaattattatgaaattacaaaaaagtgaaactcagctcaagacacctaaaagaagtggaatgttgtatcacaccataagcttctgctgcttattagctatagatggcaaatcatcaggagtctaggttagctagagcagtggtccccaacctttttagcatccgtgactggtttcatggaaggcaatttttccacagagcggggtcaggtggggggtggtggtttcaggatgaaactgtttcacaaggagcacataacttagatccctcacatgtgcagttcacgatggggttcacgcccctatgagaatctactatggcgctgatctgacaggagacagagttcaggcagtaatgcttgctcaactgctgttcacctcctgctgtgtggcccagttcctaacaggccacagaccagtaccagtccatggcccaggggttggggatgcctgatctagaggacaaggcaaagaggaaaacatatccatggatataaataaatctgcttctctacaacagggtttgtcaacctcagcactactgacatttcaagccagaaaattatttgttctagaggacagttctgtgtattgtaggatacttagcagcatccttggcctctacccactggatgccagtagcacctctccaggcctcccagctaccagttgtgacaaccaaaaatgtcttcaaacattgccaaatatccccttatgaaagcttcatgccaggaaatctgatgttttaattgctggtaataactcaattattgatattttctgttacagattggggcaaaatatgctttcttactcattcatgaatgttaagtttaatatcctgttataaaatgtaaaggacataaccagaaatcacatctgccactatgcctgtacacacacagacagacacaaagacacatacacagatctggtagctgttgaaacagatccagagattttctatgagaaaagcatcatcccgactgtattcttagtctcactaagtcttagtctcacttagtctcactaatctcacgcagtaaagatggaagaaacagtaagaaggatctattctacggatccctgcttctcatgaggaaactaagagtcaaagacaaaccaagattagctacaaatagattttatcttcactgaaagtaacaaaaagactcagtacatatgaatagaataaacagcccagaaatagaaccacacaaatatagtcaactgatctttcaaaaagagcaaagacaattcaacactgaaaggatatttttttcaataagtggtactggaacaactgtatatccacatgtaagtaaataaataagtatgtaaataaataatctagacccggatcttacatctattacaaaaatcaacccaaaatgaatcacagacctaaaggaaaaacaaaaaactataaaactcctaggagataacataggagaaaagctagatgaccttgggtttggtgatgacattttacatacaacatcaaaatgatccatgatcatgaaagaaaaaatgatccatgaaagaaaaaatgatccatgaaagaaaaaattataaattgaacttcattaaaatctaaaacttctgctctgtgaaagacactttcttaagaaaataaaaagacaagccacagattaaaataaaatctttgcaaaatatatatctaataaaggaatggtatctgaaatagacaaagaactcttaaaatcaataataagaaaacaaacaacctgatttttaaaatgggcaacagatctgaatagacacctcaccaaagaagagctacagatggcaaataagtacatgaaaagatgctccatatcatatgtcacctgggaattgcaaattaaagcaacaatgggccctgcgtggtggctcatgactgtaatcccagcactttgggaggccaaggtcggcggatcacctgaggtcaggagttggagaccagcctggccaacatggtgaaaccccatctctactaaaaatacaaaaactagccgggcgtggtggcgcttgcctatagtcccagctactccagagggtgaagcatgagaatcacttgaactcaggaggcggaggttgcagtgagctgagattgcaccactgctttccagcctgggcaacagagcgagattctgtctcaaacaaaacgaacaaaccaagcaacaatgagatatactctactactatatacctattaaacaggaaaaatctaaaacactgactacaccaaatgctggcaaagaggcagagcaacagatactccaattcattgctggggaacataaaatgacccaaccattctggaagacagtcttgcagtttcttataaaactaaacaatacccttaccatatgacccaacaactgtgccccttgatttttacccaagtgagttgaaaacgtatgtacacacaaaaacctgcacatagatgtttatagcagctttctttcttttttttttcccccttgggacagagtctcgctctgtcacccaggcaggggtacagtggtgtgatcttggctcactgtaacctctgcctcccaggttcaaaaaattctcctgcctcagtctcctgagtagctgggcattacaggcacccgccaccacacacagctaatttttgtatttttaatagagacagggtttcaccatgttggccaggctggtctcgaactcctgactgcaagtgatccgcctgcctctgcctcccaaagtgctgggattacagacgtgaaccaccgcacctggccagcagctttattcctaattgacaaaacttggaagctactgagatgaccttcattaggtgaatggataaataaaatatttatccattttatatccagacaacagaacactattcagtgcttaaaagaaataagctatcaaccatgaaaggacatagaggaaccttaaatgcatattactaagtgaaagaagccaatctgaaaaggctacatactgtgtgattccaaccgtatgacactctggaaaaggtaaaactatggagacactaaaaagatcagtggttgccagaggtttcgggaatagaggaataaacatgtgaagaatgggggatttttaaggcagtgaaactattctatatgactgtaatggtgaatacatgtcattatacacttgtcaaaatccatgaaatgtactacaccaagagtgaatcctaatgaaaactgtagattttaattaatgtatcaacattggcttatcagtgataacaaatgtaccacactaatgtaacatgttcattacaggagttaattatatgttaataataagctggttgagggtgggaagtcagtgatggctagatggcaattatctgtaatttctgcctaatttatctgaatctaaaactgcagctctgggcgcactgcctaggagttagccgggctccacaaggagcagtaaaaaacagacaaatgtaaaactgctctaaaaaataaagtctattaatttttttaaaagtatattgtggcagcacagacaccagcccacatgcttgggaatcagtatttctgaaaagggatcctccatttttaaaaagagggctgcctgaaaatatttctacaagaagttgcatgtatccacataatgtgcttcttcttaacacccaatagtctttacaattatcaagggcaggccgttacctgctgtgtcttaataattatggtttaaaatacaatagaatttcattagatcttcttggcttaattcccaaacttcttcaattttaggttcagaaaataactgataagagctgggagatctattaatgtttaataataaagtcatcactcctaatacgcccatagccatttatgataaaagaaatgcacagttaaattaggcttataaaatggcaaggcaaagtacatctgataatgtagctctgaacttacacatctatacatgcatagtcaacaggcctttatcagcgtttactaatcctgcagaaccattgctctcaatcaaccattcagtaactcaattaagtaaacacaggatgggcagcaggtacacaaactgcatgatgccattcaatggatataatagagccactgagttgaagataaggaagtggttggatctggggggtaaatctacgtgggagaggcttcaggaaggtagggcaggaatctgctgttttatgttaaagctttgtagttatttggtttttaaattatgtgcaagaatcaccttgaaagtgataaagatttcttaaggatggctaaaataattgtgtaattaaataaaaaatgcttataatttaatttttataaaacaagataaaaagttttatatacattatgattacataataaaaatgtttggaagcaatattaaaatcaaattaaactgaatcaaaaatttttacataaacacttgtatgcttctgtggtgagttggtttttctttttcttcattgctaaagtgtccacaatgagctataatgatttacttttataaaaatatagttgtaaaaattaaaaatcaaacacagcaagacacttatttataccagtcaccagcgtccagggtgtcctcaggaacctggccataactgtggacaggcagcacctctagatggtgtttccgttccattctgatgccttggtgtcacgggcggcctgcttcctcacccatgaaaaaggtaataataacgcccatccccgcatggctgtggtggagtttaaatgggttcacagaagacaaagacatagaaagcccttctcccttaaggcctggcatattcatgagcttgtggtaaagtatgaaggtcaaaagccaggcaataatttgaacctagccaaggattttaaacgcaaaagaaatatggtggaatgagaattggaaaacattctggtggagcgcagtggctcggacctgtaatcccagcactttcagaggtgaggcgggaggactgcttgagccaggagtacgagaccagcctgggcaacataatgagactcgtctctacaaaagaaaatttaaaaattagccaggcatggtggtgtacgcctgtggtcccagctactccagaggctgatgtgggaggattgcctgagcccaggagtttgagggggcagtgaacggtgattgcaccactgcaccctagcctgggcaacagagtaagaacctgtctcaaaacaaacaaaaaaagaggccgggcacggtggctcacgcctgtaatcccaacactttggaaggctgaagcaagcggatcaccccagtttggccaacatggtgaaatcccatctctactaaaaatataaaaattagccaggtgtggtggtgggtgtctataatcccagctacttgggaggctgaggcaggaaaatcgcttgaacctgggaggcggaggctgcagtgagccacgatcgcaccaccgcactgcagcctgggcaacagagagactccatctcaaaacaaacaaaagggaaaccattctttggactaagcttgaagccaaagcagtgacttaccaagtgacatatctgcagtatatgatactagaagtgaactggagagtcactgtaaactcactctaggagtttaaaggtgaaaaagaggaaatggaaggtcatggctggatccagttattttcttaaaaatgaggatgcagctgggcgcggtggctcatgcgtgtaatctcagcactttgggaggccaaggggggtggattacgtgaggtcaggagttcaagaccagcctggccaacatggtgaaaccccgtttctactaaaaatacaaaaataagccgggcatggtgggtggacgcctgtaatcccagccactagggaggctgaggcaggagaattacttgaacccaggagatggaggttgcagtgagtcaagattacaccattgcactccagcctggatcatagagcaagactccatctcaaaaaaagaaaaaaaaaaatgaggatgccaagaagtgccataaaattgagaaggaaaacagatggtatagaacaggcctgtaccatgattcccaaactgtgttttgaggccctcccggtgccatggtgaagtcacagaggtgccctgggatatttcaaattttcaagggaaacacaatgacatctctcacacatcgtgagaggtactcactcaaggtagtgctgtttcaacattagatcatgctatcttcctttcaacagcagcatatatttgtaaaactgggttttctgtggttgctataataaaaaaaaaaaagaaagaaaaagaaaaaacaagtaccaccagaaagtgagaatgtgggtgtccaatatgattctaaggttggagaagttgtgcggcagggtgtacacatcccatttgagaataactgtggtcatttaagaataatataaaaacatatttttctttcaatgtatgtgtatttttttttttcaaatggcactaagtcattataacaaacatttattaagttgtttgatcttggccgggcgcggtggctcacgcctctaatcccaacaattgggaggccgaggtaggcaaatcacttgaggtcaggagttcgagaccagcctggccaacatggtgaaacctcatctctactaaaaatacaaaaatcaggtgggtatggcagcaggcgcctgtaatcccagctactcaagaggccgaggcaggagaatcgcttgaacccaggaggcggaggttgcagtgagctgagattgtgccactgactccagcctgtggggacagagcaagaccctgtctcaaaaaaaaaagttgtttgacctaaatattcactaactgaaactctcctgtattttttctgctgtcgggttgccttgaaaaaattatagacactcagaatgctgtgaagcaagcatatttgggaatctgtaagcaagaaatttttggaagtgggaagcagtggatggcaggttaggacagagcaggtgtagcttgacagagaggcggaaactgagcagatgtgctgggaaggggaagcagttctcctggcaccgggacccacctgctttatctctcacattgcccgcactgccattaggagaaactactgactctcagaaaggatcagtaccttagaggccacccccaattcgtttcaaaaaatttttaaattacttaaatagtatgacagacaattctattgaatttccatcttccttactgaaaattgttttttctcaccattccagtcacccgaggctcaaaccctcagtcatcctgtcactttcgcttcacattttactcattaggtccaccctccctcaagccctgtcagttttgcgatataattgcacttggattctaggctcggaaaggcatggagcagatagtgagttttgatttcttttccctatattggtctcctttctctactaaaccctacctaatattaattgctacaacagccctgctacgtctaaccatcccaggttgctgaggttgcctcccagctggcaccctcccgttttgctacactcatctttatgaattctattttcatgttacttcatattcaaaattcttttttttttttgatggaatttcgctctttttgcccaggctggagtgcagtggcacaatctcagctcactgcagcctccgcctgccaggttcaagtgattcacctgcctcagctcccccaatagctgggattacaggcgtgtgccaccactcccggctaattttgtatttttagttgaaatggggtttcaccatgttggccaggctggtcttgaactcttgagaattcacccgcctcggcctcccaaagtgctgggattataggcatgagtcaccacacccagcctacgtttataattcttcggagactgtcttctacctattatttacctgattagataaattaatacactggcacacatagaaccaggctttaaaattgtgatgtcagccaggtgtggtggctcatgcctataatcccagcacttggggaggctgaggcaggagaatcgcttgagcccaggagtttgaggctagtctgggtaacacagtgagaccccctctctataaaaaatttaaacacattagctgggcgtggtggcacatgcctgtaggcccagctactcaggaggctgaggtgagagaattgcttgtgtctgggaggttgaggctgtagcgagctgtgtttatgccactgcactccagactgagtgagagtgagactctgcccaaagaaaaaataaaaatgaaactgtgatctcccttatccattcattctccacccactcctttccttatgcttccctacagcacccacggaagaatgtacctattttatcatctgtccatcgctcctctgttactgtaagctccatgagagcagggattcctgtcagttttgcacacccttgtattatcagtgcaagaaacattgttcggcacttgtaaatgctctgtaaatatctgtggaataaagagtatccagtggagtaaatgctcaatacattttaaacattattattactaatataccatcatttgtaatactgctatcaacactcctgccaacaggacaaaacctgaaatattcaacctgcccctccctgcctctcccaggctggcctcaacctgcctcaccatcacctgtgttactccaaatgttagactcctaattggcacggtggcatcacctgccttttcatcttggctcatgcactcctacccatttatgcaaatctcacctgtcttccaaacgtaactcaaaacccatcttgcctggtctccagcagcccacatccttctcccctcagatttcttctctccacaatcaatttggaatttcgtcccaagccccaggacatctcttatagtacaactttatgtctgttatgtacaactttatgtctgttatgtaactctagaggtgtctatcagttagtacgtgtatagctctctcctcgatagactgtaaaccccttgaggacagaccctgtctatcccgcttctttgcagacaacttgcttcaataaaacatacacagtgggccacctggttaccaagtcagcaacaaagcaccagttgttgaattaaagtgaacttagaaattccacgtgcttattccatagtttgtggtcagcagcaattccgtaatagaatatattttaattattagctacaatgtatttggtttataatagatgattaataaatgttaactcccaaaattgttttcgtttcagacgtatctagacgatgaaaacaaacaaaatgtcccccattatctgataatctgtgatcattcaaagaatgtgggctgaaaatcttttacttaaaaatcttcctggctgggtgcggtggttcacgcctgtaatcccagcactttgggaggctaaggcgggcagatcacctgaggtcaggagttcaagaccagcctagccaacatggtgaaaccctgtctctactaaaaatacaaaaattagccaggcgtggtagcacatgcctataatcccgctgaggctgaggcaggagaattgctggaacccggtgggggggtggtggggaagggcaagggggggaggggcggagcttgcagtgagccaagatttatgccactgccctccagcctgggcaacagagtgagatactgtctcaaaaaaaaaatttcctgaagtaaagcagcaaaggactttggaatgaggggaaattgggtctatccttgttcttccatttattaacagacatataaacataagcaagttacttcattaaacttcattttcctcatctgtgaagggtagataacagtcctccatcaggggtcaccatgaggattaaatgaaatagcatatgttaaatgctctctcagagcaagtaaacaatgcgttacttcccattatccgcccccgtcctccaaaacaaggaaagccaggttatagtgacatgatcacacttaagaaatgttgctgtgacattctgcacaaacaaaattgttaaacaaaaattgctaaagaggctttatgaatgggaatcctgctaacaaagaaagcggtgtaattttcacagcactccagataatgtctgatgcgtccccaagtcccccagctctacctcaattctatccctctcttcactttactggaattctagtggtccaggtttcagattcttccccaactcacactggccttcccacaattaatctgctgaattattttactatatttacagcccatgctcacatatttggcagaaaaaacagttctatgtttgcagatgtctttttcaattggcattgacttttttctctgattttgtcactgagtaaaaaatgaggaattctgtttttcagcctcaagccatgttaagttggaggacagacaaagaagcaaagagtggggtcacgtgacaaagctcccacccccactccccccgcccaacccttcactttcccaggtacaaagaattataaagttgcctccaagcagcgtctttgcctggagagccatttcccgagcctcacacctcagccaatgcacgttcaactgctcacccagacgggtgcattctccctccccagggcccgttgtggctttttggatgccccagctggcctcccgctgagaagggaagggacttggcctggtgagaggaagattagggttttcaagcagccccaggaggaggaagttacttgttcccacgggcatgggtttttctttttgtgggtgtgttttttctttctgagaccaggaagctaaatttgcctaatagttaagaccaaaaggattatagattctttcttcaaaccccagcctgtggcccatcccatccctccatggtaaaatcagtcaatttctaaatcaccctgacagagggagggtttccagacccggcagtgaagcgtattctattccattatttctatgaaaagcactatattaacagagcattcatatttatgttcctcattcttctcactcaaactctacactaacttaaagtacgaacactaaattatatcccaatttcctaaaaaggcaaaaatcaaaacttcatttccctctccactctttctcttcttttcaataaacaactgttaggtttagattctcacacatgttcacttttcctatttcctttaattagccctgaaagcccagcaagtcctgcttttcttgaaataattgagatcctctttctcccctattatttctgcttaaaatgtgttatctcctggtaacacagaccaacattcaacagtcctgcctgtcgggaatgtctgactactatcacttttttttttttttttttttttttgtagagacagggtctcccatggtctcgaacaactgagctcatgcaattctcctgcctcgaactcccaaagcactgggattacagctgtgagccactgcacccagcccattctcgctgctgaagtccacctcccctgggtaccacctccgtgaaggtggagaaatctcaccaccacttttgcacagcccatcatgggcacccatcaggacccgatcagaatccccgtgtagttggctggcccagtggtggggtttgccaaagaactgtgggcatacactgagcctgaatccctcagcctcagtgactcatttggacaatgaggacactgccccctaccccgcaaggccatagtaaggattaaccgagacaaccccgaacacctggcaccactcaatctgggagggctctggtcaacatcacttatgtgtggtgatggacaaaatgcattattattccttaacaatcgtttctagatctgtcttcttgattcacttaaacatatattttgctgcacattttgtatttgtcctgttgatgcagaaggcaatgattcttaatcctggctgcacattagagcttgtaagaaaaatgcaaatgtcccggctccatagcccagaccagttcaatcagagtctctgggagttaggcctgggcatcagtgtttttacatctccacaggggaacaaacacataggcttctgcatggtgataagtgcaattaaggaaataaaggcaggttaacaggagagggaggaatggggtggtctgtctcagaggcctgtgggaggctggcttgctcccactgaggccttgtggaaggaaacagccacacaaagtgtcctgtctgagccagagacagacagcagagagtggagacctgtctgagtcgagccagcatcagcgctgggagatctgcaagctgctgctgccgctaccacagcccagaccaccctggttccttctcagtgcagccttgcttagtcagctcttcattcaattttgtgcagtttggtgcaatatctcagcttccttccaataaatcccccatttctgcttaagctagcaagaggaggtttccacagaaacccaaagaacttctatgaatacaacatgtcattaagtttaaattaatctttttacacagtcatgtttgacaaacacagaacttcacatcaggagatttgggaaaatctattccagcctctgccattgaatatctcaagcatattccttaattttttcagggtcaaacaagatatctatcccttttaactctaattctataaatctttgaatctgggatccttcctgaacatattatgcaatagacactggaagagtaagtgcctaaaaggcagggccctgtctccatgtgagtggaggacacccagtaggagcttacttagggaactctttttctttctttcttgctagcccctgtcacagttcattagcactatcctccccttccctactccctccactccacaagttggacaaaatgctgttagtgtatccaggatataaacacttttaaactgcagtttttggtggttcatgcctataatcccagcactgtgagaggctgaggcaggaggatcagtttagtccaaaagttcaagaccagcctgggcaacatagcaagaccctatctctacaaaaaactaaaattagctgggtgtggtggtgtatgcctgtagtcccagctacttgggaggctgaggcgggacgatcacatgagcctaggagttcaaggctgcagtgacctacaatcacaccactgcactccagcctgggtgacagcgcaagatcccatctttctctctctctctctttttttttttttttagacagaatctcactctatcacccaggctgatgtgcaatagtgtgatcttggctcactgcagcctcgtctcctgggttcaggtgattcttgtgcctcagcctcctgagtagctgggattactggcatgcaccacggcgcctggctaatttttgtatttttagtagagacggggtttcaccgtgttggccagggtggtcttgaactcctggcctcaaataatccacctccctcagcctcccaaagtgctgggattacaggtatgagccattgtgcccagccccatctgtttttaaaaaagagaaaaaaaaatagcagtctcaaaataataactatccctgacctcactcatcacccaaaattaacaaaaatctgatgttttatttttctgtctgacacacacacagcctaaggtgccattgcagtattgggcatggggtaaagggcaatgctgccattgccacacacatttccagtatggagccagagagaggtcagcctggctccactcccaggggaggagggagaggcattgtggaattcaggacatggtgagggatttatattttccttcacatctggcactcagggatcatcgtcaaacacaggctggccatctaaaacagttgttgcagtgtagctcacatgctgtcctcggtgtagatacatgtcttcactgcttttgtgaaccatattcgcacgcacccttccagagcagtattatgtatggaaatctttcaaaatcacccaaagtaacctgacaaagtccagaagaaattgaaggtttagccttagaagcaccatttttaagttagaaaacagggccatttaacagtaggtaataggaaacagccttgtcagttgcagtttactgatgttaaaagcgtaacattgaacacagtcactattttttctcaatattcactttcccctcaaaaaacaaaataagtgtctctttaaaaccaatcaaataccaccattttttttagccaatgatcatctcaataaaaattttttctgaagatttagttaaacatgaaactatcatgagataatttaccataaaggggaattatcaaattgacttactcaaagttctatatttactgaacttaagatagctctaatttttataccattattttatatgcttttaaaacaatggctaccctttaaaatattaagatgccattaattgtgagtttagaggtattaaaatagaaacatactcatgtcatatcttagagcttatggtgatagtaagtacaggcttattgaatatccattcctttttttcagaaatcatcatctttgttctctcttatcctcgatgtctctcgctcgctcgctctctttctctcttctctctctctttccttctctttctctctctctctttggcagcactgcttcatatgttttatatgtaaggagaatttacttatttttcttaattttttattattttttgagacggagtcttgctcttctcgcccaggctggagtgcaatggcacaatctcagctcactgcaacctccgcctcctgggttcaagagattctcctgcctcagcctccaaagtagctgggattacaggcatccgccaccatgcccagctaattttttgtatttttagtagagacggggtttcaccatgttgaccaggctggtctcaaactcctgacctcaggtgatccgcctgccttggcttccgaaagtgttgggattacaggcatgagccaggcccaagatcacaaaatttaaagatccaggacctcaaatacttctttgtttggttctggtttcacagaagtatttttttaaagagtttaataaaatatatttaaacaaactatagttaatataaataaacctggatagcagaggcttaatggtatctggaattgcggtgtgtatgttcacaagacatcaggcaaagaaccttatgcgttggaatatggaaggcaggcagcatccattctagaatgccagagaggttggacagctgcaacacagcgatggaaactgaaataaaagagattttcaaaagaaagagcagataatagataaggctttaacacagggtacaagcagcgtcatcagcccatggttaatacagtaagtattataaaatgtaacgttttaatctaattaacaaaaccatttgtactctcccagtctaattcacaattattaaattaccctaattcaaagcaggtgaaaccccaggcacacaaacagatggctaagagacaatttcaagttgagttgtccctgaatcccatctgaaacacatcattagcattgctcaccttttctacatttgtttgaaacttggctctattgttacaaacaaaatgaacaggaggggtgggagtgaagagaagagcaacttccttggcctttcttattttaccaattaggtaaatctactggatattgtagtgaaactgttttctcatgtctggaagggtaaggtgagcgaggctgatgaaaaggaggtagaaggaacataatcaccactatggccattctgttatctggattcctaacaaaattcctgaaaatggaggcctcaggaaaggttccagagattggggtgttcctggaaggcatgtggatggccggcagggctgtgacctacccaggcccctagtcccagcagctgacctggcttgattcactgcatagtgcctctctgagctctgaaggaactgtacacggctcacatgagcaaataagtgccttctccttttttcttcttccactctcactttaggggagcatttgcaccctaaagtatgggggccatctcagtatatttcatgtcaaatacataaagtgcttacagggagcagcaggactggcccccttagaatacagtgtcccccttccccttagattagtgcctgagtcctcaatctcttctccactccacggccgacctccagaccctcctacacctcacaggccatcctaaaaaccctctgacctggcctgcctttctcttatttcccatgccattcatattccctcttctttctctctctgtcatacgtgcacacacacacacacaccaacaacaaccctccacctctccattctccatgctgcatgctagtccacttatctttccaaaagcagccatgatttcatctctcccctgtttcctcactgctcattgctcatctgatagagcttagcaagtcactcctccacaataacctgtcttcctggtttcataagctgctcatctcattccaactcatcctctctcgcagccataagaaagcccagtactccaaggcccttcataccttctggtgccctcgcacttgcttgcttcccagaataacccatcccataagcagcctccttcatgtttcaaatcagccatttcacagaccacatcctccataaaaactgtgccgaatccccagacaaaactacacatcccttcttccacgtctgtgcttatttcaagtagagcacttgtctcacgtcctattctactcagtaccttcagccccaggacactgccaagcccattacgggcgtttacactttgctgaatcatttcattaataaaataatttatgtgtaattatttctcctcaacccctcaaaaatctcctccagcactggtacctttaatgtagttgaaattcagcaagcacaactttaatcgttttattcaagttggtgttttctagatttgctagtcccaagaatgaccagagtatttgttaaaaatatagattcttgggccctgcagtaggcagcccctgaatgagactttacaaggaggctgtggatatttgcatttgtagcaaacaatataggtaattcttatactcacttgacttaggggacacttttgtcccctactaaaatctgaatacctttggaaagaaagcaacatagcaaactttaaaactatttatatttcaatgtgaattatttggtaaaatctttttgctaaaagattttaacagaggggcccagggaaaaggaagcgaacacaggatatatccagttttgatttttgcaaaaaggtttttggatatgaggccagtttatatccaacccctttgtaataaaaatacttacctagtaaaatggtttcaacaggcagacatcccactaactctattacatcacttgtcatttttaataagtagcatcatcaattcttctagtggttaaatattgataatacaaaatatagatttaattatgaaatttcattactatataaaagtaaagtaaaggggaataaactttttacacacgggccattttaagaaaaattataatatttgtagtccattaaaagaatacatatttttaatcaggaaatataatattgtaactgtgcttatgtgcatttcttttttccttataatgaaggaatttcttctctgttttgttttgttttgtttttttttttcagatggagtctcactctgtcacccagactggaatgcagtggcataatctcggctcactgcaacctctgcctcccaggttcaagagagtctcctgcctcagcctcctgagtagctgggattacaggcatgtgccacgatgcccagttaattttttgtatttttagtagctacggggtttcgccacgttggccaggctggtctcgaactcctggacctcaggtgatccgcttgcctcgacctcccaaagtgcgggattacaggcataagccaccatgcccagccaagaatttcttatttgggttaaaatttcaaaaactacttaagcgcaggcatggtatagtctaaccataacgttagattttataaaggttgtgactaggagtattcaagcctgatttctttccttgactggctttcattgcaaaataaccacttcattacgaaaaacattttgtttcattatgcctagcaatgcatccttaatgatacattgcaaaaggggcacacagaaatgtgttacacggtgaaatattctttagtcaaattcctttggagtacatttatttttaccatcatttcttttcttgcagcagtctagctggtgtttaaatgattatctctaaggtagcttgccccagtcacactgtgctctaagttgttcctctctccttccatttgagcagggaaacactagatgggagaaagcgcggaaagggctagaagggacgtggtagaacacaccacgacgatattgagctcactcagaatcaaacttccacatctccatttggacgcccaccacaccccatcaataagacctgtccatatcacaacacacttgaaacccactcccctcccatcttctccatctcagtaaagggcaccaggtgctgaagtttaaaaccaagaagtcgcccttgattcctctgtttctcaatcttcaaatcaaattcatcagcaagccctttccacggacatgcaaaacctatccccaatctcatgtcgctttgactccctgcctcagtctccagggcaggtgccccgcagctcataggactgcccccagggttaccagataaaatacaggacaccctagtacaatggaatttcacatatgtataaatacttccccaattcagacttctttccaccaagtagtgaatgtacatttccacctgctgcccctacgagttcagggtcaccaggagagtcatctcagtctgtacaaaagagatggttcacgcaattccttgagtatttcattgtattttaaccttgtaatttttaaaatttagtaaaaaaccggttgggtgcggtggctcacgcccgtaatcccaacactttgggaggtcaaggtgggcagatcacttgaggccaggagttcaagagcagcctgaccagcatggtgaaaccctgtctctactaaaaaaaaaaaaaaaatatatatatatatatatatatatatatacacacacacacgtatatatatatgtatatgtatatatatatacacgtgtatatatatatgtgtgtgtgtatatatatatatatacgtgtatatatatgtacatatatatacacgtatatatatgtatgtgtatatatatacgtatatatacgtgtatgtatatatatatatatatgtatgtatatatatataaattagccggacatggaggtgggcgcctgtaggcccagctacctggtaggctgaggcaggagaatcgcttgaacccgggaagcggaggttgcagtgagccaagactgcaccactgcactccagcctgggcgtcagagcgagactctgtctcaaaaaaaaaaaaaaacaaaattagtaaaaagccaaaaaccaaggtgttgatacgaaaactacaaaaagcagaggtgccacaaacctaattcaactgcaacaagatgaaaatcacaaggcaggctgaaagcagggactcttcagctataagcaaaacaatttttgacttaagcttatagccttattgggaagaaaatacataaaattaaagaacaaaagtgaaacctgaatatatcttagaaattatgtctaacaaccccaaaattcatatggaaccaaaagagagcacaaataccaaagcaatcttgtccagaaggagcaaagctagaggcatcatattacttgacttcaaattattctgcaaggatatagtcaccaaaacagcatggtactggcataaaaatagatacacagatcaatgggacagaatagagaacccaggaataaagctataaatctacggccaactgatctttgacaaagtcaacaaaaacatacactgggaaaaggatacccttttcaataaatggtgctacaaaaactggttgccatatgcggaagaatgaaactggactccatatctcttaccatatacaaaaatcaactcaagatgaattaaagacttaaatgtatgacatgaaagcataaaaatactagaagaaaatctagggaaaacacttctggacattggtctaggcaaagaattcatgactaagacctcaaaagcacaggcaacaaaaacaaaagtagacaaatgagacttacttaaactaaaaagctttgcacagcaaaagaaatactcaacaggaatgaaccgacaacctgcagaatgggagaaagtatatgcaaactatgccttcgacagaggactaatatgcagaatttacaagaaactcaaacaactcaaaaaaaaaaaaaaaaagacacacacacacacacacaaacaaccccattaaaaagtgggcaaagaacatgaatacatatttttcaaaaaaaatacatacaaatagccagcaagcatatgaaaaaaatcctcaacatcactaatcattaacaccaatcatcagaaaaatgcaaattaaaaccacaatgagacttcatcttacaccactcagaatggctattattcaaaagtggaaaaacaagaagtcaatagatattggtgaggttgcagacaaaaggcaatgcttatacactattggtgggaatacaaattagtacaactactgtggaaaacagtatggagatttctcaaataatgaaaactggaactaccattgatgcagcaatctcactgctgggtatctactcaaaagaaaagatatcattacattaaaaagaacaagtgcttgtatgtttattggagcactattcagaatagcaaagatatgcaaccaacctaaatgtccatcagcggatgactagataaagaaaatgtgctgaggagccaatggggcgaatctgccatctgtgggattatgactgaacatgtccaagtcagaatcccactcaggtggaacctatggcagcgccacagagcctccatgggccttgttggccctggatggctggtccccactgtccccactagaggactactctccccaatgccaggactggggtccagtgcagagaggccctcctcctgggaaacagggtgcagctggaaatgcagtcgcaccctggcccatcacgtggcacacattcatggggaacctggattctgggtcctagacccacctgcttctgggtcagggtttcctacttagcagagaagcaaattcgccatgatctactgaaagcccttgacactagtgtttgaaataaaaataaatgaagaaataaaagtaaagaaaatgtggggtgtgtgtgtgtgtgtgtgtgtgtgtgtgtgtgtgtgtgtgtatatatatatatatatatatacatacacaatggaataccattcagccacaaaaaagaataaaattatgtattttgcagccataaaaaagaataaaattatgtattttgcagcaacatagatggaagaggaggccattatcttaagtaaaacaatttataaagtcaaatactgcatgttctcacttataagtgggagctaaataatctgcacacatggacacagaatgtggaatgacaggcaatggagactttggagggtaagaggatgggaaaggggtgagagagaaattacttagtgggtacaatgtgtgctactcagatgatggttacactaaaagcccagacttcaccactctgcaatatatccgtgtaacaaaactgcacttgcacctcttaatattgtataagtaaaaaagaaagaaaattatatctaaaaaggaatgtgcaattataaaaggtttaaacagccttggcacggtggctcaggcctggaatcccaacactttgggaggctgaggtgggcggatcacctgagattaggagttcgagaccagcctggtcaacatggtgaaaccctgtctctacgaaaaatacaaaaattagcaggtcgcggtggggggtgcctgtaatcccagctacttgggaggccacagcaggagaatcactcgaacctgggaggcagaggttgcagtgagtcgagattgcaccactgcactccagcctgggtgacacagtgagactctgtctcaaataaataaataaataaataaataaataaataaataaatggtttaaacagcttagctgtctgggaattgatgcccttatcacctttaattctatttttagctttcaagcttaggaggaaaaaacgggttgcctgcaaatcatccatggtatggctaacgagcagtgtgacctcaagcagcatatgctaatccttcccaggattttataatcctctttattcattagaactgttggagtttctaaacaagtaaaacttgaagttttggtttcaacccttacaatctgctaaggtaataaaatgtttgcaatagtaattaagccctgtctcttatttagatatacatttttaaaaatttgtatttatttatttatttaaactacttacttatttatgtgaaacagggtctcactctgtcacccaggctggagtgcagtggccctattacagctcactgcagcctcgacctcccaggcccaagcaatcctcccacctcagcctccctagtagctgggaccacaggcgtgcaccaccacgtcctgttaatttttttatttttgtagaaacagggtctccctatgttgcctaggctggtcttgaactcctgggctcaagcaatccttccacgtgggtctctcaaagtcctgggattacaggcgtgagccactatgcccaactaagtttgattttccaccaaaaaaaaagcacctacaatgaaaggtaaagatcctaactgtgctgacatcagttctggtagatgtatgtgcccacgtaaccaccttccaaaaccagacacagagcttccttgacacctcacaggcccatggtgctcctttctaaggcatcccctctcatgaccctgttcctgaacttcacagaaatgaagtgcatattcttatctgtttgacttattttattgaaaataatgtttatcatagaatacgttataggtgaaaaaaaagtttttgagattcaatcagtgttgaatatatcagtgtctcatttttttggttgagtaataccccattttatgaatatatcaaaatgttttgaaccactcttctgataatgaacatctgagatatttccagggttgcataatattatgaataaagctgccatatatagaactggacatgtctttttgtggacctatgtttttctttctcttggattaatatctagaagtggaattccttggtcatgaggtagatgtatgtagattttctaagaaatgaccaaagggtttcctgcaagtggctatacctgtttatactcgtctccctgatccttgccagcatttggtcagtctgttttgcaagggatggcaatggctcactgagcaagcattcatgatccaggaagaggaaaaatcattctcaataggacagggttattaaggacagggctctcagcttacaggagggctgctccttcttccttcatctggaggcttacacctcagctcaactgttgttcaactgggatacatttcatttatttctctctctctctcctctctctctcttttttttttctttttcttttttttggtagagactgggtcttgctacgttgcccaagctgatctcaaattcttgacctcggccaggcgtgatggctcacgcctgtaatcccagtactttgggaggctgagacaggtggattacctgaggtgaggagttcaagactagcctgggcaacatggtgaaactctgtctctactaaaaatacaaaaattagctgggcgtggtggtgggcacatgtaatcccagctacttgggaggctgaaggtgaatcacttgaagccaggaggtggaagttgcagtgaaccgaaatcacgccattgctgggtgtgatttcactccagtctgggcgacagattgaaactctgtcaaaaacaaaacaaaacaaaacaaaacaaaacaaaaaaaacgaattcttggcctcaagcaatcctcccacctcagcctcccaaagtactggtattacaggcatgagccactgtgcctggcccacttattttcttctttatcaaagcatgttcatatactctttcacaaccaaccccgattaaatcctgatcagaaattctaacagatggcaaaagatggctcagaaatggagtaatttgcccagagtttacagggcgacctcaccatcccagccctctgacttaacaggtgctgcgtaactgcacatatcagatgaactactgtatggaaaaagaaagaaaagcaacaacacaaaactactgaatgctgtgagttatgtagaactatgctgcacttggtggtttctgactacatgctcctttctcactataggcttctttccagaagtcaagatgaatgggcaacctctgggactgccagaggtgtgtcccaggaacagaggagcgggctctttgcatgggagacactccaggcctaatgggaagtccctggagcaagggacttcatgcactcacatgatgcatatgacacagacaaggcaacgctgttttggtgagtgaggctctcccaacagatgggagaggagtgaggctctccctgctcccctgcccatcccataggcctcttaagtgtttgggggtgactaggaaaagaaaggactccaaccctgggaaactatccatcctcttaactgggacatttgtggttcccttttcccgagatgaccttgcggtagcttttctagctgggtagctagcaattctggacacatctcagcactccaggacatctgacattcaggtgggaactgactggatattaactgggccaccttaattcactgaccaccatgttttcttttgtctttctttctttctttctttctttctttttttttgagacggagtttcactcttgttgcccaggctggagtgcaatggcactatcggctcactgcaatctccacctcccgggttgaagtaattctcctgctcagcctccggagtagctggaatcacaggtgcctgccaccacgcctggctaattttttgtatttttagtagagatgtggtttcaccatgttggccaggctggccttgaactcctgacctcaggtgatccgcctgcctcagcctcccaaagtgctgggatgacaggcacgagccaccacacccagccaggtcaccatgttttctaaaatagccgaaagattcccacatagctgaatctagtaaaatgattctcatccttggtacacaagcagaatcacctggggaactctaaaaaatactgatgcttaggcccccaccattcccatttcattggttagggaacagcttgggcatggggaggtttaaatgctccccattgattctaatgagcagccagggttgagaatcactagccacactctacactacaaccttacctgcttttgaaaaagggtaaaatgcgcagtctaataatgtagggaaccttgccaaaccctgagctccaccctagaattgaaatcatagcaatggaaaccttttatgtttatcttcttttttttcttttttgagatggagtctcgctctgttacaaggctggagagcagtggtgcaatctcggctcactgcaaactccacctcccgggctcaagcaattctcctgcctcagcctcccgagtagctgggattacaggcatgagccaccgcacctggccatttctttatcttttataggtgcatgatctgcagggtaacacatacagaaattattttgtgtggcatcataattccatgccctattaaagggtggggactggaatttatccactgccaacctaaccccatatgtgaactcagaacagaaaaaatgctcagaaaaaagggtggtctgtgattgacaaatagaacccaggattgtaaatacatttgctggtgttcagtaaagtttagtaaagttgaatttcatatcatatttcatatcatattctaagaaatttattttcattccatcaacccgacagccaaataattgagttacttactcctgaatttggttacactatagccataaagaaaatgttggtatttacaaagtattagcttcagggtatattttatttaaaaaattgttcatcgtacttatttcagatttatctattaaagtcctgctattattttttttaatttcaggagtcattctacctcattgtttatcaactttcagagaacacattttttacaagtgataaagtaattctactttatgtttgaaactccaagctctacataaatgataaaacactttttttttgagactctgtcttactctgttgtaatggcacaatcacagcccagtgcagctgcaacctccgaggctcaagcggatcctcccatctcagcctcccaagtagctagctgggactacagggccatgccaccatgcctgactaatttctttttattttctgtaaagatagggtctctctatgttgcccaggctggtgtcaaactcctgggctcaagtaatcctcctaccttggcctcccatagtgctgggattacaggtgtgagctaccatggataaaacactttaaattctacatttagaataatagccaggttaatcatacataggtaacaagttactttcaaaaggaaggtctcaataaaacttaattattcagattagattatcaacatagaaacagaaagagcttacagtgaaaaatatccttccaggaaatgatttatccctgatcttaaagggaaaacttagaaaagcagcaaattttcaaactattcagctcatgaaaagttggtaatcaaaaacactcaagtagttttttagtctcttgttgaagtttatttcatctttgtctactttccctcacagggcatgatatagaagccttaatattttcattgttccctactggaatctctctgttcttttgaatacttcaaataaacacctcaaagaagagctgctcaccatgaagacaacttcccattcaagaaagtacaaggcccagcacggtggctcacgcctgtaatcccagcactttgggaggctgaggtgggcgaatcacttgaggtgaggagttcgagaccagcctggccaacatggtgaaatcccgtctctaccaaatatataaaaattagctgggcgtaggccgggcgcagtggctcacgcatgtaatcccagcactgtgggaggctgaagtgggcggatcgcgaagtcaggagttcgagaccagcctggccgatatggtgaaaccctgtctgtactaaaaaatacaaaattagctgggtgtggtggcacacacctgtagtcccagctactcgggaggctgaggcaggagaatcgctggaacccaagaggcggaggttgcagtgagtggagattgcgccgctgcactccagcctgggcaacaaagagagactccgtctccaaaaaaaaaaagcctcatgctagatgagaatgggcactgttgatcaaggtgtccaaaaatagttaatgtggctaaattgagataggttatgcttccatcacagtatgcatattgcagtggtgacaatgagacctgtaacattttgccatgcttatgagtaaaatatgtagaagaaaaatgttgactttcaaaataacatttaagtaataaggaattttcagctcttatctcataggactactgttgttgttgttttttttttgtatgctatatattgattgaaacattgtaatgtggtacatgactgtgttttggatcaggtgtcaaaatataagtgtactttgtacaattcaacttctagaaaaaaaaaattagctgggcgttgtggagcgcacctgtaattccagctactcaggaggctgaggcaggagaattgcttgaacctgggaggcggaagttgcagtgagctgaattcacgccactgcactccaatctgggctacagagcgaaattccatctcaaaaaaaaaaaaaagaaagaaagaaagtacactgttacggccaggcgcaatggctcacgcctatcatcccagcactttgggaggcaaaaatgggcagatcacctgaggttgggagttcgagaccagcctgaccaacatgtagaaaccctgtctctactaaaaatacaaaattagccaagcagtggtggcacatgcctgcaatcccagctactcgggaggctgaggtgggagaattgcttgaacccaggagacagaggttgtggtaagccaagatcgcaccattgcagtccagtctgggcaataagagtgaaactcgtctcaaaaaaaaaagaaagtacaggtacgttttttagaatgatgatacatcatcactgactcacttggtttctgcaaccagatcctttcccgtgtctgtgcagtcatgactccattgctgccctgaggtggtatactaacccaaatcacagtctgaaagtcatttttcatttaggttcttccctgcttatctttggttgagccaggaaatgcaacttgataatgactcagcacaagctattcagctgggagccggttcctagccacatccagctccgcgttggaaatatctgaagtggtcagtctaggatagagcagtgctgctgtagcttccaggtagggtttcctgaagtgtatgcccaatgctctttctgaatcagcactgaaaatattcatgcaggacctcccccggccctacctgaccccacagagcaagagcaatacattcctctgcctgtcactaggccctgggctgccaggtttgggggatactaagtccagggaaaccaccagaagtagttccagggaggccacaggtgtgcaaaagaaggatcccatgcatgagcccaagaaggagtctaaagcctggccttgagatgaaaccgttcaattaaacagttcctttcatattaaagtcaatgtaaaaataccatcacttacactgacttcatcagacaagagaacactggggttttgcttaaagaatcatctgacagcctcagtaatgaaaggaggtggaggaattctatcaattttcctgttttaaaggacgaggtgaagtcttacccattttgtaactgctattctaatcggagaactattcagaagtggaaacatctattcataaagtattacggggggaaggagagggaaaaaaaagcattatgaacatcatatgagacacacggccacgaagtgcccaagtgctgagcaaaagcactggcagtatggtttacatcacttcccaataaaccagttttgctcaggcatcatagaggcactgctgcaaatgccaccaagaaaccagaacaggaaaggggctatgtggctcactcaatccacgtcttctttgccaattatccttctttttgttgttagtaaactctcaactagaaccttaataacttttcaatgtctaagattttgtggcagcattttaagataaagagttaggagaatataagatttccaagagctattctccttaatgtggaaaaaaatgcaagctagataagtatttagcaaaagaaaagtatgagatacatactgaagagaaggtttaatccaaacataattaaatcttagctataaaaaaattattagctttgctttggcaaaagaagaaaattaaactgggcacaatggctcacacttataatcccaatactttgggaggccaagactggaggaccatttgaggccacgagtttgagactagcctgggcaccatagtaaggtcctatctccaccaaaaaaaaaaaaaaaaaaaaattagctgggcatggtgatgtgactctgaagtctcagctactcaggaagctgaggcaggaggaccacttgagccaaggagttcaaggctgcagagagcaatgatcacatcactgtacttcagcctgggcaacacaatgaaatcctatccctaaaaaaaattaaaaataaaaaagaggaagattatttttcccccaacaaatagtgacatttataatagatataaactgagttgtcagctgaagttaatgcagaaaactcaactggcttgacactcatcatgctttaaacttgacgcaatgtttaaatattcattgcaactccagtgaatttttttcttcttcatttttcccagctatggttatagtaaaaattgcaccaagttggcagaactgggggctctcttgttttggccttgctgactggttttcagtgtctgagctcttgtcttcctgaagttcgagtggcagcgcattagattcctggagcagtagttaagcttgagctaaacatcctaggcccaagttcagctccacctcgtacagtgacatgacattggcaaggtaattcatctttacttatctgcagaaaagggatgataaggctgggcacggtggctcatgcctgtaatcctagcactttgggaggccgaggcgggtggattgcctgagctcagaccagcctaggcaacacggtgaaaccccatctctactaaaatacaaaaaattatctgggcatggtggcgtgtgcctgcaatcccagctacttgggaagctgagacaggagaatcacttgaacccaggaggcagaggttgcagtgaactgagatcgtgccattgcactccagcctgggtaactgtgtaagaccccatctcaaaaaagaaaagaaaaaagaaaagaaaagaaaagggatgataactgtaccaacttcacacacttgttataaggattggttgaatacaatcttgacgagtgtttagaagaacgaatgacataccgtgtgcactcaataaatgttctctgcattatatttattatacatttcccagaagtggtcttattcctgcgtttacatttatgttaatgtataaagtctgcttgataaactaaagtcaaggacattatgttccacatattacaactgtgattttttaaaaaataacctttcaaagttaaataaaacaaacacatcagtttaggggtccggctgacttgggttccatttacttagtagttaagtgagcataatatttattatccccaagcatctgtttccttatcaataacaagaatatcaatattacaccttttaaggaattgactacctaacagaggacaaatatttaataaaggattgctattcttactatattttctgtgaaaggtaattattgcctgatacaaaagtaaaacaggtagtaactgtgctggcatcattgtaaaactcttccaactaagaaagaaacttagctgggtacagtgtctcatgcctgtaatcccaacactttgggcaatatatggagacctcatccttacaaaaaataaaaaaaattagcccagcacagtggtgtacacctgtggtcccagctacacaggaggctgaggcaggaggattgcgtgagcccagaagatcagaggctacagtgatcgtgattgtgccactgcactccaggcatcagagcaagaaactgtctatttaaaaaaaggaaatcattaaaaccagtttcctaaaatttattatagagctagaaatagattagtgactgcctaggacaaggggaagagagtagtgagggctaaaggatacagaatttctttttgtgtaataaaaatgttctaaaattgattgtggtgatgattgcacaactctgtgaacatactaaaaatcactgaattgtatactttgagtgaattatacaatatgtgaattatatctcaataaaactattattcagctgggtgtgggtggctcacacctgtaatctcaacactatgggaggccaaggcaggtgaatcacttgagtccaggagttcaagaccagcctgggcaacatggtgagactttgtctctaccaaaaatacaaaaattagccaggcatggttgcacgtgcctgtggtctcagctacttgggaggctgagatgggaggattgcttgaacacggaaggcggaggttgcagtgagccaagagtgtgccactgcactccagcctgggtgacagagggagactctgtctcaaaacaaaattaaaagtaaaaataaagctgttattataaaagacaataaaacgatcacatacagtatgaatctatttatatgacattacagaaaacacaaaacaatggggacagacaacaggtcagcagttgccaggcattgggctgggggtggcacaagaaagtttttttttggggggagggtgtcacagaacttttctgtattctcattatgaaaataattcagtgaatctgcacatgtattaaaattcatagagctatatagcaaaattttaaagttgatttaaaaaacataaaaagttttcttaaaatcgtctattttcattttaattttttccaaagaaaaaaattgcttactaatctttaaaagtttttgttgttgttgttgttgtttgtgagacagggcctcactgtcacgcaggctggagtacagtggcgcgatctcgactcactgcagcctccacctctcaggctcaagcaatcctcccaactcagcctcctgagtagctgggaatacaggcccatgccaccactcttggctaatttttttaatttcctatagagatgaggtctcactatgttgcctaggatggtctcaaacttctgcgctcaagtgatcttcctgcctcggcctcccaaagtgctagcattacaggtgtgagcaactgcacctgaccttaaaagttattctgatggctaagagtgcttatatcccattactaaagattttgaaatattttagatacatatgaagtagtagtagtaggttttctgttaaaatggaggagaaattgaggtagaaaaggcaaatttgaacagtaagacagaactgttactagactctggagatcttctgctgtcaacccctcgaaaccaccatgtgcaaagtcagaggtgtgctcagcgtcagaatgctccctcctgatctcaagtcttggtaaaacttccagaagcatgtagcttattattatgaggaggagggaagtaccttttaaggtacaacagagaaagctttactgttcttaatctaaactctgaattttcatttatatggcatgataggcgtcacatgaatcaaagttctaatgtactgttgcttgattttcagctccttatcaggagaaattaggtcaagactgtaatgcgatcaatcagaagatacctgtggatgttgaaactgcataaagccagcgttcgggtgagtaacatcgttctccaaccagctcattccattgcagttatttcctcatcatttaaatgaagtttaggccgggtgcagtggctcatgcctgtaattccagcactttgggaggccaaggcaggcggatcacctaaggtcaggagtttgagattaacctggccaacatggcaaaaccccgtctctactaaaaatacaaaaattagctgggggtgatggcgggcacctgtagtcccagcaactcaggaggctgaggtagggagaattgcttgaacccggaaggcagaggttgcggggagccgagatcacgccactgcactccaacctggttaacagagcgaggctctgtctctaaataaataaataaataaacgtgaggagcttggggggaatgcctctcttactaaattaggtatggtttttattctatgtttcttttttttaaaatttttttattatactctaatttctaagatacatgtgccgaacatgcaggtttgttacataggtatacatgagccatggtggtttgctgtacccaccatcccgtcatctacattaggtatttctcctaatgccatctctcccttaggccccccaccccccgacaggccccggtgtgtgatgttcccctccctgtgtccatgtgttctcattgttcagctcccacttatgagtaagaacatgccgtgtttggttttctgttcttatgttagtttgctgagaatgatggtttccagctgcatccatgtccctgctattctgtttctttatttataaacttgcttttgtttctttgtttgatgttgttgtttctttttcttttctttttttttcagagatgagagtcttgctctgctacccaggctggagtgcagtggcataatcatagctcactgcagcctccaactcctggactcaagggacccactcacttcagcctccagagcagctaggatgacaggggcatgccaccacgtctagctaaaacttgttttttttttttaattgctttacccctaacgagatgcttaaggatgagctaatgataaaagaaaaggtgttctctttgctatttgtcataacattttgagaagaatgaaagtgcacctggtatgtacaccaaatttccacttccccacaggactggtgatgatgcatgagtgaatctaccagaacaggccaggggtaccatgagtagggagtgggtagggatcctccaaaatcaagaaaaggctcggggtagtacagtttaatccaaaacaacagccaaaactgggtagaaaattagactcttcaaggaactcccaggtacattaccccgtttaatccttgcaacaactggatgagataccaaacaaaaatctgttattactgttttatgtattagaaaaacattagatgaaggagcttcatctaatgtcccaagactggtatttgacaaagcattgactagaagccaattcctttaaaaaaaaaattgtagagatgaggtctcactatgatgcccagcctggtcttgaattcctggcctcaagtgatcctcctgccttggcctccccaagtgctgtgactacaggtgtgagccaccacacccagcccagaaaccaattcatgactctcagtttatgactacgacatcatgctgctcctcagacaccccaaagttcacaacctggaatatttttcattagctactacagtgttactaatccatacctatattattcaccaagattagtaaggaatccagccaagttatttccccatctcttcctactttatttccaactaagggactcacttcttcacttggaaactgttctgctctggtcagtctccttagtccaatattccctccattctcaggattcactctttgttcaaatcctctgtagcatccagcagttgctaacagagacaagcagttaaaacgcagagatgccaagcatcaggaactgtggctggaggaaataaactggaggttttggggctggctgcagtggttcacgtctgtaatcccagcactttgggaggctgaagcaggtgggaagatcgcttgagaccaggagttcaagaccagcctgggcaacatagtgagatccccaactatacaaaaaattttaggcttggcactgtggctcacacctgtaatcccagcactttgggaggctgaagcacatggatggatcacttgagcacaggagattgaaaccagcctgggcaacatggcaaaaccccatctctacaaaaaatacaaaaattagccaggcaggccaggcatggtggctcacgcctgtaatcccagaactttgggaggctgaggcgggtggatcacctgagatcaggagtttgagaccagcctggccaacacggcaaaaccccgtctctactaaaaatacaaaaaaattagctgggtatggtggcaggtgcctgtaataatagctacacaggaggctgaagaaggagaatagcttgaacccacgaggcagaggttgcagtgagccaagatagcgccaatgaactccagcctgggcaacagaacaagactccgtcaaaaaaaaaaaaaaaattagccaggtgtagtggcacatgcctgtagtctcagctacttgggaggctaaggtgggaggatccctggagcccaggaggttgaggctgcagtgagccaagattgagccactgcacttaagtttgggtgacagagtgaccactgtctcaaaaaaaaaaaaattaagaaagttaatctggcacagtggcatgcacctctagtcccagctactcaggaggctgaggagggaggatcacttaagcccaggaggtggaggctgcaggagccatgaccatgccactgcactccagcctcagcaacagaacaagaccctgtctaaaataataaaaagtaaaaaataaaataaaaataagctgggggggttgggggatgcagacaggcaaactacaggctcagcatgatctcttggggtatggttattccttgcatggccaggacttcacttgtgactttttcacagaaagatgggtcccttagtgggtcccacagaaagatgaacccacagatgggtccagtgcctgtgcatgttcttcatatcttgtgtgacacaaaaatttatactctaagccaggtgcagtggcacatgcctctagtcccagctactcgagaagctgaggcaggaggatcacttcagcccaggatctcaggaccatagtgtgtgagtgataatcgtgcctgtgaatggccagtgcactctagcctgggcaacatagtgagatcttgcttctaaaaaaaaacaaaacaaaacaacaaaaataaaaggaaaattttctttaaaaaaaagttttttaaacattttaactctctgagtatatctggaacagttttgctattgagaactcatggctttgactgcaagtcatatgcatgcatattcatgagtagtgagtatccaagaagtgttttctgaatgatgcataagtgaagcatgacttcatggattaacaaatgccacaggtcaattatgcctctctctgtcacccccaacaaaaataaatatcaaaactacatgaaacagaagttgaaataacagtcagtaacaaaggtgctgtgttccttaacatcagaaaaatgagtctttattttattgaagaacagcatcactgaattcacaaacccagttagtactttcaaatgcctatgcacagactggcactgtgtcatataacaggatgtttgattacatgtgggtcagtggaaaccacccttatcacacccacatatttgcatttataaattgacttggctgtgcagaaaaaccagctatatatagcttatgccagctaattttactaattaatggatgaataaaaaggtatttaaataaggctcagatcaaactcatgtgtgagaattttagcaactgaatcgccagggtaaaacaggacgaggtcttgctctccagttgaattgcaagtaaattccacacacagagagatgccggtggtagccaaacccacagcttgaaaatctgtttatttgccaaaccaaatggctttgtactgcttttcaattccctacactttattgtttagaagcctacactggaaactaaacaaaaactagaagtagaatggcagatgcctttcaggggagtactgcttgggggagggtcaacatcattctgctttttgatcatgggggtggttacacaaggctttgcattcagctagattcttctgttttctgcacttttctatgtaagtgttatacttaaattttaaacacacatacacacacatacacacacactcctgcattgggaaaactttttaaaaaattccctcgaatcctgttagaaagaactgtgtcagaaatattctttctttggcaaggcacaatggctcatgctcgtaatcccaggactttgggatctcgagatgggaggatcatttgagcccaggagttcaaggctgcaatgaactatgatggtatcactgcactccatcttgggtgacagagcaagaccctgcctcaaaaaaaagaaagatactttcttattatgtaatttggagttaatgagtgcatggctagaaatattaatataactgtatcatcaaatcaggaagcaccttttacagccatctaatgcaacttttttatgtatttatttttttgagatggagtcttgctctgtcgcccaggcaggagtccaacggcgtggtctcagctcaatgcaaactccacctcctaggttcaagtgattctcctgcctcagcctcccaagtagctgggactacaggtgtgtgccaccacacctggctatttttggatttttagaagagatggggtttcactttgttgaccaggctggtcttgaactcctgacctcgtgatccatctgcctcggccttctaaaatgctgggattacaggtgtgcgccactgcgccctggccttaatgcaaccttttctaaaaggtcttgtaagaatctcttagcctttgctgacaaacatttaatgatggagaaggacccttctccaggacccatgcaggcagctttgatggttagaagtttctccttactttgtaatccacaatctctgcctttgtaaatttctccattatcattccctctcccacatcacagccatttcaatatactgtcagccagcaatccacgtggccatgtattaaatgacgaagagtaaagatcctgtcctctctctgccaaaaaagtgaacacccgtctgcttggtaggtaaatacccagccagaagaagggcaatttatcaaacttaatgaaatatcctcaaggacacctctgtctatttgtacacgtatctgctcatcaagcaacttttttttcattctttaaagttgtggtaaaataaacacaatataaaacttgtatcgtaactattttaagtatataactcagtggcattaagtacattcatattcttgtataaccatcacaccatccatctccagaactcttttcatcttgcaaaactgaaactctgtacccattaagcagtaacttcccattccctcccccatcaagcccctggaagacaccattctattttctgtctctatgaatttgactatttgaggaacctcacattagtggactcatacagtatttgtctttttgtgtctggcttatttcacttagcatgatgtcctcaagttttgtccttgttgtatcatgtgtcagcatttccttttttttttttttgagccggagtttcgctcttgttgccctggctggagtgcagtcgcgggatctcgactcactgcaacctctgcctcccgggttcaagtgattcttctgcctcagcctcccaagtagccaggattacaggcatgcaccaccatgttcgactaattttgtatttttagtagagatggggtttctccatgttggtcaggctggtgtcaaactgccgacctcaggtgatccgcccaccttggcctcccaaagtgctgggattacaggcatgagccaccgcacccagccagcatttcctttttttaaaggctgaataacattccattgtatggctgggcacggtggttcatgcctgcaatcccagcactttgggaggccacagcaggtagattgcttgagcccaggagttcaagattagcctggacaacatggcaaaaccttatctctacaaaaaatacaaaaaactagccaggcatggtggcatgcacctgtagtctcagctactcaagaggctgaggtgggaggatcacctgagcccaggaagtcaagtctatagtgagctgtaatggtgccaccacactctagcccgggtgacagagtaaaaccctgtttcaaaaaaaaaaaaaaaaaattccaatgtacatataaaccacattttatttcatccattgatgaacatgagttacttctaattcttggctattgtgaataatgctactatgaacacaagtgtacaaacatctctgagaccccacattcagttatttggggtatatacccagaagcagcattgctgcatcatatggcaattctatgtctaattttttgaggaactaccatatgattttccatcatagctgtagcattttacactcccaccaacaaagcacaggattccaatttctccacatccttggcaccacttattattttctgatttttgttgttgtgttcttttttaacatgacatgatttcatcatgatttctcattttttatagtagccatcctaagcaactttctttttattattgttattattattttacagacaggctgtcgctctgtcacccaggatggagtgtggtggtgtaatcattgctcactgcagcctcaatctcctgggctcaagcaatcctcctgcctcagtctcctgagtagttgagactataggattacatcactgcactctgagtaatttttatttgtttttatttattctttttttttttttttttttttgtctatagaggcaggttctcactttattgcccaggctggccttgaactcctggcttcaagcaatccacctgtctcaggctcccaaagtgctgggattataggcatgagccacttcacccagctaagcaacttgcttttgataacaataccaatgactaccatttagccatgtgcttcacttgtggctagtttggaatccactgtaacctagaacactcacaacaaactgctgaggtttaaacatgaggactctgaggctagcaaggccagcaccttgccagcagcagcccacagttaggaagggacagacctgggatttcagcccccacatgaaattaattcgacaccctgtgctctaccgccctgccctgccttacttttgaccattcagagtgatttctgggcactcatttgggtgccagggatctcctgagcaagtcgcccacccacaaccctagcaaagtctttgatcggcattctggtttgtgaacaatccagagagaacagagcacaataaactcctctaggatagaaacctcccacttatttatgttggtattcccccaaaccacccactatcatgacatacagcactcgataaatatacttgggctgggtgtggtggctcacgcctgtaatcccagcacagcactttgggaggctgaggcaggtggatcccttgagcttaggagtttgacaccaggctggccaacatggccaaaccccatctctacaaaaaataagccaggcatagtggcacacacctgtagtcccagctacttggaggctgaagtgggaagatggcttgagcccaggaggtcaagggtgcagtgagctatgactgcaccactgtactcctgcctggggttacagcctggtctcaaactcctgagctcaaattatcctcctgccttggcctcccaaaaaacaaaaaaattacgcaggcctggtggcggggcctgtaatcctagctacccagtactgaggtgagaggatggcttgggcctgggaggtggaggttgcagtgagccgagatagcaccactgcacttcagcctgggcgacagagtgagaccccatctcaaaacatacaaataaataaagaaagaaatacacgtgagggaaaatttttgttacatacattgggcatctatcaaatttgaaataatacaaaaagccagtgtggttctgaaagacaaaaatcaactgctatggtgccgcatttcattataccaacaaccataaactccatattaaaattttgcttaaaaccagggattcggccaggcgtggtggccaaatccctggtgagtgagatcgtaccactgcactccagcctggatgacagagtgagactctgtctcaaaaacaaaacaaaacaaaaaaccaaggattgtgcccattactgtatctgtcactctccaggtcacttaggggaggtgtcaagtacctacttgcagatgacgattacaagctgaagcctattgctgtcacagaaaacagaaataaatgttactttctagcttcaaattaatctctagagaagtagccttgagtcattcaactctccacctactcaaagcattaagtcagtgttcaacgtgtcttcgctcttggctatgtcataaatgccatattgtaaaagagtgtaggcaagaagttgcctgtgatggacaaaaagattagcttcatactaaaaggctccagtgattccccttcgttaatgccatgaagtccaacatttagtattagggccaagcaccctccaagcctgcctgtggagcctcccactgcctctctggaacaagctctggtaatgtggaatgacccctgcatagtcaggctgtccttcctcaggaaagccccttcttccagggtgtgtggcccactcccttcaggatttccaccaacatggcaggtgtgtaccccaactcctaacactgtgcgggactcccagtagctacccaggaaaagtttgctaaactgaatactaaacatagcagtggaaggttctttaagaaaatgttctatttacaacatttacagcgcatcagctctcctgagacaggactcttacaattatatccatcaagcgtccttcaagtcactctagctccaaagttccctgtctgcttccacctgccttctgcagtcccactggcttcattccattgctccttatatcctgccgttttaaaatcatataaggagccaggcgcaatgacttatgcctgtaatccccggatttcgggaggctgaggcagccggataacttgaggccagaagttcgagaccagcctggccaacacagcaaagccctgtctgtactaaaaatacaaaaagtagctgggcatggtggtgtgtgcctgtaattccagctactcaggaggctgaagcacaggaatcacttgaacccgggaggtggaggttgcagtgagccgagatcacaccatcacagtccagcctgggtgacagagcaagactctgtctcaggaaaaaaaaaatatatatatatgtgtgtgtgtgtgtgtgtgtgtgtgtgtatatatatatatatatatttatatttttatatgggatttatgtatatatttatatgtgggatttatatatatatttatgtatatatgggatgctcattcttttaaatataaagacgtacagaagtaaaaacagcactcaactgtcacccagaaataatcactattaagatcttagtataaaaattgtttgaaatgcagtttatagatgatgtgtccatatatgaatattttatttcagaaatggtatcatgtcatctactagtttgtgttcagctcttttgctattgtttgtttgtttgagacaaattgcacaggctgatttcgagctcctgagctcaagcaatcctcctgcctcatgctcccagagtgctgggattacaggcaagggccactgcgcctggccgcctggccgtgttcagcttttgaaaaaaaatttttcatattagtaactgtaattgaatgttttatcttttaagatatttacaaagtatcccattatatgaatgtgacttaatttgtgtttgtttaataaacctccctattggactgcccatctctgtacgttggaaggggggatgaaaatatgttgattaatggatacaaatatactgtttgatagaagaaataagacctggtagggtgactagagttcaccattaactattgtgcatttcaaaacagctagaagaattcaaaagattcagcatttaaaaagaaaacaaatatttaaggtgatggctatcgcaaatactgatttaatctttacaaattatatgatgtattaaattatcacatgtgccctgaaactatgtatatctattatgcatcagaacaaacaaactctgatggagctggccaatcctacctcagaaaagctgctccaggccacattcccctgcaagttaaggggaggggctgttccaccagattgtattttcctttttctattttgttttgcctacagatatgtgtatttgagcacaaggtaaaatgtttcttgctatgggttgtggtcaaataagtttgacaggctgggcgcgtggctcatgcctgtaatcccagcactttgggaggccgaggcaggcagatcacctgaggtcaggtgttcgagaccaggctggccaacatgatgaaaccctgtctctactaaaaatacaaaaattagccgggcatggtggtgcattggtgcatgcctgtaatcccagctactcgggaggctgaggcagcagaatcgcttgaacctgggaggaggggttgcagtgagtggagattgcaccattgtactacagcctgggcgacagagcgagactccatctcaaaaaaaaaaaaaaaaagatgtttgacagccactgttctatcccaagaatttaacaaggaatttaacaatttttaaaatagtcctgaattattatagttagtatttaaaaattattatgtagcctgaacattaaaagatttcgaaacgtctacccaagagtttcataacactttccatgacggggaatttcaaaatgtacatttttccgttgctgaaaaagtcaggcatggccagggtgggcttcgcaaagcaggctctgcccttaagactctagactgctcctctctcccaccccgccccacccctcacttaagggcaaaccgaaaatgcagagggcagcagagggtagaaagaaccccgaggaaaacgcagcattgggtggtaaggaaggtggggctgcaggcaaatgacccaggggcctggggtggaggaagtatcacttccttgcctgcctcctctggggccttttctctccagcatcgggctacagttctgctctccgtgacacagcttccccctggaaagcagaagtggtcctccctctccagagagcctctctgagataagcttctccattcccctcacctccgtgggggttaacatttacaggaagagtgggggcacccagagacccaaacaccaagaagacgtcattgcttcagtgggaggcagaatgaatacacacccatctctccagtctcactgtaccagcgcccaagggtaggcccaaagaccacacgaagacctccccaccagatgttttaccaatggagggaggaaagcggggactggaaagaagcttccaaccagtatggtctggtgtcaataaagaggagtgagcaagatttcagatgctcacagaaaggtgtgggcaggcaggcaatatgtaactttgcctgaagcatggactgtagaaaggaaatgagaccaactaaaaaactatctgagctgtgaagacttcttcccagtgttctacaacccaaaagcagagcctcaaccatcaagaaacagccacttaactggattatttaaatgtatcccgattcatgcagttctataggatgtcatttaagtagcagcattctgaacactgagcatttacaataatgaggttaactgacaatctcttcccatacacggtcagctcatgccaattcctggttgaagaagcctttgtaagatgatagggaatgaagtggggagagcagaagaaggggtactggaagcctgagggttaataaagctcagtgaaagagcaatgccatggaagagttctataccaaggaaaactgtatcctgagtgtaagtacagcttttaagaaatagctctgtggcaaataaggaatgctgccagctaattcagtaatgagagaaaatgaagtacagaacaggatgaaggggataaacaacaaaggctactacaggccctgctcttgtcagcatggatgaggctttttaatacatcatcccaagtatttaaacttgctcttctaaattacaagacagggccaggcacggtggctcacacctgtaatcccagcattttgggaggccgaggcgggctcacctaaggtcaggagttcaagaccaccctggccaacatggtgaaacccatctctactaaaaatacaaaaattagccaggcatggtggcgggcgcctgtaatcacagctactagggaggctgaggcaggagaatcacttgaatccaggagatggaggtgcagtgaactgggatcacgccaaagcactccagcctgggtgacaagagcgaactctgtctcttcagtgggaggcagaataaaaaataaagagagcccttccaactaacttaagccaaatataccccagaaaggccaggcgcagtggctcacacctgtaatcccagcattttgtgaggacgagacgggcagatcacttggggtcaggagttcaagaccagcctggccaacatgatgaaaccccatttctacaggaaatacaaaaattagctgggtgtggtggcacatgcttgtaagtaatcccaactactcagaaggctgaggcagggaggattgcttgaacccaggaggcggagattgccgtgagctgagattgcaccactgcactccaggctgggcgacagagtgagccttcatctcagaaaagaagaacaaacaaaacaacaacaacaaaaatctcaaaaggtccaagtctttaaactgacaaaatttctaatccctccattggcagatttccataccattctctacacatgggtatctcccccgaaagggaacttattcaaaataaattttagaaatgataatggagtgaacttaaagtttcacaatttaagtatcaggaaaccagacagctgtaagaaatctttcagtatttctgactggaaaaaaaaaacccaacaacttcctcttccatgttcttctcttcattatcagccttaaaagatgggaggcaatgtatatctatactgttggcctagaacagtgccttgcacacagcaggtgctcaaaatgatcaggtgttttaatgtgcgtcctatgagccctgtctccttaatttatattttcacttaaatgatgctctcactgataaaacaaccaggaggttggcaaagtttacagggagacactcccagcacaggtcggggaaagtccttttgcaaagtattactcaggtacttctgtattaaccattaagcaactgattgcccccaagacagtcactagggctgctcggacagcagccagtataggtgtcatcttagcagttactggagaccaccctttcccacccccaagcaaaaagcagtgctaaaaagcacagaaattcatttctagaagacagcttgaagtgcccagcttgatggtgcatctgctatgtcctgagcagttagccacttagttcagacaattaccaagtgataaaagaaaattaactcatgatttcacttttaaaatgtttgttaaatgacctatttatttgcttttaagcacagatggtctgcaacttaatgatttttcaactttacgatggtgcaaaagcattaggcattcaataaaaactgtacttcaagtacccatacaaccatttcatttttcacttttaatacagtattcaataaattacatgagctattcaacactttattatcaaatgggctttgtgtcagatgattttgtccaacagtaggctaatgtgagtgtttcgaccatgtttaaggtaggttaggctaatctataatgtttagtaggttaagtgtattaaatgcatcttctactttcagtattttcaacttacgattgggtttattgggacacagcctcatcataagttgagaggcatttgtaattgagcacacactacaagtccaaaggtggacaagtagaaagagccaaaaaacatacctactactcctaatggtaccgcttaaccaagcctgtgaccttagacaaccccctgaatctttttctgtatctgtccctctccatcctataggagccacaagaggttcaaatgaaattagagttcaggaagacttcagagccctgaacaaatgaaaggtgctaacaccttcaccatcatcactaggaagagccagtaaaggtctgaggctatctctgctatcaatgtgacattgatcaatgtcctgactaatgattgtggtggcctttcaatgttaacactcatccatgcatcactgagatcatcagtaggagtgagcttataacaatgttccctgctgtgggcatgtatacatggtgtccttatttataaagttagtatgggtttgtacatgaataatatgatttaattttgttaagttacaactccacatagtcatgtgaaccaactggaatctcagatgtttttggtgatttatctaaaaacaaatattgcagatagaagtaaaaggctttcaaagtcattgaacagtgctggaccacttttctgctaaaagaaagctattggagggcaggggcgggattacaaagtttcgacctacctttgtaacaccaatgactctaaaataatgtaggtcttctggattgggccaactacctttgcaggaaacatcttaaccaccccacatcaggacctctcttccagtaataagtagagaagagcggtgctgagtggagactctcatttagttacccacactaaatgtgggtaactcagtgctgggtaactttacagtcaacaaagcctctcaaatccccgttttagaaaccacaacagaaagggccagtaggcttcaaacaccttagggaccagctctatttcttcatctccctatgtcctcagttttgcatagggcaagctcacaggaggccttcattccttcttccaagtaatctcgaactgagcacctactatgtaccaggcactggggctatcattttggacaagacagatgaagcagttttcatggagcttccactgtaatggttaatacagactatcaacctgcagacaaatcaatataactctggctgctgctataaaggaaatcttggctgaataaatggcccaagtggcttatcccaaatcacagcatattaaatgatggaagaggccaaaactcaaggttcctggctcctactttctctccccttacatctgctgccctctatccttcactcagcaaaagggccacacatttacatccaaagaggcccagtcaccaaagactctatgatccagtgaaatttattcaggcctcaaagcgcttacctgcttacctccaaatgagaatggagggactaactggatcccaccctttcccaacaaagcttactcttacagagaagggcttagtaaggcctgggttcttgcagtgcctattccccagggcatggctttaggtgttggctgtacagagaaaacatccctgtagcccaccagtttattggctagttctttagggaaagtcagactactgggccctgctttgggacagcaaatccctcatgtatttattaagcagatttactaataaggtcagatggctaaatctgacatggtagtcttagcttcaacatcaccaagctgcaaagaaccaagaagcctcatttatatgaatagaattcactgcaataaataacagtaacatcgtttatgaaatgcttggtacgtactaggggctttgtgtgtactaaacagcccctgaggtatatatgctctctcaaagatgaagaaactcaaacagggaatttcaataatcggtcccctaaatatacagccagaaaagtgcaaggcctagattctgagctggcgaagaagggcgaatttgggataattaacacactcggatatacaaaaacacaaccgctaatccacatgacaggcaggctgttggaaagttacgaatgcaccgcaagcaaaagatgccaccatgggcccagagaatgcaggaaagtgggaactgactcttacgcccttcccccaaggcgaaggaaaggcaacccctgaattcttaaaataactctcgcttcctctcagccggcaaaagcaccggactgctcctcggcaggccgagaggcgccgcagcgggagggtgagttccagcctccgctgcagcccctcgacgtcacgctcctgcccggggccaccagggagggcaactgtccgcgggcctcgagagggcgccggccgagccccatctcccactgccaggagacccagggtccagaggggagatcagattgcacaatccgaggagtcctctccagtatccccgccgcgcgcccgctccgcaagggcagcggcttccccactgtgtgcgagaagcggccggagcagggacagcagccggccctcgggccctcaccgggaaggacacgcggtgagcccgcccccgcgcccgcacctcccaccgctcccctgcgcggctccgcggcggacacctgcggagcctcagtccccgcgccctggctcccccgggcccggtcacccgtcggctgaagacatctaagagccttcgccgccggagcccagaggcggtgcggccaggaagccgccgcgggccgcgccgatgcccccggccccgagtctaccctgcgcccgggactccggctggtggcacagccgcggcccctgcacccactgcccgcgtgcccgcgcccacccctgggctcgcgcagaggactcacccgctcgcggtgaaagtttcggggcggcggagctccacggtgacttgagttcagagtggggggcgcgacggggcggcccagcaggacctgcgtatggggaggctgctggccgggaacgctggacgcgcggcgaggggctgctggcgcggggccgggggtggcgcggccgccccgcttgccgcgggctggaggctgcagcgcgtctggcccggaggcgccacggccggggagcgagcggcagcgcggcccggcgcggcgggagaggaactggctgtctctgggctcggccgagaggtcccagctacttcctggtgctcggatttgcatattgagcccggagggcggggaagtgaccgtttggtccctgcccaaggtttgggtgtttggtgtggccgtcgcgcagggctttgaaggcacagaaacctggcgactcagtctgcagctccggggagtggagcagcccgggccagccttctcctgtagggacggacggaaggatctccagaggagcctgggcttcgcaggcacatcgactacacctctcgcgtctcctttcctttccttccgacccatccccaaggtattattaggaagggggtgctcccaggagcggaggaggtgcgctctgatcccttccaacattaggggttctctagagaaaggatgcgatatgccgaggtgatgctcggggtctcgctgagttcttaataatcattacaaagtaagttccttaagggtgtacaggaggctgttcagggcattgatgggggaggggatgacgtagagtcaaatggcccggcaagttctccgtttctcaaagcctcagtttcctcccaggacaatggatgtgaggatgcctatctcgtgtacctttctgtacagggcccgcactgcccgcatgcaggaatgctggcgttatttcattcctactacagctgcgtcccagcatgtagggcagtgcttgcacgtagcagctggtgcctaaccagtgtttgctgaaagaatgtgtgaccgcgtgctcaagctcatgttcaactcaataaatgccacttacaaatccaagatgtaagattaggctcgggcaccgtggatacccacttcttctaagccagattctgttcactgggatctcagcattgcattaatagcaaacatttacagagggcctgatgggccagacactgcccaaagcacttcctttctctcattaacaatgttctgaagaggctgaccccagctacagagcaataagtcaaagcagctgccttgtgacacaatctgatacagcagttatttgttactaggaaaagagtactaaagtttgcatactaattccttaaaagaagaaaggtggtttatacctgtgatgttcactggtatgtagttgttagtttactgctgaagagaaggggtgggttgcccatggaaatacccatgggaaggtgtgtgattgaaacctatcaagaatcctatggagaaaaatagaaaaatgaaaaaagaacaaaacaaaacaaaatgaaacaaaaaacaatgaatcctacagaataattcactacctgagggcctattgcctagacttttcttaagaatgacctctcaaatatattaaccctacattaaagtttctgctgctactgatgaatccttgagatcctgactaaacgtgtgtggcaaataagaataggattcaaaaacgtttggtattggttaatctatcaggaatgtctgtcagtgtttttggtaaaatgtctctcagggtaaggaacaaagcatcatcaggcttacctgagcttgcaaccagatccccaactccctaactcctcacaaaattgtcctcactaagttcatctcttcattcttatttggaaaaaaacgatacgatgctaatttccacttcttaggctgcatttatacttgctgaatacttcattcaatcattctccatatctgtctttccttcttaatgtcccatgaaagttctatttctcaggaaagttttatttaaagctcctattgataggcaactttttaaagcatctgtagcatgtatggtgtgcaacattattttaaactagacctaggtactttttagcattgtttgcccatgttaggcttgtctcctcaactactggtaaactccttttgggaaggcactgccttttatatttatctattccccactctacctcccagaaattatgttgggtgctttaaaatagttcattagtattgattgattggttaattgattgatgactgatttatctcaccagttttctctaaattgagcacagaaggcagtgatcagaggcagggtgcaaatcaattaaggctccttgacaggatagatgggcctttgaagaaaggaaggaatacgatgggcggggcaagagaaggcggtattgtaaatacgttatgtacagtgtatcctcatttttgcagatatcatatttgtgaacttatctacttgctaaaatgtatttgtaatctcaaaatcaatacttgaggtgttttcccagtcactcatagatatgcacatcccagagcagtaaaaatcttgagttgccccacgcacaccctcccagctatagtggaacaaggcgatgctctgccttcttgtattctttttgctgtccatttagtttatgctttccacacttctgtgctttttgttggtgattttgctgtttaaaatggcccccaagcgactgagctcagtggctcatgcctgtaatcccagcactttgggaggccgaggtgggaggattgctcgagctcaggagttcaagaccatcctaggcaatatcgtgagaccccccccacaataaaaaaattaaaaaaaataaaaataaaaataaatggcccgaagcgtagagcttgctaaagtgctgtctgttgttgctaagcaccagaggctgtggcgtgcccactggagaaccgtcctgcaggcaggagctactgtgctattggttgtgaactcaatgtaatgaatcagccatatattaaataaggtgtccattaaacagaaacacacaccatataaggttatgtattaatcataatgtaatgaaaatgttgtgtccagagactcacaggaacctaagaagcagtgactcggtattcgctaattcagtgttcacagtgactatagaacataactaccacaaataatgggacgcagcatacaagagcatcccatcatccactgtgggaaaatgaaatatctgacctctaggaacttcttcattagcaccgccatcatcatctcatgcgtggtcatggtgcttgaggccaggattggtcatgctgctcacctgttcatgttggtccaccaaatcatctgtgctgcaacccatcccactgccactgtgtgggctgcccctgctgcctcttctagtattcttccacctaactcttgccaacttggccacagacctaactaccaagtagcaaataatacagagtagtggtgctcaaccctgggggcacattagaatcacctgagctgctaaaatatctccatgcccaggccctacctagaccagttaaatcaaagtctttgggggatggggatggtgaggggaaccccaattatcagagactgaaaaccttgccaggtggttccaatgagcagccaagtttgggaaccactggtggagtgtgctccgccctcccctagcctggagctcacctctctgaacaacgtccaccttcctcaagccctcatgcccccaggtcctctgtctttaagagagtagcatgtggccaggggcagtggctcaaacctataatcccagcactttgggaggctgacgtgggtggatcacctgagatcaggagtttgagaccagcctggccaacatggtgaaaccctgtctctactaaaaatacaaaaattagctgggtgtggtggctcacacctgtagtcctagttactcgggaggctgaggcaagagaattgcttgaacccaggaggtggaggttgcagtgagccaagatcacgccactgcactccagcctggacaacagagtgagactcggtctcaaaaaaaaaaaaaaaaaaaaaaagtagcatggatatatgcctctgtgtgagggtaaagtggtaaggattgagaatattaaacctgtattactggggaaaatgggtgactttagcacaagtgattatagatacaaggaattaataaagaaaaacaaaaattgctaagaattattttaggctacaaaaaaattttaacaactatatgaaatattatttatacataaattgccacaaggtagattttttcttaaatacttatccaatgttaaccggtattacaggccaggcacagtggctcacgcctgtaatcccagcactttgggaggccgaggcgggcagatcacgaggtcaggagatcgagaccatcctggctaacacagtgaaaccccatctctactaaaaatataaaaaaaaattagccgggcatggtgatgggcacctgtagtcccaactactcgggaggctgaggcaggagaatggcgtgaacccgggaggcagagcttgcagtgagcagagatcgcaccactgcactccagcctgggtgatagagtgagcctccgtctcaaaaaaaaaaaaaaaaaaaaacaggtctgacatggagaaagtatttatggtcaagtaggtttggcaactttttgagctgaacccagtctcacaaagctccttgtagcacagtgatgtcactgtcaatctccatgaaggagctaagtgtatttttgtcccagatcatatacccaggtgatggatttgctgaaagctgcacctgggaccagctgtcaagcagaagacacagctattgatgtgaatagagacctctcagagccaccaagctagccgttgtttcatcatcaccctaaactaaattcagaaaagctgaatgttcatacaccgtaagacagctgagagtgggcaggaatgtcatgcatcactgccacctggcaccagcagatctcctggaactgtgctcgctgatgtgctgctgccttggccactggcataggcatcacagcaaaatagttttgtgtgtgtgtgtgtttatttattttttgagatggagtctcactgtgtcacccgggctggagtgcaatggcaccctcttggctcactgcaacctccacctcccgggttcaagggattctcctgcctcagcctcccaagtacctgggattacaggcgtgtgccaccacgcccaggcaatttttttgtatttttagtagagacagggtttcaccgtgttagccaagatggtctctaactccggactttgtgacccgcctgccttggcctcccaaagtgctgggattacaattattattttttgagatggagtttcactctttttgcccaggctagagtgcaatggcatgatctcagctcactgcaacttctgcctcccaggttcaagcaattctcctgcctcagcctcccgagtagctgggattacaggtgcgcaccaccattcttggctaattttgtatctttagtagagacagggtttcacaatgttggccaggctggtctcgaactcctgacctcaggtgatctgcccgccttggcctcccaaagtgttgggattacaagtgtgagccactgcatctggctgacagtgaaatagtttaaatcgtgagcatggaagtgacaaatgtgggtgcctgcactcctttcccagtatgacagcaaacccagcctccttcattgagctttacatcactctgtggggatggagcttgtccaaagtaacctttcatgatgaaccatcggggacccttcagggcccagtcaaggccattgcctcccatttattccttggccatgtgagccagactatgtttccattcactcatcaccccctgtgtgccaggaacaagatggggtcccatattcaattctacaaaaacagtttggtaacagccatcagatggtgatggagccagatgaggatgtttgtgtaatactaataatgattttatgttctggtaacattgtctgccttgtttgatgttatgttgttttcatgtatgtatgcgtctggtttggtggccatcttggatgttcagtttctgtctatgggtttttttgtgtgtgttttttttgtttttgagatggagtctcactctgttactcagtggggagtgctcttggttctctgtaaccctcacctcacctctcaggctcaagtgactctacagcctcagcctcagagtagctgggattacaggcatgcaccaccatacacagctaatttttgtatttttagtagagatggggtttcaccatgttggccaggctggtctctaactcctgaccttaaatgagccacctgtcttggcctcccaaaatgctgggactacaggtatgcgccactgcacccggcctgtctatctatgttgcctttgaataaacgcagcttcaaatgctgcttctagtttttggcttggcaggtggcattggctgaatccctcctagtgctcaagcctcatagtatgatgtagttattaccacttcttgtatgtggccagcattgctgtaagacaagagatatttcacagaaatcaataggacctctcagagccaccaagctggttgttacttcaccgtcaccccaaactaaattcagaaatgctggatgttcataccccgtaagacagcttgaattatgggtaaagaggtgtaggtatgctttaatatgaacctcattggctggttttcccagacccataagctagtgatagcatgtgcccagcaacagtgctaaggacaccttgatggcttcattatagtcactttccattccttacttccacctccagaaaagtccagaaaatgcagagcattgctccaggtgactcagtaaaatctcagccacactctacaaagtaaagctcatcccagggtaggctacggttttctctgactccgggcacgtcctttcgttgatctgaatggtttatgtgtgtctgtcccacggaagtcctgtttatctcgattagttattcttaagctccaagaataccactatcctaaaaaatacaactgtcctatgacatatggtatttggttcttagttagggattcttcaaatcagattgtggacaggatgcaaagttacaagtggaaaaacttgcactcataaagtcagaaagggagctgtattcatttaccttccaacccagcaggagagttttgactgaactcatatgattttgaacctgaattgcttgcttacatgacccaaggatgtcagaagaggttctttctgtacctgtgattactggaaaaactactcaacaggactctgtttgctcatctataaaatgacccacatctagctcacacaggtgctgggaaggtagccaagtggcccagtgttcccaatctaaggagaaaagtgatggcatgaggtggaggaggaggtggaagcaggagtaggaggttggctaatactatgagtatacacgatattgagagtattttggggtggctccggccacaacgacagacacctttatgcctttgttttcactcactgcctttcttgacctccatcctcacacagctgggtcttacccatctttcaaggacagtgtaaatgccacctcaccaaggaggccttcagcagacacagcatatctcttccttccttgaaatcccacagaaaggtcaggcacaatgacttacacctgtaatctcagcacttcgggaggcagaggcaggaggattccttgagctcaggaattggagaccagcctgggcaatatggtgaaactccatctctacaaaaaatacaaaaggccaggcgctgtggctcacacctataatcccaccactttgggaggctgaggcaggtggatcacttgaggtcaggagttcgagaccagtctggccaacatggtgaaactccgtctctaccaaaaaatacaaaatttagttgggcttggtggtgcatgcctgtaatcccagccactcaggagggctactcaggaggctgaggcaggagaatcacttgaacccaggaggcggaggttgcagtgagccaagatcatgtcactgcactccagcctgggtgacagagcgagactctgactccaaaaaaaaaaaaaagaaagaaagaaatcccatggaaagtgatgtctagttcttgttgacacttgtcagtttatacctttattctagtattgttatttatctacacagagttcttaataccagatggtgaactcctgaatgaggatctgggcctgtctgtttggtttcatttttattattgtttgttaccctaagcattaagtgcagtgcacgtaagcagaaggtactcagagcgagtttgctaaatgaaaggttagtatggtgtaaggggaaacgcctactgtgtcggaacccagagaggcaatattttgcttggtttgtggtgaaaaacctaatggtgattgagtcttcaatctgcctcacgggctgggcaagtcacttctgtacattttatacacacacataattctcacagtaatctttgaggaacatgctattaaccctgtcttactttcaatgcaactgagtcacagaagggttaagaaagttttccgagatcacacaggttactcacagctagtgagtgatcgagtcaagatctgactgtgggcttagctggccaggcctatggcctttctgctgtgctgccacactccctctcctcatctccttccattgtacagattcttctggagctttctgggatgttagctaccttgagagcactcatacatacgtcctgtgtattccataaagtgggaactgtggaaatattcaggctcaaccctaatacttgcggggcgatttgttgagagtaaaaatggaggtccaagtaccatgtgtctcatctgtgtatatcaagtggcaaaggttttgttgttgttgtttgtttgttttgttttgttttgtgagacggagtttcactcttgcatgatctcagctcactgcaacctccacctcctgggtccgattctcctgcctcagcctcccgagtagctgggattacaggcgcctgccaccatgtcctgctaatttttgtatttttagtagagacagggtttcaccatgttgaccaggctgaccttgaactcctgacctcaggttatccacctgcctcggcctcccaaaatgctgggtacaggcatgagccatcgcacctggccttcaagtgacaaagtattaaataaaatatggtctatccttctacttggacaaatgtaccctttttttttttttttttttttgagatggagtctcacgcccattgcccaggctggagtacagtggtgcaatcactgctcactgcggcctcaacctcctgggcttaagtgattctcccacctcagcctcccaagtagctgggactacaggtgcatgtcacacatcactaggcccagctaatttttgtattttttgcagaaactgggtttcaccatgttgcccaggctagtctgcatctcctgggctcaagtgatctgcctgccttggcctcccaaagtgctgggattacaggcatgagccactgcccccagccttaaatgtacctttttattggtcaaggctctccagagaaacagaatccataggagatatctctattcagaagagagagatttattctaaggaattgactcatgtgattttgggggtagtgagtctggaatctaccaggcaagctagcacgctggagacccaaggctgagttgatgtggcaactcaagttcagaggcagtctactgacggaattccctcttcttcacagaacctcagtctttttctcttaaaacctttagctgattggattgtacccacgcacgttatgcagggtaatctgccttgcttaatatttactgaattaaatgttaatctcatctacaaaatacctggtctgcccatgaacacatgggcagggactttggggtttgtggtgcatagagcatggcctgggaaggttcatgagctccagaagaacacaaatccacagacttctctcctgacaggagggcctacgcagagcagggccttttatgatgtagggaatggggtcagtgttcttcttgtaagctccaggaatggaacacattcgtgccagcttctatggttgggcacgagaacctgagattccaagagcttccatgacatgcctcagggactgagctcattcggctcagggcaagcaccacaactgtcatttttacaggttaaaaataattgaacattggcaaatgtacatggttcaacatagtacaaaagtattgtacttaatgctaaatgtatttttattataaatataggccaatctttgaataagctccctgagaaataaaacaaaaataaaatcttgacaaaagtagactgatgatgaaattcaattactatgtaatctgtgttggagcttagtaaatgaaaaattgactggaccaatagagatacggtcaggaacatagaaacacaacttctgacttggtttaaactcctacctcttatttttctggtcttggtgggctttaaggctagttggggccacacagatggattttgtttaagtggttggaatgcttgggctggaaagaaacaaggagtaagatggtggttgtaataatattggggtaaaagagactaatattttacaaattattttagaatgtataatttcaataattttgataaatctacaaggccgagagggtggatgtgatttccacccagtttttcaacaagggagaaagtaactaacatttattgtgtgataggtatcatccaaggggcattacatgtatgtcatttaatcctcaaagccataccagtgtggctactctgtacctcattttacggataaaatgataagacttgctcaaggtcacatagctggtgagtgaggggctgcgcctcaattcaggcctcctgagccagaaccagggtgcttcccaacagtctctgtgccatcgttcaaagatgagccacctcatctgtcatctctttatctcacatgcacatgcacatggccacatcccaagtcagggtttcacaggcacatccaagaggatcctgagcttttaaagttgaggacagacccttgcccacctttacattcatgtgcctggctcggtgccaggcacaaaggtgtccagtgaatgtctgttagatgaatgcatttttcaatgtattaaagattaattttataaacaaaataattttatggttttataaattgattaaaggctaagttctctagggtgtcagttagccacaaaaataacctttctctgagtaaaagaatattgtatgggaattataagatgtcagtgctggccctagctccaccatgtattatctgtgaaacccagatttgctcatttttaagaggtggggttggtccggatggcatctgtagtaacctccaggtctcaaatgctatacttcttagaggagcactgagtgtgaggactttcgttgccagttcagggcattttacctaagaaaatgattcaccatgccctggagcaatgaactgggcagtgtaaagcagtaattttgcttaggccaattcggaggctgcttttcaataaggaaaaacacttgaggggcatctgcatacaaaaaaaggggtaagaagcttgccaggatgaagaaacttaaacctccaccatcctttatgagccattccaactagatacccatgctaaacaaaagaaaatcttacagggactagaaaactaatatgaaatcttctgaagttccttgaagaatattttgtgatctgaacctgaaaacttagaatactgctaaaatatatggctgaataaggcacatttctgataaagtgcaaactgttgtaagagagtatttttcaattttaacttatccctttggcagtacccagttttcaaccctaagagaaaggtgtcccagagagatgggttcaggaaaggattgagaactaatcaaataagctatagtaatcaaataccttatgctaaacacagattgaggggaaaaaattcaaattttaggacaatgtattaaaataaattaaagaggccgtactatgtatgtatatatagatatacacatatacatccacgtatgatgatacatgtgtcacttaagtgtggaaacaggaataagaaatggggacccctgagaaggctctattggaaatagctggaggcttcagactaagtttctgagcagtaggttttaagtccgaggcagtaattggcaaagctcttctgtcaagaagcagagaattccaagacactacatgttttcagaaaattggattgtttccagctttttcattaatttacacattttgcatgcctggcattgtactaatcgaacggtggcctagccaacataaacaatgttgaatggctaataactagctatagggttggtgataaaatggaagtaagtgaaagctacaaatatcagccaggctgtgtggttcacgtctgtaatcccagtatttgtactcaggaggccgaggtgggaggatcccttaagcccagaagttcaagaccagcctgggcactatggcaaaacccatgtctactaaaaatagaaaaattagccaggtgtggtggcacaggcctgtagtgcaagctacttgggcggctgaggtgggaggatcacttgagtccaggaagtggaggttgcagtgagctgatgtcacaccactgcactctagcctgggtgtcagagcaagaccctgaagaaaaaaaaaaagaaagaaagaaaagaaaaagaaagaaagaaagaaaggaaaggaaaaaaaggaagctacaaatatcagagaccctttctgtacataattattctgcatttgccacacaaagctcttagatgtcacctctttttaatataagcttcatctgtttgaagctatgatttatttggggggatactgtggttcctctcacagactgaaataatctctgtatgataatactacaacttatttttttctttccaataaaaatagttgttttgttgccatttttcactttaatgtcatagctagagaatcagagagctaagaaagtgcaggagaaggaacacagcagctgccaccacagctaggtggatactgggtgcttcacatgctcattttttgtttcttctcactataaagaaactgaggtgcagagatgtaaaatatcctcatctgatatcattaaactagcaaatctgttgttctgggatggaatatagcctttttttttttttttgagacggagtctcgccctgtcacccaggctggagtacaatggtgagatctcggctcactgcaacctccccctcctgcattcaagcaattctcctgtctcagcctcccgagtagctgggattacaggtgtgtgccaccatgcctcgctcatttttgtattttagtagagacagggtttcagcacattggtcaggctggtctcgaactcctgacctcgtgatccacctgcctcggcctcccaaagtgctgggattagaggcgtgagccaccacgccagcattttttttttttttttaagtgacaggatcccagccttggcaacatggtgaaaccctgtctctactaaaaatacaaaaaattagctaggcatattggcgtgcacctgcagtccagctacccaggaggctgaggtgggagaactacttgagcctgggagggcgaggctgcagtgagctgtgatcatgccactgcactccagcctgggtgacagagtgagaccctgtctcaaaaaaaaaaaaaaaaaaaaaaaagaaagagacacaggatcttgccctgttgtacaggctggagtgcagtggcatgatgactggtttgctgcaaccttcaactccttggctcaagcagtcctcctgctcaggtgcctaagtagctaggactacatgcatgtgccaccatgcatggctaatttttgtagaggcagggtcttgctatgttgctcaggctggtctcaagctcctgggctcaagctgtcctgccaccttggccttctaaagtgctgggtttacaggcgtgagccaccaatgcctttttgaccccagagctcatgatcctccagttaccacatgcttgctttttctcattacgtgccaaccacaatacttgctctatgttgttgtattttcttctcacacaatgatttgtaattggtattgttaattcctaggttgcagatgaggaagtaaagctcagagaagtaacttgccaaatatcacacagccagtaattgtcaggaaaaaataattctcaaattcatgtattgcctttgtgtaatatattaatatcaacagtcaaatgtttaatcacagtgaactgattacgcattcatcctttgtcctatggattagagtggtgaatgtaactttctggtatgaaataaaatgcttaagaaatttgagaacacttgtgaaagtgaagtgaaacctaattgttaagactattaagtctctatcatcatcactagctaaagaaaccacaaatttaattctttaagcaaaaccaaaataataattcattaatttgttaattatttcttctttctatacatattagctgacttttgtcctattcttgcaaatcctctaccattttggctggtagcccagtaagctgttgcctatttatctgatgattctctcaccattctattgggaagaaacagaaaatcatatcaaaatcgatatgagaactctgagtaaaatagcatgttaccaaaacaaaagtatataccttttagctttttagagaagtacctattttcccattaaaaacaaaaagtaatagccaaaaccaatgtcaagagtgggtgtgaggccagatgcggtggctcactcctgtaatcccagcactttgggaagccaaggcgggtagatcacctgaggttaggagtttgagaccagcctggccaacatggtgaaaccccatctctactaaaaatacaaaaattagccaggcgtggtggtgcgcacctgtagttgcagctactcagaaggctgaggcaggagaattgcttgaacccaggaggcagaggttgcaataagccaagatcatgccactgcactccattctgggtgacagagtgagactctatctcaaaaataatagtaataaagagtgggactgagaggtgaggagagtacaaatgaacaaaaaatcaataaaaagaggatttgtactctaaaataaaaatctagtttgtggggattttatcagaaatagtatccttcaacagtgcgtttaaaattctttctttttgtgcaaaattgcaactataattataacgttttagtctttggctaaaatgtggttgcctttgcatctcatacagggccaaaagaaaaaattagaagcattggaaaaactgcacagtgactacaggatgtcctgttgcatttatctgagtagacagacccgtgactcactataaaacttgacacgtcggaaatggttttattgtgcatggtacttttctcttcacaaatattaaacacatagatttgaggataacaagttatatttgtccaaatataaaggtgtctgctataattctttttgaagtgagaagttaattctatgcaatgataactcagaaagtatacacagcatccagtcaagccttatctcagaactctcagaaactctcgtaattaccccttcttaagtagatgataataaatgatatgaggaagatgtctcttgattaaattgatatattttaccattgtttggcaaagtcggggtagcactcgctctattgaaaaacctctttagccttgagccatacaaaagtgatacattgcaaaaactcatcagtaatctaataataataataagagcagcttcacatgaaaaggaatggcaacactttcacattgtgataaagcaaaacaaattccatgcattcagttttctgaatcctttaaaaaaaataaaataaaatgaagaacacatacatctgttatcttcctctttttttttttttcttttttttcagagacaggactttgctctgttgcccagactgaagtgcagtggtgcaatcatagctcactgcagcctccaactcctgggctcaagcgatcgtctggcttagcctcctgagtagctgggactacaggtgtgcactaccacacccagctgtttttgtttacttttaatttaatttaatttaatttaatttgctgctgttttgtttttttgtagagacagtgtctccctatgttgcccagactggtctcaaactcctggcttcaagtgatcctcctgtgaaatgctgggattacaggtgtgagccattgtgctgggctgctttcttccctcccctcccctcccctcctcttccctcccctccctctcccctccccttccctcttctctttcttttttttttttttttgaaagggaatctcattctgttgcccaggctgaagcgctgtgttgagatcttggctcactgcaacctccacctcccaggttcaagtgattttcctgcctcagcctcccaagtagctgggattacaggcacacgccaccacacctggctaattttttttttagtacagacaaggtttcgccaggctggtctcaaactactgacctcaagtgatctgcccaccttggcctcccaaagtgctgggattacagatgtgagccactgtgcccagcctcttttttgtttttttaatatcggtttacaccatcttatttagggagcctaaaatgtctcctctgcataaatgcttcccaattaccttagctcaaacaatctttttcctttcagtgtacatttattgccaaacaactctttaaaaaagaaaaagaaacaaacaatttaacaaagaaagatttcccttagtcactgggattgttaaatgataatatttggccctagtttcttccatttttcatccttacgcaaacaaatattggcatatttgtgtgtgtgtgtttgtgtgtatctgtacaggtatgtgtgtgtatgtagagtgtgtgtttaggggtgtagggatggtatttgtatgtgtgtaggtatatggtattttgtgtgcaaatacaggtacgtgtgcagagctgtgtgtatggagtgtgcatacagggatatatatatacacacacaggtgtatgtagaatacctgtgtgggtaagagtgtgtgtgtgcagggctgtgttgtgtgtaagggtgtttgtatgtgtgcatgcaggagtgttggcatgcaaggatgtatgtgggaggtgttatatgagtatatgtaggtttgagtgcatgcagataccagtgtatgtgtgtaaggatgtgtgtgtgtgtgtgtgtgtgtgtgtgtgtggagtgtaatgtctgggaaagaagttttgaaaagtgggccagccatgggaaaggattgaggtgaaattagaaaataaaggtgacaggtgggaggccagattgtgcagaatcttcgatatggaggcaaggagttgacatgccatttaatgagcagtggagagtcactgctggtggctggcaggggcagggaggggactgattatcagaatcatcgtggggcatcactagggttgcactattcctgggcattgagggtttggatttcatggggttggggttcataagactggcctgaggattaaattagatactacacataaaggacttaacgtagcttggctcatgggaacttctccaaggagaaggagaggaggtagaagggaagaggagaaaatacagggacttgcaaggaatggttcacttgggtagagaaaagaactttgcattctgttttcaacctttcaaaccattcctactgtgtctaattcagcctcagagtctgaatgcctccagtctgcgtggaataatactcaatataactcatactcaaggaactccagaagggaaggaaatgcccttctattcacgtgggatggcactcgctgcccatcttgggtggagtgttgtccactcagactttgtcttccagccatggcaggtgaggggcagctcactgatggggggaggaaatgcaaatacaactgcaggacaaagagtccggctccacaactccacatcagccctatgtgcaaaatgaaataaaaagaaagaatgacagctgtgtgacttatcatatcaggataaggataggggagtgtttgtccacaggaaatggactgtagttcctccaatttctaccttatgctcttatagcagttactttatcttttatctattgttatggactgaatgtttgtgttgtctcaaagctcatatattgaaatcctaatcccagtgtaatggtattaggaggtggggtctgggaaggtaattaggtaatgagggtggagccctcatcaatgggatttgtgccctgatgaaagagacctcactaggtgcagtggctcactcctgtaatcccagtactttgggaggccgaggcggaaggattacttgatcccaggagttcaagcccagcctgggcaacataatgagaccttgtctctttagcaaaatcataaaatgaggcaggaggattgcttgagcccaggaggtcgaggctgcagtgagctgtgattatgccactgctctcctacctgggtgacagaacaagaccttgcttcagaaaaaaaaaaaggaagaaagaaaagaaaagaaagagacctcagagctctgtccctccctctttctgccaagtgaggacacagcaagaagacctggtgcatcctagaggcaaggtgcccaagtatttgcatacattatctctttttcattcccatatttattcaacgtgaagttattgttaagtgtttgctgtgtgccaagtgctctgacaggtagaaatgatccccgttttgtagcagaggaaatggaatgtcggtgagattgagcaacttgccgctaacaacacagctggcaggtgagagtgcagctcacagcccagctcacagcccagctcatcgactgagcactcccccatcccaccccacccccagcttggtcagctgacctgatgcacagaaactttgtgaaaaatctgtcctgacaaacaaaaattagctttggccagaagatcaaataacacacttgcacacttgtgttatccagtgtgaaatgcaaatgaatgtggtgacatccccaaacaagacacctctgcagctgcctttataacattatataatataatgttatagggtaacagtctctacactacatactgcctccttaggatacagaaacccaagcgggagggaatactctttggtctttttcaaacaccgtgacacaaggagaaaagagagactaggaaagaaaagaaacaaagtatctggttaaccatcaaatcaagctataaattcattagaagataattttttttctaaaaataactttctggttgactttgaggttgctgacaagtttcagtctaatgaatactagtttctaaaaaaagtcagatgaaaataaatgaagaagaaattttgtttctgatctggtttaaatgttttttttaaaagtgaaactttggattaaatgtagaactggcaatactgtgaaacaaagaagacaatttttgagaacttgaactctctcagtactgtatctgcttagcttgagggtcaaagtaataaatttttaataaattaacaaattataagaggacacttttcaggatattaaatttatttatttatttatttatattttttgagacagggtttcactcttttgcccaggctgaagtgcagtggtgtgatcatggctcactgcaacatctacctcctgggctcaagcaatcatcccacctcagccccccaggtagccaggactacaggtgtgcaccaccatgcccaattaatttttttattttcagttgcccaggctggtctcaaactcctgagctcaagccatctgcctgcctcagcctcccaacgttctgggattataggtgtgagctaccgtgcctggctaaaagtaaaattttaaattataagttgactataagcacattatacataccatggtaaataaagctgccgctggcctgaggatgtcctggaagggaaagaggtcatgaattgagggtgagctcgacccactgcacagtttagactgggctccccactcatcggggtgggtgggaatcataatcttgtttgtttcttaatttttgttattgtttgcttcttttaagcctagatcctctggatttagaacacacagacttttgccaaattgcgtgctgatttttctggtaaaccaaatgttataagctgagtaatgtctgactaaaattcctatgttgaagtctaacccccggtaccttggaatgtgaccgtatttggagaaatggcccagtctgcgatactttgctgtggcagccctagaaaccaatatgccaaccaacccataaacagacaaaaaaccaggaaagaactaaatgcacaggattccaaatccagtggcagcatctcccctatcatgtccttcttccaggataaaaatacacagatggagacacaactgtaaatgtagtgcatgtacagacacaccctccctctgctcacaaggaggccctaggagatgttgcacctcaatcaaaatgaacacccagtgcccaggtcttgacttctaagaagatacaggactccctggggaagaggccaatgctagggctggggccaggaaggtacaagatgagcttggaacacccagttgaggctgaaagtgaggactcaaaacatgatgggaacagtcccaaaagcataccagggctaccctaaaaaagctcccacttgccaaagctgggtcaatttaaccagctttggttaaattataaaataatgtaagtagcaaattataatggagtgtaacctgtagaagaaaatacaaatcataagtctatgctgacatttacttctatttatttatttatttagagacagagttttgctctgtcgcccaggctggagtacagtggcgtgatcttggctcactgcaacctccacctcccaggttcaagagattctcatgcctcagtctctggagtagatgggattgcaggcacgcagcaccatgcctggctaatttttttttattttcagtagagacaggggtctcgctatgttgcccaggctggtctccaactcctggtctccaatgatctgcctgtcttggcctcccaaagtgctgggattacatgcatgagccaccgcgcctggcctgtgctgacatttaaatgggaagcttgatgaggaacagaatacttacctactttcaaagcttctcttcagaaaatacttagttataaaggggaaaagagttaaccattcagtggaaaaacctggccgacatcaatttcaaggggtcaaaataatcatcagacaaactggtattgctaccatcttctaggacacacagaaagacatggcatcaccaaggaagcagagcctgagtcaatcataagaagaagcatcagacaggcctgagtgagggactttcttcgaaataactgacctgtaatcttccaaaggtcccaggaactgtcctagtctaaatgatactgaagagatcttcgctcaatgcaatgcctgactctgatctggaagtcttgctctaaaggacatggttgggacaactggcaaaacctaaagggtatctgaagattagatggaagtaatacatcagtgttaattacccaatttttatccttgttctgtggttttgtaggaattgtgtagaaagtattaaagggtgatagcgttttaaaataggatttatttatttatttatttagagacagagtctccgtctgtcacctaggctggagtgcagtagcacaatcatggctcactgcagcctctgcctcccaggctcaagtgatcttctagcctcagcctcctgagtagctgggactccaaggcatactccaccatgcctggctaatttttgtgttttttgtagagatggggttttgccatgttgcccaggctggtctccaactcctggactcaagcaatcctctcacctcagcctcctaaagtgctgggattatagttgtgatcaactgcacctggccaggtttcatttttatttaggtataatgagggcaacagatcaggagacaaagagttttttggctaggtgcagtggcttatgcttgtaatgtgttctgggaggccaaggtgggagaaccacttgagtccaggagtttgagaccaccttggtcaacatagcgagaccccagctctgcaaaaaaattttttaaattagggcaggcgtggtagctcaagcctgtaatcctagcactttgggaggccgaggcgggcagatcacaaggccaggagttcgagaccagcctgaccaacatggtgaagccatgtctctactaaaaatacaaaaattagctgggtgtggtggggcttgcctgtaatcccagctactcaggaggctgaggcaagagaatcgcttgaaaccgggaggaagaggttgcaatgagctgagattgcgccaaaaaagaaaaattagccaactgccaggcatgatcactcatgcctgtaatcccagcactttgggaggccaaggcgggcggatcacctgaggtcaggagttcgagaccagccttgccaacatggcaaaaccccatctctactaaaaatacgaaaattaaccgggcaacagagtaagactccatctcaaaaaaaaaaagaaaagaaaaaaagaaaagaaacaagagagagaataatttccgactgtacccactttctctgctatatttaacttctggagggcaaggagaatgtcttgttcactctgcattccccaagggctcagaactttcaaacacatagcgtgaactcaataagggttgaacaaaagagtgaatgcatcagtgagaaaacgaaaaggtcaggccaggggcagtggctcacacctgtaatctcagcactttgggaggccgaggtgggcacatcatgaggtcaggagattgagaccatcccggctaacacggtgaagcttcgtctctactgaaaaatataaaaattagccgaggccgggcacgatggctcatgcctgtaatcccatcactttgggaggccgaggcgggtggatcacgaggtcaggagatcgagaccatcctggctaacatggtgaaaccccgtctctactaaaaatacaaaaaaaaaaaaaattagccgggcgtgatggcgggctcctatgctcccagctacttgggaggctgaggcaggagaatggcgtgaacccgggaggcggagcttgcagtgagcggagatcgcaccactgcactccagcctgggcgaaagagcgagactccgtctcaaaaaaaaaattcgcctagcatggtggcatgctcctgcagtctcagttactcgggaagctgaggcaggagaatcgcctgaacccaggagacggaggttgcactgagccgagattgcgcctctgcactttagcctgggcaacagagcaagcctctgtctaaaaaaaaaaaaaaaaaaaaaggtcttgctgttcactctgtgtggttagctcctgaccagtttctgttctctctgttcatgtggtggacagtttggtgtcacagcttggggttttctcctaaatcacagtctacttagtttttcaagattctctaggctgggcgaggtggctcacgcctgtaatcccagcactttgggaggccgaggtgggcagatcatctaaggttgggagttcgagaccagcctgaccaacatggagacaccccgtctctactaaaaatacaaaattagccgggtgtggtggagcatgcctgtagtcccagttactcaggaggctgaggcaggagaatgacttgaacccgggaggcagaggttgcagtgagccgagattgtgccattgcactccagcctgggcaacaagagcagaactccgtctccaaaaaaaaaaaaaaaacaaaaaacaaaaaaaaaacaaagtctctagcaagtacaagaggtcaaattgtaaaactgtcgtaactctgattctccctctttcttcttttgccactggacatttacttagaccctaaaaagtacaaaatcagatttaatcttatgggtgttaatagcagcctttcattctgttctgtactggacaagtggccataatttctcaaatgagagttagcatttggcaaacagaaaattttacattagtgagcctaggagtttgaggctgcattgagctatgatcatgctaacctgggcaatagagtaaaaccctgtctcaacaaataaatagataaataaataaaaattttattttattattgttatgattttcttttggagatggagtttcactgtcgcctaggctggagtgcagtgtcacaatctcggatcactgcaacctctgcctcccaggttcaagtgattctcctgcctcagcctcccactagctgggactacaggcatgtgccaccacactcagctaatttttatatttttggtagagacggagtttctccatgttggccaggctggtctcgaacctctgaactaagatgatctgcctgccttggcctcccaaaattctgggattacaggtgtgagccaccacacctgcccataaataaaattttaaaagaaacattttaattgggactttctgtcacatctggtcatcgtatcacaggagctgcagtggcgtctcttgcatatgctatggaacagaatctccgtggagcagggagggctgccatgagaccagctttgaggctgacagaaggaggaagggaaggtgtgcccagggagggacatcatgttccgttcccagtgaccaatcctctatgggtcttgaggatggttgtggactgagggagaagggggagaagaaggaaggacacccccaagctgaatgggaaagaaaaggaagccctggggtcagaaagccatctggctgggaccctgctccctggtcggttactgttcaaagatgaccataggttgggacacggaagacagctaatcaggggcccttgtgccaccctcagagataccaagaatgacagaaactgatgaatggaatgttgtatgaggggcggggtgtggatagatgattttgtcactttagaggcattcatggaaggtagaaccataatgaactatgtaggaagttaaagagtgcggagatgcaagaatatttgaaggtgaggccaaagctgggttactcaccaggcagcctgtgcaggtgcacgggccatgagcaaagtagggaggggcagaaagaagggaaatagaaggtgggaagcaagagagggaaagcattacctcgaatttaacaatcagaaaatctaggccaggcaagcgggctcacgcctgtaatcctagcactttggtatgctgaggcaggaggatctcttgagcccaggagttcaagaccagcctggacaacataccaagaccctgtctctacttaaaaaaaaaaagaatatctagaatgaaggtatgttgtttcagcatacatatatttttgtattttatggcttattcttctatcccttgggttttgccccacatgtgaagtgtgctgaggggcagaccaaaggagaagggaatgcagccaccagggacagaccctgtcagtgagggagatgtccacctgtctgacctggcaaggcatggtttatgtttacaaaggtcagttcaggctgggcacggtggctcacgcttgtaatcctagtgctttgggaggctgaggcaggaggatcacttgagcccagaagtttgagaccagcctgggcaaggcaacatagtgagatcttgtctctacaaaaaataaatttcagtttggcctttcaatcatttcaaaatagaactttacacctgggatacaggttttaaaaaatattggaaaagcttcactgtcttcaagactttgcaaaattaaacagctacttgagaggtgttatgatctttgtctaatttgccttgtctaactgttgcaggatgtgtgtgattatataactaggaatgtgagacactattccttgaaacaatttttggggaaaagctacagccctgtggtgactgaaaacagacagcaaggtagcatgtcctgaagacacacagggcaagagagggctggctggcagacctccctctgtcactgcagccatcctgcaggctccccattgaggaaccagttcctccaagagacttccacattttcctggagattccacatccccaccccacccctaccccacccctaccccacccccaccctgctgtcagcctgggggttgggagacttctggtaggtcctgtggctgaacttgagctgggcctaatccactttactgcgctctccatggaagggccctgcagggtaagcaggcttaggattggctggcttggatgatttcaggggcctggggaatatgggctgttccatagttgtttggtccctggccctgggtgattagaacaggtacataatggccccaagtgtgagagccctttaataaaggaggtggcggggagagggctctggattggatgctgtgttcttctccatgtatgaccaactgcagcctctcccaaaaccttagcagatggtttgctttgtgctccattccggaaataacttttggccacttggaaaggagccggtcaattatcaggagccatctgtatttgggtgttgctgtctgtttcctcacgcttctatgaccaggaactgcctcggcctgtgctgcgctgtctgtggtcggtataaatctagtttcctacagtcttttcagactatactgctttaataaatctacagctgtctttataccaggtccaaataggtaaatgaaaagttaaaacatttaatcaggccagatgtggtggctcacgcttataatcccagcattttgggaggtcaaggcgggtggatcacttgagcccaggagttcgagaccagcctgggtaacatagggagatctcctctctagaaaagaataataaataaataaataattttaaaaagaaagcttttttttggagatggagtttcgctcttgttgcccaggctggagtgcaatggcatgatcttggctcactgcaccctctgcctcccaggtttgagcaattctcctgccttagcttcccaagtagctgggattacaggcacccaccaccatgcctggctaatttttgttttttttagtagagacagggtttcaccatgttggccaggctggtctcaaactcctgacctcaggtgacctacccgccttggcctcccaaagtgctggaattataggcatgagccactacacccggcaaggaaatatttaatcagattttttggtagacataatggaccattcagatgttcccagacgcccagctattcagcatcatgtagatgcatcctaagaggagcttgttgtcctaacagcatgtcagttatctctctgtctcttatcatcaggcattctaaccccctaaaaggtagagaaaatcttatcccatgggcaagtcatagtaaaaagattacactttatgggcaccaaaagaatatgaagttaattttcctcctttatatgcaccaaaaaaatatgaattagatagcccagatgtggaggcttgtgcctgtaatctcagctctttgggagactgaggtgggaggattgcttaagcctgggagtttgaggctgcagtgagctatgatccagccactgcactccagcctgggcgagagagtgagaccctgtctctaaaaatactttgaaaaatgaaaacagtacaacttagacagaaatccagacaaggctagagccgagatgcctgctccttgaggtcgccattgctctcctaagctctgtgccgaaggatgttccgaggccctgtggctcttctacgtagacacgcacaggctatgtaggtagaatggggtccttaactatccagcaataaacaatcatcctggatgttgcattttcctcagaaaagacctgaaagggatatggagtttgagtgggggaaggagatgttacaatcctcatggtagggctttggagattatggaacagtgaagccggcctgtaaggggcagaccattcctcccttgtagattgtagctgatcactgccgtctatctgattggctgtgaatgaggtgactgcctccaaatgaagagtagaacagatcgggaggtggggggcgcaatcagaggatttttgtccctgtggttccaggaagtaggcacattgctgaagaaggccaggcaggagataaggccattgtttgagcagcaacatccaacagaaatatatcaccagccacacatgccatttttcatgttcttttcttttatttttttgagacagggtcttgctctgtcaccctggctgccaggctggagtacagtagtgtaatcatagctcactgcagccttgaactcctgggctcaagtgattctcccacctcagcctccaaagtagctgggaatacaggcatgtgccacctcgcctggcaaatttttaattttttgtagatacagggtctcagtatgttgcctaggctggttcatttttcatgttctaatagccatgttaaaaaaggaaaagaagtggccgggagcggtggctcatgcctgtaatcccagcactttgggaggctgaggcgggcggatcatctaagatcgagagtttgagaccagcctgactaacatggagaaaccccatctctactaaaaatacaaaaaaaaaaaaaaattagctgggcgtggtgatgcatatctgtaaccccagctactagggaggctgaggcaggagaattgcttgaacctgagaggcagaggttgtgatgagctgagatcacaccattgtattccagtctggggaacaagagtgaaactccatctccaaaaaaaaaaaaaaaaaaacggaaaagaagcgcgggccgggcacggtggctcaggcctgtaatcccagcactttgggaggctgaggtgagcggatcacaaggtcaggagttcgagaccagcctggccagcatggtgaaaccccgtctctactaaaaaatacaaaaaactagccgggcatggtggcatgcacgtgtagtcccagctccttgggaggctgaggcaggagaattgcttgaacccggcaggctgaggttgcagtgagcccaggttgtgccattgcactccagcctgggcaacacggagagactccatctcaaaaaaaaaaaaaaaaaaaaaaggaaaagaagctactttcattgtaccatatatttacccccaaacacctaaaatattattatgaaatgtgttattaatataataataatttttttttggtagagatggagtcccactatgttgcccaggctaatctcgaactcctgggctcaagcgatcctcctgcctcagcctctcaaagtattgggattacaagcatgagctaccatgcctggctaggaattattaatattcatatattttacactcattttttgtaccattacttcaaatcgagtgtgtgctttctttttttttttcttttttgagatggagtttcaatcttgttgcccaggctggagtgcaatggtgtgatcttggctcactgcaactctgcctcctgggttcaagcaattctcctgccttagcctcccaagtagctgggattacaggcatgcaccaccacacccggctaatttttttggtatttttagtagaaacggggtttctccatgttggtcaggctggtctcaaactcccgacctcaggtgatctgcccgccttggcctcccaaagtgctgggattacaggcatgagccacagtgcccggccctgagtgtgtgtttttgtcttggactggccacatgttgcccttagctgcatgtggctttagctgtccttacaggcaggctattccaggggcagaggtgaccacccttgtaaccctgagcttccagctcctcttctggaagcagaaagcagagactatgtcattagtactatcagaaacctggttctttggactgtagttacgttttatttttgacgaggtcttgctcaaggctggagtgcagtggcatgatcatagctcactgcggcctccaactcctgggttcaagtgatccacgcaccacagcgtcccaaaactctggaattaaaggcatgacctctgcgtcctggcctgtaattactctttaagtagttcctcctttggttgccctcctttttgttttataaatgccaggctgctgtgttgtgctgagtaaatcttgggagttcattgtatgaacttttcatgggaatacttcaaataatgtagagagaagcagcctcgggctgcagacatgctgggtggatgaggagggaatcccagagagacacactggattctgagcctcattggaggtttcctggcccctgcctacttggggctcttccagcattttcagtggaagcccaggagagcagccaaatcaatgcccttccaggagatcagataagtgggacatggaaatagaggaacatggagggaaaggggaataaaataaatggtaattgatggaagataaagacccagaaacagcaagcttaatagtaacacaaagccaatagaagccacatgccgggactgctgctcaatgtggcacaaataattcagagtatgctacaaaccactggaaagaaaagagaaaggtagatcaataattataatcatcaaaggtagatcaataatatatataataatacataataagatcaataattataataagtgcggtggctgacacctgtaatctcagcattttgcgaggccgaggcaggtggatcacaaggtcaggagttcgagaccagcctggccaatatggtgaaaccccgtctttactaaaaatacaaaaattagccaggcgtggtggtggacccctgtaatcccagctactggggaggctgaggcaggagaatcacttgaacccgggcggcagaggttgcagtgagctgagattgtgccactgcactccagcctgggtgacagagtgagactctgtctcaaaaaaaaacaaaaaaaaccacaataataataataattacaataaaagaaaccctcttgaagggcagagcagccccctgggcttttctggaccattcagtgtgaatgaacacagggtgacctggcaattgcaaaacacctaggattattgtcaccaacaataaaggaaataattatactaatcaattgtttgagcaaagcttttgacacctcccccggtaatcaaaaataggctcacaggtaatttagtgtataaaaacaaaacaaacaaaaaatataatcctagaaagaaaatccttttgtccaaacttcattgcagaccaggcatttgaaaacgtcataaactgtaaacagccatttaaagtgcttctggggagcaggccttgggatgagagcacttttttggaaaacagcaatttagaactgaggcagcttgctcagaagttccaatccagcgaaaagcccccagtcgctagcagagcaaatggagacctcctgcccttttgacttcagcaccgggacttgcatgcaggtctgtcagtgagctttcctgcaaacctcagagaagagctcaggttccgacaatgccctgcccttctccccaggagaagcttcccttggtttgtggagtttttctcctctgttcgttccgcagaactcccctgagttcacattcacggggaacaacagagcctcgttaagcgagttacaatgggaagctggcagggatggccgggataccgtgatatcccactggggtttccctttgaaatcttcctatggtctgagaccaaaaacagcaatcaatgagatgactggaaggaagagaagctgcatgaaaggcattcagaaacctacacttatctcctggggcccccaccctgtcccttgaagccatcacagggttctctggagagtgtatggcagttggagccactatcttcctcctcactatctctccaaccctgccccgattcctcatgactcagcctgaggcggggagattaagaattcagactgtgggccgggcgcggtggctcacgcctgtaatcccagcactttggaaggccgaggtgggcggatcacgaggtcaggagatcgagaccacgctggctaacatggtgaaaccccgtctctactaaaaatacaaaaatattagccgggcgtggtggcgggcgcctgtagtcccagctactcgggaggctgaggcaggagaatggagtgaacccggaaggcggaggctgcagtgagcagagattgcgtcactgcactccagcctggacagagggacagaaccagactccgtctcaaaaaaaaaaaaaaaaaaaaaaaaaaaagaattcagactgtgtaattacccctttgttaaaacatattgctacacatttcacgtatatgtgtagatgtatataggttttactttaattatgcaaaaaagcgtccacttcttatcggtaaaatattctacatctgcacatggagacagatgcttaatggaaagggatatgggatcattttttacagcccacagacactttgatttcttgctcagaggacttcattcactcagtgcctttcatgaggtcaactttgttctagataccttgctaggcgttggggacacaaagatgactctcgctctatctgggtggacgacagcacttcatccaccaacagaagggcccggagagttttagggttaaatgtgcacgttagggccgggtgtggtggctcacacctgtaatcccagcactttgagaggctgaggcagctggatcactttagatcagaagtttgagaccagcttggccagcatggtgaaaccgtgactctactaaacatataaaaaaagttagccgggcatggtggcttacacctgtaatcccagctactcaggaggctaaggcaggagaattgcttgaacctgggaggcagaggttgcagtgagccgggatcacaccactgcacgctagcctgggcgacagagaaaggctctgtctcaaaaaaaaaaaaaaaaatgcaccttagataaagtaaaaataaggtaggtgtgctgttgcattgcagatatacctagagaggtacataacatatttttggtgaggttggaaagataaaggtagggtaattacagaatatagtagaaacttctaagctagaagttgaggagcagttttggatttttgttttaagatgcaaggtacttaagatcaggtgctagtgactgaagctggacatctaaaaggtaatttattttttatttttatttattttttttttgagacagggtctctcactgtcacccaggctggagtgcagtggtgcagtcatagctcactgcagccccaacctcccgggctcaggggatcctcccaccttagcctcctgaatagctgggacaatgcctggctagtttttgtattttttgtagagacaggatttcgccatgttgaccaggctagtcttgaactcctgagatcaagcaatcttccagctttagcctcccaaagtgctgggattacaagcatgaggcactgcgcccagccaaaaggtaatttaataataaccacaaggtcatccaaattagaaacaagacaaaacaatgcaatgaggggtggggggcaacgaatttgaaagttctttaaaagcatcagctatcttcctgaaggttatgaaaccgcctttgcaaaattataactgagacaatgaaagagatctaacttaaccgactccatcttgcttctaacctccaagctgtctttgttcattcctgggcattggctgaaccaactttgggagaaacttagtttgtagtttatagtttgtttatttatttatttggagacagagtctcactctgtcgtccaggctggagtgcagtggcatgatctcagctcactgcgccctccacctcccaggttcaagccattatcctgaatcagcctcccgaatagctaggagtacaagcatgcaccaccacacccggcaacttttttttaatttttagtagagacagggttttgccatgttgcctaggctggactcaaactcctgagctcaggaaatccacccgcctctgcctgccaaagtgctaggattacaggcatgagccactgggcccggcggtttttttttgtttgtttttgttctttttttgaagagtcttgctgtgtcgcccaggctggagtgcagtggcacaatctcggttcactgcaacctccacctcctgggttcaggcaattctgcctcagcctcctgagtagctgggattacaggcgcacatcaccacgcccggctatttttttgtatttttagtagagagggggtttcaccatgttggccaggctgttctggaactcctgactgtgatctatagtttaaacaaagacggcaacagccctttcccaaagcagacctccttcttgcctggggactagattgcctttgcaagactaacattagtcacaggattagaaattatggtttaggagtcttgcagctggaggctacaagattctgaccctccctaaactgctcctgagatcagtgcttgagatattttgcagaccctgcacttgatggatcagctggcaccacccagactgataaactggctcctctgatcttgtggcccctacgcaggaactgactcagtgcaggaacacagattcgtctccctgtgatttcatccctgaccaatcagcactcctggctcactggcttccctccacctaccaagttatccttaaaaattctggtcgctgaatgctcagggagactgatttgagtaataataaaactccgatctcccgcacagccagctctgcgtgaattactctttctctattgcaattcccctatcttgatgaatcggctctgtctaggcagcgggcaacgtgaaccccttgggcagttatatttagagtgttctctgtcttcagcattgtttttagtttgcatcacccaaccctgagtggggagggaagtggtgctaggtcttctcagtgtctcgggtgggtgtgggggcttccacggaggctgagatctaattttattttcccaaacacttaactattggagaaaaaaaatccctttattggcatataaatcctttctgacttcaagtgtttagcagctactctcatttgtttactgagtttgtgatgagagaaaagagggcttgcagccccaaagaaaggcgggagggacccagtagagctgggggctgcccgccccctggccttcgcataaagagcgccttagcaggtctactgagaacgggccagctgttagccagctggtcactcaggaggaagcccggggaagagagaagtgagaacgtttcagccaagtaactaagtgcacagccaccctgaccgtggccggaaaaccgcgaatggaaaaagaaaaactgccccgagaaactctgagaacttgcagtaagtgcacaatatccgtaccagagcttaatttctttacctgaacttaaaaacatgcacatactttgtattttaattgcagcgacctctctcctgtgctagcccgaggtccggagtcttacaaagggcttagttgagagtgcacgatttgtgagtcctcttcaacttggtgtttaaggagagcgggggtgagctcagtgaatgcggtttagcaggagagcatttgaatcccacagaggagccagcctatgccctagttctaaggcgcctctggtcctgtgtgaccgcgcccgggaggcaacagctctgagctcagtttcctcctcagcaaaatgtagccaatgacggcgttgtgaagcgtgaatgagatcaggtctatgaacgtcactttgtaaacccttaaagcaccatttcaatctttcattaaatattaactgaaaggagcactcacatagaaatgtcagtgtctacccccaactaactttcccctcttttctcacttcttctttttcacagaaccaggctacacatcttgtcattaaatttaatcaataaacatctagtgagttctactcaagacactgagctaattggtttatgttcagcaccgtcaagaaccttaccatctagttgggaaaaaccacacaaaacacattcgaagtttttaatggcactcatgtcaaattatgaaagaagaatggaagaggtctctccttagcaaatgattaattactgcgttagtctgctaggagtgccgtaataaagtagcacagacggggtggtttaaacatcagatattaattttctcacagttctggaggccagaagtctcagctgaacgtgttagcaggattggtttctcctgacacttccgtccttggcttgtagacggccatcttttctctgtcttcacagggtctttcctctgtgtgccccagtagctccccctgtatccaaatttcctctccttataaggacatcacttatatgggattagtgctcacctcattaccccattttaacttaattacctctctaaagaccctatctccaaatacagtcacattctgaggtcctgagggtcaggatttcaacatatgaatgtttggggagacagaattcagcgcataatcgttaccaaaccagaggtttagatgctgtcaggggagttttcttacagaaagagccaaacatgcaatccttcttgggtgcttttgatgacaggctgactataagttagagctatttgtactagtgctacaaatttggctatcttctagttaaataaattcaataaatcctggtttatttttacgtctacaggatattgtattaaatattcaaattgaataaaaatatttaaatcatcatttgaataaactacttctcttgtgaaaaacaatacatttattgcaacttaattatttttatcttttccctctctccctcccttattcctttggaatgaggaggccccgtgggctgtggagatttttgcccacgaatgggctgtggtaactacagccacagactacaaatgctgaaccgaggggtaggagaggcgtggaacctcttaatagaggaggtggcccttaaactggggaccaatagattgatatattcagaaattcatttctgccagattgttgttatgactgcacagaagttctccactgagttttacatagcatctctacagtacttctacatagtattgctccacgtggatgaaccacagtctatttaaaaatccatggttttgttgggaaattggtatgggggagtgaagcacaggtgcacgtgttaggaccccaaagatggtgaactgtgcctgaggagggcgaagccagaggacactctggtggaggtccgttgcagtgctgtcgtgcaaatccatcgtccgacctgggtataggggtgaaagactaattgagccatctactagctggttccctcagaagtttcccttaggatagttggtgctctcccaaaacccaacccacacagttttatccagtaaagttaatattagaggtcctggggccaaaatgatctcaacctattctcaaactttaaatgggtaagattattattgttgttatttttattttatttatttatttatttatttatttattttttgagaccaagtcttgctctgtcgcccagactggagtgcagtggcgccatctcggctcactgcaagctccgcctcccgggttcacaccattctcctgcctcagcctcctgagtagctgggactacaggcgcccgccaccatgcccagctaattatttgtattttttagtagagacggggtttcaccgtgttagccaggatggtcttgatctcctgacctcgtgatccgcccgcctcggcctcccaaagtgctgggattacaggcatgagccaccgcacctggcccttgtttttgtttttgagatggagtctggctctattgcccagactagagtgcaatgacgcgatcttggctccctgcaacctccacctcctgggttcaagcgattcttctgcctcagcctcccaggtagctgggactacaggcacccaccaccagacccagctaatttttgtatttttagaagaaacagggtttcaccatgttggccaggctggtctcgaactcctggcctcaagtgatccatctgcctgtctcagcttcccaaagtgctgggattataggcatgagccaccacgcctggcctgaatgggtaagattattaaaaaaaaaaaaaaaaatcatggctttggggtatctaggccattttcaactcttgactcaaaagagtgctcagtggacatttatttattttattttacttttatttatttatttattttgagacagagtcttgttctgtcccaggctgaagtgcagtggcatgatcttggctcactgcaacctctgcctcctgggttcaagcgattctcctgccttagccttccgagtagctgggattacaggcatgcaccaccatgcccgcctaatctttgtagttttagtagagacggggtttcatcatgttggtcaggctggtctcgaactcctgatctcgtgatccaccccctcccctcccttggcctcccaaagtgctgggattacatgcgtgagccaccacacctggactcggtctttttattttatttttttaatttttattttttgcgatgaagttttgctctttttgcccaggctagagtgcaatggagcaatcatggctcactgcaacctccgcctcctgggttcaagcaattctcctgcctcagcttcccaagtagctgagattacaggcatgtgccaccacatccgactaatttttgtatttttagtagagacagggtttcgccacgttggccaggctggtctcgaactcctgacttcaggtgatccacctgccttggcctcccaaaatgctgggattacaggtgtgagccaccacgcccggcctcaatcttttataaatgtgaaaaccattcttaacttgctggccatctaaaaaaactgttgggcaatgactcagactgtgggccatagttggctaatccctgacttagaaatgagaaatttgaaaaatttactgctatgctctgcttagttacaagaaatggaaggacggttgcttggtgcaaggtatataagatatggcatgccagccacagggtgaagcttcttgttgtaagtatttctagagactggcgatggccatgggtgagtctggacctgctgggtgccctggctgcactggcctgttgcatgacagctttcaacaagggccacaacatgttttgagtgaaagaacacctgcctacaatgctacccagacgcaaaggcagaaagcatagcaaatgaaatctttccatccaagcaggcttttccggtagtgaaactaaaaaagcagcagtacataccaatgaatgttttaacatttctacttgaggacatgatacggtgaccacagctggagcttgtccaccctgaatgtacagtggaaactagcaggcttcctaggcaggggagctattcatgaacaagcccatggatggggcccttaactgcccttcatattggctctgcactgagtcccatttcttttcttttcttatttttttttttttttttttgagaaagagtctcgccctgttgctccggctagagtgcaatggcgcgatctcggctcactgcaacctccatctcccaggttcaagcgattctccttcctcagcctcctgaatagctgagattacaggtgcatgccaccacacccagctaattttttgtatctttaatagagtcggggtttcaccgtgttggccatgctggtctcaaattcctgacctcatgatccgcccgcctcggcctcccaaagtgctgggattacaggcgtgagccaccgctcccagcccccatttcttaaaagttgtttaggaagtaacaggaacaataagctgtgttttcttagcacttccatgccagtacagcatcattaccatgtcacggtttaagcagaggagattcagggagacactgaaccacaaacacaggtattaatgactctaaagttgtttgtttgtttgtttttgatagagtcttattctgtcacccaggctggagtgcggtggagcgatcttggctcactgcaacctctgcctcccaagttcaagcgatccttccaccttagcctcccaagtagctgggattacaagcatgtgccaccacacctggctaatttttgtatttttagtacagatggtctttcaccatgttgaccaggctggtcttgaacttatgacctcaagtgatccactcgcctcggtctcccaaggtgctgggattataggcgtgagtcaccacgcctggctgatgactctaaagtttttaagcaccatactacacaatctcttaatttatcctcctgattattttgtaaagtgggattttttttttgttttttttgacggagtctcactctgtcgctaggctagagtgcagtggtgcgatcttgactcactgcaacctccacctcccgggttcaagcgattcttctgcctcagcctcccaagtagctgggactacaggcgtgtgccaccatgcccagctaatttttgtacttttagtagagatggggtttcgccatgttggccaggatgatctcaatctcttgacctcgtgatccgcccaccttggcctcccaaagtgctgggattacaggtgtgagccactgctcctggcctaatttttgtatttttagtaaagatggggtttcactgtgttggccaggttggtcttgaacttctgatctcgtgatctgcctaccttggccgcccaaagtgctgcaattataagcatgagccaccacgcctggccgggaattgttgattctattttcttttcttttttttttgagacagagtcttgctctgtcacccaggctggggtgcagtggcacaatctcggctcactgcaagctccgcctcccgggttcatgccattctcctgcctcagcctccccagtagctgggactacaggcgcccgccaccacatccagctactttttgtatttttagtagagacggggtttcaccgtgttagccagcatggtctcgacctcctgacctcgtgatctgcccgcctcggcctcccaaagtgctgggattacaggcgtgagctactgcgcccggccgattatattttcatgagcaaagtgtcagagaccctgaatgaattatcgaaaatcatagagtaagtgcccaaactggatcaaattacagtcttaaagccccaagcccaatatcaagaagtgtttatgattaaaacaatgtgatcaggaaggtgaaggttttaggattcaaaataacaaaacacagtaactcaattttttaccttttctcttggttttcttgttctccacctttcatctgtttgtttttttttgagacagtatcgctctatcccccaggctggagtgcagtggtgcagtcttggctcactgcaacctccgcctcctgggttgaagcaattctcctgcctcagcctcccaagtagctgggactatgggcacccaccaccatgcctggctaatttttttttttttttgagacggagtttcgatcttgttgctgaggctggagtgcaatggtaggatctacagctcactgcaacctctgcctcctgggttcaagcaattctgttacctcagcctcccaagtagctgggattacaagcacgtgccaccacgctggtataatttttgtatttttagtagagaccatgttggccaggctggtctggaactcctgacctcacgtgatccacctgcctcggcctcccaaagtgctgggattacaggtgtgagccaccacgcctggcctgcccagctaattttcaaatttttcatagagacagggtttcaccatgttggccagctggtctcaaactcctgacctcaagtgatctgcctgcttcggcctcccaaagtgctgggattacaggcatgagccaccgttcccggttgttctctcccattcaccacattctaagacaccacaactctaatagaatgcctctaaatactaatttgcaaaaagaaatcataaaattcttaagaaggtagttttcacataatgaaaatggaaggtttcttattagcattcttgcaactgtctttgatccctcattttttctttataatggctaatacttagcgagtgcttaccaggtgccagacacacagagcactttacaaacattaatcacgtaatccttatgaccatttatgagacaggtgtcattatctttatttttcagatggggaaaccaaggcacagagagagagagactcatttctagactcacctctgtatcccttaggtctaaggcatagagggtcatcagttcaagtctcatttgggcagaagtcatagtgattctggagacagagtacgtcggtatgaatcctggctaccatacttactaactagctctttatttatttatcattatttatttatttatttattttttattcatttattttttgagatggagtctcgctctatctcccagactggagtgcagtggcacgatctcagctcactgcaacctccatctcccgggttcaagcgattctcctacctcagcctcccaaggagctgggattacaggcgtgcaccaccactcagctaatttttgtatttttattagagacggggttttgccatgatagccaggctggtcttgagctcctgacctcaggtgatccacctgcctcggcctctcaaagtgctgggattacaggcgtgagccactgcaccaggccatatttatttatttagagacagggtcttactttctcacccaggcaggagtgcagtgatgcaattatgtctcactgcagacttgacctccagtgctcaggtgattctcccacctcagcctcccgagtagctgggactacaggcgtgcaccatcaggcttggctaattttttttgtttgtttgtttgagacagagtttcgctcttgttgcccaggctggagcgcaatggcgcaatctcggatcactgcaacctccgcctaccgggttcaagcgattctcctgccccagcctcccaagtagctgggactacaggcatgcaccaccatgcctggctaatttttttgtatttagtagagatgggatttcatcacattggtcaggctggtctcgaactcctgacctcaggtgatccatccccaccttggcctctcaaagtgttgggattacaggcgtgagccactgcgcctggccaggcttggctgatttttttatagtattttttgaagagatgagctctccccatgttgcctaggctggtctcaaactcttggactctaatgatctgcccatcttggcctcccaaagtgctgggattacaggcgtgagccaccgtgcccggcctatgctttacattaaagatatcatttacttaagtcatccattaattagtaaagattttagaagatatttttcaaaatgatgcaaaattcaaaaaataattgataaaatgcgaattacattttaatgtctcataaatggaattactaagaagcagtaatgaatatctgatgacagttcaaatatctacctttcataccctgtgtagtattttgtccattttttctatgcttcgtttcatattatatatactgtgtgatggttttattttcatataatacaaaatctcaaatcagatggtgtcctacctgttatagttgtctggtaaaatgcaactagaaaacatttctctctagagagaactttgacaccagatggtgctactatcactagcaatctgataagaaattaaaaaaaattaaaagccctttgtgtgaaagtcagttctggggagatgagccacagttgtttcaaactggtctgcattttctccaagacaagagtatagggctattccaaggaacagtatctttcctagcataggtgttaacttttgtggacatgtatacacgtagtaagtctgcagtggggcacaaagagcacaaattgcctgagtgtgctggggtcctaacgggtagaccttgaaattacccttttcagaccactcttggctcccctggggcctctcatgtttgtctgaggtaaccaaggacagagtaggacaatatagatgtgctggcccctacaaaactgagcagacagtaatcacaggacccgctctggtcaagcatgaggtggtagccagacataagaaaggggccatagagtgctctacttcgtgccatagaatatgccttagagattcttttgaactgtaaacatttcgaaaaaaaaaagcatgttgttttcacaatctttttttttttgagaccaggtctcactttattgccaagaccgaaggacagtggtgtggtcacagctcactgcagcctcgacctgtggggctcaagcgatcctcccacctcagcctcccaaataactgggaccacaggtgtgagccaggatgcccagacaatttttgatttttttgtaaaggcaaagtcttgctatgttgcccaggctggtctcaaactcccgggctcaaactatcctctcacctcggcctctcaaagtgctaatattacaggagtgagccaccatgcccagtcttgttttcatagtcttaatagtcatactgtgtaaggactacataacactgcactgaatggaggtaataaaatttacttagctgtacctctccatggaacatttgggctgtcggactgaggctggactggaccaagggctaaagaccaagaggccaggatggatcaagtcagtgtctggaggctaactggaaatagacagctcggtagccagtctgatcaatgcatcactgtggagagtcgagtagggacaaggcaacagacagatgccagggacaccagacaagaaactgcatgagaatgaaagatacctacttggatcctccagctgtgaccacaggcttggtttggaagtgagctttaaagagctctcagccatcttgcatagcttttgcaaaaggcacttgtgatagcatggccagtgagtgggtgaatgatttttatcataatagactagcatttgctcattcatccaagacccacatccagtctacatccaaaaagaaatggctgtaacaattacagtagtcccccttatctgtgggagataagttccaagaaccatagtggatgcctgaaaccttggatagtactgagccctatatatactatgtttttctcctttacatacacatctatgataaagtttaatttataagttatgaactggatgagatcaagagcaatagctaataataaaatagaacaattataacacatactgtaataaaagttttgagaatgtggtcgctctctcaaagtatcttattgtactgtactcacctatgttctgacctcaattgacggagactgaaactgtggaaagtgaaaccacaggtaatggggaaactactgtttaacaaaacctttagcatatacatttactattttaaatgtcagttacataattctcaattatttgaaggattaaaaaaatcatgaattaatttcaattgggactggagtaaattgggcattgttgcaaccttaatttagaacttcctaaactcttactttaaatacaggaaaagactgatcttcatattcctaggtctaaggaggatttagtgtttcatcctccatgctgtaaaatgcaaatttggtaatttcactttctgggttaaagtcctgtggttccctatcacccaaggatgaagtccaaattcctaagcattgtagctttaattataaagtagcaaaagcctcaaaatgcaaaatagcatgctttttgttctccagaaatctagatctgcattatctaaatggaattctttagcaaatagctacatgtggctggtgaaaatatgaaatgtagctggtataaattgagatgtgctgtaagtttaaaatacatagcgcatattggctggatgtgatggctcacgtctgtaatcctagcactttgggaagccgaggaaggaagatcacctgaggtcaggagttcgagaccagcctggccaaacatggcaaaaccctgtctctactaaaaatacaaaaattaaccgggcgtggtggtgcttgcctgtaatcccagctactcaggaggctgaggcaggagaatcgcttgaacctgagaggcagaggttgcagtgagccaagattgtgccactgcactccagctgggtgacagatggagacgctgtctcaaaaaaaaaaaaaaaaaaaagccctgtcgcggtagctcacgcctgtaatcccagcattttggggggctgaggcgggcagatcacctgaggtcaggagtttgagagcagcctggccaacatggcgaaaccccgtctctactaaaaataaaaaaccaaaccaaaacaaaacaaaaaaaactagccgggtatagtggtgcacgcctgtaatgccagctacttgggaggcagagggcagaagaatggcttgaacctggaaggcggaggttgcagtgagcccagaccacgccattgcactccagcctgtgcaacggagagagaatctgcctaaaaaaaagaacaaaaaacaccaaaaaaaaaaaaatagtggatatcaaaaatttatttaaaaagaacagtaaaatctctcattaataattgtttatactgatttcatgttgaaatgatattttgggtagagttaaatatattattaaaattaatttcacttatttaattttttaaatgtgactacttgaaaactcagggtgcagcataccaaacacaccaacatgacatgtttatgcatatgtaacaaacttgcatgttgtgcacatgtaccctagaacttaaagtattaaaaaaaaaacaaaccattgatttttacatttaaaaaaaaaaagaaaattcaaagttacatttatggctcacattatatccctatcaaacagtgcagatatagatgattcccattaatattcttttttttttttttgagatggagtcttactctgtcgcccacgctggagtgcagtggcgggatctcggctcactgcaacttctgcctccccggttcaagcaattgtcctgcctcagcctgctgagtagctgggactactggcaggtgccaccacacccagctaatttttatatttttggtagagatgggttttcactatgttggccaggctggtctcagactcctgacctcaagtgatctgaccctctcagcctcccaaagtgctgggattatatgtgtcagccactgtgcccggggcctccattaatattctttagtattttcactaggcacctcacttaattacagaatgcctatcccattcaaagcttgagaatcaacctattttctcttgctgaaaagatagctttgttttaccaggtttcaagaaactgataatgggttgggaagagtaatgcattgcccatggggctgatgctaatctgcccctctcctgtgagctctgcctccagaacctctctcaaatctgcccctctcctgtgagctctgcctccagaacctctctcaaatctgcccctctcctgtgagctctgcctccagaacctctctcaaatctgcccctctcctgtgagctctgcctccagaacctctctcaaatctgcccctctccagtgagctcggcctccagaacctctctcaaatctgcccctctcctgtgagctctgcctccagaacctctctcaaatctgcctctctcctgtgagctctgcctccagaacttctctcaaatctgcccctctcctgtgagctctgcctccagaacctctctcaaatctgcctctctcctgtgagctctgcctccagaacctctctcaaatctgcccctcccctccttccccagggctggctgagcacccctcagggtgcatatggtctccctgcttctcctctggtctaattcttttgtgttagagtgacctctctaagagggggctctttacctggaaactgcaaaccactaagaggatcatggacagaatttaggctgtctgtgaacgtggatgtcaacacatcaaacaaaaaagcagcttttagcagtatctctgctttgccactgacagaaaggtagatattttagggtaatattctagctgctgtaataccaccactgaaattgtagtactagtagaccctccagtagatgttatttaatgtgttaatgaaaaagcacacatttattataccacacattttcttgacattttcatagctaaggctttttattcttatttatttatttatttttgagacggagtctcactctgtcacccagcctggagggcaatggcgtgatctctgctcactgcaacctctgcctctcgggttcaagccattctcctgcctcagcctcccaaataggtgggattacaggcacctgtcaccacgcctggctaatttttgtatttttagtagagatggggtttcaccatgttggtcaggctggtctcgaactcctgacctcatgatccgcccgcctcggcctcccaaagtgctgggattacagatgtgagccactgcgcctggcctctaggcttttattctatgcatttatttattacttattttattttatttgagatggagtcttgctctgtcaccccagctggagtgcaatggcatgatctcggctcactgcaaactctgcctcccaggttcaagcaattctcctgcctcagcctcccgaatagctgggattacaggcgtccaccactatgcccggctaattttttttttttttttttgtatttttagtagagacagggtttcactgtgttggccaggctggttttgaactcctgacctcaagtgatccgcccacctcggcctcccaaagtgctaggattacaggcataagccaccgtgcctggccttattctatgcatttaaaagcattttttttttttgagacagagtctcgctctgtcgcccaggctggagtgcagtgacgagatctcggctcactgcaacctccgcctcctgggttcaagcaattctatgcctcagcctcccgagtagctgagattacagggacctgccaccacgcctggctaattcttttgtatttttagtagagatggggtttcaccatcttggccaggttggtcttgaactcctgatctcgtgatccacccacctcggcctcccaaagtgctgggattacaggcgtgagccactgtgcccggcctaattttatttttttttctgacttggagcccttaggcttcaccaagctttcaaagcctgaaagcctcccactgccttcaggaggcagccagtactcactcctccgctcagcacaggagaccctgctatctcatcagccataacttcggtgacttctcagtgaattcagcatttactgatgcccacatatgctcagccttcatttgcctttgtgccttctaaatctgtttccttccttggaatgtcttatcccaaccttttaacctggaaactttctacagtctcctatgataatgtaccatacagttcaaagggcagacttttcatgtacacggctcctggagggaacaatatgggtctttatctttattgtccatcgcccattccaagtttccctgaagtgtggtatgcactgttcttttttttttttttttttgagacggaggcggttctgttgcccaggctggagtgcagtggtgtgatctcggctcactgcaacctccacctcctagttcaagcaattctcctgtctcagcctcccgagtagctaggactataggcgcgtgccaccatgcccagctaatttttgtatttttagtagagacggggtttcaccatactggacaggctggcttcgaactcctgacctcaggtgatccacctgctttagcctcccgaagtgctgggattacaggtgtgagccactgcgcctggcctgcagtgttcttttagataacaaattaatgaacatttttcatattaatatgcaaatattaagagagtgatttcttatttatggcagtatttgtaattcctttacaaatcacttttaagtttaaaaattaagtaaacaacagtatatattgacacagacatggcaacatgatgatggcagcagataactactgcgtctacttctgagtagttggagatattctgtgtactatttttcagacttttttataagtcagaaattattcaaaaatgaaaagttaaaaaaagaattaaagcaagtatgagataatgtattaactttatattaagtcaaaatctaaataaacagctattttctgggtaggcatggtggctcatgcttgtaatcccagcactttagccaaggccggtggatcacttgaggccaggagttcaagaccatcctgtctaacatggtgaaaccccatctctactaaaactacaaaaaactagctgggcgtggtggcacacacctgtagccccagctacttgggaggctgaggcatgagaattgcttgaacctgggaggtggaggctgtagtgaactgagatcacaccactgcactctagcctgggtgacaaagagagactccatctcaaaaaaaaaaaaaaaaaaaagaaagtattaaagaaaatgctggcatttgactaaatcagaataaataaataaatagaaagaaaaagttaaaaaaaaaaaaaagctacactcctagcctgggcaacatagtgagaccctgtctctaccaaaaaaaaaaaaaaaaaaattagcaattagctggacatggtggcatgtgcgtatggtcccagctactctgaaggctgaggtggaggattatttgtgcctaggaggtcgaggctgcagtgactcagttacttgtgactgcaccactgcactctagcctgagtgacagagcaagaccctgtcttaaaaaaaaaaaaaaaaaaagccagacccggtggctcacgcttgtaatcccagcactttgggaggccgaggcgggaggatcacgaggtcaggagatcgagaccatcttggctaacacggtgaaaccccgtctctactaaaaatacaaaaaaaattagccgggcgtggtggcgggtgcctgtagtcccagctactcgggaggctgaggcaggagaatggcgtgaacccgggaggtggagctagcagtgagccgagatagcgccacggaagtcccgcctgggcgaaagaggaagactccatctcaaaaaaaaaaaaaaaaaaaaaaaaaaaaaagctgcaatcaaaacatgttgctcagaaagttaccaggcccaatcttcatgcagtcccaccaagagttcagtgaacagacattgccaatgttatttaggacacagaactctattgagcactatattcactatttgggtgatgggatcagtagaagcccaaatctcaacatcaggtaatatatccatgtaacaaacctgcacatgtacctgtgaaatgaaaattataaaaaaaaaagggggggggggaggggaagaaaataatgaaagacttactaaagcattctgccagaatagtttaacttggatgccaaaaccaggcaaggtgctcaaaagcaaacgtaaacaaaacaaaacaaagcaaatgtatcaaatgaaatccagtagtgaactatttaatacattatgtacagtcggatttaaaggaacacaaataggtttaaataatttgaaaattttaaagtaacttagactttcctacattaaataagaaagtagaacccgattgaggaagcctaacataaaaaaaaaaataggagaggcaatatcttaattttgatgtgaactacataacatttaatagaaatgcttctaacctctcactgcaaaataaaaaggaacaagtgctgttattacaatattcctggcaatcctaaccaatgcaacaggaaaaggataaaaattaagaggcttacattttagcaaagagaaaaactcatgtgtagatgtcatgactgcttatttagaaaatcaaagtgtgttatcaattaaaaactatgggagtggccgggcgcgatggcccacacctgtaatcccagcactttgggaggccgaggcgggcggatcacgaggtcaggagatcgagaccagcccggctaacacagtgaaaccccgtctctaccaaaaatacaaaaaaattagctgggtgtggtggcgggctcctgtagtcccagctactcgggaggctgaggcaggagaatggcgtgaacccgggaggcggagcttgcagtgagccgagatggcactactgcactctagcctgggcaacagagctagactccgtctcaaaaaaaaaaaaacaacaaaaaactatggggcggggggtggggggccgggtgctgtggctcacacctgtaatcccagcacttttggaggccgaggcgggcggatcacgaggtcgggagatcgagaccacggtgaaaccctgtctctactaaaaatacaaaacattagccaggcgcagtggcgggcgcctgtagtcccagctactcgggaggctgaggcaggagaatggcgttgtagtcccagctactcgggaggctgaggcaggagaatggcgtgaacctgggaggtggagcttgcagtgagccgagatcgcgccactgcactccagcctgggcgatagagtgaggctcagtctcaaaaacaaacaaactaaactatggggtggagggagggggtaacaaaaattactagaattattaaacgaggtcagtaagatgactgaaagacacaaaaatcaaaagcatttttatttgggacagaatcttgctctgtcacccaggctggagtgccgtggcgcagtcttggctcactgcaacctccgcctcccgggttcaagaatttctcctgcctcagcctcctgagtagctgggattacaggcttgcgccaccactgtaatttttgtatttttagtagagacggggtttcactggtcaggctggtcttgaactcctgacctcagttgatctgcccacctcgggctcccaaagtgttgggattacaggcgtgagcccccatctccaaatacatacatatcaatctgtactatcatttttaatagacatggtacaaatatatgagaattgctgtaaacattaaccaattatggttatgtacttattttttgagataggggtctcacaggggaatgaaaatgtgggagtactttaacaattttattttttttgagacagtctcactctgttgtccaggcaggagtgcagtggcacaatctcggctcccctgcaacctctgcctcctgggttcaagcgattctcctgcctcagcctcctgagtagctgggattacatgtatgtgccaccatgcctggctaatttttgtatttttagtagaggcggggtttcactatgctggccaggctggtctggaactactgacctcaggtgatccgcccacctgggtttctcaaagtgctgggattacaggcgtgagccacggcgcccagcctttttatccattttatgtatttaagattttttggaacctgattcacacaatagactagaacacttcttaaatcctattgagtactgacaatcttaacagtctattagttttatccctgtgtgattagtgggcagcaatatcttagttttagctaaccaagcctaagcttcctgctcttctcatgtaaaatcatgtatattcaggaagatgagttagctttattaatatacaagtaagaaaatgagctgtggaaagaagtatatgcaagaaaggttaagtgacaaaaaactaaaaaaaattagtgattttggaaatctgcctttacttcacctgccatctccctagtgactttaagaatgctacataaagccaggcgcggtggctcacgcctgtaatcccagaactacgggaggctgaggtggttggatcacaaggtcaggagatcgagaccatcctggctaacacggtgaaaccccatagctactaaaaatataaaaaattagctggatgtggtggcaggtgcctgtagtcccagctactcgggaggctgaggtaggaggtggaggttgcagtaagctgagatcacgccattgcactccagcctgggcgacagagcaagactctgtctaaaaaaaaaaaaaagaatgctacataaagtttaaaaaccaagaacatttggacagattttgaaattgcgaattaatgaaaaagtaatttcacctttattgatcagctcagttgctactgtactgcagagcaatcagataaattgtaaactttcaagacactaaaattagcatattccaacaaagttatttatataacagctatttttctagtctaattttctagtatttattggaaattccccaaatttgtttcaattctctaatcttatcccagccagtatcatgttctgtttaatgtttaacacgctcatattattaaaatatagaagtcccaggtctaaccttcaatggacaagtgttgtatgtcactatttttttgcctacttgtaaaaatgactgaacaaactttttagatgaataattaagcaacacatatccctcttgctttcattgccattttaataaaaaaatagaaactcctatattacaaaatatattgcgtattgatgtcacaatggaaattttacaaacacaaactttataaaagggtgttaagccaattccagatgaagcaaagacatgaaaacaggaaagttctagaaaaagtctaaataaatgtattcatattctatgagtgaaaaagataattcaaaaggatgtccgtaaggctgggcacagtagctcacgcctgtaattccagcacttggggagggtgagatggggggactgactgagcccaggagtttgacaccagcctgggcaacatggtgggacctcgtctctacaaaaaatacaaaaattagtacggtgtggcagtgcccacctaaagccccagctactgaggagactgagacagaaggattgcttgaactcagaaggctgaacctgcagtgagctgtgatcatggcactgcactccagcctgggcggtaaaagtgagatcctgtctcaaaaaaaaataaattacaaaggactcatatatattaattttgtataattcctctaaaggaattattcaaagatctcaaaatatttcactgatatttaagtcctgtaagtttccatttcatacggtgatataagaccatgggtagaaaatatcagtgaataaaggaagacatgccaaatgtctcatctgaatattttgaccactccatgatcttttgtcctcgcactgctgcatagttaggtttcagaggctggtcttcgaattaggtcaccaaaatatgcagtgattgtcttcaatttgttgttaaggaagttctgttctatttccacttgccctccaggcccagctaaggatgccacctaaaaagatgaacagaagaaagtcacacacacacctaagcataatttcaaaagtctttcacttgtactgctttatcaaataataggaagactctggcttttaagtcctttgaataatgaggcatctcaaatgttcactgctacttcccaggcatcgatgcaatatttggaggtgggaatacttgttgtttcagatccttttgatattatgggaaagttcatggtgctatgctgttgtataggacttgaactctccaatgccatagctaaagaactcaaaggaagaaaaaaatgaacaaatccttcccaaacatccggtttattaaaaatttttatttttatttttaaacattagaggcagggtctcactgtgttgcccaagcttgtcttgaattcctggtcttaagcattcctctcacctctgcctcccaaagtcatgttatttatttatttattttgttttttgagatagagtcctactctgtcgcccaggctgcagttcagtggcacgatctcggctcactgcaacctccgcctcccgggttcaagcaattctcctgcctcagcctcctgagcatctggaattacaggcacatgccaccatgcccagctaatttttgtattttttttagtagaaatggtgtttcaccaagttggccaggctggtctcgaactcctgacctggtgatccgctcggcccggcctcccaaagtgctgggattacaagccaccgtgcccggcctaggtttatttttaaaatattattactatttttctcaccttccttcctcctcctccccaggtttatttcttaacaaaaatatttaaagattatttccctttcctgaaagcctttgtcctgttcatcttaccatctgcatctcaccctctctctcattcattcaacagtatttactgagttcctgctacataatcctagctctgatccaggccaggatccctactctcatgaggagtctagcctctagtgacttttccttctctcactggtctcctttcttttctcccaccttggatcacttcttctacctgttgcccttttgtgtattttaaatgctttctacaacaattgtggtaaaaatatattgctctttatgaggtctactagaatatagtgactaactcctttattaggaagctgaaagcattgctccctccccacttccacagttagcatcaaaaggatgctagccaaatgaccagcatttgacagggggatggggcagttagctgctaaagattcttttgttccaagacagtaatgcttgcactcaccttggtacattgtaaactagaaatgttttaccctaagttcttatacagataaggttttgttttggttcagtactttcttcacccaacctctcagaaatcttttaaatagcaaatacgttactttctaactcatgattacttcaccagagatcagtttttcagggcagtaagaggaaagatcaagagaagagagaatgcagtggagagtcctggcaatcagagaatagaagacacagcaatagaaaaaaggacacccaaagtattagggacaggcataaacaacgagaaacaagcattttctgtaactgtaatcagcaaaattatagttgtgtattttgtaaagagaggcagtcagtctggtttggttttaagggttgagagaatgtaatctcagtggtattatgcataccaccccagcttctggtagaagggaagatttattgtgcatttccagtgagtatgtaaaaagtcagacacagaaatagggaagaagcaaccagtcattctatggtaccttattatgtgaaaataagtcaaatacagtattccttataatgattaaaagcaccccccccactatttttttttgtaaggaatacttaaagtaaaactagaaatttaaaaatagcaaagtagtctcagagtagcaattattaggaataaatgttttaaattctattttggtatctttatgccagtctggtttttttacaagaaagggggaaaaaaaaaaaaggtattttgtctttagccaataaaaggcattttgaacagtttatcattttttcacaataatttttctttgagttgagatataactcaaatgtagcatttctgctgtgccctttaacaaataacttaaaaatccccagttctaccaataatttgctgcattaatttaaaaagccatttttaggccgggcgctgtggctcatgcctgtaatcccagcactttgggaggtcgaggagggtggatcacgaggtcaggagatcgagaccatcctggcaaacaaggtaaaaccccgtctctactaaaaatacaaaaaaatttgctgggggtggtggcgggcgcctgtagtcccagctattcgggaggctgaggcaggagaatggcgtgaattcaggaggcggagcttgcagtgagctgagatcgcaccactgcactccagcctgggcgacagagtgagactctgcctcaaaaaaaaaaagaaagaaagaaaaaaaagccaatattgttgttgttaagaaagccattaaaataggctttttttggctttctaaatccgaaaatatgaatactgtctatctccctaaaagagcattttaaagaaaagaaaaaccatttctgaagtttacttgaaaaaaaaaataaaggtactataaatatagttattattattattattttttgagatggagtctcgctctgctgcctaggctggagtgcagtggcgtgatctcggctagtactgcaagctctgcctcctgggttcatgccattctcctgcctcagcctcccgagtagctgggactacaggcgcccgccaccacacccggctaattttttgtatttttagtagagacagggtttcaccgtgttagccagggtggtctcgatctcctgacctcgtgatccacccacctcggcctcccaaagtgctgggattacaggcgtgaaccaccgtgcctggccgatggttattatttcttatataagccataaatagaattccataaaaaccaatttctgagaaggtagggtagttttgtttttcttaatacctttcagagggttgtctagatgtgggtgcttgggattaaaataaaaaacggaggtaagatgggggttttctcagaaatcatgagttctgaagtctgtgcaaaacagttcataaattactttactcaataactattattgagagcttataggtatctgttctaggcagtgcagtatagcaaaaaacaaacaagaaatccccacctccatgaattttacattctggctgggagagtcagatgacggacaaataaaacaaatattgttcctgataatggagaaggtggtgtatctatgtgacaaaatatagtgagaaaactcttatgtacttagtgtttagaaacaatttttttttaaaaacttaacatgttactttcaagtgttctgattacgaataatttctaactttagcctttttgtacgttttcacattttcacaactacatatatgttaaaagttagaattttggagttggaaagacttataaatttagtagttcaagcaaataactttgcttcaggcaaatttaatgaatgccattaatttctagctgtccttatttttcactagaaggtttagctgacaaaatattatttggttccctccgctttcttagaatttagtaaattgtgagaggaagagagggtaggaaggacagagagagggagaacggcaggtacaaacagataaagattagcagctctggattactttccacatttttggcatattacctaaatatattaattttaccaaaataaatcccaattaatttggaccagaactagaattaaggactgaactcttgactagatttgacatcaggcagtaaagctgttctttggggactggcaaaggagaaacagaaaagatcacaatgaaggtgttaaagcacagaagatttaagcatcaaataaaaaggtaaacattcaaaactgcactttttttttgttttgttttgttttttgagacgagtctggctctaccgcccaggctggagtgcagtggcatgatctcggctcactgcaacctctgcctcctgggttcaagcgattctcctgcctcagcctgccgagtagctgggattactggcgcacctggctaatttctgtatttttagtagagacagggtttcacgatgttggccaggctggtctcaaaactcctgacctcaggtgatccgcccgcctcggcctctcaaagtgttgggattacaggcttaagccactgcgcctggagcaaaactgcattttttcaagagaaactggatttaaagtaaaagtgcacgataaccgaatgagagaaaataacctcccaactgcatcctactggaagaatagataataattactaatgacagatacactttattacaagaaagacacagaaacatcagagcgctgaaaaatgccctattactacatggaagagattatatctatgatacgcagaaatgtggttagaagataattgtaccaggtaaaaataaagagcattttatgtggccttcagatctgtgtcaaatacaatgctagcagcagcaagcatctgaaatactggctgtgactacaaacctaatgtcacacaggccaagaaagtgctttgccactagttatttttgtccctcttttcttttgctagatccaactaataaccacatgaaaataaaacaaagttggcaaaacatttctggtaattaaaagttctctgggaggtgaaagaagaaaatattttttagtcagcagaaaatcaggcatcattcaaacttaaaaatattcattaaattagttccatttctctgcacatggtaaacagaaatgagatttctatgcttccgtaaataaatcttaaagccaccatcatcaacaggagcacctggtggcaggtcaaaaaaattctcccaggcagccaggcacagtggctcacacctgtaatcccagaactttgggaggctgaggcaggcagatcacctgaggtcgggagttcgagaccagcctgaccaacatggagaaaccccgtctctactaaaaaaaaaaaaaattagccgggcatagtggcatatgcctgtaatcccagctacttgggaggctgaggtaggagaattgcttgaacccgggaagtggaggttgtggtgagctgagatcaggccattgcactccagcctgggcaacaagagcaaaactccatctcaaaaaaaaaaaaaaaaaaaaaaaaatctcccaggcatcacaagctaacctacactggtgtcctaagaggaggctaccaggccgggcacggtggctcatgcccgtaatcccagaactttgggaggctgaagtagaaagattgctttagcctagaagtccaacctgggcaacatggtgaaaccctgtctctacaaaaaataaaaaaattagccaggcatggtggcccatgcatatggttccagctactctgaaggctgaggtaggattgttttagcttgggaggttgaggctgcagtgagctgtgatcatgtcactgtactgcagccttgtggacagagcgaggctctgtctccaaagaagaaaaaaaccccaccaaaacccaaaaacaaccagttcactaagctctgtccttcactcattctcagaaccagatactggaaacatgaagactgttcaattcaacaaattacagaaagcacaggctatcagcacaggactggcaggtagcactatgtgtggcccagaagagctggatatccctctaatttctatctccatcatttacggagaaaagttacccacagtagtgaaggagatacaagctatatttagaagcatttcagcaaagctgcatttgtgtcttcccaagtcaactatgaggccaatcagttccacaacccctctagagcatgacaatatatatggtttaagaacaaacgattctgctctaattgtgcagcagcaaacttcccaagggaaaagctggctagtaaatattaggaactgatcatatatttatcctgaattgcagtgtatgattaagcactgatttcaccaagaacagagttggtctagaaaactgggaatcaggctgggcgtggtggctgacacctgtaatcccagcactttgggatgctgaggtgaagaatagctggccaacatggtgaaaccccatctctacaaaaaaatataaatattagctgggtgtggtggtgcacacctgtagtcccaggtactcgggagaccgaggtgggagaattgtttcagtctgggaggctgcagagagcagtgatcacaccactgccctccagcctggccctgtctcaaaaaaaaaattggtaaccagttactgaaagagtcaaattctacttattgctccaatctcatggtgagctaatattgtgataacagataaatctgtttcttcttggtaatgaacattgtgtcagtgggagacagcaaggtgtaataagactgtcgctgtgtggcagaatgaccaggcaaatcaccatacctccatagccttcaatgaaatcaggtgctccagagacacactgctccagaatgtcatgatgagtcaatgaaatggctaagaggacactgtacaccctaagtgtgtgtgccagctcctatgctgtagatgtatagtgtatgaatgcagggtattgttagtgtagtaggatgaatgcacttgtgagtgctctgagttgtaaaggctattttcaagtaactagtttggagtctactattccaacatgtaccagaagcccaatccagttcttagccttctttttattctctcatgcttctggttcctaaacctggactaatgttctctacctctacgagagagctaccattagattagatttccactatattaacttaattcagaaaaatttgacacaaattaaaaaaatctagtatttagaaaccagttaggacccaatcttctatttattaaatacacagatactataactacttagaatatttgcaaagaaatctgtgataaatttttattgtatttagccaaaaccactacaaatgctaataaactgaatttagttatatttaaaacattttccacataacaatattacctaatagtttttcatatttattcaaattcaaatacattattcaaatacttacaactctattcttatatatggtaagttttagaaatattacctataaataagtacctcattcctatcgacctatctatctatctacttacctacctaccaaaataaagtattagaagtgactgttacaatgaggaacatttcccttcctagacatcagagaacaaaatataaaaagacaattggagcctcaagtactctgccaactaaggagagatgagggccaggcgtggtggctcatgcctgtaatcccagcactttgggaggccaaggcgggcagatcacgaggtcaagagatcgagaccatcctggccaacgtggtgaaaccctgtctctactaaaaatacaaaaattagctgggcgtggtggtgtgcgcctgcagtcccaactacttgggaggctgaggcaggagaatcgcttgaacccgggaggcaggcagaggttgcagtaagccgagatcgcaccactgcactctggcctgggcaacagagcaagactctgtctcaaaaaaaaaaaaaataaataaaatagagatgaattccattattaaatctaacatcaattttatacttagcatttatttaagtattagttaatttgtaaacttctccgctttatgaggaaacaaaaaaggtaggatttgccttgcccaaatgttacatgacatgtgcctggggaagccatggaagggtggagtatgtctgccagacaaaggccaggaattaacatgatacagttcattttttaaacataattcttagggatgttgcttaatgttgccctgctctctgtattggtaagatacataccatgacaaagataagtaacttaacttatcttacctgaaaagtacaaacgctattgcttttctgagactagatgttttatgtctaattataaaattaaacaaggatgagaagctgccatatcaaacagaccagcagacttcctctcttcattcctgccttgggctaagagacaagactccttatgtcagagattccacccacaaggatttcacacaaaggtggtgtttgataattgctgcctgcatcttcctttgggaaggactaagtccttgtctgttcagagatttccatctattaatatgttcaataggtttaccacttaagagagaaacctttcatgtttgtgaagcatggtagatccaaagctagaattagctggaattctctggctgtccaaggaaagaaagaaaggaaagcatggcaagatacaggcatcaagcagataaaatatgcttactctgggctttttgaattggatttgagatcccagcactttagaaggctgaggcaggaggatcacttgagcccaggaatttgaggttgcagtgagctatgatcatgctactgctactgcactccagtctgcgtgacagagagaaaccctatctccataaataaataaataaaataaataaataaataaataaataaataaaaattagtccttgggtttcaacagcaatttcaagagtaaggtatataccttacctccaaagagcctctcacaaaaagatatttttctaaattactcagttcttagcaagaaatgcatgtcttggaatcatctccaaatagaaaaaaggaatagaagattgaggtaataagggacctaggaaaacatttcactagatacaggaaacaattagcatcacctcttacatggaaatatttgagataagttgtatttgtattgaatccatgctgtttaggatgatataaatattatctaagagtacaatctccctggatgggaaattaactgtccttcatatgagaaaaatgcagcctgagtttcaaacagcagacagtgacacacttgggatcccaaaccatttgcaaattcaggttgttgtaacattctaaaagttccttaacaaaaaaaagttaattttctttgcaactgtgatttctctcaatttttagtactgtctcctctcattaaaaaggtcctacatacggatacattttcttcattaaaaaaaagttgatttcccactgtaatctgatcaaaatttgatagcaaaaaaaattaatacacttgaaatctcatcagtctttcttggtttcattattccctagtgtcattactccctctagaggcaaaaaaacatgaatcaccaactccaattctggaaagtttttgaaataaaccaaaaacacaaattaaaaagttaactttctctttgtgaaagtgttaatgtaactagaaaaagttaatcagcataaattgcatgataaacagtgaatttcctctcgttattcaagtactcaaatgaggctgtctacatttaatataaggatcacatcgtctctgatgagacgatgtagagcataatattcatatatgagaaaaacagaaattatggaattgttaggctcaaccttatctggcagtctagaaagcagctctgaatcatatgagaagtagatataaaatacctggtgtttaatatgaactttaattcagaaaactcatgaagcaaagagatggttcgtttttctttctttctttttttttttagacagtctcactctgtcacccaggctggagtgcagtggcgcaatctcggctcactgcaacctctgcctcccgagtagctgggattacaggcacgccgccaccatgcccggctaatttgtgtgtatgtgtgtgtacatatatatatatatatatatatattttttttttttttttaagtagagacggggttttgccatgttggccaggctggtcaactcctggcctcaagcgatctgcctgccttggcctcccaaagtgctgggattacaggtctgagccatcacacccagcccctttttcttataactggtataacacatactattaatattttgggaggcctaatgtttagacacaatggatcataaacggatgaaaggcagcaagatctaatttcattgcttaacaataagagctcaggcttgttaatttcatttactcttcttttaacccagagaatctcaactctggagaacattacaaccactttgggcagtttaaaaattaactttcgggcctcatcctagaaaacagagaatctttcccagtggattacagtatcagtagtttttttaaaaagctactcagctgcgcgtggtggctcacacctataatcccagcacttagggaggctgaggcaggaggattgcttgagcctttaattttgagaccagtgtgggcaacatggtgagacctcatctctataaaaaataaaaaaactagctgggcatggtgctgtgtacctgtggtcccagctactcagcaggctgaggtgggaggactgcttgagcccagttagttgaggctgcagtgagccgtgtttgggccattgcactccagcctgggtgatagagcgatatggtctcaaaagaaaaacaaaaacaaaaacaacaacaaaaaaagcgctactcagcaaaaactataaaactctaaagcacaggcaacacaatgaagaaagataaattggacttcataatagaatagaaatgcctgagttggaaaacctgatagaggtaatgtagaaggaaaaaggaagcgacaaacactagaaaatatagataggaaagactaacattctcttataatagatgtcccagagaaaggaaaaaagacaatgagaacatagttgaagaaataatggagatagcatctcctgaataaaaaacacaaaaaacatcagaatactagggctagttctgatagtatcaaataagagagaaaggaaaaacccagttagcaacattacagtgaaattcagaatatgaaagataaaagattttcaagagtatctggcacaaagcagatatgtagtaagtgtttgtagaaaaatcaacaaaaacatgaaatgaagatatcatcaaatctctctattttttttgagacagtctcactttgttgcccaggctgaaacaaataaaaaaaggctagtttaactatgcagcatttgaagcacaacacattcaatcatcctttcaatatgtctagtcaaagcctagagtgctctatttgtgtctatgtatgccaggagctatggaaatgaagacgatgtaatagacatggatctaacttccagatacttccagctttctaagaatagtcagtgagtcagggacttctcataaacacattctctggtaaggatatgtcctgtatcttttaccgtacttgtaacagttaagacagacactcatggaagtttttcagtaatggcttacctggtcaatatcagcatttcttggaagaaaataaacaatattattagtgatcttcttagaaagtctgaaaatttcaaaggtggacagcagttaaggaaaaataatgaaaacagcagctaatttctaatcagaaagagatgtgttttacttatcataactcaaatgtatttcaaaatgtaactaaaaggcaaaaacttaaaccctctaatggaacctaaaagagaattacatacaaaattttgtaattcagcaaaatattgtctccataattcaaagactgacagttgaatcgatttcaattatcctagtggtaggtactaggcaagagtcagaataaagtaaccatggccattgcagtctacactctaaattttattttttaatttttaattttattatttttgagacagagtctggctctgcttatttatttttgagacagggtctggctctgctgcccaggctggagtgcagtagcgtaatctcaattcactgcaacctcctcctgggttcaagagatcctcatgcctcagcctcccgagtacctgggactacaggattacaggcatgtaccaccacatgcccagctaatttacactgtaattttttttttttttttttttgagacagagtcttgctctgtcacccaggctggagtgcagtggcaggttcaagctattctcctgcctcagcctcctgagtagctgagattacaggtgcccaccaccatgcccagctaatttttgtatttttagtagagatggggttcagccatgttggccaggctggtcttgaactcctgacctcaaatgatttgcccgccttggcctcccaaagtgccaggagtacaggcgtgacccagcatatcccggagtacactctaaattttaatagcaatttagaaatatctttcctgttaagagtcaaacagatgattacggtggagaggcaggtggcgaaagtgacaaataacaaacgttactctatgaaataagggtttgcttcgacgtcttggaagagtaacaggaaaaatgtgaagatacacagcaaattagccagcatcagtgtgaattgaagagttacagaatggttaccgacaaactccagggatactttttcaggaaattcccaattcacaagacaataagtccaatgtatgtgttctagtccttgttttataatggtaaaattgaatagcaagcaacggaaatatgacctcatatttgtcattacttttggagatcgatccccacataatttttacatacctgctttttatgtaaaataaactagaatttatcaaatgctgttaggtaatactccctctcttcactaatttctctctctccctattttttgaaatggagtctcgctccgttgcccaggctggagtgcagtggcgcaatctcagctcactgcaacctcggcctccctggtccaagcgattctcctgcctcagcctcctgagttgctgggagtacaggcgcgtgccaccatgcccggctaattttttgtatttttagtagagatgaggtttcaccgtgttagccaggatggtctcgatctcctgacctcgtgatccacctgcctcggcctcccaaagtgctgggattacaggtgtgagccaccgtgcctggccaatctctctctctcttttgagacagagtcccgctctgtcacccagggtggagtgcagtggcgcgatctcagatcactgcaacctctgcctcctgggttcaagtgattctcgtgcttcggcctcccgagtagctggattacaggcatgtgccaccatgcccagctaatttttgtacttttagtagagacggggtttcaccatgttggccaggctggtctcgaactcctgacctcaggtgatccacctgcctcagcctcccaatgtgctaggattacaaatgtgagccaccctgcccacccacccacactccctctctcactctcatgctcctttctctccacaatcattgtttcagtaaactcttaagacttccaaaggatatccatcaggagacatcattgttctaatgtcaaaggtctctgcagtggcatagtctggccctccccaaggtgggctgaggaacacaacatcagcctttaaaaaagaagccagcagcaagaaatctccacagatgaactctatcttatctgctatcccataaacttctgcattattgcgagcaagggcaatcttaacaggatcgatatcaatggcaatcactaaaagtgggaaaagaaaaaaatgagttaattgacactgattattacttatataggatagtttgtctttcgaatagtctgtcatcttctgtataagaactctttataaagaggaggagacatttggccacatatacccgagatacagacttcagtttaaattttttatttaaattagaaagattcactgtgttgtaccaagaaaatgtgactgaaggacttggctttcttgtctcattctacagcttttcctaaccaagtggcaatacttgccacaaaatatctactgagtgccaaatatgtggccaccattgtgctgtgctacagcctgagtaggaaaaggtcactgactgtatggacagcctgtaatgctaggttttggaaaagacaaacttcttcaaaatgtagcaatgcccaaaatgaacagaaattcgacaacttctaaggaaaaccaaaataaaacacatttttacaaacttatgtagccaagtgatgaagaagaaatgtaaaaacacctgccgataatgtttaatgagacattttaggtagaggaaacagatattatgttttcaccaaatggactttcatttactttattccaattttataattagcaacatagacataatttaattttagcctatccatgttttgcagtaaactactcagtatacttgcatgtgcactactgtgttaatggcagtattcttcatttgaaagggctaagtgtaaccgttacgcttacggcgtggttgtcatggaaattgttttccagaaaatgctagtttccaaatttgttaacatttttgagaaataatgtgctaaatcacttgaacaataaatataagcaccaattataacatattgcaaaatcatacatttataatggtgtttttcctgttctaaataaacattcacatttgtaagagaaaaaattcttttcagttgctcagtctgttacatttatagttcagagacagatggaaagatatgctgatgatactaaaaattcaaaagaaaaagtgactttcaacatcaaatcgattcagttaatcctattttcatctgtatttcttttggaggcagactttgtttttaggcctaataagaatggaagcattcaatctatcacataaaagaaatgctggctgggcacggtggctcatgcctgtaatcccagcactttgggaggctgaggcgggtggaccccttgagctcaggagttcgaaaccccatctctatcaaaaacacaaaaaattagccaggtggggtggcatgtgccagcagtcccagctactcaggaggtttaagtgaatcacttgggcctgggaggaggagattgcagtgagctgagactgtgccactgcactccagcttgggtgacagagcgaaacacctgcaccccacaactgcaaaaaaaaaaaaaatgctgacaaatgttaatcctcagccacactagcacacctcgacagcttaatagccacatgtggctactagctggctatcatattggacagcacagatatagaacatataagtcatcacaaagagttctaccacgcagtactgttgctaaactactggaacacatacaaaagggaaagatatctttaacaacagcaatatgcaaaactttacagattccctaaccctttagttatgcacagacagcaaatatctgagtggaaataaatctggcacaaaggaataacctcaactatatgcaatgccatctttttgtatgtatgttgcgtatatatgtaaatgtataaatacacatacagttcaagcataattgattatatgaatgtgtatataagcacacatgaacatacattatgatagggaactcaaaacagttaagtgtaattgattatatgtgtatacttacatatgtatacacaaacacactaagtcaattatactttaactgttttgaattccttgccataatgtcattagctcaactatcctacggggatactaggaggagtattgataactgccattcatggaacacctgtgaattaccacttctggtttggggggcaaattatgggaccccactacaggctaggggtggtggctcatgcctgtaatcccagcactttgggaggccgaggttgggtggatcacctgaggtcaggagttcgagaccaccctggccaacatggtgaaaccccatctctactaaaaataaaaaaattagctgggcgtggtggcaggcccctgcagtcccagctatatgggaggctgaggcagaagaatcacttgaacccaggaggtgaaggttgtagtgagctgagaatgtgccactgcactccaacctgggcgacagagtgagactccatctcaaaaaaaaaaaaaaagaaaaaaagaaaaagaaagaactatatatctacatatagacggtttgagtaatctgagaaaattttcagatttctttgctactttatcagggagcaggtgttttaatcatcgtgttacgatgtaaggccaaaatgaagatacattttacaacttttaaggaaaaatcattttttaggcaataaatattgattttagcccataggaatggcttatctggcaaagtctgagaagaactttgatagtaaatagtgaattcaaggaaaaaggaaaataaccactactctcgtaaatatgaaagtagcttgtctctaggctatcaggagaccatgtaaagttagcaaatatcatgccttggagcataggagtattaaaacgggccacatttcatcagcactgaccttaaaaatctatgtaatggtaaggcgataagatcattttcagtgcaatttgattaaagtcagtttctctgactagaccgagcaaggactgagtctgttttttctacagctactacgtgctttccaaaatcatattacgctttcctacttaacattaacttcatataaattagtaagattcagtagaaatctgttgaaatgaaactatcattgataggtataatatgaagatatttaaaaatatgcatcttcagaatgaaaactaaatgaaaactctgcttcttgtctcagtgtggtggctcatgcctgtaattccagcacttttagaggccaaggggaggattgcttaaagccaggggtttaagaccagcctggggaatatagcaagatcctgtctatacaaaaattaaaaaaaaaattagcagggcatactggcacgtgcctgtagtctcagctacttgggaggctgagatgggaggatcacttgagcccgggaagtcaaggctgcagttagtcctgatcatgccactacactttagcctgggtgatatagtgagaccctgtctcaaaaacaaaacaaacaagaaaaaagcaaaaaaaaaaaaacccctgctttatgacccagctatatcagtttgggcagaatgttcaacttcttcaacccatttctttccctggggaaaagcagattacctacttgacacagttactggcagttacacagtgggaggggttttctttcttgtgatcccaaagtcaagcaacaaatattatatgacgatgaggtaatatttaaataatatttagtctctgaaatttaaagctttttgaaaggtgtcacagaggtcatctaaattcaaggtctcctttaaattaaggttaaatggccgggcgtggtggctcacgcctgtaatcccagcactttgcgaggctgaggcgggaggatcatgaggtcaggagatcaagaccatcctggctaacatggtgaaaccctgtttctactaaaaatacaaaaaattagctgggtgtggtggtgggcgcctgtagtcccagctacgtgggaggctgaggcaggagaatggcatgcactcgggaggcggagtttgcagtgagccgagatccgcgccactgcactccagcctgggcgacagagtgagaccccgtctcaaaaaaaaagaaaaaaaaaaattaaataaataaataaataaaggttaaatgatgtccaaagtcataaagatagtgagtgccaaagtccctgtccactgttcaatactctcctgttttggtatttttggttttataaaatactatgaccttggcttcaagttattaggtgtgccagtataatcttgggcaagttatttcatctctttatgtctcagtttatctgtgaaacacagacagtaagacataattcacaagattgttgattacataaattaatacatatagttggcccttaagacagacattaggagccctgatctcttgtgaagtcaaaaatctgtataattttggactccccaaacttaacttttaatagcctactgttgacctgaagcctcacctataatgtaaacagttaattaacacatattttgtacattgtatgtattatatgtagtattactaaaataaagaaacagagaaaaaattgttactaagtaaatcataaggaaactacatttacagtactatatttattgataaggtaggtttttttttttcttttgtctttttttttttttttttttttgaggtggagtctcgctctgttgcccaggctggaatgcagctggcatgatctcggctcactgcaacctccacctcccgggttcatgcgattctcttgtctcagcctcctgagtagctgggactacaggcacacgctaccatgcccggctaatttttgtatttttaggagagacggggtctctccatgttggtcaggctggtcttgaactccccacctcaggtgatcagcctgcctcggcctcctaaagtgctgggattacaggcatgagccaccgtgcccggcctacaaatttttttaagagcctaggtctcactctgttgcccgagctagagcacagtggtgcaatcacagctcactgcagcctgcaatttctgggctcaggtcatcctctcatctcagcctgagtagctgggactataggcacacaccaccacgcctggctaatttttaaatttttttttttttttttttttttaagatggagtctcactctgttgcccaggctggagtgcagtggcatgatctcggctcactgcaacctctgcctcctaggttcaagcgagtctcctgcctcagcctcctgtagctgggattacagatgcatgccaccatgcccagctaatttttgtaattttagtagacggggtttcaccatgttggccaggctggtgtcgaactcctgacctcaggtgatccgcctaccttggcctaccaaagtgccgggattacaggggtaagccactacgcctggccaatttttaaaattttttctagagaccaggttggtctcaaactcctggcctcaagtgatcctcctgcctctaactcccaaattgctagaattatgggcatgaatcaccacgcccactcaaattttaactttttataacagatttgtgcatattttatggtagtaaatgataaaacaaactagtatctacctatgtttcatgcattcatgacattcctaccttttaaattatttcgatgtatctaggttacaagggattcatctgcgagtttcttttttttgtcacaaatctccagaacattttccaatatatttactgaaaaaaatctgggtgtaagtggactcatacagttcaaacctgtattgtccaagggtcaactgtaggaagcatttacaccagtatgcaatacaaagtatatgcttaatatatgctagctattattttatatcatttcatattaaattattaattatatattaaatctcagtaagataggctgtagcaaaatggaaacatttactgaattaactggacataaaatagttcacttccattgatggctaattacctctcattcctgttaaggcaaactgaatggtatttcctccaactccacagaatgcgtctactacaacgtcacacttgaaggactgactaacacggccagcaatgtgttcagcaatcttctcgggtgtaactgaaaaccagccctctgcaaaggaagagtatttgtgttagcatgttcaatttcaagaagccaaggcaagcataaacatagcaaacattactatatcttacatacttttaaaaagcttagtttccctttttaaaaaattataaatcacccagttttaaagaaaaaaattcaaagcttgttataaaaagcagtaccattcccaatttccacagaaatgatgacctcaattcttaacctaattcttttgtaatactgtatacctccatctttccaataactgaatttacaattttgatgtttctctttgaggctttaactactgactcctgtggaaaatagagaactagctgctctaattggctagctcccccacactccgctgagtttcccaattatttcactcctaaggttgaagcaatagcctggatttgtattggacactccagaaaaaactccatttttttttggagacagtgtcttgctctgttgcccaggctggagtgcaacggcatgatcttggctcattgcaacttccgcctcctgggttcaagcaattctcatgcttcagcctcctgagtagttgggactacaggcacacgctaccatgcccagccaatttttgtatttttaggagagacagagttttgccatgttagccaggctggtcttgaactcctggcctcaagtgatttgcctgcctcagcctcccaaagtgctgggattacaggcatgagccaccatgcctggccaaaactgcatgcttttaaagggtcggaaaaagcaaggaactggatggcaaaaggccatagggttgcagaagtacaggaggaatagcagagccctcatcattgtcagaaatgaggtcctgtgtccactctagagtaaagagaatactgacctccttctttcctagtagaaaaaatttacatttttcagaaagacaattatctgaacaaaatgatgtgctgggctgggcgtggtggatcacttgagcacaggagtttgagagcagcctgggcaacatggcaaaaccctgtctctacaaaacattagccaagcatggtagtgcttgcttgtaatcccagctacttaggaggcggaggcataaggattgctttagcctgggaggtggaggttgctgagccaagattgtgccactgtactccagcgtgggtgacagagtgagaccctgtctaaaaaaaataaaaaaaaaaagaaaagaaaaaataaaaagaaaagaaaagaaaaataaataaataaaaataatgtgcgttaattttcctgttgtctccccttaccaaaactttaaatggctcttcaatactggaaaaataagaatatacacttttatagaattgcaagtgttttccagccttatttctccttacaaattcctacgtactttttccaatacacagcattttgcaaattgttttaaaagggtcagagaacccctgaaattccatcatagatcaagaacttctgattttgatagttaataaaaaccaatatttaggtaaccccaatatgaaaacgagcatcagtctgcaagactgagttaagtaagctgttgcagatcacagcaggaaagctggagaaagaattcaaaatttgcgaagttaattcattaaattgtatccttctgcactagatatttcttcatcaggttttctgtttaataatttatcaattgcttttgctatctatcctaacgtgctacaaatacagattaaacccaagtctgaaattttaaaaatacacacacacacacacacacccccccaaattgaagggcatcctataaaataaatggccattgatcctttgaaaaatgtcagtcatgaaagacaaaaaaggctaaggaaacattccacattaaaggcaactaaagaacttgaaaaataaacgcaatgatacatgatcctatactggaaggaaaaaaaaaacgtgataaaggacaagattaggacaattaatgaaattagagtataaactgtagattaaaatattggaacaatgttaagttgataattcactattttaaacaaaaatatacttgtccttagcaaatatacactaaagtattaagaagtatgagcatgatgtgtgttaagtactgatcagaaaaagatacacacacatacatatggagagagaatgagaacgtaaacggggcaaaatgttgaaaatagctgaatctgggtaaaggggatatgaacattccctgtattattcttataactttctttttaagtttgaagtggtttcaaaataaagctaaaaaatacatatatactttacctctgtccaacttaatcccatcatcaaaacgggagaagagcctgtacctctgggcccagtattttgccagctcaggaacagcagctatttcaggaggcagaccattcacctttttgttcttcttcttgttcttcttctttttcacttcagctaaaataaaatacaaagaatatagatgctcactaagaaaaaaattagtatcacaggtgagtttatctaaaaatgaagccacatcaaataattacaagcagaacatgtatataatgacacgcccagagaaacaggagagcctgatggaaacgccactggcttcaatcaccaccagcattttgctacttattggctttgtaagcttgggaaagtaaccgagggtctttaagctccattcattcattcatttatttatttatttagagacgaagtctggctctgtcacccatgctggagtgcagtggcacgatcttggctcactgcaagctccgcctcccgggttcacaccattctcctgcctcagcctcccgagtagctgggactacaggcgcccgtcaccacgcccggctaatttttttgtattttagtagagacggggtttcaccgtgttagtcaggatggtcttgatctcccgagcttgtgatccacccgccttggcctcccaaagtgctgggattacaggcatgagccactgtgcctggcctaaactccaattattaattcctataaaaggaatgagtgggctgagcgaggtggctcacacctgtaatcccagcactttgggaggctgaggcaggtcagaagttcaagacaagcctgaccaacatggtgaaatcccatctctactaaaaatgcaaaaatcagctaggcgtggtggtaggtgcctgtaatcccagctacttgggaggctgagacagaagaatggcgtgaacccgggaggcggagcttgcagtgagctgagattgcaccactgcactccagcctgggcaacagagcaagactctgtctcaaaaaaaaaaaaaaaaaaaaaaaagaacgatggggctgggcgcagtggttcatgcctataatcccagcactttgggagcctgaggcaggtggatcactgaggtcaggagttcaagatcagcctgaccaacatggtgaaaccccatctctactaaaaaaaaatacaaaaattagctgggcacggtggcaggtgcctctaatcccagctactcgggaggctcaggcagaagaatcgcttgaacctgggaggcggaggttgcagtgagctgagaatgcgccattgcactccagcctgggcaacagagcaagactctgtctaaaaaataaagaatgaatgaatgagttagtgagtggtgttttactaacttaattcacagcagtgacaggacatatgtagaaatactgctatagacataaaagttattacttctgttagccaagacactgctttatgtggtgaattatagaaatctaaacatttcctttctatcaaggactagattaggctctagctgtcagccacattgaagtcacatggcatctgagaaactgaaacaagggtcactagctccattccacggaaaaggatctcattcattcagagagtgattttttccttaaataatttttatcagtttggttatttggtttcagtcttcagcaaattagaagcaacaacatagaaagatgagacttttttcacagttaagtataaaaacaagaggtgatggctaacaaagaataagactccattacacactcaaatggtcctcaaggatagggctatgtcctaatatgttcatctgatcattgtaaatagtaaaaagtgtcatcatgattcagtaaggtcagatctccccatccccaaacaagtgttaccaatagcaaagtaaggtgattatttgtcaacagggggtataatgacattgtgggtggaataattctttgttgtgcaagaccatcctgtacatgacctaacattaagtaactgagtactaaatgccagaattccaaatgtcattctaacaaccagcatcttccaaatgcccttgtgaaggtggtactaccccaggtaaaaataaccagcatcatgagaattggtgtttatggaagtgggatagtgcaggaaaatgggacttagcaattggagaagctggtaatgagtcctagctatgccacttacttgggctaatttttaaatttctctcagctttactttctgtatttacagaatggaaaataattccacctacttccctggctaccttacagattaaatgagaataagacacataaaagcatttcacaaacctacaaatgtcatatgaacatcggttattataacattgataataaaataaatgtaaactactgtaggtatgctgtttagaatgtaaagaaatatgtgataaaaagatcacacatacacatacacccacacttgcactaagaaaaaaaaaatcaagactcaaataagttattttccatagtaataaagaaatatgatgaaggatcacaataaatataatggtttcataataccattattaaactatataagaacttttgctataaaaattcgaatacaaaagtgcataaattgaaaatgttactcagtttattcattagagccaactgctctgaatactttttatattaacgtgccatcaaaaacacatgaccatcatcaaccacacacacacaaacacacaaaaacacaccaaaaggaaaccagcctgagcatggtggctcatgtctgtaatcccagcactttgggaggcaaaggtgggaggactgcttgagcctttggcaaccagcctcagcaatatagtgagaccctgtgtctctacaaaaaaaattaaataattaagcaggcatggtggtatgcacatgtagtcccagctactcaagctgaggtgggaggactgcttgaaccctgggggtcaaggatgtagtgagctgtgattgtgccactgcactccagcctgggcaacagaatgagaccctgtctcacacaaaaaaaggaagaagtaaaccaaagctacaccacatacgaagaaggcaaaggatggtcacctttagcccatggaactgagtaccagcagaggccaagttgaactgcagacagtggtctgcagacacaatgctcccaacagtctttccttttctacttgatcatgttcactcagccaagagtgaggagtgctgtgggtgctaagtcatagccagttagaacatgtacacttaatatgccttctctcacttaaaaataagtagcagtgactccttaggtacagaagattcacaagtgaaaactaagaaaaaacatattaacaaaaaaaattttatctaaatccattagcaagttgaagcatatttagtgttaccttcagtttctgcctggcgggagtctggcacttcttgagtaacacaatcctgctcatctggaacagttgctagcaagctgtctctttcatagttttctgtttcaagttcaccagctgaagatacgctctgacacttttcaggttctggattattagtctccagtaggtcatcaccttttttaacagacatgtcttgttcctctgaatcactacttgtggaagcgtcgtgcacattgtcatgagaagatgattcctgtgatgcttcttcatccattggtttattaacccatgtgaggaatttttctaccttaaattttaagatgacatcatgagcaggaattaattccaaactacacagaatcttcctacctattacaatttgtgacttgactgaaaagctgtgtcaaaattattggctctgaaaagcagactgcatcacacaagcttaatagtaagtaattctgacctaatcagaatgctatctgggcatgccatgaccttattaaaagttttctttaaaaggaaaaaaatatcagattaaatacttcctgctatcaataaatactaagaggataaagccaaactgtcccagtggcttttccataagggttcttaacaatttaactgtgctacagctacattacaaatgtacctacttactactgaatcattaattttagtttttcaaattatatagacctttctgcaatggtcaagctacaatctatgtaaatgttacttagcatcaaagacgaaccctaaaaagatttagggccaattcttctgtaagaatagaagcagtctaccttggaaaaaaatcaaagttgccttttaatgcaacagtagagccagtaagatatgtcccagcaaatgtttgttctttggaatgtaaacaaggctcagcatcagatcatatacttctttgcatcttctgtaacaagaatattcttcctatcttgtcttcatgaccttcctgagagtgagtaaattagaatgaagtggtagacatgatgggcaacaacccagaatcttcctctaaagaaatgtgcctataaataaattaggtatctaagttagacacaacagttacatatcttttttcctcagcaatgcttagtagctgtttctgcactcatctatagatgaatgcataggggctattatatccaacacattacggaaagtcctatggcactgactggactaatactaggcagtctgtttcagatataactagcaccacgaacatatttattaaaacatcaccagcagccaaccaacctgaaatgtaatacaaaataaaaccagctatatatatgtagcatattttacaagtatgctgaagttagggaggacactgtattgaggggaaaaagagtcatctgactagccttaaaaaacgaacaacagagacagagacatcatggagtctaagattatggtctactgtcctgaagagctgctggagatgtgattgacatgaaggttctgctcattagaaagtgttttttaccccttagaatatagaaaggtaattcatcacaccaagcaattttctaactacttttgtttcccaggtacttggatggcaggtatagcaaagtccaatatatgaatatttcaggtaataatgtgaatcaataaattggcctgtgtttcatgctatcagggtgaacttgttacaaggtgtaaactgtgactgatcacttctggaagaactcagaattaaagcttataatataaagctgtatcagaatttggactattaggatcctactggtatggtttggctgtgtccccacccaaatcttatcttgaatcatagttcccataatccccacgtgttgtgggaggaatcaggtaaagatagttgaatcatgggggcagtttctcccactctcttcttgtgatggtgagttagttcttatgagatctgatggttttataaggtgcttccccctttgctgggcactcatttctctctctagctgccatgtgaagaaggagttgtttgcttccctttccaccataattttaagttttctgaggcctccccagccctgcagaactgtgagtcaattaaacctctttccttcaaaaattatccagtcttgggtatgtctttattagcagcccgaaaacagactaatacacctacctagcctaaaacagatgagaaattgttccttgttcttgtctgttactacataggcaaggttgtgttttttttttgagacagtcttactctgtctcccagcctagagtgcaatggcacaatctcagctcactgcaacctccactcccaaatttcaagcaattctcctgcctcagcctcccgagtagctgggattacaggcacccgccatcatgcctggctaatttttatatttttagtagagatggggtttcaccatgttggccaggctggtctcgaactcctgacctcaggtgatccatccgcctcagcctccctaagtgctgggattacaggtgtaagccaccatgcccggccaaggttgcgtgtttttttttgagatggagttttgctcttgttgcccaagttgaagtgaaaaggcgcgatctcggctcactgcaaccttcgcctcccgggttcaggcgattctcctgcctcagcttcccaagtagctgggatttcaggcaagcgccaccatgctcagctaattttgtatttttagtagagacagggttttccatgttggtcaggtttgtctggtactcccgacctcaggtgatccacctgcctcagcctcccaaattgctgggattacaggtgtgagccaccacgcctgacggctgcgttttttaaataatctcagacactagtactttgtagctttcaaacacttccttttcaccctacctcaacttgccttttttcagatttagtttacataatatatgccttcaagaagcaagtaaaaaaaaaaaaaattatgtaccttgatacctggtagaaacaagcttcctatttgaatacaacaattttaagaggtcaaacttacaaaacatgattctatctacaaatatgcaacaaaatttatacataccttactcagaattttgcttttcttgaaaaatggtttttctgactctttggtaaagaagatgtgtttgtttttcatctttatttgtctgcgcatgtctaggtacttagacttaagcttcacattcttctctaaatacctgacctgccgatgactgaaatttgggattccaccatatctgtgacagatcaaaagaagttattcacaacttaaattctaggattatatttccttataggaaaagaggatgaaactaacattttcactcatttttacaccattttaattccctttttaaaattatgaaaatgataaaatcaagttcaacttgcagaagagttgatggtctaactttcatgttaattctaaagcaaatgtgtaacaccaaagttgggaagtgcaacatacctaatatagaggagaaagtagaaattacatagtagactgcaataaataaatagtaaaacaaacaaacaaacaaacgaaaaccctatccccttgttagggaaatataaatatataaatattttcaaggtggggtgatgtcaaagctctttaacacatttctggatacatttgaattatctaaatcaatctgagcttacaaaagaagcgaacccttcgtatcaaaataattagagaatgcctgatttaaccatctgaaattttccagtcctaaaagatatgtatctgctatgaagcctcctttgattatattaagcctcaattaactccctctccgacaaactgttagggacttaagagtgtcacgtacactttagcatttataaaatccagaatagtctgggtttaaatatcaggtctcactttgtagccatattataaacttcttttaataaacaggatattttatccttctcttttaagtagaattgggtgctctaaatagaagagtagttaagtagttaagtctcaggcaagaaaagaaatagatttctacaagtgtatactatcaaaatctacccaaggattcctcaataaaattacatgtgcatagtaatacatcctgccaggaatttaaaaacctttttacatgctaaactttatgatcttagccttaaatgattatatgaactattgattatattaactataattgccttgaaacacctaagggagaaaaactgataatctttagaaactacaagagtcaacatgtacttctgaactaatttgaattcaattcaattttgagacattcctcgtgtccctttcttgctgatttcataatgtgtaggtatctacttaaatttcacaacttatgaaacagataagtaggacctgaaatctcaacccttagaaacatcaaagaacccagtcatggtttagattactggtgtaagctgtctttagcaggctttacctactcagtttagtcttcatgtcatgtacgataaaatctctagttgttctaacacaactacccatgttcctttatatttaatgcttgggcatttaatcccagagaaatcttatgtcttttatacacaaatgttcacagcagctttgtttacttttgaaatttcattttatttttttattttctgccttgctatgttacccaggctagtctcaacctctggcttcaggcaatcttcttacctcagcttcccaaagtgctgggattacaggtgtgagccactgcacctgcactgttgctttatttgtaacagcaaaatgccacaaataatccaaatgcccttcaattgttgcatggttgagcaaattttggtacatccatacattggaattctattcagcaataagaaggaataaatggactgggcgtggtggctcatgcctgcaatccctgcactctaggaggctgagatgggaggatcacttgaggccaagagtttaagaccagcctgggtaacatagcaagaccccatctccatgaaaaaacttaaaacagcactgcactcctgcctgtgttacagagcgagaccctaaggaaggaaggcaggcaggcacatgtaataatctggagggaccttctagggcattatgcttagatcttaggaaacaatttagatgttgttaattaggataactgataaaacacaatgccctcatttacagaattagctaaatgtttctaccaacaagaaagagtgtctatggtacattgttgagggagaaaagcaaactggagagtatgtataatatgacctaatttttctgttttacacaaagagaagcaaaatgtgtgtacgtggtgaggcgcggtggctcacgcctgtaatcccagcactttgggaggccaaggtgggcggatcacctgaggtcaggagtttgagaccagcctggccaatatggtgcaaccctgtctctactaaaaaaaataaaaaattaaaaaataaataaataaataaataaataaataaataaataaataaattagcagggcatggtggtgcatgcctgtagtcccagctacttggggggctgaggcaggagaatcacttgaacccaggaggcggatgtcgcagtgggccgagatcgcaccactgtgctgcaacctgggtgacagagtgagactctgtctcaaaaaacaaaacaaaacaaacaaaaaatgtgtgtacatgtatgtagtcatagcacacaacagtaaatttggagggacgtatgccaaactcttttttttgagagtctcactctgttacccaggctggagtgcagtgacattatcatggatcacagcaccctcaacctcccgggctgaagtgatcctcccatctcagcctcctgaggagctggaaacacaagcatgcaccactaaacccagctaatttttaaatgtgttgtagagatggggtctccctatattgcccagcctctcaaagtgccaaactctttaaaacactgcctacatttgagaacgactggaagaaaatattttttactttttacttgttatttggtttgttttaattttactctttcataatagaaaaagaggttattgcagatttcaacgtttcatggcatgtttgctatggtcttcagatgtacacatgtgagacactaggtgagaagagaaccatgcttagaagtcacaagagctgggcctaaattccctctccagtaaagtcaaagcctctgtgagctgcagttgctttatctatgaaatggaataacaacactggattgctataaagtgcaactgagttaaagcatatgaacacatcaaatgtacagggatatctctaccacaattctaacaagaagtgatttaatgataacatctcaacacatgatcatttattagttatctgagtatcagagtgctctaagatagttgaagtggttaggacttctggaaaacaatagcttttatgaataattacttttgtcctgagccatacttgaatcctagaagggaaccactgtcatcaaagtctgaagctgggttttcatcaatgtccagttcatggctaaataaagaaaaggtaaacagtgataagccattaccaccagagcccactagttcagataaatataaaaaaggcaatataattgacccatgcaaacttcccaacaccatagtcaaccatagttcacaaatccaggaaaccaggaaaaaaaatactttaattagaattttccaaataaggtaagtacccaggagcatttggaacacagcaataagtattccagtcttaattcatgtcaagtaatgatttctattcctaggtatggtacaaatataatctctcaattttcctttggaaaaacatcagtaaaagctgcagccttaaattaatctcctaaagtgatttctccagaggtttcccttaaaaacaaacaaggaaacgtttgataagtatcaatacaatccaaaaaaattttaaaagaaaaaaattactaaaaagaataaggttgtgatacaagtgatattaggtaacccaggagaatccacctactagagatcttttagtaaccagtgaccatccctggccgtaattccaagtaaaggaaaatggtgagaaataattcttacaaagtttgtccttcaaaataagttacgctcattgaacacacatgatctttgcctccagtaactagcaggaactggggagttaacaagcaatgtctaccatacagaggtagcacagtagaggcacgcttgtttttccagaagctatatggagtacaagtgtcttacgtggagcaccccatatccagtcaaagaaatgaagtgtccagctaacttccaagttcagtaacgatccattctttccttgttacttacagtgttagcatctgactacatcccaccttacattattctctacgtgtttaattatgtgtacaagtgtataaaccttcaagaaaaaaatgtagagatgaataccaaaaaaaagaacttttatttggttataaaacacaaaaacagaactgcagtttgggacataagcacagaatgaggtggcctcatgaaatgtccaaagaagagaggaaaaggttagagtttcactgaggagagaggtcacgcacgttgtttagaaagttagttcactggtgctggcagcatcttacaacaaacagctggtgagttttgattaggagagccagtggttgttaggtggaagactgaaggctacaaattcaggaataacttacaaattacaacaaaggactgcttcaggagtggcacaaccactgccacttctgggcagagataaacttgtaaactgagagttatggttaggcctctgcagtttcgaattgggcttgtgagactgtgtcaggcacatgttcctgtataagcagctatctgtctttgtgtgatgctttggcttggaaaaatttcttgtgacagttcctgttatcaagcaaatagtgcatgagagtctttcatggccttccctggctacattttaccagtgtctgacacaagtgactctgttttgactcttacaactttcacagtggtaaacagaaattgcactgcttcgagtagagttgatctgggctctggctttagagctgccattgctgtgaagaaaactgagcaaggtactcattcctgtttgcctcagtgttcctatctatacaatgagagcatgaccttgaggtctttttcagcaatacaaatctgggataaaatatatccagacttttgaaaaacattagaactggtgagagagagagagagagagagaagatatgaatttaattattagttaaaagttatgatccattgatagctactaagtgtcagatatattacatgctagctcctgtctacccaataatcacaaaaaaggcatcagtccaatcttatagatgagaaattaagaaatgtacttaggccgggtgtggtggctcacacctgtaatcccagtgctttggaaggccaaggcaagtggatcgcttgaggccagaagtttgagatcagcctgggcaacatagcgagaccctgtctctataaaaaattttaaaaaattaaccaggagtggtggcacatgcctgtagtcccagctactcaaggcagaaggatcaattgagctgaggagttcaagttcaattgagctgaggagttcaagatcatagtgagctatgatcataccatgtactccagcctgagcaataaagcgagaccatgtcttaaaaaaaaaaaaaaagaaatgtacttagagtttgtgagtggcagagttcggccttaaccacaggtccaggtccaaaggctcagaatcttaccagtagatcctgcctcttcttacatgattactggtgttgtgaacataacaatcatttaataaccactggcttatgcatacaaatgcttactctaacacgctctggtgagaggggtctcatgtaacttacctcctcttcagtttgcttggcttatgctcaggtgggtcttcctcactttcatctccatcagtaccactggcatgtggtctgtctttgcttgtgtttgctcctgaagacttctgtgaatctaaagaggcagaaatattgcagtagcttttcccccttggtttgacaacttttctgctatttgcaaagccaatgaaaatattttagcaccttttctacaatgaactgtacatgtgcatcaaagtgccagctcacatctacctgtttgtgtctacatgcagagtctgagcattttaaaaatcttaactataaggtttgttaaattaaatttagcctaaaactgtccatgtatacattttaagctcagcttaaaggtttttccacatgtagtgaactgtaacctaactggtggtataaaaagactgtaacctactcttgtatcaagcacagttttggctaatcacaggcagccaactattcaaatggtgttcaagtaaggcaaaagctgggctgtaaccaatccagctgtttgtgtacctcacttccaccttctgtacgttgtacatcatttttctttttctgtccataaatgttatcccaatcatgtggcagccctggagttgctctgaacctattctggctcttggggctgcctgattcgagaatcgttctttgctcagttaaactctattaaatttaatttgtataaagttttttaacaggtttaaaaatgttcatgatctaactacttaaaaaaaaacacaacaaactaaaactacttcttcatagctgcccaaatctatacaaattaacccacaaagctgcctttgtgatagctgttagcagtcacattcttatggaattttaattgaaatgaccgtaaagggggcataatcacattgattaagaagaatttctcttcatttgcctttaatatagtatgaaagtaacattaataatgagagtaacaaagacaatactatctttaagaagagttctttaacttatggtgacaaaaactgaggcacaaatctgtctttgttctctattttgtcagactggaagaaagtgggtggctgagaccaggaggaaaaagatgtctgcagggtagacagtgggaagctttgactctttcagaaaagcaccaaccacaaagagtagaaaacatacttacatagaagcataacctataccttaaattttgaactcaagggaggatctaaaacaaatcaaaatctaaaaccctcaaattcatcctgatggcagacaaactggagttattacagaaatggacaatctataaacgtaagtaagggaacaagcaacagaaggcagagactgtgagacttactgtgtctgccaaagaaaaaagcaggcagatgagggaggagaaccaaagagggtaaatgagactgtaagtcagaggccaaaacaatgcccaatatgtatattataaactttatggtcaaaatatgtacagaaaggtatcaggagagtatagaagagtatcagaatactaatataaaatcttcagataaaaaaggatggctgtgcatggtggctcacgcctatgcactttgggaggctgaggcgggaggatcacttgaggtcaggagtttgaaaccagcctggccaacacggtgaaaccccgtatctactaaaaataaaaaaataaaaaaattaactgtatgtggtggcacacacctgtagtcccagctactcgggaggctgaggctggagaattgcttgagccccggaggcggaccttgcagtgagctgagatagtgccactgcactccagcttgggtgacagagtgagactccatctcaaaaaaaaaaaaaaaaaaaggatgaaaatgtatcaccaaaatagaaaggaatgagaatattatactgaggtaggctccacttataagaatctaagaatatagagaaaaaaagcaagttgacagagctctttagaaactgtcagctggatgaagtattatcagcaatctgagataagcaaagcaaatctgattgttaagtaaaaggtcaaaaccatttgtttcacaaaagagtaaggctgagaagaaagttcctctacactagatgtcttcagcatgtaacatcaggatgccaaaacttcaggatatgcagatgtcacagaaatttcctcctctcccattttcctggtgctgttaattggcagaagcctctccccagcaagagcactgatagggtttggatgtttgtcccctctaaacctcatgtttaaatgtgatccctaatgttggaagtggggtctggtgggaagtgtttgggtcatggggcagatccttcatgaatggcttggtgcctccccatggtaatgagttactgcaatatctgattgctaacaagagcctgggacctcccccctctctcttgctctctctcttgccatgtggcatgcctatgcccctttcaccttctgccataattggaaggttcctgaggccctcaccaggagttgatgctgagactatgcttcctgtacagcctgcaaaactgtgagccaaaataaacctcttttctttaaacattcctcagtctcaggtattcctttacagcaatgcaaagtaaacattctcatcgcaccgtactcagattaactcctttcaaaacacttctcattggcattctctagtcctgagtggcaccagtttgcacaattagctttttttggatcagttattcaggaatcaatcaagttccttatcctggatattttattgatgagaatcctgaaattaaccattccagttataaattaatattgtgccccattgagatctttttttcacagcaagacttcacttgtcctaagtgaattccacatatgactcataagacatttgcctaatgtcaaatgtacattctcaattgctttctaacatgcctcttataaattcatttgctgtggaaaactaaaagttccaagctcactttatattagatttctcaagagattccacattttccttcattagccaccaaagtacaagagaaggaatgatacttttgagaactgcatgcaaatattcttattaatagaaaaggataacttgtgatatgaaaacaggatggattaaatcatttcattattatatgaaatgtacttggtattatttaactctacaataaaggattagtagttattttgctaaatgaattcattttgctaaccattacatggcagaataaatcttggttaatcttaccctcagctggtgggtctgtgtttgtattccccgatgagttgctttcctcattggttcctccattacgtggttcactttggccggaagcagggcactctcttttgctactaacttcacttagctgttgatgaccatcatgacttgtacaggaatctaattggctctgtgttacttcctctgaattcagttttatgttactaattccatcaagtatttcactatgatccttatcacttgtaccagagctatcattatcaaccataataggtgaagatggaaaagatactaagtcaactttcatgcatttttcatcgttcttgtcatcagcttctgttttagatgtgtaagtatctgtatcacagctttgcgaggcatcaaaagtccaaccctgagcttcccaatactgaaattgttccaaataataccaataaagttgactataatgttgctcccattcttcctttgtatcaggaaagttccaaggttcagaagatagtgcttgacccggatgtttttcttgccaactttgccacaatagtcctcctccatattcattccaatacttttcccatttctctgtaatttctagctttggagataatgtgttttcaactggaggattttctgtctcagtatcttttttgctttgaagttcatatgttctggtgttctcatactgttcaattgaagatggatcatctgaagccaaaatgtcgtcttcttcatattcttttctccaagattcttgcacaatttcatctaagtatttcttttgatgttttttcttttttattttaactttatttctagtattcatagatacctatatcagataaataacttgaatataaaatgcactttatgaactataaaagaaagtagaatccaagtctattttgacattaaattaaacaaaatactctaaagccccactatgggcttttagctccacaaacaagtgatatactatctaagtttccaagaggaggaacagtcacacttgggacatattacaactttcagttctggttaatttataaatacatacatttaaaaagaggtagagagcgctgatcgtgaatgcatataatctgataaaaagtaattccttgcgatattttataaagaaaacgaagagagagaaataataaaagtaaattaatatttacctcaaaatccttatgtgcagttatcctaccaaattgaagtggcaatcccatacttctcatgagttcagcctcagaatcaagttcactttcatccaggcctatgcctttgctgtcatgtgattctgcagtaccacaggaataaccaccttcctcttcttctgtcgcctggtctcctatagacaccatttattagcttagttttaaggaaaacttaagaggcaggttttcttattctcatttaaaaaaacctgagatccagagtgacccgcccaaaataaaggtatgcaaatgttacaattttttttattttttagagatgggggtctcacaacactgcccaggttggagtataagggctatctgcaggtgtgatcataatgcactaaaaagctctgaactcccgggctcagtcagtcctcttgcctcagcctcacaagcagctaggattacagttgcatgccaccttgcctggcctgcaaagttacaatttttaaaggaaaaaagaaaacaaacaaacaaaaaaacaattgacggtatgctgggcacggtggctcacacctgtaatcccagcactttcggaggctgaggcaggcggatcacttgaggtcaggagtttgagaccagcctggccaacatggtgaaaacccgtctctaccaaaaatataaaaattagctgggcccaggctgtaattccagctactcaggaggctgaggcaggataattgtttgaacctgggagacagaggttgcagtgagccaagattgtgccactgcaccccagcctgggtgagagagcgagactctgtctcaaaaaaaaaacaaaaaacaaaaaacaaaaaacaccaattgacagtaatatggagtagtaacaaaaaaagccaatgaagaatcaacggaagaatcaatttcttattgggctctgcctgccactattcatccagctgcccatttaacttactcttattgagcatctgctctatgttgggcactgtgcaaaatactgaggatggaaggaagacagaacaggccatgtaaccctggtacgtggagaaaatacttatatgttcacattctttatctgcaaaattactgaactaagacagtgaatttaaaaatcatgatttgtgaagttattttaggaggtattaacaagagcgggtgagaagcatgtcttcatgcccctcacagacacttcaatacaactgggatttaggttccgggattctgtggaagagctgatttaacaaaaggattctgctattagaagtttgaaatcctggggttgggcacagtggcttatggctctaatcccagcattttgagaggctgcttgaggccaggagtttgagataagcccgagcaacacagcaagaccctgtcactacaaacaaaaccaaaaaccaaaaaacaaactaccctggtgtggtggtgcctgtagttcctgctacttgggaggctgaggtgagaggaatgcttaagcgatcaccactcagacaacagagcaagaccctctctctctctcaaaaagagaaaggtttgaaatcccctaaactggaagatttttttcaagctctctgaatctaattcttatagaaaaaccatttattacaacaaagtaagttttactttacaccaatatggttatctaatagtaattataccattaattgaacatatgccattcaagtgctctattaactttaaaatgtgagataacaaaaaatcattttatgtattagcacactcaattagcattgggctgcactccagcttgatggtctcaggcaacagaacagtaaatcttatatagccaaaatgaagactgataaagagaaatctcttgtgtgcaaattctgctacaagagggagaaagatacctttctaaataatgaaggggattccactcactactccattcaaatatacttacgaaagctcaggagacagcaaaatggagaaaatgagtaagctaagttttatgttcctttcattgtatgcatattttcttatgctcgaggagcctagtgttataggaactttatttgtatagcatagcactgaaacacaaaccaaacattccatccagtgatcaactgaagaaacagtaatttgttccatcgccatgagagttaaagggtttccgaaggtaactatgacactgtacttgggctgactttggcttctaactccacgctgtaattttctctgtgtctaggaacacaaaaggtctatgtggaaggacaaatgcagtagggaagtttgagacatcaaagtgaggctcaatgtacagtataaattatatttgttacttgggaaagttaaccaagatgacagtctctttacttcaggcaaaagactatagatgttcacctggaatttttatcccgtagcatttttttatcctgcagcatttacctacataatacacttctcatgaagtgtattatgattagaatttacatattctaaattggagatggagtctcactctgttgcccaggctggagtgcagtggcacaatctcagctcactgcaaactctgcctcctgggttcaagggattttcttgcctcagcctcctgagtagctgggattataggtgcccaccaccatacctggataatttttgtatttttattagagatggggttttaccatgttggccaggctggtctcgtactcctgacctcaagtgatctgcttgccttggcctcccaaaatgctgggattataggcatgagctaccacgtccagccagaaaccacatattccctacttttcaaagctttaaattccaggttgctcttatctaatggtggcaactcaggaaggaacactacacacagcaaaagaggctacattaagtcagcacaggagctatagaaaaataaaagatctgctttcatagagattccagaaccatgttataaaagaaaataaaaattgtctgattacagaattatttggcatttatattttatgctaacaaatcaatacaatattataacaaattatacataaattaacacattatggctaatttcagcaaaaaaggtttatagaattttatactatattacctgaattgttgccactgtctctgatgtaatagccttttaatcccaaattgtacaattttcgatccctgcgaagcagattaagcagttcatattgaaatgaattaagccatcagtttagagggaaagaaatctcgagccatcatagattaattaataaaaagcttatgaaacagaaaagcaatctgctaatacatttccctcacttataatgtagaggcaataatacatttgtcatggagttgttgtaaggactgatagtatatgtaaaatatttagcacaatgcttaatacatggtagcatttattactataactgctagttactaaaattaaggctgggcacagtggctcccacctgtaatcccagcactttgggaggctgaagtgggcagatcacctgaggtcaggagtttgagaccagcctggccaacatgatgaaaccccatctctatcgaaaacacaaattagctgggcgtggtggcaggcgcctacaatcccagctactagggaggctgaggcaggagaattgcttaaatccgggagacagaggttgcagtgagctgagatcacaccactgcactctagcctgggcgacaagagcaaaaatctatctcaaaataaataaataaataaataaataaataaataaataaataaataaataaagtgtaagttcttctggacatctgatattccttatgatacataaacaatggggtgttctctaaattttgttgtgaataacagtggagagatttagcttacatacatataaatcaatatgcagacactaatccatttgctaataagacaaactatccattgtgaacagtagacaaggtattcatggtctgagcaaaggttaccaaaataaacctaacagaatacttaagaaaaagtggaagactctgcttttaatatcttcctgactataaattcagctgtaataaactccactgtaactgccacaagaaggccaattgcaagtggaagctaacataacttttctaatggagctacaataaatttaactaaataaaattctagtgtacttgctctatttcacatcctcttgcttcttttttggagatactatattaaatgtaaagtaggtggtgactcagcaggatgatgaacaggcaagggggtgacgtgcaaatgtgctttggcagcaactggtccccaggaacatgggcactgggggtgggaagcaggagctgcacagggggcatcactggtctgcccgccagcacctaaagatatgaggcactgaagtgtcaggtggaggagaggaccagtatggttggttgcaagttggttataatgagcaaatatgtaaatgtgttgaagataatgttagccaagattccccttgttgaaggttacttatttactttattattttaaaatttattttaatgtttttaaataaaaaatttttggtttttttttaaatagaaatgaagaaggttatttaaaagcaaggtgggctcaagacattatctgataggaaggaaggctagaaagaaccctcaggtgttgggccaacattagaagtactgttataaactcatggcatttaagtttatatacacagcagacataaaaatagtttttaaaagtgttacatatgtgtgtatatatacacagacacattcatgtatgcgtgtaaaacacacatatataacactgctcaactctctctactaagagggcacaggagcaataagcacaatgagcagccagcttttggtttctaaatactattttccacgaaaggaaccaagttgcccggagaaatgacaattccaggttggagcatgaactgtaaaagataaacctgggtcatcttatgccagaagattaggaagcactcaacactagaaataccaaaaggacacaggagccacttcaaagagttccccactgggaactctggccaaatcagagacaatatcagcaccaaattaatgttagcaatggaatataacctgttagtaaactaagaatctagattccatgttaacataaatgaataaatacatggggagagaagggaaagcgctccttacagaatactaactaataaatgcagtaggagcgaagaagacaagaactactccttggcatccttcacagaggtagctgattcagggggaagttatcaatgaaggccaaggcttgtgggtttactggaaaacaggatactgatataacactggaatatgcccccacaaaatactgattaatacaaagggaactattgtgactttacagtagaaaacctggcattcagcaacatcacccatgatggtatactgtctacaaaaagtgcctcctgaggccacacagagctgggcatagcatcatttctgtggcactcctgctaaggcatgacccaactattcctgatgaaacatcagacagacccaggttatgcaaggtcagaccccacctctagaaaaaacaaaaaaaattagtcaggtgtagtagtgcacacctatggttctagctatatgggaggctgagctgggagaatcacctgagcccagaaggctgaggctgcagtaagcaatgatcatactactgcactccagcctcagcagcagagcgaatctctgactcaaaaaaaaaaagaaaaaaaaaaaaaaagccatgccagacatggtggctcatgcctataatcctagcactttgggaggctgaggtgggtggctcacttgaggctaggaatttgagaccagtctggcacacatggtgaaaccctgtctctactaaaaatacaaaaattagctgggtgtggtagtgtgggcctgtagtcctggctacctgggaggctgaggcatgagaactgcttgaacccagagggtggaggttgccactgcactccagcctgggcgacagagcaagactctgtcttaaagaaaaaaaaaaaaaggaaaaaaaagaatacatatgaaattatttgtaatgaaaagcatacatgtttcagttcaaaaagatgaaattcaatggaattgataagatggtcattaaatagtgttttaaaagtcattctgattcaaatccaattgtgtattattttctagaggtgtgagttgagaaatcttctcaactttttatatttcataacttatattctttcttctcttttagaaggagggcttcctaaaagtagcatcagtacccgatcatactgtgggccgtgaattaagcatgtagcctgggctgccactgactgcaaggtgactttcctttcctggcctgcatctcctacaaagagccaggtatttttttctagtgagctttttggtcccaccacagtgctagcctgtaagccttctggaggatgggcccagcttaacaggtcaggtcctgagaaagaggcagaaaggcctttcctcatgggtgagaggtaaatcagatgtccaaggttattaccgtatgttccaaggctgctgataatttgaggcagaaagatcagcaacaagagattaccagctgttatatttttatgaaaactgtttcttacttaaaactttcaagtatctccaagagcattcatcaccgggaatgatgtatttctaatgcctggcatttgaagaatttccattatacttgctaagtagtctctataaagctgtctttgagtttgattagtgtaccttaatccacagagcataatgactctggatgaattgatggcctcaaaaatagaagatttgttgtaatttctggacctggcacacgtaaggactaaataaatcttaactactgacttgaattgtaaagcagaagacccaacctggattgtagtaatcccactaagatactgctctccttagagccctaagcagccacctgaattcttatcctcatacctctgaatatagcaatcatcactatgatgcctaagtcatctttctattgcagcacctgacactcgggcggctgaaataaatgctgaatataagattcttagcaatatctgaagtacaagtagcaattcctcttttgcaaagggactgtgaatcagcttcattttcaagtagtaaaatggaaaggaagccctttctgaaaggttaaaaggggatcacagaaataatatgtgacttgcctaaggccacacagctgagttttgtaaaagcacacagatatgctgtctataaggtcactactcttttaaattctgactcaggaacctgttggggggaaaacatatcaaacaatttcgttgtatcatatatgaaagactagagatcaccatcagacactgtcacatgagtatgatcagagggaaatataggctcttcagtttccagaatagtcctctatctccacgaatctatattaatgttaaagtgtctgaacagaacatcagttgttctagtgactttagactacttaaataccttaatgagggagattcaatgaaatcatgttctatgccaactggtagcatagtagaaaggctctcatgtcatacagatgtgcggtagaattctaaggctgccatttactggctatgtgattttttttaggccactactgagactctgagacagggtctcattctgtcacccggattggagtgcagtggtacgatcatggctcactgcagcctccacctcctggactcagcctccagaatagctgggactacaggagtgccccaccatgcctggctaattttttgtatttttagtagagatggggtttcgccatgttgcccagactggtcttgaacttctgggctcaagcgatttgcctgtctcaacctcctgaagtacttggattactgatgtcaaccaccacaaccagccaagactcaattttctacttgaataagtgaggataacaagacatacctcagggggctgctagaaggcttaaattaaaaaaaaaaaaaatttaaattattctgacatttgttaaaatggtaaagaaaggctttatttaggataattgcgatagtaatcaacataatggtagccacacgcatgtaatcccagcactttgggaggccaaggcgggcagatcacttgatgtcaggagttcgagaccagcctccaaggtaaaacctcatctctactgaaaatacagaaattagccgggtatggtggtgcactcctgtaatcccagctacttgggaagctgaggcacgagaattgcttgaatctgggaggcagaggttgcagtgagccgagatcatgccactgcactccagcctgggcgacagagtgagactctgtctcaaaagagaaaaaaaaaaagataatggcaatggagagaaattttgctcaacttcaaatatagcaaagacagctgaagatagccaatgggcaaaattcagggggcgtggtcagtggatggaaaattactaaggggagacatccagcatggggggattgttgctataccaacagtattcttgctgagggcaggccagagcgatcagatacgaagggtgggaagatcttgctacatggacttagcaggattctggctaaaaaaaaaaaaaaacacctgtaatcccagcactttgggaggctgaggtgggaaaactgtttgtacccaggcattccagaccagtgtgggcaacaaggtgagatccctgtctctacaaaaaataaaaaaatcaggcaggtatggtggtgcatagctgtggtcccagctattcaggaggctaaggtaggaggattgcttgagccagggaagttgaggctgcagtgagctgtgattgtaccattgcactccagcctgggtgacaaagtgagacccagtctcaaaataaacctcccaaaacccaaaaaacaaaacaaaaaaaacacccaagaaccaccccgccaccaccaaaaaaaaaccaacaaacaaaaaaactaatggacttagaaggattcttgttaaaactggaccagacaggttgaagacaagtcccaaatgacaaggcctaatcaaaaggagagctcactcagagcctgtgtaaagtctggttaaggagagtctctgtcagtacaaaatgtagaacaattagtggtggcacttaataagtcatctaagggaggagaccacccctcatattgtcttatgcccaatttctgcctccaaagaaagaagtaaaaactaaaaggcagaaatgaaatccataagcagacagcccggcgccacaccctgggcctggtagttactgatccacccctgacctaatcggttatttgcataaaaaaggcactgggccgggcacagtggctcacgcctgtaatcccagcactttgggaggccaaggcgggtggatcagaggtcaggagacagagaccatcctggctaacacagtgaaaccccgtctctattaaatacacaaaaaattagctggacattgtggcgggcgcctgtattcccagctacttggtgggctgaggcaggagaatggcgtgaacccagggggtggagcttgcagtgagcagagatctcgccactgcactctggcctgggcaacaaagcgagactctgtctcaaaaaaaaaaaaaaaaaaaaagacaccgtgaagacccctgtccagttcggttcctttctaattagtggtgtacgcagcccccagtcacgtaccccttgcttgctcaatcaatcacgatcctctcatgcagacccccttagagttgtgagcccttaaaagggacaggaattgctcgctcgggttgctcggctcttgagacaggagtcttgccgatgctcctggccgaataaaccccttccttctttaacctggtgtctgaggggttttgtctgcggttcttcctgctacagtcaatcgatccaagggatttagtcaattctagtggatacttttagtcctcattttgcttgatattttggcagctttccaatgttgattacaccttcactgaaacacttggtgacagtgtacttaggcttgtctgtttcctagttaatccatctcttcttgtctttaaaaagtgctggaattcctcaaaggtgggtccccctcccccacccttcattcatgggttcaatcactatctaaggctaaatggctcaagtttttaaaactagcccagacttctccagcgctccaaatcaatatatctaactgcttataccatctactcttggttatctcatagatacccccaaatcaaaatgtataaaatcaaactcataatctcttctccgttgacccaaaataaaattcatgattctccagtgatccctacctcagtgaaaggcagcaatacaattcagtccccacccccagcaagttttctcccatctttctttctttctccatttctgctgccatttatcctaatccaagtcactataatctcttgcctaaacaatggtcttaagtggttctcttcctccaaacaatcctccacactgccgcctgagtagtattttaaatatgcaaatctgatcatgtcattccccagcttaaggttttcctatagtctacaaagtcctgcaggttttgaccctgcctactgtgcacaccctatcttctttctttcccagttttttttcttttctttcttttcttttcttttttttttcgggacagagtcttgctctgtcatccaggcacgacaatggctaactgcagcctggatctcctgggctcaagagagcatctggcccaagcctccagagtagctgggactacaagcatgtgccaccacagctggctaatttaaaaaaaaaatttcagtacagatgaggtgttgttatgttgcccaggctagtctccaacttccgagctcaagtgatcctcactccttggcctcctaaagtggtgggattacaggtgtgcaccaccgtgccctccctcccagccattttgttcagtttcccaggctttcttttgctccctcaaggcctttactgagttcagcctgcagagaacgctttctcccactgaaactcaccccaatttatcccccttagacctctttccaaaagctacttcctcaaagaaacctctgtgacccttgggaagttatttgttttcacagcatcctgcacttctctctcatagcacttataactataatcaaccaagtaataatgtaaccagttatcttaatactgatcttctctgctaggaagtaaactcaatacatgtaaggacagaatgtcttgtacctcagtgcctaccagcatacttgatatacaagagcataaaaaacattttctgaatgattgtatgattacatgcatgcctgactaaaaaggcgacaatactttttacattaaatttaacaagtttaacttctttttgcgttttcaaattactccacaaaattacacgagaaaaatgaattccttcaaacttaaatcctactatattaaactatattaaaagtctatcacataaaattcacttccacaaggatttaactctaacaaaataagcatgtgagattaaatatacccgttatagagatgaaaaaaaggttcaagtggtttaactcacgcatttaatataaaggaaacaattcctagaattcaagtctcttggcggtattattcctgcatactagctatttttcatgtggggcaatttttacctgattcaaagatttccccaaaataaagatatcacaaaaactgatgtaaatatggttaagtcttttatctcaactagtttgaagttttaattatgtgcatttgggcttaagtagatttttaggtttaaaaaaagacgttaaccaaagcaagtcactcaactttaaactttcactttatgacatcatatctactagagatttcattttacatgtcgctttcccccaatttttaaatgtcccctcgatttacaaatatgagatgacatatagcacctacgacattttttcatagcagcctaccaagtagatgtagtccttcacataaaaataatggcagtgggcgaccactccgtcatgtctgagggttttgtttttcttaaacatcaccgtgcccaagggtcagatgttctggctgctttcagggatcaattacaaccacgtttgcaatacctacatttctgggtaatgactgtgctagaacatgaagagattctcttttctacttactccacaaatgccctggagcaaaggcacagtatcttacaatcctcccgctcctcagtgaagagaaacatttccgccacgcggctccacttctcgcagcacattttaccacttcggaggcagctctgacgtacgcagcccgggtcgcgcctgccgggcctccgctcggttactggagacttaaaacaggcctcgcccgcaagccctggatgaggagatagaaactggcccgcggccgctcgcgccaggaactagcccggacgctgccgccggaagtggctccgaggggccacgggaaggggcgggaccacgggcggtgtcacccccatcgcggcccgggctgcagcctccgcttggcttctttcacacgggttgcttcggaggaatccgccgtgcaaatctgtccgcccccttggccactgatcccccgaagagcttctgtcgccgctctagggtaaaggagagctgcccctgcccctgcccccgcctatccgaggtgcgtcggcccccgcgcttctccagcccggcatcgggtctggcgggactgggagcggcggaggccgcgcgcgtccggtctcccaggagccgcctcggaatccccagtctcttcgcgggcaggctcggcttcccgccggcctcagtgttgcgccccagcgccagagtggccgggtgtggagatcctccctcctctgggtcaaagagcctgctctccccgcccctcttctcccctggcgctctaaaccttcggttcggcgcgtttcatcctaatgcatctggtggggcgaggcgtctctagcctctgtagaggcctctaggcttgcccagtggcttcgctggttccgggacccaggtcgagcctccggtgtaaaagccaaggggaagtgtgtggtgatcccgagagtgtagggggaaggccgcgtgggtagagcgttcgtatccagaccgatcaggcttggaatcctggtcctaccttgtgacctgggcaggcaacttcacctttctgagcttacctttgatcacctgtgaaatggggttgattaatagtaactactacatgaggctgcagggaggactaaacgaaatgatgtatattacacctttcctttgtgcgcttaatcgttggcgtcggtttggaccttgtaggcgttaagtgctgtctcggtccctggggtttgcagcctgtaaaacaggtctctgccttcaagttactaattatctaataagaaaaaagttcatgtgcgcataaataggatggagaaaatggaaacagcagcgttgctggatctgttaaaaagcagttagtatttggtgtgtggtattcatggaggctgcacgggcaacttcttcagagacaagctcagacttgaaagttggagcgcttcggacgtggttggtgctacgaaatgccaaaatagtattggtacttcttcagaactatataaaaactgagctaaaattcatgcattttgaatggtattttcaatagtattttggccacatttactagttagtggaaatattcattttcaaatgtttattcccacctccttactgctgccctactttgaggtcggagataggctgattttgttggttcaagaaggtaagagtgctgcacggttacagatataccagtatagatgtgcagcattattgggatgagtagctgggttacgataataagggtagaacagtaattatgaaacagagtggaagattttagctagaagttgggcattctaggattaaaaaaaagtcttgaagggtaatttgtatcagaacctcgagtttaaagcttgaaataaaaaaagtattaaagctaaaaggattagatttaccagctatcttgacaaatactaaattgcagctttattcatggtttgaagtagaagtcctattcagtaaaacagtgctgtatctctgttaaaccattattaaatatgcagaaaggaaacaggaaaacttgtttttcagtgactatatcttttaattggcattttcagaatgttctcaatgtgacttatatactaaaagtaaaatggaaaagtactggtaatgggtgttcaaaatagatatactttctttttttatttatttattttttttgagacaggatcttgctctgtcacccaggctgtagtgtagtggcgcgatcgcggctcacagcaacctctacctcccaggctcaagcaatcctcctacctcagccccccgagtagctggggctacaggcatgccaccatgcctgcctaattttgtgtgtgtgtgtgtgtgtgttttgtagagacaaggttttgccatattgcccagactggtcttgaattcttgggttcaagtgatttgcctgcctcagcctcccaaagtgttaggattataggcatgagccactgagcccagcctaaaatagatacactttcttaaggttttattgaaagccaaaaagttctctgttctgatttattagtttttcaaatactctgattttaaacatctgtaaggtccatatcctcaccatcaatgtcaatatggtgatatttgagcttgtcaactttttttttttttttggagacggagattcatttttgttgcctaggctggagtgcaatggtgcgatcttggctcaccgcaacttccgcctcctgggttcaagcgattttctgcctcagcctcctgagtagctgggtttacaggcatgcgccaccacacctggataattttgtattttttgtagaaacagggtttctccacattggtcaggctggtctcgaactcccgacctcaggtgatcctcctgccgctgcttcgcaaagtgctgggattacaggcatgagccaccgcgcgtggccttgtctacttgtttttacaaaagttacgtgatttccagatagggaaattaagatactttatatgtgatgcattagactgtgtttattttagatttttaggagcagggggctgtagaaaataacctggaattttgataactaaaatattgtatttcccccaaaacatcttctagtacatagagttcccccctgcccccgagacagggtcttgctcagttgcccaggctggagtatagtggtgtgatcattatagcttactgcagtcttcactccacgggctcaagcagtcctcctgccttggccttccaagtagctgcaacttacaggaacgtgccaccatgcctggcgaacctttttttggtagagacaaggtctcagtatgttgcccccaggctggtctcaaactcctgaactcaagcagtccttccaccttggcctcgaaaactgttggaatcacaggcgtgagccaccctggcccatgtcatttttcttggattaaaataaagtgataagaagtcagttacacccccaggtaaattcagggggaaggggaagggccaggcagaagtatagggaagacagaaattgcaatcttaatattttctttctgtcaagagagaaaaagagagatatatcaaagaaagtgtgttggaagccagaccaggaggtaagatgactagattttaattttagttcttggcttttactgaattaatttagtaattttaagcaagcaaacttggctttctgtgtttactttttttgttatctgcacaattaaggggttgcattgagatttaaagacactttgtcatattatcaaaagtaagtgctacattttatctactgaaaggcattttattcaaattcctctgatagttctctagtagcaggttttaggatactgttttgatccagatttctgttaaagatttagatgaggccaagcagggtggctcacgcctgcaatccgagcactttggaaggtggaggcgggcggatcacttgaggccaggagttccagacaagcctggccaacatggcaaaaccccatctctactaaaaatacaaaaattagctgggtgcagtggtgcttgcctgtagtcccagctacacgggaggctgaggtatgagaactgcttgagcctgggaggtggaggttgcagtgagctgaaatcgagccactgcagtccagcctgggtgacagagtgagactctgtctcaaaaaaaaaaagttttagacgaacacagttcagttgtatatttagccagcatttgttaggtgcctactatgtgccagaaagtaagtgtttttcgtatgttttcttagataactattttatttactgatatatttagaatagtatctggcacaaacttctcagggccatccacttgtacattgtatcctgtcccctttggcctagtcaaggacagagctctaacaattatctcctctcctacatcatctgtttctcactcccattgatgaattatcgttgacattgaacatgggtgtattttctcctatctttttttttttttttttttttttttgagatgggagtctcattctgtccctgaggctggagtgcactgtcagaatctcggcttatataacctctgcctcccgggttcaagcaattctagtgcctcagcctcccaagtagctgagactacaggtatgcaccatgatgcctggctaatttttgtgtttttagtagagacggcattaccatgttggccaggctggtctcaaactcctgacctgaagtgatccacccgccttggcctcccaaagtgctgggattacaggcatgaaccactgtgcctggccctttttttttttttttttttttttttttttttttttttttaaaagacagagtctcttgctctttcactgaggctggagtgcagtggcttgatcattactcactgcagcttctacctcccaggctcaagtgatcctcccccttcagcctcccaagtggctgggatcattggtacatgtcaccatgcccggctaatttttaatttttttttctttttgagcagagtctcactctgttgtccaggctggagtgcagtggcatgatctcggctcactgcactctccacctcctgggttcaagccattcttgtgcatcagcctcccaagtagctgagattacaggaatgcatcaccacacctggctaatttttgcatttttagtagagatggggtttcactgtgttgcccaggctagtcttgaactcctggcctcaagtgatctgcccctacttggcctcccaaagtgttgggattacaggtgtgagccaccgcacctggcctaaattttttttttagagatgagtttttactatattgcctggctggtcttgaacttctggtttcaagcgatcctcttgcctcagtctcccaaagtgctgagattacaggtgtgagttaccatgcctggcctctgtcttaaagcaacagctgcagcagcagcagcaaggaacaactcacctgtgttcccttctagatacccacctgtttctttcctcctttgtgagtttttatatttgctgtttccaattcattttctcccattcattcttgaacttcaagggtaaaggggagctgcttctgtcctctcctgtccaaagtgttttgtttcctactatacaaaaacagctcttaccagggtttgccagtttcctttgtgttgtaaaatccagtgatcaattatcattttttttttttttttaaagctgtctcctacaggaaggaagtgatctcttaccttactaggcctgtcagcagcatttaaaacagcagaatatgccttccttcttgaagctcttacttccaggatactgcttctgcaaggaggaggctggcttgccattgtcttcccaacttctaaaatgttgggtgcctcaagactcagtcctggaaattctttttttttgttgagactgagtttcgctgttgttgcccaggctggagtgcagtggcatgatctcggctcactgcacctccgcctcccaggttcaagcgattctcctgcctcagcctcccaagtagttgggactataggcgtgcgacaccacgcccggctaattttgtatttttggtagagacggggtttctccatgttggtcaggctggtctcgaactcctaacctcaggtgatcctcccgcctcggcctcccaaagtgctgggtttacaggtgtgagccactgtgcctggccagaaattcttttttactctatattcgttccctggagggatcttctctagttccatggttttaaatattgtctatatgctaccaattcctagatttatatcagggccatccacttataccctgtatcctcctcctgatcttcctgctaaactgctgatgtccggctgcctacttaatgtcttcacttggatgtccaatagacatcttaaacttaacatgtttaaaatggaattcctgatctccatcccaccctcaaacctgttcttcctgcaactcttattttttcacaagcaagcgaataataatgatgatagcaatagttaatgcttatatgtgataagcattaactatgcttagatgcttataagcaatagttaatgcttataagtgaataataatgatgatagcaatagttaatgcttatatggtagaaagcactcttctaagagtttttttacacgtaatcttcacgatagtcttattaggtagatgctatttttattttacaaatgaagaaactgaggcagaaagatatttaagtacttgctaggtcacatggtaagtggcagagcctcccaggatacaaactcaggttgccaaaacaccatataattatctcagattctacgtcaatttattagcaaatcctgttggcattatcttcaaaatatatccagaaactgaccatttcctactgccttccctctgccacatgtatgtaagccactttcatcacttattgcaaatattcctatttgctccctgtgtagtatattctcaacacagcagccacttaaaacccaagccaggcaacctcacccttctgctcagtgccctctgatgccttctccatctcacttagataaaagcgtgcatagaggccgggcacggtggctcatgcctgtaatcccagcactttgggaggccgaggcgggcggatcacaagttcaggagatcgagaccatcctggctaacatggtgaaaccctgtctctactgaaaatacaaaaaattagccgggcgtggtggcgggcacctttagtcccaactactcgggaggctgaggcaggagaatggtgtgaacccgggaggcggagattgcagtgagtggagatcacgccactgcactccagcctgggcaacagagccagactccatctcaaaaaaaaaaaaaaaaaaaagcgtgcatagaaagctctacagtgttgcctctctggtccatttccaagtgccctccttgtcactttctcagcctggcatccttgctgctcctcaggccacaccaagtgcacatattttgcacttgctgttcatttagctaggaagtttgcttccttccttcctcaggtttctgctgaaaaatatcttttagagaagtcttcccagaatgccttatataacatagcaaccttctcctttcccctactcccatgcctgacatagaactccatattcttcttacactgctgtttatctttctccctagcacctgtcatcatctgacctagcacgtatttactagttctttgtttactgtctatcttccccctctaggatgtaagtcacttgaggttgtagactttgctttatttgctgctgaggaaatgggtggtacatattagggtacataggtgctaaataggtctttatagaatgatttatcatcttcattttcagagaaacaatgagacacagagaggtgtaacttgcttaagattgtaaagccagtattccagtatgttcagtctggctcctaagcccattcatgtcatccactgtgttcccgaaacagactgaaaggataggctatgtcttagcccacctcatgaaagttaataagcagatgagatgtataatctttgctgggctcaaaaaggtttttttcaagtgtagtttgtagagatgtagaaaatagtagcatatttttagctgaatgagtcagtatgatcgagttaccaaaaaagctgctgtgattctatgaatgatagcgatagcaaacatgtaacactgattacatgtaaggtattctaggctctttacacagattagcccatgtaatcacagtaatctcatgaggtagttaggattattatcctagttttataggttttagcaaacggaggcacaggggttaccgtacatggtgttgtcagtatcagataactggagtcagcaactcggagtggtcttgagtgctgaaagcttaagaaaccaggggctgagaagtagttgaaagaattaaggattctgactggagaaggaattttgtggctcatggcagctgtttttaaacttacaaggttgtttaatggaagagggtctgtggtacaaaactaggtccagtagggaaaagttacggggaagttaagtataaggaagacctttttagtagttctgatggatggttgctatgttgcattctaaatgatcaaacatgggtaaattatagtgatttgactttttttatttttattttttgagacggagtttcactcttgttgtccaggctggagtgcaatggtgcaatctcggctcgctgcaacctccgcctcccaggttcaagcgtttctcctgcctcagcctcccgagtagctgggactacaggcgtgcaccaccactcccagctaattttatatttttagtagagatggagtttcaccatgttggtcaggctggtctcaaacccccgacctcaggtgatccgcctgccttggcctcccaaagtgctgggattacaagagtgagccactgcgcccagctggtgatttgactttttatatggattgattagatgatgatgtaaataaagcccaagttgtgggctcaatgtttgctatgttcatctcctgtaaccataacagaccagatttctcacaagtaaatgctattggttacaaggaagttagatgaaatgtttcattcctgggagaacaagtcagtgcctgccctcctaataagccagttagcaagataatcttagaataaaaacaatacatttttcttatatgtacatctcctcctctcctttcatcctttgctttcatttattcattttttgcctcagctacaaaggacttgagataattttcagtaagattatatagtatggaataatgataaaatagttctaagaatacaatcaaagtatggagaaaggaaatcttttgtaggaaaatattatcatagggctaatataattgtataattgaatgttatgtttcaccttcaggttatctggcaaagttactaaaaagaatcatagtcagttatacatcttcatactttttctcctaagaaacaaagcttttgaaaactttgtttaaaataaattcttaggtggggctctatatagtggcactaaacagtgtagagttgtcttcaccaaagcaatggtagcatgagtgtactttttaattgtaatataccagtgtgggcctataatcatatttcttaaacatttatgaatgtctcttgctgtttattttatagaaatgctaactctaggtattaatgattatctttttttttcttagtaatttatttcttacatttgtttactgtgctttccagaatacagacattgaagtttgggacaagatatttatctaacttctggtaagtccaatgactgttttagagagtgaatttgtaaaataattatacagctatattttcagtcatttaagatttaagataaaaacatgttttcttttgtgcagagttatctcaatatggctgggcatggtggctgatgcctgtaatctcagcactttgggaggctgaggtgggtggaccacttgaggtcaggagtttgggaccagcctggccaacatagtgaaaccctgtctctactaaaaatacaaaaattagccaggcatggtggcacatgcctgtagtcccagctgctcgggatgctgaggcaggaggattgcctgaacctgggaggcagaggtttcagtgaccgagatcgtgccactgtactccagcccgagcaaaagggtgagactccatctcaaaaaagaaaaaaaaattatctcaatgtgacctggtttaagatctaatttttcaacatgatccaattaagatgtgagtcaaattattaaaatcaatctgaaattatattcctcatcagctttattttcttctcttaattatagctttattttcttctctcaattatagtgaattatttcaaaagctaaacacgacatacttcatgagcagacagatgaaaccttataattttcacatttaaatctgcaacaaagttaaccattaaccatctactcattttgggtattaagaaatgactctatatgctatttaagtagagtaaaacatttaactttatagctgtggagaaaataatactggattatttttcccttctagtgtcaaaattagcgacctgctatggcaatgaagaaagaaactgaatttgtcattttcacctgaagaaaaatgatagacaaaaatcaaacctgtggtgtaggacaggattctgtgccctatatgatttgtctgattcacatactcgaagaatggtttggtgtggagcagttggaggactatttgaattttgcaaactatctcttgtgggtttttacaccactaatacttttaatacttccttactttactatctttcttctctaccttactattattttcttacacatttataagagaaagaatgtattgaaagaagcctactctcataatttatgggatggtgcaaggaaaacagtggcaactctgtgggatggacatgcagccgtttggcatggtaagcaaggatactttcacctttgtgttgccattcatgtgtgctgcattggaactgtgttaccattccattttattgattgagaagatactgaaaaatataatagaagtgatatttactattccctgttacctgtttgcatgcattgtgcatatgtatgtgtctttcttaaaatgagttttcccatagacgttggtttccattaaacaaagaaccacgcagcactgtagactataagttgaaataagaaaatttatatttaaaacatctaaaagagtaagagactaaataaagctgtagaaaactgctttaaaaatgcttcaattatttagcatttacttctgttcaaaataagtttgactctaccagtgtggatttaaaaaaatgaaatattaaataatattacattaaatataatttgcataccaaaatttatgcattttctgttgattcattcctgaggttggtgtgtctttctgaataaataagatagcaatagatttctctccaggatagtcattcttttctttattactcgagaaattttgatgttgtatacttaatgattggtggttcttctttgtttggccatcatgaaaattgccttctggtggcctatttttaagtggttagtgtttcactctaccaacttttacttcattattccaacaatatctagtttgttggtctcttttgtttttagactacacaaaaatactgaagattcaataggcaggtaacaaacattggctttctgtattatgttgttgagcatcacagtggtttcatagaaggacatggaaggaagcacagactttaaaagacacttgaggatatatatcactgaactattaggctgctattagcagaaatcaccagtgacatgcttattagactaatgtattttcattttaatactacatacatacttaacgaacctacaaattttttttgagcacctacctatgccagacactgtcaattgcttaatataaattaactgctttaaatctcattaacataatggagtctatgtggtaccccgattatactcatgaggaaactgaggcacagagaggttgttatttgcccaaggtatactaggaatagggccaggttacaaccccagatacctgggagtatacatttgagagagctcttggaggtaaaataaacagaacttggggactgagtgtagagtcttgagggaggaggagttaaagtccagtaagatttctactgctagacagggaaacatgaactggaagtgctccaagtaatagcaaccatgtgagcagaagcacaaaggtggaaaagtactctgggagccacagttagttcatatgatacatgaaataatgtaatgtcactttataggcaattggggcctcaaggggcttgaaaatgttggactagagtggtaatgcactgatactttctcagcagggagtagtatgatcacatctgctttaagagtgacacagttaaatagccctaggttttaaggtttactggcactatagtaaactattttttttaaatatttgattagttggatttttctgctttcaaccaggtggtttggtattatgtgagtagttcaattgtttattatttaaaaaaatactctgtaaaaccagagagaaccttggtaatattctgtatagaatccttcaaaggagtttctttaatgctcagaaaatgtatttgatgggacagttgatcctcaaactcagtgactgttttgtagagcttaaagtaaattgaattacttgagttagaattatgtaattggaaagaaatgatataatatggaaacatacaaaatcttggaatatcattaaattatttttcaaaacattctcaagtccatttgagtagcttatttcatactagaatcacagttatcatacttaatttaatctgttttgtgaagaccttataaactactttagaaatgtattttctgctctggaatttagactgaagtctttattttttgaatgtttatttcgtagctgatcagacaggaatacacttaaggaaagaaacagactaatattttcagatttccatattctggactttacttttgtcttctatacaagtggcagcctttcttgagacagctgttctatgtggaggaatatggccctaagaacttactttcctttcctattacaaccactctagatttccttctttttcacgctttttctttgagcctgattctgcccttgatctctggatgctcacttttttctttttgccaggacccgtggaggctgtggtctgtaatggtgtgtctgacagtggtgacatgggaagggaatgtggatcttatggctagtgccatgatcaccattttattcactccttagaatatcctgacttcccaacttctgtgattttcctctgaggcatagttggatcttacttaagtcttttctattccctactaactgccaacagcacagcgttgctgtaagaagtaaatttgttggccttggtagccatatagagaacagcaggcaggtaatgttagctaagggaggagaaaaaacagccagcctggaactgaggctgtgttagcaggaagcttccagatgtcatcccttctccaggtccaccacctttcatcctgtcatttactcccactactgaaaccctttcttctgtttacagagacataaaatagggcttgcagtgtggaaggaaggggcagggatggcatgagcctctgatctcgcttctacaggacagagattttcttgtagtctagttcaaatctgtgattaaagtttttcccccacatgtaataagattgtacaaaatcatgtttgtttttttcatgaatagaatgaggaaatttctaaagaattgtttaaatgcataaaaatgaagtcttatgtttgtgaatgaacttaaagtttacagcattatgatatctgtgtatatataacagtgtaaatggtattttattttacattcagtcactaataatttttcctgttttaaaaatatgtattttttatttttatttatatttatttatttatttttgagacagagtctcactcttgtgtcccaggctggagtgcagtggtgtgagctcagttcactgcaacctccgcctcccaagttcgagcgattctcctgcctgagccgcccgagtagctgggactataggcacatgccaccacacctggctaatttttgtatttttagtagagacggggtttcaccatgttggccaggctggtctcgaactcctgacctcaggtgatcctcccgcctcaccctcccaaagtgctggtattataggcacgagccactgtgcccggactgtatttacttaatttttttttttttttttgtagagacagggatttcactgatttgcccaggctgatcttaaactcctagcctcaagcaatcctcctgccttggcctcccaaagaactggcattacaggcttgagccatcacacctggcctactttttttgcttttattgatgaattattttatgactcatttttaaatttactgtaggtcatgttatctcagggaagttgtaacttacttgatggcttctgcaaataaggatgttctcatacttaacctcagtaaacccattaaaattggaaaattaacattgttatgttactaatatcaaatctttagagctcatttaagtttacagcctttctatttgactctatttttttatagtttccatctttctgctgaaattcctaatctgattgtgcatgtatccaccttttccactagatcctttgcgtttatttatttatttttatttttagagtcagggtctcgcactgtcacccagcctggagtgcagtggcacgatcatagctcactacagctttcaacttctgggctcaagcaatcatcccacctcagcctcccaagtaactgagactacaggcacatgccaccatgcccaattaatttttttacatttttgcagaggtgggtcttgccatgttgcccaggctgatctctaactcttgggctcaaaagtcctaccatctcagtctcccaaagtgttgggattacaggcgtgagccactgcattcccctttaacttaatcatagttattttatagtttccatctgatagtttcaacatttgggtgtgttaattgttttagctcttgacagtgggtttttttcttgctattttatgtcatattttttattgaattctggccattgtttgtaaaagagtgacaactgaggtaaatagtatttatgactgagaattgggctcataaataggatttatgactaagaatctgtcaggccattggtggttggttggaggtggattcaatctactgagtgaactggatttggcttttattgctgcgctacagctaccttcagttcagcacagacttcagactcctactgttatcttgtgcatagtgtagggcctgaagtagagggattttttggttgtttctgctccaccttcagtcctgtatgcctttgccacaggaagagtctcttcacgcccctcctgcgaccccaggaaaaggtcgcctttgcttataactccacatgggggtacttggggactcctcacaccccttgttcatgcccctgccctcattccccctggtgctggggaataacactcctcagttttcaggtttagctaaaatcttatggggctctgtattcctggggcttaggggtgaggctttctgtgttcctttcccttttccctgtgttgggggttttgtgatggaagcaaatttgctgccactgcccccgtggtagatatctgctttatgtcacttaaggactctgggccccttagactttcctgccacccacccccgctgcccagtggtagatattttgcttcttccccttctctagaatagtggacttttgcctgacactgggctggtgagggtggaggagtgtgtggggcaggggagtggagttctgcccctcccagcattacatgccttttgcatatacccttttcttagagggagtgaaagggtttcctgcccctttcccagcgcctgacaggttttcttctatgagataaacttccaggaagtgagtggggttttgttccctgtaccattagcaggggctctctttactctccagcttcacccctaatctttcttgtgagcacacactgaggtctgtggagaagagttggagagtgactatctggggcttccagagattctacattcaatactagcctaccgtgggactttaagtattttgtaatatgtcacttcttcttaccctcttttatggcatctgcctcttcctcccattctcagtttttgtctttccttagaggggtttatcaccctttgaagttcagttcgcttggttgccttgtgacctctgttctctgacatgcttaagaaaaaaagatttgtacttcaggctttttctcatcagtagggtgggagtgatgttcccttgtggctacagagtttctcttactagtaatactgtacagtatttttaatgttttcaaccccctcctctaacagtgaaacctggctcccgttattcttactatgttgactcatttgatcagtccccctgtgtgtaacccatctcccatcattgctgccttccctacaactcatggatgccctgtccctgcttgtgccaggccaccactctgtacagtgccctgtccagctgctcaggctgtgatgccctacaccaggccacctgtctcgggctctggcccctcctgccaggctgcttgctggttgtggatgcccccatttcactcaggctctgactcccaagcttctgtgacccttgcctcctctcttccccctcagcagccccccaacctgtgataggctctcttctgtgcagtgccctcatgactctgctctggttctgactccccactccgcaccacctgtgtgagtgccttccttagtgtctggcgccaacactccaggctacctctccacacaggttgtctgcttcactgtggcccacctaatggctgtaggactgatttgttcaagaaagaaagagaagaggaaagagagggaagaagggggaaaggggaagagaaccaggaagactagtgtacttttgtgagaaaagactgagctaccaaccatcctttactgtaaacgaattaatgaatatattttaagtatttgcattttgtcattcaaacaggttatgaagttcatggaatggaaaaaataccagaagatggaccagcacttataattttttatcatggagctattcctatagatttttactatttcatggctaaaatatttatacacaaaggcagaacttgccgagtagtagctgatcactttgtctttaaaattccaggtaaactttcactgtagatagtaatgtaaaaatgttttattaacaaaaccaagttatgtggtcgtctctatcctgagctctctaacgtcatttgaaaaagctttttgccttttagggtgtgtataacactcttggaactctaatatgggaatatctaattatcctttttaggcaatatatctaaaaataaagacattgtggccaaaaatgttagctcattttttattttatacattttaaaattgtttatttttaagtgaaaattgtatttatggcatacaatataatgtttttaaaaaataattttaaattttcctttttaaaaaaatatatttttatatattaaaggggtataagtgcagctttcttatatgcatatattgtgtagtggtgaagtctgaatattctcaattagtaactttttcctttataagcaggtattgttatttttttaagttgtatcttgaactgtaacttttaaaaagcaaattgtgatttttgccaatttatattttgatttagcgcatagttgatttttcacataatgaattttttcatgaaatactgatttttggagtttaattttgttataattagagtttgataactctcagtttgatagcaaattaaaattagtaaaaaaaaaaaaagtcatgatatattttgtcttggctgggcacagtggctcatgcctataatcccagcactttgggaggacgaggtgggcaaatcacctgaggtcaggagttcaaaaccagcctggccaacatggtgaaaccccgtctctactaaaaatacaaaaagtagtccagtatggtggctaataggccttctgtaggctcagctactcgggaagctgaggcaagagaatcacttgaacccgggaggcagaggttacagtgagctgacatcatgccactatactccagcctgggcaacagagtaagactccgtctcaaaaaaaaaaaaaagatatattttgtcttcatgtaatattatactaccctaaaaagtacatacatcatgtctcttatcatgtgtaacttgcctgataagatttataacaataagattgagtactacattatttatatttgtttatttatttagatatggggtcttgctatgttgcccaggctggtctcaaattcctgggctcaagtgatcctcccactgtggccttcaaaagttttgggattacaagtgcgagccactgcacccagccgtgaagactacatttaaaaaaatctgtttataaaggctatgtgtgtgtgtgtgtgtgtgtatataaatatatataaatatatataaatatataatatataatatatgtattttatatataatataaatatatactatatattttatatataatataaatatatattacataatatatgtattttatatataatataaatatatattatataatatatgtattttatatataatataaatatataatgtatgtattttatatataatatatatataagtatgtattttatatataatatatatataagtatgtattttatatataatatatattacataatatatgtattttatatataatataaatatatattatataatgtatgtattttatatataatataaatatatattacataatatatgtattttatgtaatataaatatataatatgtatgtattatatatataatataagtatatgtaaatatatatatacacgtgtgtgtgtgtatgtatgtgtgtgtgtgtgtgtgtgtgtatatatatatatatatatatattcgagacagagtcttgctctgtcacctgggctagagtgcagtggcataatctcggctcattgcaacctctgattctcgtacctcagcctcccaagtagttcaaccgattcttgtacctcagcctcccaagtagttgggattgcagatgcccgccaccacacctggctaatttttgtatttttagtagagaagggtttcaccatgttgtccaggttggtcttgaactcctgacctcaggtgatcctcccgcctcaccctcccaaagtgctggtattataggcacgagccaccatgcctagctggccatatgtgtgtatgtgtgtgtgtgtgtatatacatatttattcattttgagaaggaatctcgctcttgttgtccaggctggagtgcaatggtgcgatcgcgactcactgcagcctccgcccctcaggttcaagtgattctcctgcctcagcctccctagtagctgggattactggcgcctgccaccatacctggctaatttttgtacttttagtagagatggagtttcatcatgttggccagactggtctcgaactcctgacctcaggtgatccgcccgccttggcctcccaaagtactgagattgcaggcgtgaggcactgcgtctagccgaccttatattttttttaaaaaaagattaattaaccaaatggtttagagtttttcaaaaatcattcttgagtaaactcattgagattaaatcttattttcttctgtcactgaatcaccagaaacaagccaagagagttgttagtatgttctctggtataaaagcaaaagctgacacatcaaccagacaaacacaaccaaacagtgaattaagtaatgatctacttcagtgcctataaacagttaatgctttgggtatccatgtgtcactaaaaagccatttgtgtataaacatattgtttatggctatgttttctgttaaaaactaacatgatcactatagtagatttggaaactaaaaaacagaacatcattataatcttaccatcttaacataatgttttcattttgatttattttacttcatttttttacttacagtaacataggcaaacagttttgtggggtttttttttaagatttaatgttagcattaatatttttaccatgttttataagaaaaacgtttagtgtatgactagaacatttacttaactgtttttctctaatggagcattaggttgcttctacttttttcgtttttagcaataataaaatgtgaatatttttagcatttgcctttttcaaaaaattttcctctttatgatctttttaatgatcataaagatcattaaagaccttaagacactaagaccttaagtctttctttgctttctgaaggaataaaatcattataattctcagcacaagagagattttagggtgggtgccatggctcacacctgtaattccagcacttggtgaggctaaggcagtaggattgctggagctcagaagattgaaaccaccctgggcaacatagcaagacctcgtttctgctaaaaataaaaataaaaaaatcagccaggcatggtggcacagtgcctggagtctcagctacttgggaggctgagtgagaggatcgcttgagcccagtagagcaaggccgtagtgagctactattgtgcccctgcattccagcctgggtgacagagcaaggccctgtttccaaaaaaaaaaaaaaaaaaagggatcctatattttatcctattatgtatgaatccttccctgttcattcattcattatgagaatgcatctagttgaatcagtgaacatttaatataatgtggggtgacaattattttcttttcttttactttttttgagacagtcttgctctgttgcccaggttggagtgcagtggcttgatctttgctcactgcaacctccgcctcacgggttaaagcaattctccctgcctcagcctcctgagtagctgggattacaagcgtgcgccaccatgcccggctaatttttgtatttttagtagagatggggttttgccatgttggccaggctggtctcaaactcctgacctcaggtggtctgcccgctcatcttcccaaagtgctgggattacaggtgtgaaccaccacacccggtctgacaattattttaatcttaatgctatttataaacttgcctcagatctttaatgagaagtttggttggtctgcatagtgttttttgttttttttctttgagacagagtcttgctctgttgcccaggctgcagtgcagtgacacaattttggctcactgcaacctccccctcctgagttgaagccattctcctgccccagcctcctgagtagctggattacaggcacgtgccaccatgccaggctgatttttgtatttttagtagaggcagggtttcaccacattggccaggctggtcttgaactcctgaccgcaagtgatccacctgcctctttctcccatagtgctggtattacaggcgtgagccaccgtgcctgaccagtctgcgtagtttttaacatcaaatgcatcgctatatttcccattctcagggaataatgtatactaacatcagtcctcagaagtttcagtatgggatttgattaaaatatttaattactaacaggattatttctttcatccataaacagttgtaatttgaaaagctgtataatattaagacttggaattagctgctcttgagtcaagcactctcactttattaaaatctcttaatgatgtttgctattttaaaaatttaattatagaattagcattatctaacttttaatttaaatagatttgatcatggtcttgccagttttggaagagctaagcgtagtttgattcacattctcctgagtgattctcttttttatagggttaatggaagattagatttctgattgctgatgggatatcatttctgaatcccattagaatcctatctctggaatctctgccctgccacttcttagatgtgtgactttgagcaagctattattctgtaagcccaagttttctcatttgtaaagtggggagaagaatacctgtttgtcaagattgttgtgagttttacttaagaatttgagccagagaagcttaatatcaagcttaagttaacctttagagtttttgttgattttaacaagtaaaaaagtttactgatttaagtataaatagtacactatgtagtttgtgaagatttttcttttgaagaattaaacaagaaatacttatgttgtaacttcatactcatgtttcttacagggtttagtttattactggatgtgttttgtgctctacatggaccaagagaaaaatgtgttgaaattctgaggagtggccacttgttagctatctcaccaggtggagttcgagaagccctaattagtgatgaaacttataacatcgtatggggtcatcgcagaggctttgctcaggttgcaattgatgcaaaagtggtgagttatataaaattaattattgaagtaaagcttagtcaaatttaaaataaatggttaagtaatgagccaaattgtataagaacttagacactttcgctgcaataccttaaaaattagtatgtaggccgagtgtggtagctcacgcctgtaatctcagcactttgggaggccgaggtggatgtatcatgaggtcaggagtttgagaccagcctggccaacatgatgaaaccccatctgtactaaaaatacaaaaattagccaggcatggtggtgcatgcctgtaatcccagctactcgggaggctgagggaggagaattgcttgaacccaggaggtagtggttgcagtgagctgagatcgcaccactgcactccagcctgggtgacagagtgagactgtctcagaaaaaaaaaaaaaaaaaagactggagaatttgaatgtcattttgttcttttactttgccttcggaaagtatttctcagccttttggtctccatattgtactttagcctatgaaacacttatctattacctatggaaagtttctgcttcgttttctgtttattttaattctgattgtcagaaaaagaaattacgtttgatagagtattgttctttttccaggccagttagagaatattgtcagtaacttagttttttagttgtatagaaaatgaattatggcacattttacttatttcccagtcttaagcactgacatagaaaacaaaaacctagactatatgaataatatagaatacaaaaggatatatgcagttatacaaaaattaagaataatcaatataatgtatatgaaaacaatgataaaaactaaaatcctaatcaaagatggttcagctttcaagtttatattttgaaagaattgttttttcaaaagtttaagataaagatgacaaaaatagatttaaaatcacataaatatgccacgggattccaattcctcatccttaagtttacattttcggcaacatattgcttggaagaaggactgaaagagataaatgatgcagagaaaataactctttctaatcctcgagtttccagataattaatttaaataaactagataatttaattgagatttttatataaaactttagaaagtttaagaaatacctttttataagtttaaaaacaaacagaaatcttataagtttgataaatattgtgccaccccaacatctcatagcacttttaaacactttatcttatactcctattcagtaaggaggtgatgcatataattattccaatgttgcagatgaggagactcaggcacagagagatggagggatggaagggatctggtcattcagcaaggcagcagtagagcagagcacacagttcagaacttgaggcaaccggtactgttgccattcctgactacattgcacagtgagtctgctgacgtctaagactgaaggggcttcccagatacgtgagggaagtctttcagtaatatatatgtgtatatatatatagttgtaagtggcacaagtatcccctctggaaaatcccttaataggcctcacaaaattataagctgtgaacaaaatttaaggctattgttacaggattaaccacatttaaattgtaacatagtaatagaaattaaaggggaaaaagcctgattgtcaaggcaagttcacaaaatattggtagaaaatattaaggtttattcgaaagtgacagactttttgatataatacaaatcaccctggtttgtgaattcttgagacttacaactttggtaagaacttttagataacttcatgtctacataagttgaggagataactagtccatttactttaattgtaaagtaaatagcagatataaaagaaaatattttgtttcaataaaattatatgaattatctcactttatccataaaaactttgagatagagataattattatttattcccatttgatgcatgaagaaagtgaggcttaaacatggtaagtaacttgtacaatgtctcccacatttttagtgacagagttaagatgtcaagaccccagtaatagtttttaaaaatgtacattctcagctggacatgtgggttcatgcctgtcatcccagcactttcggaggccaaggcaggaggatcacttgaggccaggagttcgagaccagcctgggcaacatggtgaaacctcatctctgctaaaagtacaaaaattagctgggcatggtcttagctactcagatggctgaggttggaggatgactggagcctgggaggcagaggttgcagtgagccgagatcacaccattgtgttccagcttgggcgacagtaaggctctgcttcaaaaaataaaaaaattatacatatatattctctctctctctctccccaccttcctccctcccctccctccctctctcccttgcacacatgcatgcacatacattacatacagacacacattgatatatgtaagcaattgaattctaggttgctgatgcaactccttcatgtacaaagtatgcatgaaagaaggaatgaaggctgaggtgggcagatcacttgaggttaggagatccagaccagcctggccaacatggtcaaactctgtctttaataaaaacacacaaaaaagttagtcgggtgtggtggcacatgcctgtaatcccagctacatgggaggctgaggcaggagagttgcttaaaaacccaggaggcggaggttacagtgagcctagatcatgccactgcactccagcctggacgacagagcaagactccaccttaaataagaggaattaataattatattgcaagatcaagttgggataaagtaatgcttgtggctttaaggatgaaagcttgcgtgccagtgtagaaaccatattcccagactatagtgaggaatactgagcctgctagggtaagctttcttttatttagtgggccctagaaatgtccagcttctagaaaggtagctatttaaggaatgatatttcaacaggaaagtctttaaatgcatacatcaatacagaattctgggatcatgccagtcagtagatataaatccagccatactggttggaagtggcaaatggtatatgcatgagctgaaatatttctgtttcttcttggacattttaggttctcaactataaagacattttataactatttaaaacattaggatttcagccatactaatattttattttacaaaactagttttattttacaaaactagtagtgatttccagataacagtctgaattttttactgaagaattc

SEQ ID NO: 9 - RNA, 177 nt, Homo sapiens
  source 1..177: mol_type = mRNA
atgttagtataagtaacatgaaaacattctcctcctcataagcctacatcagaccaaaatattaaactgacaattaacagcccaatatctacaatcaaccaacaagccattattacccccactgttaacccaacacaggcatgcccacaaggaaaggttaaaaaaaggataaaatga

SEQ ID NO: 10 - DNA, 2515 bp, Homo sapiens
  source 1..2515: mol_type = genomic DNA
gcgctttacggccgcggggacggagcgagccggcgccagggcccctcgggccgggaagaggggaaggggagcgaggttgatgcccggcggaggggcgagcgcggcgtctggccggcttctcaccgccgcggagcaaagagggtcccgggaagcggcagggtcggcgtccaggagcggcttcgggggctccggcggcggcagaggcggagcaagcggccccgggtccgggagcggaggcccggggggccccgcgggcaggatgagcttgaccccgaaggagctctcgagcctgctgagcatcatatcggaggaggcgggcggcggcagcaccttcgagggcctgtccaccgccttccaccactacttcagcaaggccgaccacttccgcctgggctcggtgctcgtcatgctgctccagcagcccgacctgctgcctagcgcggcgcagcgcctcacggcgctctacctgctctgggagatgtaccgcaccgagccgctggccgccaaccccttcgccgccagcttcgcgcacctgctcaaccccgcgccgcccgcccgcggcggccaggaacccgaccgccctccgctctcaggatttttacctcctataactccaccagaaaagttttttctttcccagctgatgctggcacccccacgggaactcttcaaaaagacgcctcgccagattgcactgatggacgttggaaacatgggccagtctgtggacattagtgggcttcagttagccttggccgaacgccaatctgaattgccaacgcaaagcaaagcgagcttccccagtattctcagtgacccagacccggattcttctaattctggatttgacagctcagttgcctctcagatcacagaagctttagtcagcggaccaaagccacctattgaaagccattttcgaccagagtttattcgtccaccgcctccactccacatttgtgaggatgaacttgcttggctaaaccccacggagcctgaccacgcgatccagtgggataaatcgatgtgtgttaagaatagcactggtgtggagatcaaacgaataatggccaaagccttcaaaagccccttatcctctccccaacaaacacagctacttggtgagttggaaaaagaccccaaacttgtctaccatattggcctcaccccagccaaacttcctgaccttgtggaaaacaaccctttagtcgctatagaaatgttgctgaaattaatgcagtcaagccagatcactgagtatttctctgtcctggtcaatatggacatgtctttacattcaatggaagttgtaaatcgactaactacagctgttgatctacctcctgaatttattcacctttatatatcaaattgcatctctacttgtgaacagattaaggataaatatatgcagaatcggttggtgcgtcttgtgtgtgtgtttctccaatccttgatccgtaacaaaattattaatgtacaggatttgtttatagaagtgcaggcattctgtattgaattcagtaggatacgagaagctgctggtcttttccggttgttgaagacattggatactggggaaacaccttctgagaccaaaatgtcaaaataatacctcatcagaaccatcccatccattcactgttcagctgtactgtgatttagtttttacaccgttaaaaccctgagtggattgcttggtttaatgcatataaacagtactttatctacttaaagcaaagttttgctttcttgaatgactttttctgtgagatgaatttttgataagaactagggaaaacatgtcttttaggtgtcttgctgatgactatccataggaggaatggctatcccaaaaaaagttccgcaaaaaagtagatgagtttcttttttttttaagcactaaagaacaaaatgcatttttcattaatacaggcttctgatgaaccaggaatcctgttttcgtaaagttccaatgttgatgagagtaaattcttaagcatttgtcctagaggtgaaagcagctgaatgtttctgaaccatcaagaggcaaacaaacaggagtttgtttcttgaacctgcttatgcacacagctcttaactcctcatgaggcacacagctcttaactcctgatgaaccaaggatttactcataactttctccttgtcatggaggcttaatagacaacagaataaatgcatttcttgggcctcttataaacttgggaattcttagaaagctgcttctattaccaggctgtaatagctggtatagttttttttttttctcttaagatgttctgttattagtctgagacagccatttttttgttttaaggaaaaatatcagtcagtgctccgggaggtaatttcctgtggggtctgcaccctcctgtctgggtggtggatgtgggtttgagaagtaggagagcagggtggtaccgtgtgggctcttaccctttatgtgattttggacaacagtgccttccattaaagttctttttatcaactgtta

SEQ ID NO: 11 - DNA, 690 bp, Homo sapiens
  source 1..690: mol_type = genomic DNA
gtcatggttacctccccacctccccagctgcaaggaggattcatccggcaaccagttgaggctagattctcagcagctttattgaatattgagaggtggtctcacctcccactggacatgtgtcctcaagttcataccagaacttctttgggacagacagacagacaggtccttctccaggaagctcaagtggccttccacgtcccccgcatgggggcccagtttctgcctgagctgttccatctgctgcaccagggctttgttgaagttttccccgtagggctccacccggcgtcggaactcctccacctgctggtccaggtgcccacccagctctgccagtgacttctgcagcccctcggtgttgcccctcaggttgccacgcacgtcctcggccaagggcgccagcctctgccgcagctcctcggcactggccgagatcctggccttgagctcctcggcgttcttcttcatctggaaggtcaggccctcaagctggtggttgagcttctcctgcgtgtcctgagcatagggagccaggctgcggcgcagctcctccacggtctggtcaatcttgactttgaattcgtcagcgtagggcgtaaggcgtcccttgagctcctccacgttctggtcgatcttggccttgagctcgtcggcgtggggcctcagcgaggcctgcaggctgtc

SEQ ID NO: 12 - DNA, 491 bp, Homo sapiens
  source 1..491: mol_type = genomic DNA
aatgctcttcaccagggaggacacgaaggatccctcagccccactgagcacagacaccaggtttccagagggccgcttctggcaggccagctttgagggcaacaaagagaatgagagggagcctgagacctgctgagggagggcagggcaaagagaaaagtgttcgctcatcctgctgagattcaaggagagtcacccctggctagaagctacaatcatccaagtgggaagtaatgtctaccgatgttctgatttaagtgaatgaaggtcttctttccacctcgggaaatcttgagggttatgtaacagcagctttgaaaggcttgtgagatgtactttgtgctagtccatctcaaatcttcaccggctgtaccaagaacagaaaaaaaaaggaatttggtgagtactttataaagctaaatttactcaattaatttcacctctgattatgtttttcatgtatcttgggacattagaatgtcctgtattta

SEQ ID NO: 13 - DNA, 3493 bp, Homo sapiens
  source 1..3493: mol_type = genomic DNA
attcaggggtgtaccaggccccagaggcagcggaaagggagactgtggggaactaggagcaacagcaggcatggaccaaagcagtgaaggatgtatgaaaaagattagcagtgtgaatcttgacaaacttataaatgacttctcacagatagaaaagaaaatggtagaaaccaatggaaagaacaatatactggatattcagttggaaaaaagtaattgcctattaaaagtaatgcaagcaaaggaggtctccattaaagaagaatgtgctactcttcataatataataaaagggctacaacagaccattgaatatcaacagaatttgaaaggtgaaaatgaacaactaaaaataagtgctgatcttataaaagagaagttaaagtctcatgaacaggaatataagaataatattgccaaacttgtaagtgaaatgaaaatcaaagaggagggatataagaaagaaataagcaaactttatcaggacatgcagagaaaagttgaattaaatgaagaaaagcacaaagaactaatagagaaaaaggagatggaaatttcagagttaaatgcaaagctaagaagtcaagaaaaagaaaaacaaaatgaaataatcaagctacaactagaatttgatgccaaactagcaagagttcagactaaatcaaaatcatatcaggattctactgttttgccacaaagtatctacagaaggaaacttcagcattttcaagaagaaaaaaacaaggagattgcaattcttcgtaataccattcgcgatttagagcaacgcctttctgttggcaaagattctcaccttaagcgtagacggttttgagcttagcagtaaattcattagttggtatttatttaaaagcaatcgaaagtttcacttctgttttcaatatatgcatacagcctacataacaaacgaagtcagctttaaggtttttattgaatttatttggacaaatccgtactgtatccaattctgtactgcattcttgcttaatagtattaaccataaaggaggtcaggtttatagggtttggtttacctattaaaccatcattgactatggaaatacttactaagcaatatagagacagacaatattatctttccccttatatcttttaagacagccactcaagttttagaagagtatgatacaacattagagaaagaaagatacaaaggctttattcatgtgtgatagtaaaaatcaggatgagtcttagatatacaaaagataaatggatatttaaaatagttatatatgcttttttagcaaaatattcacgtgttaagtatttctggatcttaaaatacaaaatccacttattttattagttaaaagtagaagattgaaaagacaaagtaatattgttttgagagataagtaaagaaaaaaatggaaagcatgtctttgttgttcttcctccattctaaactgctaattatccaacagaaacccctaggtcatagtttacgtttctattctcattaaagcaaatagaacactggaaaaagaaaaaaaaagacatattaaccaaaagctgcaatcacctttcagttgctccatcataaaaaatatggtttgagtcaatatttctatgacataaaatttaaaatctggaagccaattcagtagatttctccatgtttgcacaaatctaatttctatttttggttcactaatttggtagtttataaaaatgctggaaatgaaattgtgtctagactaaattggggagtatgtcctattttaaatatttagtttgaaggtcattctcacttttttgcctgattctttcagcgtcaactggcactggcttctgtgggtataagctgctgacttatttgtcaggcagctggaggcaaacgtcactgacaagattcagttatgttctcctctgcccgaagtccctgtcagctacataaagatgggaggttttctttacactgtcaggtgattgcagaccctgttttttcaaatatcagatgtcgacaatggcagcatcagctcctaggagccaactctgaatctgtgggcaatttacagagtaattgatttatacatctctttcgacagagcttcttttgtaaatcttgtaaatcttcacttgctggcatatctcggttctctgggtgaccctgcctatgctgacccttgtgcttatggtggtgatgaaggcctggaggagcaggatgagtaggagcatttggtgctcctggttgctgattctctgaaagctcactgctgccaaggtgctgatgtccatgattgtgatgatgctcatgatggtaatgaggcgatggagtttcaactgttttatccacagtagccaaagatacacgtttacaaaagtcttcatctttgagagtctggaacaaatttataaatcactttttaacaagcttatagagataggaataatgcgtgaaaaatgatttgtagagctaacatgtgaaattccaactagttaattagaatcgagctggctttgtattatattaatgagaaagagtctgatgttagtctcaacacctagacattttattaaaggcttaaaaagaaagccaaaccactgtacttatatttgcagaagcaaatgaagtaaactggcaaaaggaacttggattgcaggaaagtcaaagtaatatcaaatcctaaattcagttgatctgggccgagcatggtggctcatgcctgtaatcccaccactttgggtggccgaggggggcagattacttgggctcaggagtcggagaccagcctgggcaacatggcgagatcctgtctctgcaaaatatacaaaaattagctgggtgtggtgtcagatgcctgtagtcccagctacttgtgaggctgaggtgggaggatcgtttgagcctgcaagatggaggttgcagtgagctcagatggtgccactgcactccagcctgggtgacagagtcagaccctatctcaaaaaaataaaaaataaataaaaaatcagttgaactatatggaaacttctgtacctgtgtcaacggtgcatcttatctaatactatatgatactgtaatctaatattatctctgataattcaaaaatgctaataaactctgctcaaatgtgttagtttttcatacttcatcctatgccaggaaatgttttgaacttcaccaaagctttgctaatgtctccctaccataaggctgtcagctgaaaatagcaaattactgcccgactcccattgaccataatatccattccgcatgatcctagttctttaatttactggaagtctttactgttttgtggtacctactaatcagagactcaaagcctatagatacagaaatgtaacagtaaatctcaacctaaggctaatttgcaaataaaattacaaaatcaacttagaacccaaagactcactaagtgatatgcttttgatgttttgaaggttttaaaaggttataaattccattaaattttagcatacaacta

SEQ ID NO: 14 - DNA, 744 bp, Homo sapiens
  source 1..744: mol_type = genomic DNA
atggggaagcgggacaatcgggtggcctatatgaacccaatagcaatggcgagatcaaggggtccaatccagtcttcagggccaacaatacaggattatctgaatcgaccaaggcctacctgggaagaagtaaaagagcaactagaaaaggaaaagaaaggctccaaggctttggctgaatttgaagaaaaaatgaatgagaactggaagaaagaactggaaaaacacagagagaaattgttaagtggaagtgagagctcatccaaaaaaagacagagaaagaaaaaagaaaagaagaaatctggtaggtattcatcttcttcttcatcaagctctgattcttccagcagttcttctgattctgaagatgaggataagaaacaaggaaaacagagaaagaaaaagaagaaccgttcacataaatcttctgaaagctccatgtcagaaactgaatcagacagtaaggatagtttaaaaaagaaaaagaagtcaaaagatggaactgagaaagaaaaggatattaaaggactcagcaaaaagagaaagatgtattctgaagataaacctttatcatctgagtccttgtcagaatcagagtatattgaggaggtgcaagcaaaaaagaagaaaagcagtgaagaacgagaaaaagcaacagaaaaaacaaaaaagaaaaagaagcataagaaacacagtaagaagaagaaaaagaaggctgctagttcaagtcctgactcaccgtaa

SEQ ID NO: 15 - DNA, 690 bp, Homo sapiens
  source 1..690: mol_type = genomic DNA
cccaaagtgctgggattacaggcgtgaaccacagcgcccggccaagattaacattcgagtcagtgggctggggaaggcagatccacccttaatctggtgggcacaatctaatcagctgctggcaattaaagcacgctgaaaaacgtgaaaaggagagacaggcccagcctcccagcctacatcttcctcctgtcctggatgcttcctggcctcgaacatcggactccaagtccttcaattttgagactcagactggctctccttgctcctcagcttgcagacagcctactgtgagaccttgtgatcatatgggacttgctcctccttgtcttctgccatgattgtgaggcatccccagccatgtggaactttttggagcaattgtggcagtgagatcattccaactcgtggatcacagatacagcagcgagttcctgaatcccagacccagtggtggtcctctaactgaatgattatagcaaatgttgcagcatccctccaggataacttcagattcctggcatcattctagaaggcttagcccataattcattcctttggcccttccaataatcagaaagcacaagctcgtgaacatcttgggaatctgcaggtttgcttatcatctgttaaatattaaaaagagtactttttaaattcctaataaactggttaatgcatttagacaga

SEQ ID NO: 16 - RNA, 2068 nt, Homo sapiens
  source 1..2068: mol_type = genomic RNA
ctgcagggtccggaaaagaaagagaaacggcccagcgcggtcgctcacaaaattcagggcggggctgccctcggcgcccgaaagctttttgttttttgtttttttttgaaacggagtttcgctcttgttgcccaggctggagtgcagtggcgcgatctcggctcacagcaacctccgcctcccgggttcaagtgagtctcctgcctcagcctcccgactagctgggattacaggcatgcgccacccccggctaattttgtatttttagtagaggcggggtttctctatgttggtcaggctggtctggaaatcccgacctcaggtgatccgcccgcctcggcctcccaaagtgctggaattacaggcttgagccaccgcgcacggctttgcccgcaagcgttttgaattttgtggcccggaattcactgcgaggactgggatcacccatcacccttccctggtctacggaaaatgaaacgtgtttactgatacagaaacggaataacggcactttgggctggggagggccgagctgcctcaggcttctggtctccagctgcgcggcactcacacctgccgctgtgaaaatgcagacccgcggggcaggaattccgagtccaggctggagcgcgatctggaatctgactcgctggaaacagcaccgcggtggattcggacctggggttggggcctggagagacagaggctgccactgattctctgaattccagcttcccttgtcagtcacgcagctgaaagcaggaagaagctttctcttctgcacttggaacaacaaggtgaagatgacagagagtcccacatctaccatggaaaaggcaatggtccctaaatcatgccaagttttctaatgacatgtgttttcccgcaacatgtaactatatgtcctcccttcaagcaccagttatcacagtacaaacacagcaacagcataaaggaatgactactgagcctgaagctctgatggatgctctgagttgagactttgttttatacttaggtacctgtgagtcattgcagagttttgcctgactccaccatgacccaatttgatcaaggtctatatgctatgagacctctctagttcctcatcagaaattataatgaataccatgtccagctcctgaggtgcctgatggcaacgagcggcagctcctcttggggcaacactttgggggtgaccaaggcctcacagacctcactactgctgggctacatggcccaaggctcctgggcgcaatggatcggtggtcttggattcgcctcggacatttaatgacatttgttcttgggctttcacaagtggtctctggttgagatggaaggaaaggacgcagggacacaaaatctctgactgcccagggaccccctcaccgtcctcacttccactcaagacgacatggccgttccactaaattcccgaggacatggccaacatgagtaacaacacagggaagtgccttacacagagttccatggaagcctggctagtaccctctagaccatttacctacacctgttgggatctctctagcatgaaatgctccagaggcagtcatgccataagcttggatcctccagccattgagtgtgagggtcttagcccggtctaaatgagcactgaattctagagagaccaagggactgtctaagttctgaattctgagtcctgaatcctggaagttggcttctggccagggagcaggtgttactgcctctgatccacaaaagcagccccacagctcaagttgctcttctggatttctcccctctcctggttcctggccaggcaaatcttcactgctttgttagcccatccatttgctcaaccagatttttcaaatatgtatgttttcctccttttcccattgtactcagtggaaggaatggtctaaattacctaatcagcaattcctggaaaccaagccaccatgtacatacaatcttatacgttaatgtcacatacaagaaagaaaatgcagaggtaccagtagtggctacctaagcagtgtaatgtagaggtaataaaaacgaattgttggatagtatc

SEQ ID NO: 17 - DNA, 291 bp, Homo sapiens
  source 1..291: mol_type = genomic DNA
acaaggtgtggtggtgcacgcctgtagtcctagctacttgggaggctgaggcaggagggtggcttgagtccaggagttctgggctgtagtgtgccatgtcaattgggtgtcagcactaagtttggcatcaatatgggagcaggggaccactaggttgtttaaggaggagtgggtgcactggctcacaccagaaatggagcaggtcaaaactcctgtgctgatcagtagtgggatcacacctgtgaagagccactgcactcctgcctaggcaacatagtgagatctgtctct

SEQ ID NO: 18 - DNA, 1215 bp, Homo sapiens
  source 1..1215: mol_type = genomic DNA
ctggtgcttccgaccgctgcgcgcggctcctgggctgtcacagtctcccgttgccgccgtcatgtcccggcagctgtcgcgggcccggccagccacggtgctgggcgccatggagatggggcgccgcatggacgcgcccaccagcgccgcagtcacgcgcgccttcctggagcgcggccacaccgagatagacacggccttcgtgtacagcgagggccagtccgagaccatccttggcggcctggggctccggctgggcggcagcgactgcagagtgaaaattgataccaaggccattccactgtttgggaactccctgaagcctgacagtctccggttccagctggagacgtcactgaagcggctgcagtgtccccgagtggacctcttctacctgcatatgccagaccacagcaccccggtggaagagacactgcgtgcctgccaccagctgcaccaggagggcaagttcgtggagcttggcctctccaactatgcagcctgggaagtggccgagatctgtaccctctgcaagagcaacggctggatcctgcccactgtgtaccagggcatgtacaatgccatcacccggcaggtggaaacggagctcttcccctgcctcaggcactttggactgaggttctatgccttcaaccctctggctgggggcctgctgaccggcaagtacaagtatgaggacaagaatgggaaacagcccgtgggccgcttctttgggaatacctgggcagagatgtacaggaatcgctactggaaggagcaccactttgagggcattgccctggtggagaaggccctgcaggccgcgtatggcgccagcgcccccagcatgacctcggccaccctccggtggatgtaccaccactcacagctgcagggtgcccacggggacgcggtcatcctgggcatgtccagcctggagcagctggagcagaacttggcagcggcagaggaagggcccctggagccggctgtcgtggacgcctttaatcaagcctggcatttggttactcacgaatgtcccaactacttccgctaggcccatcgtttctcaggctgcccaaggctcttctgtaacatcttttgttactcacattttctttaatttagaactgcctcagtaaattcttagggatggaagtatttggacaaaaacctaatagaagagtcaccaccaaatgaagaataaaactccca

SEQ ID NO: 19 - DNA, 402 bp, Homo sapiens
  source 1..402: mol_type = genomic DNA
gctggggtctcaggaggcagcgctctcaggacatctccaccatggcctgggctctgctgctcctcaccctcctcactcagggcacagggtcctgggcccagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctccacagtg

(19) United States
(12) Patent Application Publication
(10) Pub. No.: US 2026/0218302 A1
(43) Pub. Date: Jul. 30, 2026

(54) DIAGNOSING UPPER GI PATHOLOGIES

(71) Applicant: Sheba Impact Ltd., Ramat-Gan (IL)

(72) Inventors: Bella UNGAR, Ramat Gan (IL); Shomron Silan BEN-HORIN, Ramat-Efal (IL); Ella FUDIM, Herzeliya (IL)

(73) Assignee: Sheba Impact Ltd., Ramat-Gan (IL)

(21) Appl. No.: 19/403,108

(22) Filed: Nov. 27, 2025

Related U.S. Application Data
(63) Continuation of application No. PCT/IL2024/050541, filed on May 31, 2024.

(30) Foreign Application Priority Data
May 31, 2023 (IL) 303353

(51) Int. Cl. C12Q 1/6883

(52) U.S. Cl. CPC C12Q 1/6883; C12Q 2600/158

(57) ABSTRACT

Methods of diagnosing upper gastrointestinal pathologies are provided. Accordingly, there are provided methods of isolating an RNA sample from an upper gastrointestinal tract of a human subject. Also provided are methods of diagnosing an upper gastrointestinal pathology comprising analyzing an expression level of at least one gene associated with the pathology in a sample of an upper gastrointestinal tract secretion of the subject.

### RELATED APPLICATIONS

This application is a Continuation of PCT Patent Application No. PCT/IL2024/050541 which claims the benefit of priority of Israel Patent Application No. 303353 filed on May 31, 2023. The contents of the above applications are all incorporated by reference as if fully set forth herein in their entirety.

### SEQUENCE LISTING STATEMENT

The XML file, entitled 105493SequenceListing.xml, created on Oct. 29, 2025, comprising 238,509 bytes, submitted concurrently with the filing of this application is incorporated herein by reference.

### FIELD AND BACKGROUND OF THE INVENTION

The present invention, in some embodiments thereof, relates to diagnosing upper GI pathologies.

Many upper gastro-intestinal (GI) pathologies, such as eosinophilic esophagitis, gastritis, enteritis, celiac disease, gastric lymphoma, gastric carcinoma, pancreatic and biliary cancer, are currently diagnosed and monitored using GI biopsies.

Eosinophilic esophagitis (EoE, an eosinophil rich, Th2 antigen mediated disease), for examples, is of increasing pediatric and adult worldwide prevalence, wherein symptoms reflect esophageal dysfunction and include heartburn/regurgitation, vomiting, dysphagia, episodes of food impaction, chest pain and even abdominal pain. Endoscopic findings, such as concentric rings, can be suggestive, however rare and non-specific. EoE diagnosis is based on the presence of esophageal eosinophilia greater than or equal to 15 eosinophils per high power field under routine light microscopy following hematoxylin and eosin staining of a biopsy sample. Disease monitoring includes repeated gastroscopies with multiple biopsies, even in pediatric patients. (Gonsalves NP and Aceves SS (2020) J Allergy Clin Immunol. 145(1): 1-7; Furuta GT and Katzka DA (2015) N Engl J Med. 373(17): 1640-8). Another example is celiac disease (an immune-mediated enteropathy triggered by gluten), a relatively common condition affecting ~1% of the world population that can develop across the lifespan. The incidence has been increasing rapidly over the past four decades worldwide. Clinical presentation of celiac disease is highly variable and includes classical and non-classical gastrointestinal symptoms, extra-intestinal manifestations, and subclinical cases. The disease is associated with a risk of complications, such as osteoporosis and intestinal lymphoma. Diagnosis of celiac disease requires a positive serology and villous atrophy on small-intestinal biopsy. Repeated upper endoscopies with multiple biopsies are required in cases of symptoms exacerbation and clinical worsening. (Pinto-Sanchez M I et al. (2021) Nat Rev Gastroenterol Hepatol 18(12): 875-884; Catassi C et al. (2022) Lancet 399(10344): 2413-2426).

In addition, in the interest of predicting patient prognosis or response to therapies, RNA transcriptomic are widely used in analyzing the GI biopsies. Nevertheless, association of biopsy transcriptomic with therapy selection, diagnosis and prognosis is limited, probably due to the fact that biopsies provide information on a specific (approximately 2 mm) area.
Background art includes:
Ungar et al. (2022) Gut gutjnl-2021-325516;
Dan S et al. (2023) Cell Mol Gastroenterol Hepatol 16 (1): 1-15;
International Patent Application Publication Nos. WO2023/002491 and WO2006038634; and
US Patent Application Publication Nos. US20200138416 and US20160010081.

### SUMMARY OF THE INVENTION

According to an aspect of some embodiments of the present invention there is provided a method of isolating an RNA sample from an upper gastrointestinal (GI) tract of a human subject, the method comprising:
(i) collecting from the subject a sample of an upper GI tract secretion;
(ii) freezing the sample in liquid nitrogen following the collecting until the sample is completely frozen, wherein the freezing is done no more than 2 minutes following the collecting; and
(iii) extracting an RNA from the frozen sample using a TRIzol®-based extraction method.

According to an aspect of some embodiments of the present invention there is provided a method of isolating an RNA sample from a saliva sample of a human subject, the method comprising:
(i) collecting from the subject a saliva sample having a volume of at least 0.8 ml;
(ii) freezing at least 0.8 ml of the sample in liquid nitrogen following the collecting so as to obtain a completely frozen sample, wherein the freezing is done no more than 2 minutes following the collecting;
(iii) incubating the frozen sample with an RNA preservation solution so as to obtain a completely thawed RNA preservation solution-sample mix; and
(iv) extracting an RNA from the thawed RNA preservation solution-sample mix.

According to some embodiments of the invention, the upper GI secretion is a saliva sample of the subject.

According to some embodiments of the invention, the saliva sample is obtained from the subject following at least 6 hours fasting.

According to some embodiments of the invention, the saliva sample is obtained prior to performing a gastroscopy in the subject.

According to some embodiments of the invention, the upper GI secretion is a stomach and/or an intestinal secretion sample of the subject.

According to some embodiments of the invention, the intestinal secretion is a duodenum secretion.

According to some embodiments of the invention, the duodenum is the duodenum blub.

According to some embodiments of the invention, the collecting is during a gastroscopy.

According to some embodiments of the invention, the collecting is performed prior to passage of an endoscope in the upper GI tract part the sample is obtained from.

According to some embodiments of the invention, the collecting is effected following a first washing.

According to some embodiments of the invention, the first washing comprises injecting sterile liquid into the upper GI tract and collecting a fluid sample thereafter.

According to some embodiments of the invention, the collecting is effected by suctioning a fluid sample from the upper GI tract.

According to some embodiments of the invention, the sample of the upper GI tract secretion comprises at least 1 ml sample.

According to some embodiments of the invention, the sample collected from the subject has a volume of at least 1 ml.

According to some embodiments of the invention, the sample of the upper GI tract secretion comprises about 5 ml sample.

According to some embodiments of the invention, the sample collected from the subject has a volume of about 5 ml.

According to some embodiments of the invention, freezing the sample in liquid nitrogen is effected for at least 30 minutes.

According to some embodiments of the invention, freezing the sample in liquid nitrogen is effected for up to 120 minutes.

According to some embodiments of the invention, the frozen sample is kept at −80° C. until the extracting.

According to some embodiments of the invention, the TRIzol®-based extraction method comprises a sample to TRIzol® ratio of 1:1-1:3

According to some embodiments of the invention, the TRIzol®-based extraction method comprises mixing the frozen sample with the TRIzol® until obtaining a completely thawed TRIzol®-sample mix.

According to some embodiments of the invention, the TRIzol®-based extraction method comprises adding TRIzol® to the frozen sample at a ratio of 1:1-1:3 and mixing until obtaining a completely thawed TRIzol®-sample mix.

According to some embodiments of the invention, the frozen sample is kept on ice throughout the thawing process.

According to some embodiments of the invention, the method comprising mixing the thawed TRIzol®-sample by vortex to homogenize the TRIzol®-sample mix.

According to some embodiments of the invention, the method comprising keeping the thawed TRIzol®-sample mix at room temperature for 5-10 minutes.

According to some embodiments of the invention, the method comprising adding the thawed TRIzol®-sample mix without the upper layer of fat and the lower level of sediment to an equal volume of ethanol and mixing to obtain a TRIzol®-sample-ethanol mix.

According to some embodiments of the invention, the method comprising loading the TRIzol®-sample-ethanol mix to an RNA-binding column, centrifuging the column and discarding the flow-through.

According to some embodiments of the invention, the loading is effected multiple times, each time with up to 1 ml of the TRIzol®-sample-ethanol mix.

According to some embodiments of the invention, the loading is effected multiple times, each time with up to 0.7 ml of the TRIzol®-sample-ethanol mix.

According to some embodiments of the invention, the frozen sample to the preservation solution ratio is at least 1:1.

According to some embodiments of the invention, the frozen sample is kept at 4° C. throughout the incubating.

According to some embodiments of the invention, comprising centrifuging the thawed RNA preservation solution-sample mix to obtain a sample pellet and adding to the sample pellet at least 500 μl lysis buffer to obtain a lysed sample.

According to some embodiments of the invention, the method comprising adding to the lysed sample at least 500 μl ethanol to obtain a lysed sample-ethanol mix.

According to some embodiments of the invention, the method comprising loading the lysed sample-ethanol mix to an RNA-binding column, centrifuging the column and discarding the flow-through.

According to some embodiments of the invention, the method comprising eluting an RNA from the RNA-binding column by adding a total of at least 25 μl of nuclease-free water and centrifuging the column.

According to some embodiments of the invention, the method comprising eluting an RNA from the RNA-binding column by adding a total of at least 30 μl of nuclease-free water and centrifuging the column.

According to some embodiments of the invention, the method comprising eluting an RNA from the RNA-binding column by adding a total of at least 40 μl of nuclease-free water and centrifuging the column.

According to some embodiments of the invention, the eluting is effected in at least two rounds.

According to some embodiments of the invention, the centrifuging is at 4° C.

According to some embodiments of the invention, the centrifuging is in 10,000-11,000 g.

According to some embodiments of the invention, the method further comprising analyzing an RNA expression level in the RNA sample following the extracting.

According to an aspect of some embodiments of the present invention there is provided a method of diagnosing an upper gastrointestinal (GI) pathology in a human subject in need thereof, the method comprising:
isolating RNA sample from an upper GI tract of the subject according to the method; and
analyzing an RNA expression level of at least one human gene in the RNA sample following the extracting, wherein the expression level is indicative of the upper GI pathology.

According to some embodiments of the invention, an altered expression level compared to an upper GI tract secretion RNA sample of a control subject not suffering from the upper GI pathology is indicative of the pathology.

According to some embodiments of the invention, no significant change in expression level compared to an upper GI tract secretion RNA sample of a control subject suffering from the upper GI pathology is indicative of the pathology.

According to some embodiments of the invention, the pathology is selected from the group consisting of eosinophilic esophagitis (EoE), peptic ulcer disease, eosinophilic gastritis, collagenous gastritis, esophagitis, enteritis, atrophic gastritis, celiac disease, primary sclerosing cholangitis, primary biliary cirrhosis, gastric lymphoma, gastric carcinoma, hepatocellular carcinoma, pancreatic cancer, biliary cancer and gastric/duodenal neuro-endocrine tumor.

According to some embodiments of the invention, the pathology is eosinophilic esophagitis (EoE) or celiac.

According to an aspect of some embodiments of the present invention there is provided a method of diagnosing celiac in a human subject in need thereof, the method comprising analyzing an expression level of at least one gene selected from the group consisting of AP006216.3, AC011754.1, CCDC152, AKR7A3 and IGLV2-14 in a sample of an upper GI tract secretion of the subject, wherein the expression level above a predetermined threshold is indicative of celiac.

According to some embodiments of the invention, the method further comprising analyzing an expression level of at least one additional gene selected from the group consisting of AC122718.1, AP001011.1, MICB-DT, RN7SL288P and AC092746.1 in the sample of the upper GI tract secretion of the subject, wherein the expression level of the at least one additional gene below a predetermined threshold is indicative of celiac.

According to an aspect of some embodiments of the present invention there is provided a method of diagnosing celiac in a human subject in need thereof, the method comprising analyzing an expression level of at least one gene selected from the group consisting of AC122718.1, AP001011.1, MICB-DT, RN7SL288P and AC092746.1 in a sample of an upper GI tract secretion of the subject, wherein the expression level below a predetermined threshold is indicative of celiac.

According to an aspect of some embodiments of the present invention there is provided a method of diagnosing eosinophilic esophagitis (EoE) in a human subject in need thereof, the method comprising analyzing an expression level of at least one gene selected from the group consisting of MALAT1, AC092746.1M AC114760.2, RGPD4-AS1, FAM133B, TMEM52B, AC018607.1, AC114801.3 and CNOT11 in a sample of an upper GI tract secretion of the subject, wherein the expression level above a predetermined threshold is indicative of celiac.

According to some embodiments of the invention, the method further comprising analyzing an expression level of CHST7 in the sample of the upper GI tract secretion of the subject, wherein the expression level of the CHST7 below a predetermined threshold is indicative of EoE.

According to an aspect of some embodiments of the present invention there is provided a method of diagnosing eosinophilic esophagitis (EoE) in a human subject in need thereof, the method comprising analyzing an expression level of CHST7 in a sample of an upper GI tract secretion of the subject, wherein the expression level below a predetermined threshold is indicative of EoE.

According to some embodiments of the invention, the analyzing the expression level comprises analyzing an RNA expression level.

According to some embodiments of the invention, the RNA is obtained according to the method.

According to some embodiments of the invention, diagnosing the pathology comprises determining the severity of the pathology.

According to some embodiments of the invention, the expression level of the at least one gene in a saliva sample of the subject correlates with the expression level of the at least one gene in a non-saliva upper GI tract secretion of the subject.

According to some embodiments of the invention, the expression level of the at least one gene correlates with the degree of histological inflammation.

According to some embodiments of the invention, the analyzing the expression level comprises performing whole cell transcriptome analysis.

According to some embodiments of the invention, the analyzing the expression level comprises performing RT-PCR.

According to an aspect of some embodiments of the present invention there is provided a method of treating an upper GI pathology of a subject in need thereof, the method comprising:
(a) confirming that the subject has the upper GI pathology according to the method; and
(b) administering to the subject a therapeutically effective amount of an agent useful for treating the pathology.

According to an aspect of some embodiments of the present invention there is provided a method of treating an upper GI pathology of a subject in need thereof, the method comprising:
(a) receiving an identification that a subject has an upper GI pathology according to the method; and
(b) administering to the subject a therapeutically effective amount of an agent useful for treating the pathology.

According to an aspect of some embodiments of the present invention there is provided a method of treating celiac in a subject in need thereof, the method comprising:
(a) confirming that the subject has celiac according to the method; and
(b) administering to the subject a therapeutically effective amount of an agent useful for treating celiac.

According to an aspect of some embodiments of the present invention there is provided a method of treating celiac in a subject in need thereof, the method comprising:
(a) receiving an identification that a subject has celiac according to the method; and
(b) administering to the subject a therapeutically effective amount of an agent useful for treating celiac.

According to an aspect of some embodiments of the present invention there is provided a method of treating eosinophilic esophagitis (EoE) in a subject in need thereof, the method comprising:
(a) confirming that the subject has EoE according to the method; and
(b) administering to the subject a therapeutically effective amount of an agent useful for treating EoE.

According to an aspect of some embodiments of the present invention there is provided a method of treating eosinophilic esophagitis (EoE) in a subject in need thereof, the method comprising:
(a) receiving an identification that a subject has EoE according to the method; and
(b) administering to the subject a therapeutically effective amount of an agent useful for treating EoE.

According to some embodiments of the invention, the subject shows symptoms of the pathology.

According to an aspect of some embodiments of the present invention there is provided a diagnostic kit for diagnosing celiac, the kit comprising at least two agents capable of specifically detecting expression products of at least two genes associated with celiac, wherein at least one of the at least two genes is selected from the group consisting of AC122718.1, AP001011.1, MICB-DT, RN7SL288P, AC092746.1, AP006216.3, AC011754.1, CCDC152, AKR7A3 and IGLV2-1, wherein the kit comprises no more than 20 agents capable of specifically detecting the expression products.

According to an aspect of some embodiments of the present invention there is provided a diagnostic kit for diagnosing eosinophilic esophagitis (EoE), the kit comprising at least two agents capable of specifically detecting expression products of at least two genes associated with celiac, wherein at least one of the at least two genes is selected from the group consisting of MALAT1, AC092746.1M AC114760.2, RGPD4-AS1, FAM133B, TMEM52B, AC018607.1, AC114801.3, CNOT11 and CHST7, wherein the kit comprises no more than 20 agents capable of specifically detecting the expression products.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a Tape-Station figure, depicting sample concentration and base-pair length, demonstrating intact RNA successfully extracted from a gastric secretion sample of a human subject during gastroscopy.

FIG. 2 is a gel demonstrating RNA extraction from sputum samples. A1—eosinophilic esophagitis (EOE) patient; B1—healthy control patient; C1—healthy control patient; D1—healthy control patient. Tape station analysis determines amount of RNA in EOE, celiac and control samples).

FIG. 3 is a gel demonstrating intact DNA amplified from RNA samples extracted from sputum samples. A1—EOE patient; B1—healthy control patient; C1—healthy control patient; D1—healthy control patient; E1—celiac patient.

FIGS. 4A-B demonstrate altered gene expression detected in sputum samples of celiac disease patients prior to gluten avoidance (n=7) as compared to controls (n=7 healthy patients+9 EOE patients). Shown is hierarchical clustering (FIG. 4A) and a summing table of the statistically significantly altered genes (FIG. 4B, p<0.05).

FIGS. 5A-B demonstrate altered gene expression detected in sputum samples of EOE (n=9 as compared to controls (n=7 healthy patients+7 celiac patients). Shown is hierarchical clustering (FIG. 5A) and a summing table of the statistically significantly altered genes (FIG. 5B, p<0.05).

### DESCRIPTION OF SPECIFIC EMBODIMENTS OF THE INVENTION

The present invention, in some embodiments thereof, relates to diagnosing upper GI pathologies.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Upper gastrointestinal (GI) tract fluids are associated with inflammation and immune activity in various upper GI pathologies, and thus may enable diagnosis, staging and assessment of response to therapy.

Whilst reducing specific embodiments of the present invention to practice, the present inventors have now established novel and unique methods for extracting RNA from upper GI samples which was further amplified and sequenced (Examples 1-2 of the Examples section which follows). Furthermore, deep sequencing analysis revealed several genes having significant altered expression patterns in samples obtained from celiac disease and eosinophilic esophagitis (EoE) patients.

Thus, according to an aspect of the present invention, there is provided method of isolating an RNA sample from an upper gastrointestinal (GI) tract of a human subject, the method comprising:
(i) collecting from the subject a sample of an upper GI tract secretion;
(ii) freezing said sample in liquid nitrogen following said collecting so as to obtain a completely frozen sample, wherein said freezing is done no more than 2 minutes following said collecting; and
iii) extracting an RNA from said frozen sample using a TRIzol®-based extraction method.

According to an additional or an alternative aspect of the present invention, there is provided a method of isolating an RNA sample from a saliva sample of a human subject, the method comprising:
(i) collecting from the subject a saliva sample having a volume of at least 0.8 ml;
(ii) freezing at least 0.8 ml of said sample in liquid nitrogen following said collecting so as to obtain a completely frozen sample, wherein said freezing is done no more than 2 minutes following said collecting;
(iii) incubating said frozen sample with an RNA preservation solution so as to obtain a completely thawed RNA preservation solution-sample mix; and
(iv) extracting an RNA from said thawed RNA preservation solution-sample mix.

As used herein, the “upper gastrointestinal (GI) tract” includes the mouth, esophagus, stomach, and small intestine (duodenum, jejunum, and ileum) and comprises both the lumen or cavities and the lining cells and tissues.

As used herein, the phrase “a sample of an upper GI tract secretion” refers to a secreted or shedded biological sample collected from the lumen or cavities of any part of an upper GI tract of a subject. Such secretions may include substances secreted from the glands and cells within the upper GI tract; and also exfoliated cells and tissue fragments. Thus, samples that can be used with specific embodiments of the invention include a sample obtained from the mouth (e.g. saliva), a sample obtained from the esophagus, a sample obtained from the stomach and a sample obtained from the small intestine (e.g. duodenum; e.g. the duodenum blub).

According to specific embodiments, the sample is not a small intestine sample.

According to specific embodiments, the sample is a saliva sample.

Methods of colleting a saliva sample are known in the art, and include for example a swab sample, a sputum sample, and a sample obtained during gastroscopy.

According to specific embodiments, the saliva sample is a swab sample.

According to specific embodiments, the saliva sample is a sputum sample.

According to specific embodiments, the saliva sample is obtained prior to performing a gastroscopy in the subject.

A non-limiting example of a saliva collection procedure that can be used with specific embodiments is described in details in the Examples section which follows, which serves as an integral part of the specification.

According to specific embodiments, the sample is a stomach, an intestinal secretion or a combination of both.

According to specific embodiments, the intestinal secretion is a duodenum secretion.

According to specific embodiments, the duodenum is the duodenum blub.

Methods of colleting such samples are known in the art, and include for example gastroscopy. A non-limiting example of a gastroscopy procedure that can be used with specific embodiments is described in details in the Examples section which follows, which serves as an integral part of the specification.

According to specific embodiments, collection of the sample (e.g. of a gastric secretion) is performed during gastroscopy prior to passage of an endoscope in the part the sample is collected from.

According to specific embodiments, collection is effected without a washing step.

According to specific embodiments, the sample is obtained following an initial wash (e.g. using sterile water, saline or PBS). According to specific embodiments, such a sample comprises both the GI secretion(s) and the washing liquid.

According to specific embodiments, the sample is suctioned from the subject during gastroscopy.

According to specific embodiments, the sample is obtained from the subject following fasting for at least 1 hour, at least 2 hours, at least 3 hours, at least 4 hours, at least 5 hour, at least 6 hours of more.

According to specific embodiments, the sample is obtained from the subject following at least 6 hours' fasting.

According to specific embodiments, the sample collected from the subject has a volume of at least 0.5 ml, at least 0.6 ml, at least 0.7 ml, at least 0.8 ml, at least 0.9 ml, or at least 1 ml.

According to specific embodiments, the sample collected from the subject has a volume of at least 0.8 ml.

According to specific embodiments, the sample collected from the subject has a volume of at least 1 ml.

According to a specific embodiment, the sample collected from the subject has a volume of about 1 ml.

According to specific embodiments, the sample collected from the subject has a volume of at least 2 ml, at least 3 ml, at least 4 ml, or at least 5 ml.

According to a specific embodiment, the sample collected from the subject has a volume of about 5 ml.

Following collection, the sample obtained from the subject undergoes a freezing process.

According to specific embodiments, the sample is first cooled in liquid nitrogen to allow rapid freezing of the whole sample. Following complete freezing of the sample, it can be kept e.g. at −80° C. until further use.

According to specific embodiments, the freezing process in liquid nitrogen is dome no more than 10 minutes, no more than 5 minutes, no more than 2 minutes, nor more than 1 minute or no more than 0.5 minute following collection of the sample.

According to specific embodiments, freezing the sample in liquid nitrogen is done no more than 2 minutes following collection of the sample.

According to specific embodiments, freezing the sample in liquid nitrogen is effected for at least 10 minutes, at least 20 minutes, at least 30 minutes, at least 40 minutes, at least 50 minutes, or at least 60 minutes.

According to specific embodiments, freezing the sample in liquid nitrogen is effected for at least 30 minutes.

According to specific embodiments, freezing the sample in liquid nitrogen is effected for up to 360 minutes, up to 300 minutes, up to 240 minutes, up to 200 minutes, up to 150 minutes or up to 120 minutes.

According to specific embodiments, freezing the sample in liquid nitrogen is effected for up to 120 minutes.

According to specific embodiments, the frozen sample is kept at −80° C. until extracting.

The steps involved in RNA extraction from a sample typically include the following:
1. Sample lysis—A lysis buffer that disrupts cell membranes, denatures proteins, solubilizes cellular components, including RNA is added to the sample, to allow the release of RNA. Of note, the lysis buffer typically comprises agents that preserve the integrity and stability of RNA by preventing RNase activity. Alternatively, such agents are added prior to or following the addition of the lysis buffer. Thus, for example, according to specific embodiments, an RNA preservation solution is added to the sample prior to lysis, as further described infra.
2. Phase separation and RNA extraction—The mixture is vigorously shaken to form a biphasic solution comprising several distinct phases, one of them containing the RNA and the others contain proteins, lipids and the like. In several assays this step involves the addition of chloroform to the homogenized sample prior to the shaking. Following careful separation of the phase containing the RNA from the other phases into a fresh tube, isopropanol or ethanol is added to precipitate the RNA, and the mixture is incubated at low temperatures to enhance precipitation. It should be noted that some RNA extraction kits do not require chloroform, phase separation and precipitation. For example, there are kits in which following sample lysis, ethanol is added and the mixture is loaded into an RNA-binding column (e.g. silica membrane column, magnetic beads column). The flow through is discarded and the RNA eluted by nuclease-free water or an alternative buffer.
3. RNA purification—The precipitated or eluted RNA may be pelleted by centrifugation and washed with ethanol to remove impurities. The RNA pellet is then air-dried or dried under vacuum to remove residual ethanol;
4. RNA resuspension—When an RNA pellet is obtained it is dissolved in an appropriate buffer or nuclease-free water to obtain a purified RNA solution.

According so specific embodiments, the sample collected from the subject is mixed with or incubated with an RNA preservation solution.

Thus, according to specific embodiments, the frozen sample is incubated with an RNA preservation solution until a completely thawed RNA preservation solution-sample mix is obtained.

As used herein the phrase “RNA preservation solution”, also known as an RNA stabilization solution, refers to a buffer used to protect RNA molecules from degradation, which typically contains RNase inhibitors, stabilizing agents, buffers to maintain stable pH, antimicrobial agents and the like. Such solutions are well known to the skilled in the art and include RNAlater, TRIzol®, RNA preserve (e.g. Norgen, Cat. NO. 17260) etc.

According to specific embodiments, the RNA preservation solution is RNA Preserve solution commercially obtained from Norgen (Cat. NO. 17260).

According to specific embodiments, the sample to RNA preservation solution ratio is 1:0.5-1:10, 1:1-1:10, 1:1-1:5, 1:1-1:0.3, or 1:1-2.5.

According to specific embodiments, the sample to RNA preservation solution is at least 1:0.5.

According to specific embodiments, the sample to RNA preservation solution is at least 1:0.1.

According to specific embodiments, the RNA preservation solution is added to the frozen sample and the mix is incubated until obtaining a completely thawed RNA preservation solution-sample mix.

According to specific embodiments, the incubation/thawing process is effected while mixing the mix. The mixing may be effected by flick-flack mixing, pipetting or by vortex. According to specific embodiments, the mixing is effected by pipetting.

According to specific embodiments, the RNA preservation solution-frozen sample mix is kept at 4° C. throughout the incubation/thawing process.

According to specific embodiments, the incubation step is effected for at least 12 hours, at least 15 hours, at least 20 hours, at least 24 hours.

According to specific embodiments, the incubation step is effected for up to 24 hours.

According to specific embodiments, following thawing, the thawed mix is kept at room temperature for several minutes until reaching a room temperature.

As described herein above, to extract RNA a lysis buffer should be added to the sample.

According to specific embodiments, the lysis buffer is added to the frozen sample.

According to other specific embodiments, the lysis buffer is added to the thawed sample or sample mix.

According to specific embodiments, following incubation with the RNA preservation solution, the mix is centrifuged, the supernatant is discarded and lysis buffer is added to the pellet to obtain a lysed sample.

Suitable lysis buffers that can be used with specific embodiments are well known in the art and are also commercially available (e.g. from Norgen Bioteck Corporation, ThermoFisher, Sigma-Aldrich, Invitrogen, Qiagen, ZYMO etc.).

According to specific embodiments, the lysis buffer comprises TRIzol®. It should be noted that TRIzol® is endowed with both lysis and RNA preservation properties.

Thus, according to specific embodiments, extraction of the RNA is effected using a TRIzol®-based extraction method.

As used herein the phrase “TRIzol®-based extraction method” refers to an RNA isolation method which utilizes the reagent TRIzol® (also known as TRI Reagent®), which is a monophasic solution composed of phenol and guanidine isothiocyanate in a water-based solution.

Thus, according to specific embodiments, extraction of the RNA is effected using a monophasic solution composed of phenol and guanidine isothiocyanate in a water-based solution.

The steps involved in the TRIzol®-based RNA extraction method (can also be referred to as guanidinium thiocyanate-phenol-chloroform RNA extraction method) typically include the following:
1. Sample homogenization in TRIzol® reagent. When the TRIzol® reagent is added to the biological sample, it quickly denatures proteins, disrupts cell membranes, and solubilizes cellular components, including RNA. This step allows for the release of RNA. Of note, by including guanidine isothiocyanate, TRIzol® helps preserve the integrity and stability of RNA by preventing RNase activity.
2. Phase separation—The mixture is vigorously shaken. The shaking of the TRIzol® mixture (which contains phenol)-treated sample forms a biphasic solution. This step leads to the formation of a biphasic solution where the sample separates into three phases: an aqueous phase containing RNA, an organic phase containing proteins and lipids, and an interphase. In several assays this step involves the addition of chloroform to the homogenized sample prior to the shaking.
3. RNA extraction—The aqueous phase, which contains the RNA, is carefully separated from the other phases and transferred to a fresh tube. Isopropanol or ethanol is added to precipitate the RNA, and the mixture is incubated at low temperatures to enhance precipitation. It should be noted that some RNA extraction kits do not require chloroform, phase separation and precipitation. For example, there are kits in which following sample homogenization, ethanol is added and the mixture is loaded into an RNA-binding column. The flow through is discarded and the RNA eluted by nuclease-free water. According to specific embodiments, this step is repeated multiple times.
4. RNA purification—The precipitated or eluted RNA may be pelleted by centrifugation and washed with ethanol to remove impurities. The RNA pellet is then air-dried or dried under vacuum to remove residual ethanol;
5. RNA resuspension—When an RNA pellet is obtained it is dissolved in an appropriate buffer or nuclease-free water to obtain a purified RNA solution.

TRIzol® and specifically RNA extraction kits comprising TRIzol® can be commercially obtained from e.g. ThermoFisher, Sigma-Aldrich, Invitrogen, Qiagen, ZYMO etc.

According to specific embodiments, the TRIzol®-based extraction method comprises a Direct-zol RNA miniprep RNA extraction kit, commercially obtained from ZYMO research (e.g. cat no. R2052).

According to specific embodiments, the TRIzol®-based extraction method is effected according to the method described in Example 1 of the Examples section which follows, which serves as an integral part of the specification.

According to specific embodiments, the frozen sample is mixed with TRIzol®.

According to specific embodiments, the sample to TRIzol® ratio is 1:0.5-1:10, 1:1-1:10, 1:1-1:5, 1:1-1:0.3, or 1:1-2.5.

According to specific embodiments, the sample to TRIzol® ratio is 1:1-1:3.

According to specific embodiments, the TRIzol®-based extraction method comprises mixing the frozen sample with TRIzol® until obtaining a completely thawed TRIzol®-sample mix.

The mixing may be effected by flick-flack mixing, pipetting or by vortex.

According to specific embodiments, the TRIzol®-frozen sample mix is kept on ice throughout the thawing process.

According to specific embodiments, following thawing, the thawed TRIzol®-sample is further mixed by vortex to homogenize said TRIzol®-sample mix.

According to specific embodiments, following thawing, the thawed TRIzol®-sample mix is kept at room temperature for 5-10 minutes.

According to specific embodiments, following mixing and thawing, the thawed TRIzol®-sample comprises an upper later of fat, a lower level of sediment and an intermediate layer (which comprises the RNA), and the method comprises adding the intermediate layer without the upper layer of fat and the lower level of sediment to an equal volume of ethanol and mixing to obtain a TRIzol®-sample-ethanol mix.

According to other specific embodiments, extraction of the RNA is effected using a non-TRIzol®-based extraction method. Typically such methods include lysis buffers which are not TRIzol®-based.

Such methods are known to the skilled in the art and kits for effecting these methods can be commercially obtained from e.g. Norgen Biotek Corporation.

According to specific embodiments, the non-TRIzol®-based extraction method comprises a Saliva/Swab RNA Purification Kit, commercially obtained from Norgen (e.g. Cat. NO. 69100).

According to specific embodiments, the non-TRIzol®-based extraction method is effected according to the method described in Example 2 of the Examples section which follows, which serves as an integral part of the specification.

According to specific embodiments, the lysis buffer contains β-mercaptoethanol.

According to specific embodiments, at least 500 μl, at least 600 μl, at least 700 μl or at least 800 μl is added to the sample.

According to specific embodiments, ethanol is added to the lysed sample.

According to specific embodiments, at least 500 μl, at least 600 μl, at least 700 μl or at least 800 μl is added to the lysed sample.

According to specific embodiments, following the addition of ethanol the mixture is loaded to an RNA-binding column, the column is centrifuged and the flow-through is discarded.

Such columns are known to the skilled in the art and are also available in commercially available RNA extraction kits, such as described herein.

According to specific embodiments, loading of the mixture to on the column is effected once.

According to other specific embodiments, loading of the mixture to on the column is multiple times (e.g. until all the mixture is loaded on the column).

According to specific embodiments, each time the column is loaded with up to 0.5 ml, up to 0.6 ml, up to 0.7 ml, up to 0.8 ml, up to 0.9 ml or up to 1 ml of the mixture.

According to specific embodiments, loading is effected multiple times, each time with up to 1 ml of the mixture.

According to specific embodiments, loading is effected multiple times, each time with up to 0.7 ml of the mixture. Following, according to specific embodiments, the method comprises eluting an RNA from the RNA-binding column.

According to specific embodiments, the RNA is eluted to a low binding polymer tube.

Eluting may be effected by any elution buffer (e.g. such as supplied by the commercial kit) or by other RNA-free fluid such nuclease free water.

According to specific embodiments, eluting the RNA is effected with nuclease free water.

According to specific embodiments, the elution buffer is warmed to e.g. 37-80° C.

According to specific embodiments, the elution buffer comprises nuclease free water wormed to 70° C.

According to specific embodiments, eluting the RNA from the RNA-binding column by adding a total of at least 25 μl of nuclease-free water and centrifuging the column.

According to specific embodiments, eluting the RNA from the RNA-binding column by adding a total of at least 40 μl of nuclease-free water and centrifuging the column.

According to specific embodiments, eluting is effected in one round.

According to other specific embodiments, eluting is effected in at least two rounds.

Throughout the extraction process, the method may comprise a centrifugation step.

According to specific embodiments, the centrifuging is at 4° C.

According to specific embodiments, the centrifuging is at 20° C.

According to specific embodiments, the centrifuging is in 10,000-22,000 g.

According to specific embodiments, the centrifuging is in 20,000-21,000 g.

According to specific embodiments, the centrifuging is in 10,000-15,000 g.

According to specific embodiments, the centrifuging is in 10,000-11,000 g.

According to specific embodiments, RNA concentration and integrity is determined following extraction. Such methods are well known in the art and are further disclosed in the examples section which follows.

According to specific embodiments, the RNA is kept at −80° C. until further use.

According to specific embodiments, the method further comprising analyzing an RNA expression level in said RNA sample following said extracting.

Methods of analyzing an RNA expression levels including e.g. whole cell transcriptome analysis and RT-PCR, are well known in the art and further described in details hereinbelow.

Furthermore, specific embodiments of the present invention suggest that upper GI fluids are associated with inflammation and immune activity in various upper GI pathologies, and thus the RNA samples obtained according to the methods disclosed herein may be used for diagnosis, staging and assessment of response to therapy.

Thus, according to an aspect of the present inventions, there is provided a method of diagnosing an upper gastrointestinal (GI) pathology in a human subject in need thereof, the method comprising:
isolating RNA sample from an upper GI tract of the subject according to the method disclosed herein; and
analyzing an RNA expression level of at least one human gene in said RNA sample following said extracting, wherein the expression level is indicative of the upper GI pathology.

As used herein, the term “subject” refers to human being at any gender and any age. According to specific embodiments, the subject is an adult (e.g. older than 18, 21, or 22 years). According to other specific embodiments, the subject is a child (e.g. younger than 18 years).

According to specific embodiments, the subject is at risk to develop the pathology (i.e. upper gastrointestinal (GI) pathology, as further described herein).

According to specific embodiments, the subject shows symptoms of the pathology.

According to specific embodiments, the subject is diagnosed with the pathology.

According to specific embodiments, the subject is diagnosed with the pathology according to the methods disclosed herein.

As used herein the term “diagnosing” refers to determining presence or absence of a pathology (e.g. upper GI pathology), classifying a pathology or a symptom, determining a severity of the pathology, monitoring pathology progression, forecasting an outcome of a pathology and/or prospects of recovery and screening of a subject for a specific disease.

According to specific embodiments, diagnosing the pathology comprises determining presence or absence of the pathology.

According to specific embodiments, diagnosing the pathology comprises determining the severity of the pathology.

According to specific embodiments, the method of diagnosing comprises monitoring disease state in a subject, comprising repeating the analysis following a predestined time interval.

As used herein, the phrase “upper GI pathology” refers to a medical condition or disorder that affect an anatomical structure or organ located within the upper GI tract. According to specific embodiments, the upper GI pathology is associated with inflammation.

According To specific embodiments, the pathology is non-cancerous. According to other specific embodiments, the pathology is cancer.

Non-limiting examples of such pathologies include eosinophilic esophagitis (EoE), peptic ulcer disease, eosinophilic gastritis, collagenous gastritis, esophagitis, enteritis, atrophic gastritis, celiac disease, primary sclerosing cholangitis, primary biliary cirrhosis, gastric lymphoma, gastric/duodenal neuro-endocrine tumor, gastric carcinoma, hepatocellular carcinoma, pancreatic cancer and biliary cancer.

According to specific embodiments, the pathology is celiac.

Celiac is an autoimmune disorder characterized by an inappropriate immune response to ingested gluten. Upon gluten consumption, individuals with celiac disease experience an immune-mediated attack on the small intestine's mucosa, leading to villous atrophy, crypt hyperplasia, and increased intraepithelial lymphocytes. This pathological damage results in malabsorption of nutrients, which can manifest in various gastrointestinal and extraintestinal symptoms, including diarrhea, weight loss, abdominal pain, and nutritional deficiencies.

According to specific embodiments, the pathology is eosinophilic esophagitis (EoE).

EoE is a chronic, immune-mediated inflammatory disorder of the esophagus characterized by the infiltration of eosinophils into the esophageal epithelium.

According to specific embodiments of the diagnostic aspects disclosed herein, the expression level of the at least one human gene in the sample is indicative of presence of absence of the pathology.

Thus, according to specific embodiments, an altered expression level compared to an upper GI tract secretion RNA sample of a control subject not suffering from the upper GI pathology is indicative of the pathology.

According to additional or an alternative embodiments, no significant change in expression level compared to an upper GI tract secretion RNA sample of a control subject not suffering from the upper GI pathology is indicative of the pathology.

According to specific embodiments, the method comprises ruling in or ruling out of the upper GI pathology.

According to specific embodiments, when altered expression level compared to an upper GI tract secretion RNA sample of a control subject not suffering from the upper GI pathology and/or no significant change in expression level compared to an upper GI tract secretion RNA sample of a control subject not suffering from the upper GI pathology is indicated, the method comprises classifying the subject as having the pathology.

According to specific embodiments, when no significant change in expression level compared to an upper GI tract secretion RNA sample of a control subject not suffering from the upper GI pathology and/or altered expression level compared to an upper GI tract secretion RNA sample of a control subject not suffering from the upper GI pathology is indicated, the method comprises classifying the subject as free of the pathology.

According to specific embodiments, the expression level of the at least one gene is determined in a saliva sample of the subject and in a non-saliva upper GI tract secretion of the subject.

According to specific embodiments, the expression level of the at least one gene in a saliva sample of the subject correlates with the expression level of the at least one gene in a non-saliva upper GI tract secretion of said subject.

Once the classification is made, additional tests may be made in order to corroborate the result or to further classify the pathology using a state of the art technique. Such methods are known in the art and include e.g. biopsy.

According to specific embodiments, the expression level of the at least one gene correlates with the degree of histological inflammation.

Specific embodiments of the present invention further contemplate making appropriate treatment decisions based on the diagnosis.

Hence, according to an aspect of the present invention, there is provided a method of treating an upper GI pathology of a subject in need thereof, the method comprising:
(a) confirming that the subject has the upper GI pathology according to the method disclosed herein; and
(b) administering to the subject a therapeutically effective amount of an agent useful for treating the pathology.

According to an aspect of the present invention, there is provided a method of treating an upper GI pathology of a subject in need thereof, the method comprising:
(a) confirming that the subject has the upper GI pathology according to the method disclosed herein; and
(b) selecting a therapeutic agent based on said expression level.

According to an aspect of the present invention, there is provided a method of treating an upper GI pathology of a subject in need thereof, the method comprising:
(a) receiving an identification that a subject has an upper GI pathology according to the method disclosed herein; and
(b) administering to the subject a therapeutically effective amount of an agent useful for treating the pathology.

According to an aspect of the present invention, there is provided a method of treating an upper GI pathology of a subject in need thereof, the method comprising:
(a) receiving an identification that a subject has an upper GI pathology according to the method disclosed herein; and
(b) selecting a therapeutic agent based on said expression level.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

According to an aspect of the present invention, there is provided a method of monitoring efficacy of treatment in a subject diagnosed with the pathology, the method comprising analyzing an RNA expression level of at least one human gene in the RNA sample following treatment, wherein a change in the expression level following treatment is indicative of efficaciousness of the treatment.

Agents useful for the treatment of upper GI pathologies are known in the art. Non-limiting examples include corticosteroids, immunosuppressants, biologics (e.g. antibodies, anti-TNF agents), proton pump inhibitors, dietary management etc.

Notwithstanding the above, the present inventors identified 10 genes having significant altered expression patterns in sputum samples of celiac disease patients, (prior to gluten avoidance), compared to controls (healthy patients and eosinophilic esophagitis patients); and 10 genes having significant altered expression patterns in sputum samples of eosinophilic esophagitis patients, compared to controls (healthy patients and celiac patients) (Example 2 of the Example section which follows).

Specifically, the following markers were up regulated in celiac patients: AP006216.3, AC011754.1, CCDC152, AKR7A3 and IGLV2-14;
the following markers were down regulated in celiac patients: AC122718.1, AP001011.1, MICB-DT, RN7SL288P and AC092746.1;
the following markers were upregulated in EoE patients: MALAT1, AC092746.1, AC114760.2, RGPD4-AS1, FAM133B, TMEM52B, AC018607.1, AC114801.3 and CNOT11; and
CHST7 was downregulated in EoE.

Hence, specific embodiments suggest diagnosing subjects and making appropriate treatment decisions based on expression levels of these markers.

Thus, according to an aspect of the present invention there is provided a method of diagnosing celiac in a human subject in need thereof, the method comprising analyzing an expression level of at least one gene selected from the group consisting of AP006216.3, AC011754.1, CCDC152, AKR7A3 and IGLV2-14 in a sample of an upper GI tract secretion of the subject, wherein said expression level above a predetermined threshold is indicative of celiac.

According to specific embodiments the method further comprising analyzing an expression level of at least one additional gene selected from the group consisting of AC122718.1, AP001011.1, MICB-DT, RN7SL288P and AC092746.1 in said sample of said upper GI tract secretion of said subject, wherein said expression level of said at least one additional gene below a predetermined threshold is indicative of celiac.

According to an additional or an alternative aspect of the present invention there is provided a method of diagnosing celiac in a human subject in need thereof, the method comprising analyzing an expression level of at least one gene selected from the group consisting of AC122718.1, AP001011.1, MICB-DT, RN7SL288P and AC092746.1 in a sample of an upper GI tract secretion of the subject, wherein said expression level below a predetermined threshold is indicative of celiac.

According to specific embodiments, the method comprises ruling in or ruling out celiac.

According to specific embodiments, when expression level of the at least one gene selected from the group consisting of AP006216.3, AC011754.1, CCDC152, AKR7A3 and IGLV2-14 above a predetermined threshold and/or expression level of the at least one gene selected from the group consisting of AC122718.1, AP001011.1, MICB-DT, RN7SL288P and AC092746.1 below a predetermined level is indicated, the method comprises classifying the subject as having celiac.

According to specific embodiments, when expression level of the at least one gene selected from the group consisting of AP006216.3, AC011754.1, CCDC152, AKR7A3 and IGLV2-14 below a predetermined threshold and/or expression level of the at least one gene selected from the group consisting of AC122718.1, AP001011.1, MICB-DT, RN7SL288P and AC092746.1 above a predetermined level is indicated, the method comprises classifying the subject as free of celiac.

According to an aspect of the present invention, there is provided a method of diagnosing eosinophilic esophagitis (EoE) in a human subject in need thereof, the method comprising analyzing an expression level of at least one gene selected from the group consisting of MALAT1, AC092746.1M AC114760.2, RGPD4-AS1, FAM133B, TMEM52B, AC018607.1, AC114801.3 and CNOT11 in a sample of an upper GI tract secretion of the subject, wherein said expression level above a predetermined threshold is indicative of celiac.

According to specific embodiments, the method further comprising analyzing an expression level of CHST7 in said sample of said upper GI tract secretion of said subject, wherein said expression level of said CHST7 below a predetermined threshold is indicative of EoE.

According to an additional or an alternative aspect of the present invention, there is provided a method of diagnosing eosinophilic esophagitis (EoE) in a human subject in need thereof, the method comprising analyzing an expression level of CHST7 in a sample of an upper GI tract secretion of the subject, wherein said expression level below a predetermined threshold is indicative of EoE.

According to specific embodiments, the method comprises ruling in or ruling out EoE.

According to specific embodiments, when expression level of the at least one gene selected from the group consisting of MALAT1, AC092746.1M AC114760.2, RGPD4-AS1, FAM133B, TMEM52B, AC018607.1, AC114801.3 and CNOT11 above a predetermined threshold and/or expression level of CHST7 below a predetermined level is indicated, the method comprises classifying the subject as having celiac.

According to specific embodiments, when expression level of the at least one gene selected from the group consisting of MALAT1, AC092746.1M AC114760.2, RGPD4-AS1, FAM133B, TMEM52B, AC018607.1, AC114801.3 and CNOT11 below a predetermined threshold and/or expression level of CHST7 above a predetermined level is indicated, the method comprises classifying the subject as free of celiac.

According to an aspect of the present invention, there is provided method of treating celiac in a subject in need thereof, the method comprising:
(a) confirming that the subject has celiac according to the method disclosed herein; and
(b) administering to the subject a therapeutically effective amount of an agent useful for treating celiac.

According to an aspect of the present invention, there is provided method of treating celiac in a subject in need thereof, the method comprising:
(a) confirming that the subject has celiac according to the method disclosed herein; and
(b) selecting a therapeutic agent based on said expression level.

According to an aspect of the present invention, there is provided method of treating celiac in a subject in need thereof, the method comprising:
(a) receiving an identification that a subject has celiac according to the method disclosed herein; and
(b) administering to the subject a therapeutically effective amount of an agent useful for treating celiac.

According to an aspect of the present invention, there is provided method of treating celiac in a subject in need thereof, the method comprising:
(a) receiving an identification that a subject has celiac according to the method disclosed herein; and
(b) selecting a therapeutic agent based on said expression level.

According to an aspect of the present invention, there is provided method of treating eosinophilic esophagitis (EoE) in a subject in need thereof, the method comprising:
(a) confirming that the subject has EoE according to the method disclosed herein; and
(b) administering to the subject a therapeutically effective amount of an agent useful for treating EoE.

According to an aspect of the present invention, there is provided a method of treating eosinophilic esophagitis (EoE) in a subject in need thereof, the method comprising:
(a) confirming that the subject has EoE according to the method disclosed herein; and
(b) selecting a therapeutic agent based on said expression level.

According to an aspect of the present invention, there is provided method of treating eosinophilic esophagitis (EoE) in a subject in need thereof, the method comprising:
(a) receiving an identification that a subject has EoE according to the method disclosed herein; and
(b) administering to the subject a therapeutically effective amount of an agent useful for treating EoE.

According to an aspect of the present invention, there is provided a method of treating eosinophilic esophagitis (EoE) in a subject in need thereof, the method comprising:
(a) receiving an identification that a subject has EoE according to the method disclosed herein; and
(b) selecting a therapeutic agent based on said expression level.

According to an aspect of the present invention, there is provided a method of monitoring efficacy of treatment in a subject diagnosed with celiac, the method comprising analyzing an expression level of at least one human gene AC122718.1, AP001011.1, MICB-DT, RN7SL288P, AC092746.1, AP006216.3, AC011754.1, CCDC152, AKR7A3 and IGLV2-1 in a sample of an upper GI tract secretion of the subject following treatment, wherein a change in the expression level following treatment is indicative of efficaciousness of the treatment.

According to an aspect of the present invention, there is provided a method of monitoring efficacy of treatment in a subject diagnosed with EoE, the method comprising analyzing an expression level of at least one human gene MALAT1, AC092746.1M AC114760.2, RGPD4-AS1, FAM133B, TMEM52B, AC018607.1, AC114801.3, CNOT11 and CHST7 in a sample of an upper GI tract secretion of the subject following treatment, wherein a change in the expression level following treatment is indicative of efficaciousness of the treatment.
MALAT1—Metastasis Associated Lung Adenocarcinoma Transcript 1 (Non-Protein Coding RNA), lncRNA, Gene ID: 378938. According to specific embodiments, the MALAT1 cDNA sequence is as provided in SEQ ID NO: 1.
CHST7—Galactose/N-Acetylglucosamine/N-Acetylglucosamine 6-O-Sulfotransferase 5, Gene ID: 56548. According to specific embodiments, the CHST7 cDNA sequence is as provided in SEQ ID NO: 2.
RGPD4-AS1—RGPD4 Antisense RNA 1 (Head To Head), lncRNA, Gene ID: 729121. According to specific embodiments, the RGPD4-AS1 cDNA sequence is as provided in SEQ ID NO: 3.
AC092746.1—ENSG00000255801 (from geneSymbol), lncRNA. According to specific embodiments, the AC092746.1 cDNA sequence is as provided in SEQ ID NO: 4.
AC114760.2—STK17B-Homo sapiens serine/threonine kinase 17b, Gene ID: 9262. According to specific embodiments, the AC114760.2 cDNA sequence is as provided in SEQ ID NO: 5.
FAM133B—Family With Sequence Similarity 133 Member B, Gene ID: 257415. According to specific embodiments, the FAM133B cDNA sequence is as provided in SEQ ID NO: 6.
TMEM52B—Transmembrane Protein 52B (Located in extracellular exosome), Gene ID: 120939. According to specific embodiments, the TMEM52B cDNA sequence is as provided in SEQ ID NO: 7.
AC018607.1—TGS1-Homo sapiens trimethylguanosine synthase 1, Gene ID: 96764. According to specific embodiments, the AC018607.1 cDNA sequence is as provided in SEQ ID NO: 8.
AC114801.3—Pseudogene, see https://genome (dot) ucsc (dot) edu/cgi-bin/hgc?hgsid=2270118158_wf2BUqaino357ZXNPP8owXcXuarm&db=hg38&c=chr4&1=7800 8511&r=78008688&0-78008511&t=78008688&g=wgEncodeGencodePseudoGeneV37&i=EN ST00000510231.1. According to specific embodiments, the AC114801.3 cDNA sequence is as provided in SEQ ID NO: 9.
CNOT11—CCR4-NOT Transcription Complex Subunit 11, Gene ID: 55571. According to specific embodiments, the CNOT11 cDNA sequence is as provided in SEQ ID NO: 10.
AP006216.3—APOA4-apolipoprotein A4. According to specific embodiments, the AP006216.3 cDNA sequence is as provided in SEQ ID NO: 11.
AC011754.1—REG3A-Regenerating Family Member 3 Alpha, Gene ID: 5068. According to specific embodiments, the AC011754.1 cDNA sequence is as provided in SEQ ID NO: 12.
CCDC152—Coiled-Coil Domain Containing 152, Gene ID: 100129792. According to specific embodiments, the CCDC152 cDNA sequence is as provided in SEQ ID NO: 13.
AC122718.1—ZSWIM6-Homo sapiens zinc finger SWIM-type containing 6, Gene ID: 57688. According to specific embodiments, the AC122718.1 cDNA sequence is as provided in SEQ ID NO: 14.
AP001011.1—SMCHD1-Homo sapiens structural maintenance of chromosomes flexible hinge domain containing 1, Gene ID: 23347. According to specific embodiments, the AP001011.1 cDNA sequence is as provided in SEQ ID NO: 15.
MICB-DT—MICB Divergent Transcript, lncRNA, Gene ID: 102725068. According to specific embodiments, the MICB-DT cDNA sequence is as provided in SEQ ID NO: 16.
RN7SL288P—RNA, 7SL, Cytoplasmic 288, Pseudogene, Gene ID: 106481000. According to specific embodiments, the RN7SL288P cDNA sequence is as provided in SEQ ID NO: 17.
AKR7A3—Aldo-Keto Reductase Family 7 Member A3, Gene ID: 22977. According to specific embodiments, the AKR7A3 cDNA sequence is as provided in SEQ ID NO: 18.
IGLV2-14—Immunoglobulin Lambda Variable 2-14, Gene ID: 28815. According to specific embodiments, the IGLV2-14 cDNA sequence is as provided in SEQ ID NO: 19.

As used herein the phrase “predetermined threshold” refers to an expression level of a gene that characterizes a sample of the same origin obtained from a subject not suffering from the pathology assayed under the same conditions. Such a level can be experimentally determined by comparing samples with normal expression levels of the gene (i.e. a sample from a subject not suffering from the pathology of interest e.g., a sample obtained from a healthy subject or from a subject having an upper GI disease distinct from the pathology of interest) to samples derived from subjects diagnosed with the pathology. Alternatively, such a level can be obtained from the scientific literature and from databases.

According to specific embodiments, the predetermined threshold is derived from a control sample.

According to specific embodiments, the control sample is a healthy control sample.

According to specific embodiments, the control sample is of an upper GI disease which is distinct from the pathology of interest (e.g. when diagnosing celiac the control sample is of EoE, when diagnosing EoE the control sample is of celiac).

According to specific embodiments, the control sample is obtained from the scientific literature or from a database.

According to specific embodiments, the increase/decrease above or below a predetermined threshold is statistically significant (e.g., P<0.05).

According to specific embodiments, the predetermined threshold is at least 1.5 fold, at least 2 fold, at least 3 fold, at least 5 fold, at least 10 fold, or at least 20 fold as compared the expression level of the gene in a control sample as measured using the same assay, as further disclosed herein.

According to specific embodiments, the predetermined threshold is at least 1.5 fold as compared the level of the gene in a control sample.

According to specific embodiments, the predetermined threshold is at least 2%, at least 5%, at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600% as compared the expression level of the gene in a control sample.

According to specific embodiments, the expression level of at least 1, at least 2, at least 3, at least 4, at least 5, at least 6, at least 7, at least 8, at least 9 or 10 of the markers disclosed herein is determined, each possibility represents a separate embodiment of the present invention.

According to specific embodiments, a change in expression levels, as defined herein, of at least 1, at least 2, at least 3, at least 4, at least 5, at least 6, at least 7, at least 8, at least 9 or 10 of the markers disclosed herein is indicative of the pathology, each possibility represents a separate embodiment of the present invention.

As used herein, the phrase “expression level” refers to the degree of gene expression (e.g. mRNA or protein) in an upper GI tract secretion sample.

It should be noted that the expression level can be determined in arbitrary absolute units, or in normalized units (relative to known expression levels of a control sample). For example, when using RNA chips, the expression levels are normalized according to internal controls or by using quantile normalization.

Expression level can be determined using any structural, biological or biochemical method which is known in the art for detecting the expression level at the transcript or the protein level.

According to specific embodiments, the RNA or the protein molecules are extracted from the biological sample of the subject. Thus, according to specific embodiments, the method further comprises extracting RNA or a protein from the biological sample prior to the determining. Such methods are known in the art.

According to specific embodiments, the expression level refers to RNA expression levels and analyzing expression level is effected at the transcript level using RNA or DNA detection methods.

According to specific embodiments, the RNA is obtained according to the methods disclosed herein.

It should be noted that the extracted RNA can be further processed to a cDNA. Methods of and commercially available kits for converting RNA to cDNA are well known in the art and include e.g. the use of the enzyme reverse transcriptase.

Thus, according to specific embodiments, the methods disclosed herein comprise synthesizing a cDNA from the obtained RNA and amplifying the obtained cDNA. Such methods are known in the art and also described in the Examples section which follows. Commercial kits are also available from e.g., Biolabs (e.g. NEBNext® Single Cell/Low Input RNA Library Prep Kit for Illumina®, NEB #E6420S/L).

According to specific embodiments, more than 10 ng RNA extracted according to the methods disclosed herein are used for cDNA synthesis.

According to specific embodiments, at least 5 μl, at least 6 μl or at least 7 μl of the RNA extracted according to the methods disclosed herein are used for cDNA synthesis.

According to specific embodiments, about least 7 μl of the RNA extracted according to the methods disclosed herein are used for cDNA synthesis.

According to specific embodiments, at least 10, at least 15, at least 20 cycles of cDNA amplifications are performed.

According to specific embodiments, about 20 cycles of cDNA amplifications are performed.

Once obtained, the RNA or cDNA can be characterized for the level of various RNA or cDNA molecules using methods and agents known in the arts.

Thus, according to some embodiments, detection of the expression level of the gene is performed by contacting the sample of an upper GI tract secretion or extracts thereof with a probe (e.g. oligonucleotide probe or primer) which specifically hybridizes to a polynucleotide expression product of the gene. Such a probe can be at any size, such as a short polynucleotide (e.g., of 15-200 bases), an intermediate polynucleotide of 100-2000 bases and a long polynucleotide of more than 2000 bases.

The probe used by the present invention can be any directly or indirectly labeled RNA molecule [e.g., RNA oligonucleotide (e.g., of 17-50 bases), an in-vitro transcribed RNA molecule], DNA molecule (e.g., oligonucleotide, e.g., 15-50 bases, cDNA molecule, genomic molecule) and/or an analogue thereof [e.g., peptide nucleic acid (PNA)] which is specific to the RNA transcript. According to specific embodiments, the probe is bound to a detectable moiety.

Oligonucleotides designed according to the teachings of the present invention can be generated according to any oligonucleotide synthesis method known in the art such as enzymatic synthesis or solid phase synthesis.

According to specific embodiments, the contacting is effected under conditions which allow the formation of a complex comprising the specific mRNA or cDNA sequence present in the sample and the probe. The complex can be formed at a variety of temperatures, salt concentration and pH values which may vary depending on the method and the sample used and those of skills in the art are capable of adjusting the conditions suitable for the formation of each nucleotide/probe complex.

Non-limiting examples of methods of detecting RNA and/or cDNA molecules in a sample include Northern blot analysis, RT-PCR [e.g., a semi-quantitative RT-PCR, quantitative RT-PCR using e.g., the Light Cycler™ (Roche)], RNA in-situ hybridization (using e.g., DNA or RNA probes to hybridize RNA molecules present in the cells or tissue sections), in-situ RT-PCR (e.g., as described in Nuovo G J, et al. Am J Surg Pathol. 1993, 17:683-90; Komminoth P, et al. Pathol Res Pract. 1994, 190:1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, CA)], RNA sequencing, whole transcriptome analysis, deep sequencing etc. Such methods are well known in the art and disclosed for example in International Patent Application Publication No. WO2022/153323, the contents of which are fully incorporated herein by reference.

According to specific embodiments, analyzing the expression level comprises performing whole cell transcriptome analysis.

According to specific embodiments, analyzing the expression level comprises performing RT-PCR.

According to other specific embodiments, determining the expression level is effected at the protein level using protein detection methods. Such agents and detection methods are known in the art and include e.g. antibodies.

Thus, according to some embodiments, detection of the expression level is performed by contacting the biological sample or extracts thereof with an antibody which specifically binds to the polypeptide of interest.

According to specific embodiments, the contacting is effected under conditions which allow the formation of a complex comprising the polypeptide of interest present in the sample and the agent e.g. antibody (i.e. immunocomplex).

The complex (e.g. immunocomplex) can be formed at a variety of temperatures, salt concentration and pH values which may vary depending on the method and the sample used and those of skills in the art are capable of adjusting the conditions suitable for the formation of each complex.

Non-limiting examples of methods of detecting the level of specific protein molecules in a sample include Enzyme linked immunosorbent assay (ELISA), Western blot analysis, immunoprecipitation (IP), radio-immunoassay (RIA), Fluorescence activated cell sorting (FACS), immunohistochemical analysis, in-situ activity assay (using e.g., a chromogenic substrate applied on the cells containing an active enzyme), in-vitro activity assays (in which the activity of a particular enzyme is measured in a protein mixture extracted from the cells) and molecular weight-based approach.

The antibody or probe used by the present invention can be any directly or indirectly labeled antibody or probe. According to specific embodiments, the antibody or probe is bound to a detectable moiety. The detectable moiety used by some embodiments of the invention can be, but is not limited to a fluorescent chemical (fluorophore), a phosphorescent chemical, a chemiluminescent chemical, a radioactive isotope (such as ^{[125]}iodine), an enzyme, a fluorescent polypeptide, an affinity tag, and molecules (contrast agents) detectable by Positron Emission Tomography (PET) or Magnetic Resonance Imaging (MRI).

Specific embodiments of the invention, further contemplate diagnostic kits.

According to an aspect of the present invention, there is provided a diagnostic kit for diagnosing celiac, the kit comprising at least two agents capable of specifically detecting expression products of at least two genes associated with celiac, wherein at least one of said at least two genes is selected from the group consisting of AC122718.1, AP001011.1, MICB-DT, RN7SL288P, AC092746.1, AP006216.3, AC011754.1, CCDC152, AKR7A3 and IGLV2-1, wherein the kit comprises no more than 20 agents capable of specifically detecting said expression products.

According to an aspect of the present invention, there is provided a diagnostic kit for diagnosing eosinophilic esophagitis (EoE), the kit comprising at least two agents capable of specifically detecting expression products of at least two genes associated with celiac, wherein at least one of said at least two genes is selected from the group consisting of MALAT1, AC092746.1M AC114760.2, RGPD4-AS1, FAM133B, TMEM52B, AC018607.1, AC114801.3, CNOT11 and CHST7, wherein the kit comprises no more than 20 agents capable of specifically detecting said expression products.

According to specific embodiments, the kit comprises at least two, at least 3, at least 4, at least 5, at least 6, at least 7, at least 8, at least 9 or at least 10 agents, each possibility represents a separate embodiment of the present invention.

According to specific embodiments, the kit comprises no more than 20, no more than 15, not more than 10, not more than 5 agents capable of specifically detecting expression products of genes associated with the pathology (i.e. celiac or EoE).

According to specific embodiments, the kit consists of two agents capable of specifically detecting expression products of genes associated with the pathology (i.e. celiac or EoE).

According to specific embodiments of the kit aspects, the at least one gene comprises at least two, at least 3, at least 4, at least 5, at least 6, at least 7, at least 8, at least 9 or 10 of the recited genes, each possibility represents a separate embodiment of the present invention.

According to specific embodiments, any of the above described kits may also comprise additional components such as a primer, an adapter polynucleotide, an enzyme (e.g. a reverse transcriptase, a ligase, a DNA polymerase, RNA polymerase, RNAse H, DNase, exonuclease and the like), RNAse inhibitor, DNase inhibitor, a protease inhibitor, a labeling agent, a linker, a secondary antibody, reagents and buffers.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques.

### Materials and Methods

Upper GI sample collection—At the beginning of the gastroscopy the physician inserts the endoscope (washed and sterilized before every use, including a QC step). Immediately at the beginning of the procedure and prior to insertion of the endoscope into the stomach, gastric secretions (about 5 ml) are suctioned. Sterile collecting tubes are used, which are connected to the vacuum passage on the scope from one side and to the vacuum tube on the other side. Immediately after disconnection, the tube is sealed with a sterile cap and immersed in liquid nitrogen for 30-120 minutes. Following, the physician inserts the scope into the duodenum, the endoscope's tunnel is cleared with water against the duodenal bulb and the fluid is suctioned. Afterwards, the walls of the duodenal blub and second part are flushed with water, which is then suctioned again. The duodenal fluid is similarly collected using a designated tube which is sealed. Immediately after retrieval the sample tubes are put into liquid nitrogen for 30-120 minutes. Following, the samples are placed in a −80° C. freezer until processed.

Saliva sample collection—Saliva samples are collected from patients fasting for at least 6 hours. The patient is asked to spit, using a designated straw, into a 2 ml collection tube, until the amount of liquid saliva reached the 1 ml line. Discard the straw, close the collection tube tightly using a sterile cap and immediately put the tube in liquid nitrogen for 30-120 minutes. Following, the samples are placed in a −80° C. freezer until processed. If gastroscopy is to be performed, the saliva is collected prior to the beginning of the gastroscopy.

RNA extraction from gastroscopy wash samples—Extraction was performed using the Direct-zol RNA miniprep (ZYMO Research) RNA extraction kit (Cat NO. R2052), according to the manufacturer's instructions with the following modifications:
The ‘wash’ samples were put on ice. TRI Reagent® was added to the frozen sample in a ratio of 1:1-1:2.5 (sample: TRI Reagent®). This mix was thawed gradually by flick-flack mixing while keeping the sample on ice during the thawing process followed by mixing by Vortex until most of the particles were dissolved.
The mixture was kept at room temperature for 5-10 minutes.
The sample, without the upper layer of fat and the lower level of sediment was added to an equal volume of ethanol (95-100%) and the mix was mixed thoroughly.
The resultant mixture was added to a designated Zymo-Spin™ IICR Column. Each time, 760 μl was added to the column, centrifuged at 10,600 g for 30 seconds at 4° C. and the flow-through was discarded. This step was repeated until loading 4-8 ml of the sample in total.
DNase and RNA pre-wash and wash steps were then performed as per protocol.
In the final step, the column was placed into a fresh 1.7 ml low binding tube, RNA was eluted either in 1 step using 25 μl of 70° C. warmed DNase/RNase-Free Water followed by centrifugation of the column at 13,000 g at 4° C.; or in 30-40 μl of DNase/RNase-Free Water in two steps-specifically, in the first step about 25 μl of water were added and the column was centrifuged at 10,600 g at 4° C. and in the second step about 15 μl of water were added and the column was centrifuged again. In case not all the water were transferred, another 10 μl were added and the column was centrifuged again in the same manner. The purified RNA sample was stored at −80° C. until processed.

RNA extraction from saliva samples—Extraction was performed using the Saliva/Swab RNA Purification Kit (Norgen, Cat. NO. 69100) according to the manufacturer's instructions with the following modifications:
24 h prior the purification, the saliva sample were put on ice and RNA Preserve (Norgen, Cat. NO. 17260) was added to the frozen sample in a ratio of 1:1 (sample: RNA Preserve). This mix was thawed by up and down mixing using the 1 ml pipette, while keeping the sample on ice during the thawing process. The sample was incubated 20-24 h in 4° C.
After 24 h, the sample was thawed completely and incubated at RT until it reached to room temperature. Then, the sample was centrifuged at 10,000 g for 10 minutes at RT and the supernatant was discarded completely by pipetting.
800 μl of PBS was added to the sample pellet following by 800 μl of Lysis Buffer A supplied with b-mercaptoethanol. The mixture was vortex for 10 second and 800 μl of ethanol (95-100%) was added and the mixture was mixed by vortex.
The resultant mixture was added to a designated Norgen Column. Each time, 600 μl was added to the column, centrifuged at 20,800 g for 1 minute at 20° C. and the flow-through was discarded. This step was repeated until all the mixture was added. DNase and RNA pre-wash and wash steps were then performed as per protocol.
In the final step, the column was placed into a fresh 1.7 ml low binding tube, RNA was eluted in 30 μl of 70° C. warmed DNase/RNase-Free Water. The column was centrifuged for 2 minutes at 420 g, followed by 1 minute at 20,800 g at 20° C. and the purified RNA sample was stored at −80° C. until processed.

Bulk RNA sequencing—was performed using the NEB E6420 NEBNext® Single Cell/Low Input RNA Library Prep Kit for Illumina. The library preparation was done according to the manufacturer's Protocol for Low Input RNA: cDNA Synthesis, Amplification and Library Generation (New England BioLabs Cat NO. E6420) with minor modifications: 7 μl of total RNA was used regardless the RNA concentration; in the cDNA Amplification section, 20 PCR cycles were performed instead of 9; and magnetic beads were used for cleanup of cDNA (Beckman Coulter Cat NO. A63881). Following preparation of cDNA libraries for each of the samples, they were pooled together and sequenced using an Ilumina NovaSeq X Plus 1.5B-100 cycles.

### Example 1

### Extraction of Intact RNA from Upper GI Tract Samples Obtained During Gastroscopy

Specific embodiments of the present invention suggest that upper GI fluids (e.g. from the stomach and duodenum), obtained during gastroscopy are associated with inflammation and immune activity in various upper GI pathologies, such as celiac disease and eosinophilic esophagitis; and thus may enable diagnosis, staging and assessment of response to therapy. Currently, the only method to diagnose eosinophilic esophagitis and celiac disease is via biopsy.

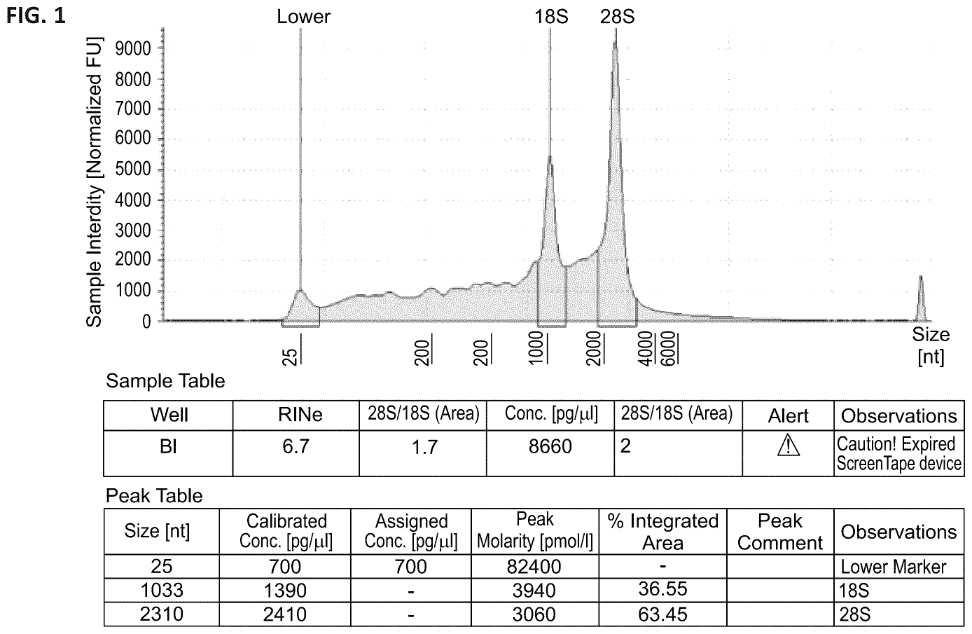

Importantly, not only that RNA sequencing of upper GI secretions has not been previously performed, the present inventors uncovered that obtaining intact RNA from such samples is not trivial. However, the present inventors have established a novel and unique method for RNA extraction from upper GI samples (FIG. 1) which was further amplified and sequenced. Comparing the obtained transcriptome to RNA samples obtained from lower GI samples (as disclosed in Ungar et al. (2022) Gut gutjnl-2021-325516; and Dan S et al. (2023) Cell Mol Gastroenterol Hepatol 16 (1): 1-15), indicated that the depth of sequencing and the variability of transcripts was satisfactory (Data not shown).

### Example 2

### Extraction of Intact RNA from Saliva Samples

Specific embodiments of the present invention suggest that saliva sputum samples are associated with inflammation and immune activity in various upper GI pathologies, such as celiac disease and eosinophilic esophagitis; and thus may enable diagnosis, staging and assessment of response to therapy.

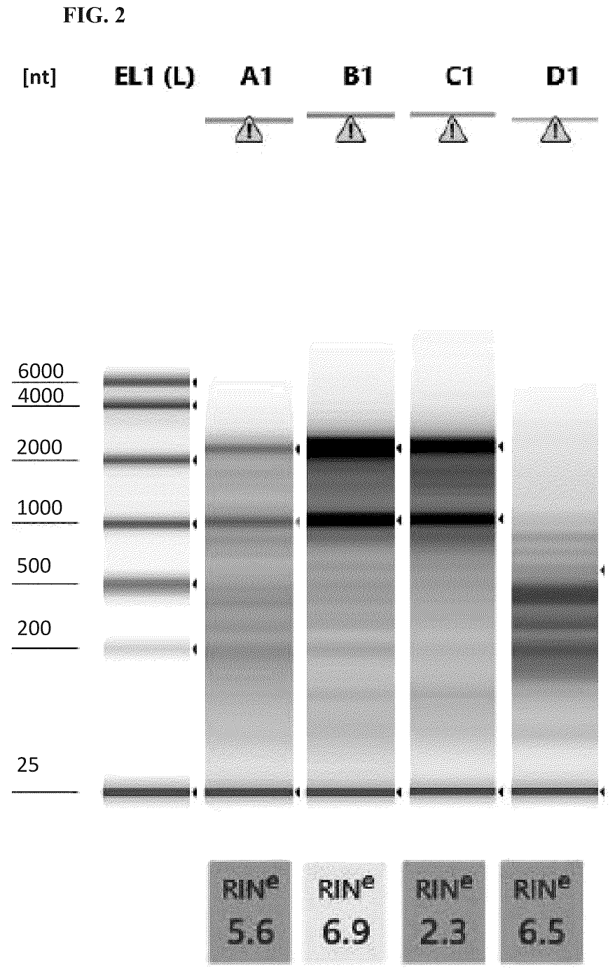

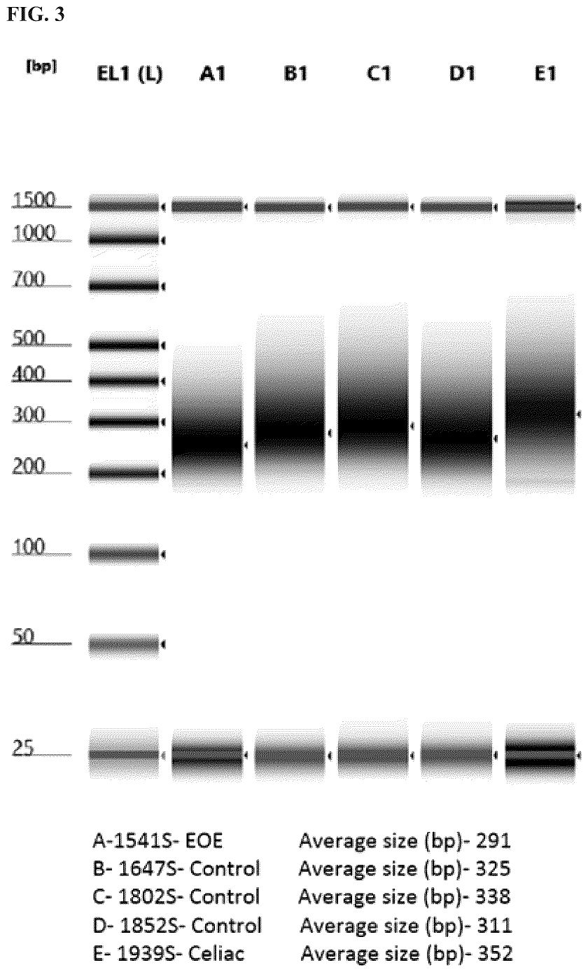

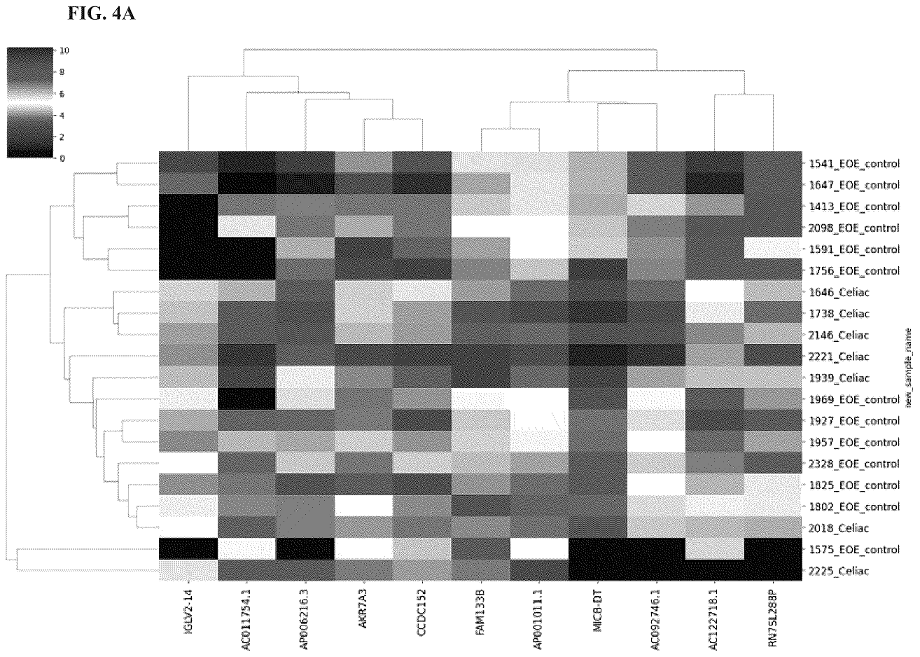

Importantly, the present inventors uncovered that obtaining intact RNA from such samples is not trivial. However, the present inventors have established a novel and unique method for RNA extraction from sputum samples (FIG. 2) which was further amplified (FIG. 3) and sequenced.

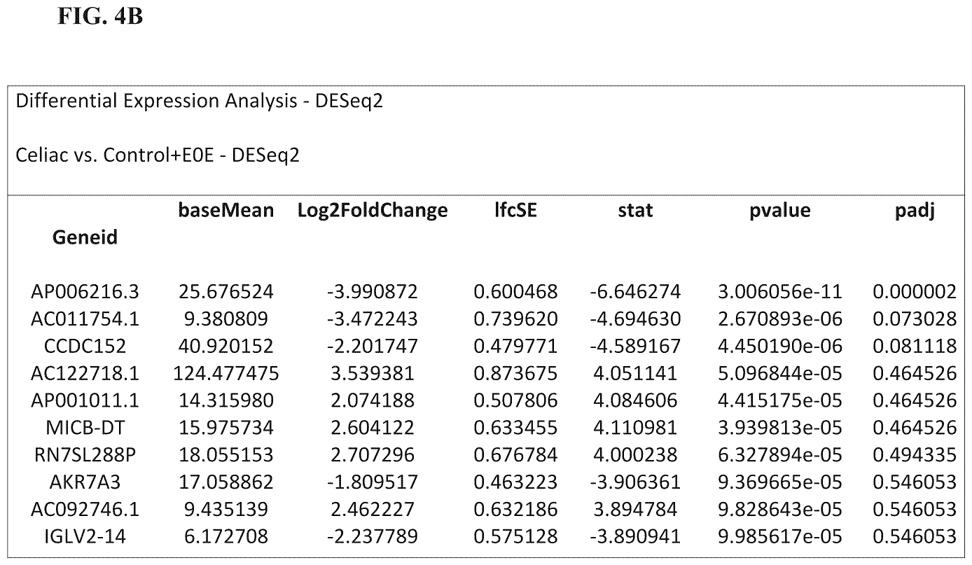

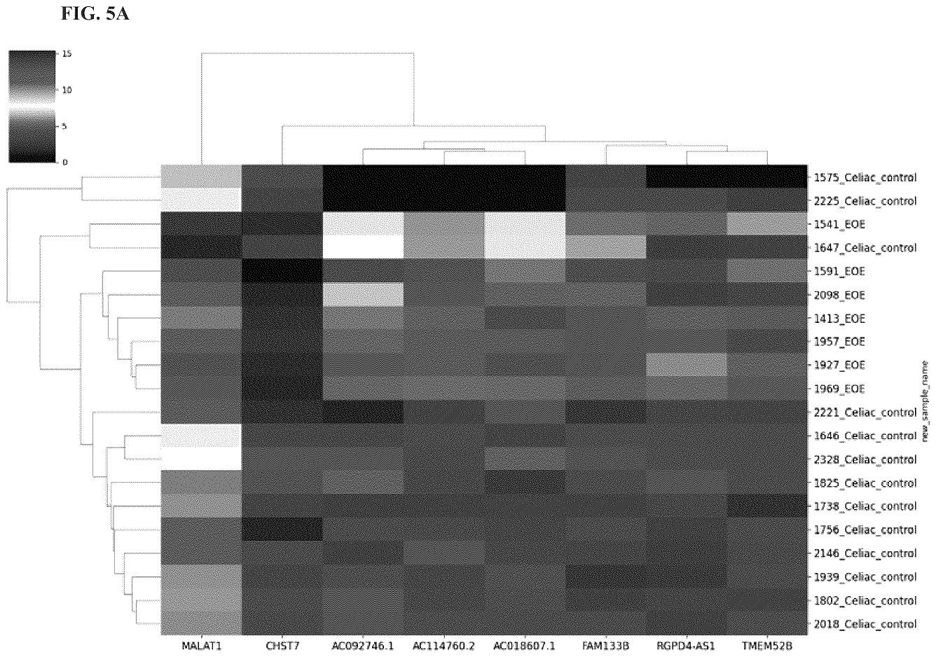

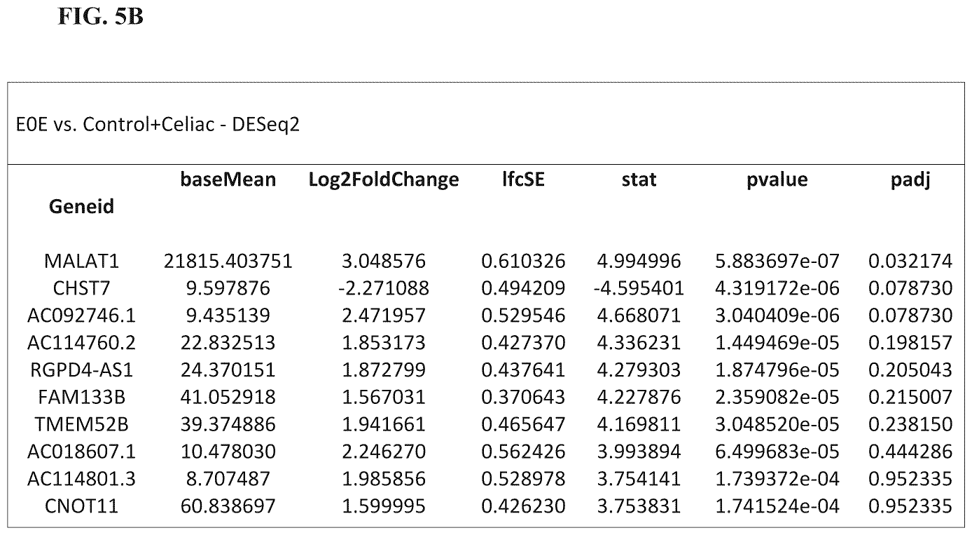

Furthermore, deep sequencing analysis revealed 10 genes having significant altered expression patterns in sputum samples of celiac disease patients, prior to gluten avoidance (n=7), compared to controls (n=7 healthy patients+9 eosinophilic esophagitis patients) (FIGS. 4A-B); and 10 genes having significant altered expression patterns in sputum samples of eosinophilic esophagitis patients (n=9), compared to controls (n=7 healthy patients+7 celiac patients) (FIGS. 5A-B). Interestingly, up-regulation of MALAT1 and FAM133B gene (3.04 and 1.56 log 2 fold change, respectively) was detected in eosinophilic esophagitis; which is in line with the work of Rothenberg and colleagues that showed upregulation of these 2 genes in histological samples of eosinophilic gastritis patients (Caldwell J M et al. J Allergy Clin Immunol. 2014 November; 134(5): 1114-24. doi: 10.1016).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of diagnosing and treating celiac in a human subject in need thereof, the method comprising:
(i) receiving an identification that a subject has celiac according to a method comprising:
(a) analyzing an expression level of at least one gene selected from the group consisting of AP006216.3, AC011754.1, CCDC152, AKR7A3 and IGLV2-14 in a sample of an upper GI tract secretion of the subject, wherein said expression level above a predetermined threshold is indicative of celiac; and/or
(b) analyzing an expression level of at least one gene selected from the group consisting of AC122718.1, AP001011.1, MICB-DT, RN7SL288P and AC092746.1 in a sample of an upper GI tract secretion of the subject, wherein said expression level below a predetermined threshold is indicative of celiac; and
(ii) administering to the subject a therapeutically effective amount of an agent useful for treating celiac.

2. A diagnostic kit for diagnosing celiac, the kit comprising at least two agents capable of specifically detecting expression products of at least two genes associated with celiac, wherein at least one of said at least two genes is selected from the group consisting of AC122718.1, AP001011.1, MICB-DT, RN7SL288P, AC092746.1, AP006216.3, AC011754.1, CCDC152, AKR7A3 and IGLV2-1.

3. The diagnostic kit of claim 2, wherein the kit comprises no more than 20 agents capable of specifically detecting said expression products.

4. The method of claim 1, wherein said upper GI secretion is a saliva sample of the subject.

5. The method of claim 4, wherein said saliva sample is obtained from the subject following at least 6 hours fasting.

6. The method of claim 4, wherein said saliva sample is obtained prior to performing a gastroscopy in said subject.

7. The method of claim 1, wherein said upper GI secretion is a stomach and/or an intestinal secretion sample of the subject.

8. The method of claim 7, wherein said intestinal secretion is a duodenum secretion.

9. The method of claim 8, wherein said duodenum is the duodenum blub.

10. The method of claim 1, wherein said analyzing the expression level comprises analyzing an RNA expression level.

11. The method of claim 1, wherein the expression level of said at least one gene in a saliva sample of said subject correlates with the expression level of said at least one gene in a non-saliva upper GI tract secretion of said subject.

12. The method of claim 1, wherein the expression level of said at least one gene correlates with the degree of histological inflammation.

13. The method of claim 1, wherein said subject shows symptoms of celiac.
